SEQUENCE LISTING (123 sequences)

SEQ ID NO: 1 - protein, 9 aa, Lymphocytic choriomeningitis mammarenavirus
KAVYNFATC

SEQ ID NO: 2 - DNA, 27 bp, Lymphocytic choriomeningitis mammarenavirus
  source 1..27: mol_type = unassigned DNA
aaagctgtgtacaatttcgccacctgt

SEQ ID NO: 3 - DNA, 1818 bp, synthetic construct
  misc_feature 1..1818: note = Description of Artificial Sequence: Synthetic polynucleotide
  source 1..1818: mol_type = other DNA
atggacaagatcctgacagccagcttcctgctgctgggcctgcatctggctggcgtgaacggccagcagaaagagaagcacgatcagcagcaagtgcggcagagcccccagagcctgacagtgtgggaaggcggcaccaccgtgctgacctgtagctacgaggactctaccttcaactacttcccctggtatcagcagttccccggcgagggacctgccctgctgatctctatcctgagcgtgtccgacaagaaagaggacggcagattcaccacattcttcaacaagcgcgagaagaagctgagcctgcacatcatcgactcccagcccggcgacagcgctacctacttctgtgctgccctgtacggcaacgagaagatcaccttcggcgctggcaccaagctgaccatcaagcccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagaccgtgctggacatgaaggccatggacagcaagagcaacggcgctatcgcctggtccaaccagaccagcttcacatgccaggacatcttcaaagagacaaacgccacctaccccagcagcgacgtgccatgtgacgccaccctgaccgagaagtccttcgagacagacatgaacctgaacttccagaacctgtccgtgatgggcctgagaattctgctgctgaaggtggccggcttcaacctgctgatgaccctgagactgtggtccagcagaagaagaagaggcagcggcgagggcagaggcagcctgctgacatgcggagatgtggaagagaaccctggccccatgggcagcagactgttcttcgtggtgctgatcctgctgtgcgccaagcacatggaagccgccgtgacccagagccccagatccaaggtggcagtgaccggcggcaaagtgaccctgtcttgccaccagaccaacaaccacgactacatgtactggtacagacaggacaccggccacggcctgaggctgatccactacagctacgtggccgacagcaccgagaagggcgacatccctgatggctacaaggccagcaggcctagccaggaaaacttcagcctgatcctggaactggccagcctgagccagacagccgtgtacttctgcgccagctctgacgcaggcggcagaaacaccctgtacttcggagccggcaccagactgagcgtgctggaagatctgagaaacgtgaccccccccaaggtgtccctgttcgagcctagcaaggccgagatcgccaacaagcagaaagccaccctcgtgtgcctggccagaggcttcttccccgaccacgtggaactgtcttggtgggtcaacggcaaagaggtgcacagcggcgtgtccacagacccccaggcctacaaagagagcaactactcctactgcctgtcctccaggctgagagtgtccgccaccttctggcacaaccccagaaaccacttcaggtgccaggtgcagtttcacggcctgtccgaagaggacaagtggcccgagggcagccctaagcccgtgacacagaacatctctgccgaggcttggggcagagccgactgtggcatcacaagcgccagctaccatcagggcgtgctgagcgccacaatcctgtacgaaatcctgctgggcaaggccacactgtacgccgtgctggtgtctggcctggtgctgatggccatggtcaagaagaagaacagctga

SEQ ID NO: 4 - DNA, 1619 bp, synthetic construct
  misc_feature 1..1619: note = Description of Artificial Sequence: Synthetic polynucleotide
  source 1..1619: mol_type = other DNA
atgcacagacctagacgtcgtggaactcgtccacctccactggcactgctcgctgctctcctcctggctgcacgtggtgctgatgcaaaagctgtgtacaatttcgccacctgtggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctgctacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgatcgttgctgttctgggtgtccttggagctatggccatcattggagctgtggtggcttttgtgatgaagagaaggagaaacacaggtggaaaaggaggggactatgctctggctccaggctcccagagctctgaaatgtctctccgagattgtaaagcggaacaaaaactcatctcagaagaggatctgggtggagaacaaaaactcatctcagaagaggatctgcaccatcaccatcaccattgagc

SEQ ID NO: 5 - protein, 978 aa, synthetic construct
  REGION 1..978: note = Description of Artificial Sequence: Synthetic polypeptide
MSAAPLVTAMCLLGNVSFPCDRPPTCYTREPSRALDILEENVNHEAYDTLLNAILRCGSSGSVIDDFTLTSPYLGTCSYCHHTVPCFSPVKIEQVWDEADDNTIRIQTSAQFGYDQSGAASANKYRYMAAAADHTVKEGTMDDIKISTSGPCRRLSYKGYFLLAKCPPGDSVTVSIVSSNSATSCTLARKIKPKFVGREKYDLPPVHGKKIPCTVYDRLAATTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANAETFTETMSYLWSNSQPFFWVQLCIPLAAFIVLMRCCSCCLPFLVVAGAYLAKVDAYEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 6 - DNA, 2937 bp, synthetic construct
  misc_feature 1..2937: note = Description of Artificial Sequence: Synthetic polynucleotide
  source 1..2937: mol_type = other DNA
atgtctgccgcccctctcgtgaccgctatgtgcctgctgggcaacgtgtccttcccatgcgacagaccccctacctgctacaccagagagcccagcagagccctggacatcctggaagagaacgtgaaccacgaggcctacgacaccctgctgaacgccatcctgagatgcggcagcagcggcagcgtgatcgacgactttaccctgaccagcccttacctgggcacctgtagctactgccaccacaccgtgccctgcttcagccccgtgaagatcgaacaagtgtgggacgaggccgacgataacaccatccggatccagaccagcgcccagttcggctacgatcagtctggcgccgctagcgccaacaagtaccgctatatggccgctgccgccgatcacaccgtgaaagagggcaccatggacgacatcaagatcagcaccagcggcccctgcagacggctgagctacaagggctacttcctgctggccaagtgcccccctggcgatagcgtgaccgtgtccatcgtgtccagcaacagcgccaccagctgcaccctggccagaaagatcaagcccaagttcgtgggcagagagaagtacgacctgccccctgtgcacggcaagaaaatcccttgcaccgtgtacgaccggctggccgctacaacagccggctacatcaccatgcaccggcccagacctcacgcctacaccagctacctggaagaaagctccggcaaggtgtacgccaagccccccagcggcaagaacatcacctacgagtgcaagtgcggcgactacaagaccggcaccgtgtctacccggaccgagatcaccggatgcaccgccatcaagcagtgcgtggcctacaagagcgaccagaccaaatgggtgttcaacagccccgacctgatccggcacgatgaccacacagcccagggcaagctgcatctgcccttcaagctgatccccagcacctgtatggtgcccgtggcccacgcccccaatgtgatccacggcttcaagcacatcagcctgcagctggacaccgaccacctgacactgctgaccaccagaaggctgggcgccaaccctgagcctaccaccgagtggatcgtgggcaagaccgtgcggaacttcaccgtggacagagatggcctggagtacatctggggcaaccacgagcctgtgcgcgtgtacgcccaggaatctgctcctggcgatcctcacggatggccccacgagatcgtgcagcactactaccaccggcaccccgtgtacaccattctggccgtggcctctgccaccgtggccatgatgatcggagtgaccgtggccgtgctgtgcgcctgcaaggctagaagagagtgcctgaccccttacgccctggcccctaatgccgtgatccctacatctctggccctgctgtgttgcgtgcggagcgccaatgccgagacattcaccgagacaatgagctacctgtggtccaacagccagccattcttctgggtgcagctgtgcatccccctggccgccttcatcgtgctgatgaggtgctgcagctgttgcctgcccttcctggtggtggctggcgcctatctggccaaggtggacgcctatgagcacgccaccaccgtgcctaatgtgcctcagatcccctacaaggccctggtggaaagggccggctacgcccccctgaacctggaaatcaccgtgatgagcagcgaagtgctgcccagcaccaaccaggagtacattacctgcaagtttaccaccgtggtgcccagccccaagatcaagtgctgcggcagcctggaatgccagcctgccgcccatgccgactacacatgcaaggtgttcggcggcgtgtaccccttcatgtggggaggcgcccagtgcttctgcgacagcgagaacagccagatgagcgaggcctatgtggaactgagcgccgactgcgcctctgatcacgcccaggccattaaggtgcacaccgccgccatgaaagtgggcctgagaatcgtgtacggcaacaccaccagctttctggacgtgtacgtgaacggcgtgacccccggcaccagcaaggacctgaaagtgatcgccggacccatcagcgccagcttcacccccttcgaccacaaggtcgtgatccaccggggcctggtgtacaactacgacttccctgagtacggcgccatgaagcctggcgccttcggagatatccaggccaccagcctgacctccaaggatctgatcgcctccaccgacatccggctgctgaagccctccgccaagaatgtgcacgtgccctacacacaggccagctccggcttcgagatgtggaagaacaacagcggcagacccctgcaggaaaccgcccctttcggctgcaagatcgccgtgaaccctctgagagccgtggactgcagctacggcaatatccccatcagcatcgacatccccaacgccgcctttatccggacctccgatgcccctctggtgtctaccgtgaagtgcgaggtgtccgagtgcacctactccgccgattttggcggcatggccaccctgcagtacgtgtccgatagagaaggccagtgccccgtgcactcccacagctctacagccacactgcaggaatccaccgtgcatgtgctggaaaagggcgccgtgacagtgcacttcagcaccgctagcccccaggccaactttatcgtgtccctgtgcggcaaaaagaccacctgtaacgccgagtgcaaaccccctgccgaccacattgtgtccaccccccacaagaacgatcaggaatttcaggccgccatcagcaagacctcttggtcctggctgtttgccctgtttggcggagccagcagcctgctgatcatcggactgatgatcttcgcctgcagcatgatgctgaccagcacaagaagatga

SEQ ID NO: 7 - protein, 8 aa, Gallus gallus
SIINFEKL

SEQ ID NO: 8 - DNA, 24 bp, Gallus gallus
  source 1..24: mol_type = unassigned DNA
agtataatcaactttgaaaaactg

SEQ ID NO: 9 - DNA, 1560 bp, synthetic construct
  misc_feature 1..1560: note = Description of Artificial Sequence: Synthetic polynucleotide
  source 1..1560: mol_type = other DNA
atgcacagacctagacgtcgtggaactcgtccacctccactggcactgctcgctgctctcctcctggctgcacgtggtgctgatgcaagtataatcaactttgaaaaactgggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgctacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtga

SEQ ID NO: 10 - DNA, 1842 bp, synthetic construct
  misc_feature 1..1842: note = Description of Artificial Sequence: Synthetic polynucleotide
  source 1..1842: mol_type = other DNA
atggacaagattctgacagcaacgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtagaagaagaagaggcagcggcgagggcagaggcagcctgctgacatgcggagatgtggaagagaaccctggccccatgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctcgggccaattatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctagtgctgatggccatggtcaagaaaaaaaattcctga

SEQ ID NO: 11 - DNA, 9539 bp, synthetic construct
  misc_feature 1..9539: note = Description of Artificial Sequence: Synthetic polynucleotide
  source 1..9539: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 12 - DNA, 9694 bp, synthetic construct
  misc_feature 1..9694: note = Description of Artificial Sequence: Synthetic polynucleotide
  source 1..9694: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatgccgaatcctcgccctggcaagccttccgctccctctttggcgttgggcccgagtcccggagcctctccgagctggcgggctgctcctaaagcgagcgacttgctcggggccagagggcccggtgggacgttccaaggacgagacctccgaggaggtgcacatgcttcttcatctagtcttaatccgatgccacctagccagcttcaactgcccacgctgccgttggtcatggtagctccttcaggcgccaggctcggccctttgcctcatctgcaagccttgctccaggatagaccacacttcatgcatcagctttctactgtagacgcgcatgcacgaacacccgtacttcaagtgcacccattggaatcaccggctatgatttctctcacgccacctaccaccgcgacaggggttttttccttgaaggcaagacctgggcttccaccaggaataaatgtggcgtcactcgaatgggtcagtagagaaccggccctgctctgtaccttccctaacccctccgcccccaggaaggattcaacgttgagtgcggtgccgcaatccagctaccctttgctcgccaacggcgtgtgcaagtggcccggttgtgagaaggtctttgaagagccagaggacttcttgaaacactgccaagcagaccatctgttggatgaaaaagggagagcccaatgccttttgcaacgggagatggttcaaagtctcgaacagcagcttgtactcgaaaaagaaaaactctcagctatgcaggcccatctcgctgggaaaatggctttgactaaagctagctctgtggcaagcagtgataaaggttcctgttgtattgtggcagccggttcacagggcccagttgtaccggcttggtccgggccccgagaagctcccgattctctttttgctgtccgacgccacctgtggggctcccatgggaactctacatttcccgaatttttgcataacatggattatttcaaatttcataacatgcggccaccattcacttacgctacactgattagatgggcgattttggaagcacctgaaaagcagagaacgctcaacgaaatttaccattggttcacacgcatgttcgccttctttcgaaaccatcctgcaacatggaagaacgccataaggcataatttgtccctccacaaatgcttcgttcgagtggaatccgagaaaggcgcagtctggacagtagacgagctcgagttccggaaaaaacggtcccagaggccgtcacggtgtagtaatcctaccccgggaccaagaagaagaagaggatccggagctactaacttcagcctgctgaagcaggctggagacgtggaggagaaccctggacccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 13 - DNA, 1296 bp, synthetic construct
  misc_feature 1..1296: note = Description of Artificial Sequence: Synthetic polynucleotide
  source 1..1296: mol_type = other DNA
atgccgaatcctcgccctggcaagccttccgctccctctttggcgttgggcccgagtcccggagcctctccgagctggcgggctgctcctaaagcgagcgacttgctcggggccagagggcccggtgggacgttccaaggacgagacctccgaggaggtgcacatgcttcttcatctagtcttaatccgatgccacctagccagcttcaactgcccacgctgccgttggtcatggtagctccttcaggcgccaggctcggccctttgcctcatctgcaagccttgctccaggatagaccacacttcatgcatcagctttctactgtagacgcgcatgcacgaacacccgtacttcaagtgcacccattggaatcaccggctatgatttctctcacgccacctaccaccgcgacaggggttttttccttgaaggcaagacctgggcttccaccaggaataaatgtggcgtcactcgaatgggtcagtagagaaccggccctgctctgtaccttccctaacccctccgcccccaggaaggattcaacgttgagtgcggtgccgcaatccagctaccctttgctcgccaacggcgtgtgcaagtggcccggttgtgagaaggtctttgaagagccagaggacttcttgaaacactgccaagcagaccatctgttggatgaaaaagggagagcccaatgccttttgcaacgggagatggttcaaagtctcgaacagcagcttgtactcgaaaaagaaaaactctcagctatgcaggcccatctcgctgggaaaatggctttgactaaagctagctctgtggcaagcagtgataaaggttcctgttgtattgtggcagccggttcacagggcccagttgtaccggcttggtccgggccccgagaagctcccgattctctttttgctgtccgacgccacctgtggggctcccatgggaactctacatttcccgaatttttgcataacatggattatttcaaatttcataacatgcggccaccattcacttacgctacactgattagatgggcgattttggaagcacctgaaaagcagagaacgctcaacgaaatttaccattggttcacacgcatgttcgccttctttcgaaaccatcctgcaacatggaagaacgccataaggcataatttgtccctccacaaatgcttcgttcgagtggaatccgagaaaggcgcagtctggacagtagacgagctcgagttccggaaaaaacggtcccagaggccgtcacggtgtagtaatcctaccccgggaccatag

SEQ ID NO: 14 - DNA, 8397 bp, synthetic construct
  misc_feature 1..8397: note = Description of Artificial Sequence: Synthetic polynucleotide
  source 1..8397: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcggatccggagctactaacttcagcctgctgaagcaggctggagacgtggaggagaaccctggacccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 15 - DNA, 10228 bp, synthetic construct
  misc_feature 1..10228: note = Description of Artificial Sequence: Synthetic polynucleotide
  source 1..10228: mol_type = other DNA
ggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagtaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagatgagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagccaccatgccgaatcctcgccctggcaagccttccgctccctctttggcgttgggcccgagtcccggagcctctccgagctggcgggctgctcctaaagcgagcgacttgctcggggccagagggcccggtgggacgttccaaggacgagacctccgaggaggtgcacatgcttcttcatctagtcttaatccgatgccacctagccagcttcaactgcccacgctgccgttggtcatggtagctccttcaggcgccaggctcggccctttgcctcatctgcaagccttgctccaggatagaccacacttcatgcatcagctttctactgtagacgcgcatgcacgaacacccgtacttcaagtgcacccattggaatcaccggctatgatttctctcacgccacctaccaccgcgacaggggttttttccttgaaggcaagacctgggcttccaccaggaataaatgtggcgtcactcgaatgggtcagtagagaaccggccctgctctgtaccttccctaacccctccgcccccaggaaggattcaacgttgagtgcggtgccgcaatccagctaccctttgctcgccaacggcgtgtgcaagtggcccggttgtgagaaggtctttgaagagccagaggacttcttgaaacactgccaagcagaccatctgttggatgaaaaagggagagcccaatgccttttgcaacgggagatggttcaaagtctcgaacagcagcttgtactcgaaaaagaaaaactctcagctatgcaggcccatctcgctgggaaaatggctttgactaaagctagctctgtggcaagcagtgataaaggttcctgttgtattgtggcagccggttcacagggcccagttgtaccggcttggtccgggccccgagaagctcccgattctctttttgctgtccgacgccacctgtggggctcccatgggaactctacatttcccgaatttttgcataacatggattatttcaaatttcataacatgcggccaccattcacttacgctacactgattagatgggcgattttggaagcacctgaaaagcagagaacgctcaacgaaatttaccattggttcacacgcatgttcgccttctttcgaaaccatcctgcaacatggaagaacgccataaggcataatttgtccctccacaaatgcttcgttcgagtggaatccgagaaaggcgcagtctggacagtagacgagctcgagttccggaaaaaacggtcccagaggccgtcacggtgtagtaatcctaccccgggaccataggc

SEQ ID NO: 16 - DNA, 8932 bp, synthetic construct
  misc_feature 1..8932: note = Description of Artificial Sequence: Synthetic polynucleotide
  source 1..8932: mol_type = other DNA
ggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagtaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagatgagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagccaccgc

SEQ ID NO: 17 - DNA, 1581 bp, synthetic construct
  misc_feature 1..1581: note = Description of Artificial Sequence: Synthetic polynucleotide
  source 1..1581: mol_type = other DNA
atggaaacggaagctatagacggctatatcacgtgtgacaacgagctttcacccgagcgggaacattcaaatatggctatcgaccttaccagctccaccccgaacggtcagcacgctagtccaagtcatatgacatctacaaattctgtcaagctggagatgcaatccgatgaggagtgcgaccgaaagccactttcaagggaggatgagatccggggtcacgatgaaggttcttctttggaggaaccgctcattgagagctctgaggtggcggacaacagaaaggttcaagagctgcaaggcgaaggcggcatccgactccccaacggtaagctcaaatgtgatgtatgcggtatggtatgtataggccctaatgtgctcatggttcacaagcggtcccatactggggaacgcccctttcattgcaaccagtgtggagcttcttttactcaaaaaggaaacctgctgcgccatattaagttgcattccggcgaaaaacccttcaaatgtcctttctgttcctacgcttgtcgacgcagggatgcgcttaccggacatctgcgaactcacagcgtaggcaagccccataaatgcaattactgcggacggtcatacaagcaaaggtcctcactcgaagaacacaaggaacgctgccataactatctccagaacgtatctatggaagccgccggtcaggtcatgagccaccacgtcccgcccatggaagattgcaaggagcaagaaccaatcatggacaataacatttcacttgtgccgtttgaacgacccgccgtaatcgaaaagctcaccgggaacatgggaaagcgcaaatcctccactcctcaaaagtttgtaggagaaaaactcatgaggttctcttaccccgatatacacttcgacatgaatctcacatatgaaaaggaggccgaactgatgcaaagccacatgatggaccaggctatcaataacgcgataacttacctcggagcagaagctctgcacccccttatgcaacaccccccttctactatagctgaagtcgctccagtaatatcaagcgcatattcacaagtttatcacccaaatcgaattgaacgccctattagccgggagaccgccgacagccatgagaacaatatggacggtccaatttctctcatccggcctaagagccggccccaggagcgcgaagccagtccatccaatagctgcctcgattcaacggatagtgaatccagccacgatgatcatcagagctaccaaggtcatccagctcttaacccgaaaaggaagcagtccccggcgtacatgaaagaggatgttaaggcgctcgatacgacaaaagctccaaaaggaagtttgaaggacatatataaagtctttaatggagagggtgaacaaatccgggctttcaagtgcgagcattgcagagttctgtttttggaccacgtgatgtataccatacatatggggtgccatggttatcgagatcctttggagtgtaacatctgcggctacaggtcacaagataggtacgagttctcatcccacatcgtaagaggggagcacacgtttcactag

SEQ ID NO: 18 - DNA, 1758 bp, synthetic construct
  misc_feature 1..1758: note = Description of Artificial Sequence: Synthetic polynucleotide
  source 1..1758: mol_type = other DNA
atgcacacgccgcctgccctccctcgccgcttccagggaggtgggcgagtacgaaccccaggttcacacagacaaggtaaagataacctcgaaagggacccaagtggaggctgcgtaccggactttcttcctcaggcacaagatagcaatcactttataatggaatctctgttttgcgaatcctctggagacagcagcctggaaaaagagtttttgggtgctccagtgggtccgtcagtgagcaccccgaactcccaacactctagtccttccaggagcctctctgcaaacagtatcaaggtcgaaatgtatagcgatgaagagagttctcgcctcctcggccctgacgaacgacttctcgaaaaggatgacagcgtcattgttgaagattcactttctgagccactcggatactgcgatggtagcgggcctgaaccgcatagtccgggaggtattcgacttccaaacggtaagttgaaatgcgacgtttgcggtatggtttgtattggcccgaatgtcctcatggttcataagcggagccatactggggagaggcccttccattgtaatcaatgtggagcgtctttcacacaaaaaggcaaccttctccgccatataaagttgcacagtggcgagaaaccatttaagtgtcccttttgtaattatgcctgtaggcgacgggacgcgctcaccggacatctccgaactcacagtgtatccagtcctactgtagggaagccctataaatgcaattactgtggccgctcctataagcagcaatctactcttgaagaacataaggagcgatgccacaattacttgcaaagcctgtccacagaagcgcaagcattggcaggtcaaccaggtgacgaaatacgggaccttgagatggttcctgatagtatgcttcatagttcatcagagcggccaacctttatcgaccgcttggccaacagcttgaccaaacgcaagcggtctacaccccaaaagttcgtcggtgaaaaacagatgcgatttagtctgtccgacctgccttacgatgtaaattccggcggatatgaaaaagacgtggagctggtcgctcatcatagcctggaaccaggatttggatctagcctcgccttcgtaggagctgagcacttgcggccactcagactgcctcccaccaattgcataagtgagttgactccagtcatctcttctgtctacacccaaatgcaaccactcccaggtcgcctcgaacttccgggtagccgagaggctggggagggtcctgaagacctggcagatggcggccccttgctttacagaccacgcggcccccttacggacccgggagcttccccttcaaatgggtgccaggatagtaccgatactgagtcaaatcacgaagatcgcgtcgcaggggttgtatccctcccacaagggccaccgccgcaacctcccccaacgatagttgtaggccgccacagtcctgcgtatgcaaaggaagacccgaagccccaagaaggtttgttgagggggacccctggcccttcaaaagaagttctgagagttgtaggtgagagtggggaaccagtcaaagcctttaaatgcgaacattgtaggattcttttcctcgaccacgtaatgtttactattcacatgggttgccatgggtttagggacccctttgagtgcaacatttgtggataccattcacaggacaggtacgagttttcctcacatattgttcgaggcgaacacaaagtggggtag

SEQ ID NO: 19 - DNA, 1242 bp, synthetic construct
  misc_feature 1..1242: note = Description of Artificial Sequence: Synthetic polynucleotide
  source 1..1242: mol_type = other DNA
atggaatttcctggattgggatctttgggaacgtctgagccgttgccacaattcgtagatccggctctggttagtagtactcctgagtccggtgtatttttcccatccggaccggaaggactggatgcggcagcttcatccactgcgccttcaacagcgacagcggctgctgccgctctcgcctattacagggacgcggaagcctacaggcacagtcccgtttttcaggtctatcctctgttgaattgtatggaaggaatcccaggaggaagcccttacgctggctgggcctatggcaagactggcttgtaccctgcaagcacagtttgtccgactcgagaggattcccccccacaagcggtagaagatttggacggtaaaggttctactagctttttggaaacactcaagacagagcgattgtcaccagatctcctcacacttgggccggccttgccgtcctctcttcctgtacccaacagcgcctatggcggacccgacttcagttccacgtttttctcacccaccgggtcccctcttaattcagccgcatactcaagtcccaagttgagagggaccctgccgctcccgccttgcgaggctagagagtgcgtaaactgcggagctacagcaacccctctgtggaggagagaccgcacgggccattatctctgcaacgcgtgcgggttgtaccataagatgaatggacaaaatcgccctctgattcgaccgaagaagagacttatcgtttccaaacgcgctggcacacagtgtaccaactgccagactactaccactaccctctggcggcgcaacgcttccggagatcccgtgtgcaacgcttgtggtctttactataagttgcatcaggtgaatcggcctctgaccatgaggaaagacggcattcagactcgaaatagaaaagcgtctggcaagggaaagaagaagagggggagctctctcggaggaacaggagcggcggagggcccagcgggtggatttatggttgttgctggcgggtctgggtcagggaactgcggagaggtcgcatcaggcctcaccctggggccccccggaaccgcccacctctaccaaggattggggcccgtcgtactgtcagggccggtatcacatctgatgcctttcccaggaccgttgttgggcagtcctactggaagttttcctactggaccaatgccgccaactacaagcacgactgttgttgctcccctttccagttag

SEQ ID NO: 20 - DNA, 1356 bp, synthetic construct
  misc_feature 1..1356: note = Description of Artificial Sequence: Synthetic polynucleotide
  source 1..1356: mol_type = other DNA
atgaatttggaggggggagggcgaggaggggagtttggtatgagtgcagtaagctgcgggaatggtaaactgaggcaatggctcatagaccagatcgactcagggaagtatccgggccttgtctgggagaacgaggagaagtcaattttcagaataccctggaagcacgccggaaaacaagattacaacagagaggaagatgccgcactctttaaggcgtgggcccttttcaagggtaagtttcgagaagggattgataagccagaccctcctacatggaaaaccagacttaggtgcgctctcaacaagagcaacgattttgaagagttggtagaaaggtcacaacttgacatttcagatccgtacaaggtctatcggatcgtcccagagggcgccaagaaaggggcaaaacaactcacacttgaagaccctcagatgagcatgtcacacccgtacactatgactacaccctatccctcacttcctgcccagcaagttcataattacatgatgccacccctcgataggagttggcgagactacgttccagaccagccacatccagaaatcccataccaatgtcctatgacatttggtccgagaggacaccactggcagggccctgcatgcgagaatggttgtcaggtcactgggaccttttacgcgtgtgcaccacccgaatctcaagccccaggcgttcccaccgagccttccattagatctgctgaggctcttgccttctcagactgccgacttcatatatgtctctactatcgagaaatcttggtcaaagaactgacgacgtcatccccggagggatgtcggataagtcacggtcatacgtacgacgcgtccaatttggatcaggtattgtttccttacccggaagataacggtcaaaggaagaacatagagaagttgttgtctcatctcgagcgcggagtggtgctctggatggcgccagacggattgtatgccaagcgcctgtgtcagtcccgcatctactgggatgggccacttgctctttgcaatgaccggcccaataagcttgaaagggaccaaacctgcaagttgttcgacacacaacaatttttgagcgagttgcaggcattcgcacaccacggacgaagtttgcccagatttcaggtcaccctgtgtttcggggaagagttccctgaccctcaacgccagcggaaactcatcacagcgcatgtagaacccctgttggctaggcagctctactacttcgctcaacaaaatagcggtcattttctgcgcggatatgatcttccagagcatataagtaaccctgaggactaccatagaagcattcgacacagcagcatacaagagtag

SEQ ID NO: 21 - DNA, 2295 bp, synthetic construct
  misc_feature 1..2295: note = Description of Artificial Sequence: Synthetic polynucleotide
  source 1..2295: mol_type = other DNA
atggaccatctgaacgaagccactcagggtaaggagcattcagaaatgagtaataacgtatctgaccctaaggggcctccggcaaaaatcgcaagacttgaacaaaatggcagtccactcggcagggggcgccttggatcaacaggcgcgaagatgcagggcgtccctcttaaacattcaggtcaccttatgaagaccaacctccgcaaaggcacgatgcttccggtgttttgtgtcgtggaacattatgaaaacgcgatcgaatacgattgtaaggaggaacacgcagagtttgttttggtacggaaggacatgctctttaaccagctgattgagatggctctgctcagtctgggttacagccatagtagcgccgcacaagccaagggacttatacaggttggcaagtggaacccggttcctctttcttacgtgactgacgcgccggacgcaaccgtggcagatatgctccaggatgtgtatcacgtagtgacacttaaaattcagcttcactcatgccccaaactggaggatcttccacctgaacagtggtcacatactactgtgcgcaatgcgctcaaagaccttcttaaagatatgaatcaatcttcactggcgaaagagtgtcctctctctcagtctatgataagttctatcgttaactccacatattacgccaacgtaagcgcagctaaatgccaagaatttggacgctggtataagcattttaaaaaaaccaaggatatgatggtggagatggactccttgtctgaactctcacaacaaggcgccaaccatgtcaattttggacagcagcctgttcctggaaacacagccgagcaacctccctcaccagcgcagctgtcccacggttctcagccaagcgtgcgcactccgcttccaaatctgcacccggggctggtttcaacgcccatcagtcctcaactcgtaaaccagcagctcgtcatggcgcagcttctgaatcagcagtatgcagttaaccggctgctggcacaacagagtcttaaccagcaatatttgaatcatccaccccctgtgtcacggtctatgaacaaacccctcgagcagcaggtctctactaatactgaagtgagctctgagatctatcaatgggtgcgggacgagctgaaaagagccgggatatcacaggcggtatttgcacgcgttgcattcaacaggacacaaggccttttgagcgaaatattgcgaaaagaggaggacccgaagaccgctagccagagtcttcttgttaacctccgggcgatgcaaaattttcttcaactgcctgaggctgagagagaccgaatctatcaagacgagagggaacggtccctcaacgcggcttctgcgatgggtcccgcaccgcttatcagcacgccaccatccaggccgccgcaggtgaaaaccgcaaccatcgcaacggagcgaaacggaaaaccagaaaacaatactatgaatatcaacgctagtatctatgatgaaatacagcaagaaatgaagagagccaaggtatcacaggccctcttcgccaaagttgctgccactaagtctcaaggctggctgtgtgagctgttgcggtggaaagaggacccttctccggagaatcgaacactttgggagaacttgagcatgatcaggaggtttctgtcactcccgcaaccggagcgggatgccatttacgagcaggagtccaatgccgtacatcatcatggtgatcgcccgccccacattatccatgtgcccgcagaacagattcagcaacaacagcagcagctagagcagcaacagcagcagcaacaacaggcacctccccctccgcaaccccaacaacaaccgcaaaccggcccgcggttgcccccaaggcagccgactgtcgcatctcctgcggaaagtgatgaagagaatcggcaaaagaccaggccgaggaccaagattagcgtcgaggcccttggaatcctccagtcctttatacaagatgttggactgtacccggatgaagaggccattcaaaccctgtctgcacaactggacttgcccaagtacacaattataaagttttttcaaaaccaacgctactatctcaagcatcatggaaagttgaaagacaattctggtctcgaagttgacgtcgcagagtataaggaggaagaactcttgaaggacctcgaggagagtgtacaggacaaaaataccaatactctcttcagcgtaaaactggaagaagaactctcagtggaagggaacaccgacataaacactgatctcaaggactag

SEQ ID NO: 22 - DNA, 1197 bp, synthetic construct
  misc_feature 1..1197: note = Description of Artificial Sequence: Synthetic polynucleotide
  source 1..1197: mol_type = other DNA
atgcctcagctgtcaggaggaggcggcggggggggtggcgacccggaattgtgcgcgaccgatgaaatgatcccatttaaagacgagggagatccacagaaggaaaagatcttcgctgaaattagccatcccgaggaagagggagatctggcagacataaagagcagtttggtgaatgaatctgagataattccggcgtctaatggacatgaagtcgccaggcaagcgcagactagtcaagaaccttaccacgataaggcgagagagcatccagacgatgggaagcaccctgatggcggcctgtataataagggaccctcttactcttcctatagcggctatattatgatgccaaatatgaataacgacccatatatgtctaacggctcactctctcctccgataccgcggaccagtaataaagttccagtagtccaaccctcccacgcggtgcatcctttgacaccactcatcacatattcagacgagcattttagcccaggctcacatccaagtcatataccgtccgatgtcaacagcaagcagggaatgtcacgccacccgcctgccccagacatacccaccttttaccccctgagccccggtggggttggacagatcactccgccccttggttggcagggacagccagtgtatcccatcacgggtgggtttaggcagccatatcccagctcactctccgtggacacttcaatgtcacgctttagccaccacatgattccagggccacccggcccccataccaccggcatcccacatccggctatagttaccccccaggttaagcaagaacaccctcacacagattcagacctgatgcatgttaaaccccaacatgaacagagaaaagagcaagaaccgaaaaggccacacatcaagaagccgttgaatgcatttatgctgtatatgaaggaaatgcgggcgaacgttgttgcagagtgtacgctcaaggaatccgccgctattaaccaaattctgggtcgaagatggcacgctctgagccgggaagaacaggcaaaatactatgagttggcgcggaaggaaaggcaactccacatgcaattgtatcctgggtggtctgcgcgggataattatggaaaaaagaagaaacgcaaacgagaaaaactccaggagagtgcgagtggcactgggccacgcatgaccgctgcgtatatc

SEQ ID NO: 23 - DNA, 7565 bp, synthetic construct
  misc_feature 1..7565: note = Description of Artificial Sequence: Synthetic polynucleotide
  source 1..7565: mol_type = other DNA
aagcttatactcgagctctagattgggaacccgggtctctcgaattcgccaccatgcacagacctagacgtcgtggaactcgtccacctccactggcactgctcgctgctctcctcctggctgcacgtggtgctgatgcaaaagctgtgtacaatttcgccacctgtggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctgctacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgatcgttgctgttctgggtgtccttggagctatggccatcattggagctgtggtggcttttgtgatgaagagaaggagaaacacaggtggaaaaggaggggactatgctctggctccaggctcccagagctctgaaatgtctctccgagattgtaaagcggaacaaaaactcatctcagaagaggatctgggtggagaacaaaaactcatctcagaagaggatctgcaccatcaccatcaccattgagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 24 - DNA, 7508 bp, synthetic construct
  misc_feature 1..7508: note = Description of Artificial Sequence: Synthetic polynucleotide
  source 1..7508: mol_type = other DNA
aagcttatactcgagctctagattgggaacccgggtctctcgaattcgccaccatgcacagacctagacgtcgtggaactcgtccacctccactggcactgctcgctgctctcctcctggctgcacgtggtgctgatgcaagtataatcaactttgaaaaactgggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgctacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtgagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 25 - DNA, 5960 bp, synthetic construct
  misc_feature 1..5960: note = Description of Artificial Sequence: Synthetic polynucleotide
  source 1..5960: mol_type = other DNA
aagcttatactcgagctctagattgggaacccgggtctctcgaattcgagatctagtttaaacacgcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 26 - DNA, 8885 bp, synthetic construct
  misc_feature 1..8885: note = Description of Artificial Sequence: Synthetic polynucleotide
  source 1..8885: mol_type = other DNA
aagcttatactcgagctctagattgggaacccgggtctctcgaattcgccaccatgtctgccgcccctctcgtgaccgctatgtgcctgctgggcaacgtgtccttcccatgcgacagaccccctacctgctacaccagagagcccagcagagccctggacatcctggaagagaacgtgaaccacgaggcctacgacaccctgctgaacgccatcctgagatgcggcagcagcggcagcgtgatcgacgactttaccctgaccagcccttacctgggcacctgtagctactgccaccacaccgtgccctgcttcagccccgtgaagatcgaacaagtgtgggacgaggccgacgataacaccatccggatccagaccagcgcccagttcggctacgatcagtctggcgccgctagcgccaacaagtaccgctatatggccgctgccgccgatcacaccgtgaaagagggcaccatggacgacatcaagatcagcaccagcggcccctgcagacggctgagctacaagggctacttcctgctggccaagtgcccccctggcgatagcgtgaccgtgtccatcgtgtccagcaacagcgccaccagctgcaccctggccagaaagatcaagcccaagttcgtgggcagagagaagtacgacctgccccctgtgcacggcaagaaaatcccttgcaccgtgtacgaccggctggccgctacaacagccggctacatcaccatgcaccggcccagacctcacgcctacaccagctacctggaagaaagctccggcaaggtgtacgccaagccccccagcggcaagaacatcacctacgagtgcaagtgcggcgactacaagaccggcaccgtgtctacccggaccgagatcaccggatgcaccgccatcaagcagtgcgtggcctacaagagcgaccagaccaaatgggtgttcaacagccccgacctgatccggcacgatgaccacacagcccagggcaagctgcatctgcccttcaagctgatccccagcacctgtatggtgcccgtggcccacgcccccaatgtgatccacggcttcaagcacatcagcctgcagctggacaccgaccacctgacactgctgaccaccagaaggctgggcgccaaccctgagcctaccaccgagtggatcgtgggcaagaccgtgcggaacttcaccgtggacagagatggcctggagtacatctggggcaaccacgagcctgtgcgcgtgtacgcccaggaatctgctcctggcgatcctcacggatggccccacgagatcgtgcagcactactaccaccggcaccccgtgtacaccattctggccgtggcctctgccaccgtggccatgatgatcggagtgaccgtggccgtgctgtgcgcctgcaaggctagaagagagtgcctgaccccttacgccctggcccctaatgccgtgatccctacatctctggccctgctgtgttgcgtgcggagcgccaatgccgagacattcaccgagacaatgagctacctgtggtccaacagccagccattcttctgggtgcagctgtgcatccccctggccgccttcatcgtgctgatgaggtgctgcagctgttgcctgcccttcctggtggtggctggcgcctatctggccaaggtggacgcctatgagcacgccaccaccgtgcctaatgtgcctcagatcccctacaaggccctggtggaaagggccggctacgcccccctgaacctggaaatcaccgtgatgagcagcgaagtgctgcccagcaccaaccaggagtacattacctgcaagtttaccaccgtggtgcccagccccaagatcaagtgctgcggcagcctggaatgccagcctgccgcccatgccgactacacatgcaaggtgttcggcggcgtgtaccccttcatgtggggaggcgcccagtgcttctgcgacagcgagaacagccagatgagcgaggcctatgtggaactgagcgccgactgcgcctctgatcacgcccaggccattaaggtgcacaccgccgccatgaaagtgggcctgagaatcgtgtacggcaacaccaccagctttctggacgtgtacgtgaacggcgtgacccccggcaccagcaaggacctgaaagtgatcgccggacccatcagcgccagcttcacccccttcgaccacaaggtcgtgatccaccggggcctggtgtacaactacgacttccctgagtacggcgccatgaagcctggcgccttcggagatatccaggccaccagcctgacctccaaggatctgatcgcctccaccgacatccggctgctgaagccctccgccaagaatgtgcacgtgccctacacacaggccagctccggcttcgagatgtggaagaacaacagcggcagacccctgcaggaaaccgcccctttcggctgcaagatcgccgtgaaccctctgagagccgtggactgcagctacggcaatatccccatcagcatcgacatccccaacgccgcctttatccggacctccgatgcccctctggtgtctaccgtgaagtgcgaggtgtccgagtgcacctactccgccgattttggcggcatggccaccctgcagtacgtgtccgatagagaaggccagtgccccgtgcactcccacagctctacagccacactgcaggaatccaccgtgcatgtgctggaaaagggcgccgtgacagtgcacttcagcaccgctagcccccaggccaactttatcgtgtccctgtgcggcaaaaagaccacctgtaacgccgagtgcaaaccccctgccgaccacattgtgtccaccccccacaagaacgatcaggaatttcaggccgccatcagcaagacctcttggtcctggctgtttgccctgtttggcggagccagcagcctgctgatcatcggactgatgatcttcgcctgcagcatgatgctgaccagcacaagaagatgagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 27 - DNA, 10649 bp, synthetic construct
  misc_feature 1..10649: note = Description of Artificial Sequence: Synthetic polynucleotide
  source 1..10649: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcgccaccatggacaagatcctgacagccagcttcctgctgctgggcctgcatctggctggcgtgaacggccagcagaaagagaagcacgatcagcagcaagtgcggcagagcccccagagcctgacagtgtgggaaggcggcaccaccgtgctgacctgtagctacgaggactctaccttcaactacttcccctggtatcagcagttccccggcgagggacctgccctgctgatctctatcctgagcgtgtccgacaagaaagaggacggcagattcaccacattcttcaacaagcgcgagaagaagctgagcctgcacatcatcgactcccagcccggcgacagcgctacctacttctgtgctgccctgtacggcaacgagaagatcaccttcggcgctggcaccaagctgaccatcaagcccaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagaccgtgctggacatgaaggccatggacagcaagagcaacggcgctatcgcctggtccaaccagaccagcttcacatgccaggacatcttcaaagagacaaacgccacctaccccagcagcgacgtgccatgtgacgccaccctgaccgagaagtccttcgagacagacatgaacctgaacttccagaacctgtccgtgatgggcctgagaattctgctgctgaaggtggccggcttcaacctgctgatgaccctgagactgtggtccagcagaagaagaagaggcagcggcgagggcagaggcagcctgctgacatgcggagatgtggaagagaaccctggccccatgggcagcagactgttcttcgtggtgctgatcctgctgtgcgccaagcacatggaagccgccgtgacccagagccccagatccaaggtggcagtgaccggcggcaaagtgaccctgtcttgccaccagaccaacaaccacgactacatgtactggtacagacaggacaccggccacggcctgaggctgatccactacagctacgtggccgacagcaccgagaagggcgacatccctgatggctacaaggccagcaggcctagccaggaaaacttcagcctgatcctggaactggccagcctgagccagacagccgtgtacttctgcgccagctctgacgcaggcggcagaaacaccctgtacttcggagccggcaccagactgagcgtgctggaagatctgagaaacgtgaccccccccaaggtgtccctgttcgagcctagcaaggccgagatcgccaacaagcagaaagccaccctcgtgtgcctggccagaggcttcttccccgaccacgtggaactgtcttggtgggtcaacggcaaagaggtgcacagcggcgtgtccacagacccccaggcctacaaagagagcaactactcctactgcctgtcctccaggctgagagtgtccgccaccttctggcacaaccccagaaaccacttcaggtgccaggtgcagtttcacggcctgtccgaagaggacaagtggcccgagggcagccctaagcccgtgacacagaacatctctgccgaggcttggggcagagccgactgtggcatcacaagcgccagctaccatcagggcgtgctgagcgccacaatcctgtacgaaatcctgctgggcaaggccacactgtacgccgtgctggtgtctggcctggtgctgatggccatggtcaagaagaagaacagctgaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 28 - DNA, 10673 bp, synthetic construct
  misc_feature 1..10673: note = Description of Artificial Sequence: Synthetic polynucleotide
  source 1..10673: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcgccaccatggacaagattctgacagcaacgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtagaagaagaagaggcagcggcgagggcagaggcagcctgctgacatgcggagatgtggaagagaaccctggccccatgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctcgggccaattatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctagtgctgatggccatggtcaagaaaaaaaattcctgaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 29 - DNA, 1590 bp, synthetic construct
  misc_feature 1..1590: note = Description of Artificial Sequence: Synthetic polynucleotide
  source 1..1590: mol_type = other DNA
atgggtatagtggaacctggttgcggggacatgctcacaggcacagagcccatgccctcagacgaaggaagaggacctggggctgaccaacaacaccgctttttctatcccgagcctggcgcacaagaccccacagatagaagagcaggatccagcctgggaacaccttacagtgggggggctctggtaccagccgctccaggtcgcttccttggctcattcgcctaccctcctcgcgctcaggttgcaggttttccaggccctggggagttctttcccccccccgcaggtgctgagggctaccctcccgttgatggatacccagccccagaccccagagccggactttaccctggcccccgggaagactatgcactgcccgctgggctcgaggtttcagggaaacttagagtggccctctctaaccatttgctttggagtaaattcaatcagcatcagactgaaatgatcataacaaaacaaggtagacgaatgtttcccttcttgtcttttaccgttgcaggtctggaaccaacatcacactataggatgttcgtcgacgtggtgctggtagaccaacatcactggagataccaatctgggaagtgggttcagtgcggcaaggccgagggttccatgccaggaaaccggctgtatgtgcaccccgactctccaaacactggcgctcactggatgcgccaggaggtatcatttggtaagctgaaactgacaaacaacaagggggcttccaacaatgtcactcagatgatcgttttgcaatccctgcataaataccagccccgattgcacatcgtagaggttaatgacggagaacctgaggctgcctgttccgcaagcaacactcacgtgtttacctttcaggaaacccaattcatcgccgtaaccgcctaccaaaacgctgaaatcactcaactcaagatcgacaacaatcccttcgctaaagggtttcgggagaactttgagagtatgtatgcctctgtggataccagcgtcccctctccccccggtcccaactgtcagttgctggggggagacccattttcccctctcctctcaaatcagtatccagtgccttctcgcttctatcctgacctgcctgggcagcctaaagacatgattagccagccatactggctgggaactccacgggaacatagttacgaggcagaatttagggcagtaagtatgaagcctaccctgctcccatcagccccaggccctactgtgccatattatagaggccaagacgtgctggctcccggggctggatggcctgtggctccacagtacccaccaaagatgagccctgcaggctggtttcggcccatgcgaactttgcctatggatcccggtcttgggagttctgaagagcagggttcatctcccagtctttggccagaagtgacctcattgcaacccgagcctagcgactccggtttgggtgagggggatacaaaaagacggaggatcagtccctacccaagctcaggtgactcaagcagccctgctggcgcaccatctccatttgacaaagagacagagggtcagttttataactactttcctaat

SEQ ID NO: 30 - DNA, 10087 bp, synthetic construct
  misc_feature 1..10087: note = Description of Artificial Sequence: Synthetic polynucleotide
  source 1..10087: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatgggtattgtagagccaggttgtggcgatatgctcactggcactgaacccatgccctccgatgagggacgggggcccggcgctgatcaacaacacaggtttttctacccagagccaggggctcaggatccaaccgacaggagggccggcagctccctcgggacaccttatagcggcggggcactggtacccgcagcacctggccgcttccttggatcctttgcataccccccaagggctcaggtagctgggttccccggtcccggcgagttttttccacctccagctggagccgaaggctatcctcccgttgacgggtatcccgcccctgacccacgcgctggactgtatcctggccccagagaagactacgctctgcccgcaggtctggaagtttcaggcaaactgagggtagctctttctaaccacctgctgtggagcaagtttaatcaacaccaaaccgagatgatcatcaccaagcagggccgccgaatgtttccattcctttcctttactgttgccggtcttgagccaacaagtcactaccgaatgttcgtcgatgttgtgctcgtggaccaacaccattggcggtatcaatccggtaaatgggtacagtgcggtaaagctgagggcagcatgcccggcaatagattgtacgtacaccctgactctcctaacaccggcgctcactggatgagacaggaagtcagtttcggtaagctcaaactcactaacaataaaggcgcttcaaacaatgtgacccagatgatcgtattgcaaagccttcacaagtatcaaccacgcttgcatatcgtagaagtcaacgacggtgagcctgaggcagcatgtagtgcaagtaacacccacgtcttcacttttcaggagacacaattcatcgctgtaactgcttatcaaaacgccgaaattactcaacttaaaattgataacaaccccttcgccaagggttttagagagaacttcgagagcatgtatgcaagcgtagacaccagcgtaccatctccccccggacctaattgccagctgctcggtggcgatcctttcagccctctcctctccaaccaatatccagttccttcacgcttttacccagacttgccaggacaaccaaaggacatgatcagccagccatactggcttggaactcccagggaacattcctatgaagctgaatttcgagcagtctccatgaaaccaacactgttgccctccgctccaggccctacagtaccttattatcggggacaagacgtactggctcctggcgcaggatggccagtcgccccacaataccctcccaagatgagtcctgctgggtggtttaggcctatgaggaccttgcccatggaccctggcttgggatctagcgaagagcagggttcctcacccagtttgtggcctgaagtaacttcacttcagcctgaaccatcagacagcggcctgggtgaaggcgacactaaacgacgccggatatcaccttatcccagtagtggggactctagctcaccagcaggggccccttcccccttcgacaaagagactgaaggacagttctacaattatttccctaacggatccggagctactaacttcagcctgctgaagcaggctggagacgtggaggagaaccctggacccatgggagcaggggcaaccggtagggcgatggatggtccgcggcttttgcttcttttgttgctgggggtgagcttgggcggcgctaaagaagcatgtcccaccggactgtacacgcatagtggcgagtgttgtaaagcatgtaaccttggggagggcgttgctcagccatgcggggccaatcaaacagtttgcgagccttgtttggactcagtcacgttctcagatgttgtatctgccaccgaaccatgtaaaccctgcacggaatgtgtaggtcttcaaagcatgtctgcaccttgtgtcgaagcggacgacgctgtttgtcgatgtgcttatggttactatcaggacgaaacgaccgggcggtgtgaagcctgtcgggtatgcgaagccggctctggtcttgtgttttcctgccaagacaaacagaacactgtctgcgaggaatgcccagatggtacctatagcgatgaggccaaccatgttgatccctgcctcccctgtactgtttgcgaagataccgaaagacagcttagagaatgtacccgatgggccgatgccgagtgtgaagagattcccggtcgctggataacgcgcagcacccccccagaggggagtgattcaacagcgccaagcacccaggaaccggaagctcccccggagcaagaccttattgcgtcaacggttgcgggagttgtgaccactgttatggggtctagccaacctgtagtgaccaggggaacgaccgataatctcataccggtatattgctcaatacttgctgccgtggtcgttggcctggtggcgtacattgcctttaagagatagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 31 - DNA, 10614 bp, synthetic construct
  misc_feature 1..10614: note = Description of Artificial Sequence: Synthetic polynucleotide
  source 1..10614: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagtaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagatgagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcgccaccatgggtattgtagagccaggttgtggcgatatgctcactggcactgaacccatgccctccgatgagggacgggggcccggcgctgatcaacaacacaggtttttctacccagagccaggggctcaggatccaaccgacaggagggccggcagctccctcgggacaccttatagcggcggggcactggtacccgcagcacctggccgcttccttggatcctttgcataccccccaagggctcaggtagctgggttccccggtcccggcgagttttttccacctccagctggagccgaaggctatcctcccgttgacgggtatcccgcccctgacccacgcgctggactgtatcctggccccagagaagactacgctctgcccgcaggtctggaagtttcaggcaaactgagggtagctctttctaaccacctgctgtggagcaagtttaatcaacaccaaaccgagatgatcatcaccaagcagggccgccgaatgtttccattcctttcctttactgttgccggtcttgagccaacaagtcactaccgaatgttcgtcgatgttgtgctcgtggaccaacaccattggcggtatcaatccggtaaatgggtacagtgcggtaaagctgagggcagcatgcccggcaatagattgtacgtacaccctgactctcctaacaccggcgctcactggatgagacaggaagtcagtttcggtaagctcaaactcactaacaataaaggcgcttcaaacaatgtgacccagatgatcgtattgcaaagccttcacaagtatcaaccacgcttgcatatcgtagaagtcaacgacggtgagcctgaggcagcatgtagtgcaagtaacacccacgtcttcacttttcaggagacacaattcatcgctgtaactgcttatcaaaacgccgaaattactcaacttaaaattgataacaaccccttcgccaagggttttagagagaacttcgagagcatgtatgcaagcgtagacaccagcgtaccatctccccccggacctaattgccagctgctcggtggcgatcctttcagccctctcctctccaaccaatatccagttccttcacgcttttacccagacttgccaggacaaccaaaggacatgatcagccagccatactggcttggaactcccagggaacattcctatgaagctgaatttcgagcagtctccatgaaaccaacactgttgccctccgctccaggccctacagtaccttattatcggggacaagacgtactggctcctggcgcaggatggccagtcgccccacaataccctcccaagatgagtcctgctgggtggtttaggcctatgaggaccttgcccatggaccctggcttgggatctagcgaagagcagggttcctcacccagtttgtggcctgaagtaacttcacttcagcctgaaccatcagacagcggcctgggtgaaggcgacactaaacgacgccggatatcaccttatcccagtagtggggactctagctcaccagcaggggccccttcccccttcgacaaagagactgaaggacagttctacaattatttccctaactagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatgggagcaggggcaaccggtagggcgatggatggtccgcggcttttgcttcttttgttgctgggggtgagcttgggcggcgctaaagaagcatgtcccaccggactgtacacgcatagtggcgagtgttgtaaagcatgtaaccttggggagggcgttgctcagccatgcggggccaatcaaacagtttgcgagccttgtttggactcagtcacgttctcagatgttgtatctgccaccgaaccatgtaaaccctgcacggaatgtgtaggtcttcaaagcatgtctgcaccttgtgtcgaagcggacgacgctgtttgtcgatgtgcttatggttactatcaggacgaaacgaccgggcggtgtgaagcctgtcgggtatgcgaagccggctctggtcttgtgttttcctgccaagacaaacagaacactgtctgcgaggaatgcccagatggtacctatagcgatgaggccaaccatgttgatccctgcctcccctgtactgtttgcgaagataccgaaagacagcttagagaatgtacccgatgggccgatgccgagtgtgaagagattcccggtcgctggataacgcgcagcacccccccagaggggagtgattcaacagcgccaagcacccaggaaccggaagctcccccggagcaagaccttattgcgtcaacggttgcgggagttgtgaccactgttatggggtctagccaacctgtagtgaccaggggaacgaccgataatctcataccggtatattgctcaatacttgctgccgtggtcgttggcctggtggcgtacattgcctttaagagatagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 32 - DNA, 2124 bp, synthetic construct
  misc_feature 1..2124: note = Description of Artificial Sequence: Synthetic polynucleotide
  source 1..2124: mol_type = other DNA
atgcagttgggggaacaacttctggtgagtagtgtaaacctcccaggagcacacttctactcccttgagagtgctagggggggcggtgggggtggaggcggaggcggcggtggtggaggcggaagcgtttcactcttgcctggagccgctccttcaccacagcgacttgatcttgacaaggcctctaagaagtttcctggtagcctcccatgtcaagcaggcagtgccgagcctgccggtgccggcgccggggcacctgcagcaatgctcagcgatgcagacgcaggtgataccttcggaagtaccagcgccgttgccaagcccgggccacccgatgggaggaagggctctccctgtgctgaggaagaactgccatcagccgcaaccgcagccgccaccgccagatatagcatggattctctgtcctccgaacgctactacctccctagtcccgggccccaaggttcagagcttgctgcaccttgctccttgttccagtatcccgccgccgccggggctgcccatggccccgtgtaccctgcaagtaatggggctcgctatccctacggctctatgctccctcccggtggctttcctgccgcagtttgccctcccgcaagagctcagtttggacccgcagccggctctgggagtggcgcaggtagctctggcggaggcgccggaggtccaggcgcatacccttacggccaaggaagcccattgtacgggccctatgccggaaccagcgcagccggatcttgcggaggtctgggcgggttgggagtacccggctctgggttccgcgcacatgtatacttgtgcaaccgacccctgtggctcaaattccaccgacatcaaaccgaaatgattatcacaaaacagggacgacggatgtttccttttttgtctttcaatataaatggcctgaatcccaccgcacactataacgtctttgtcgaagtcgttctggccgatcccaaccactggaggttccagggcggtaaatgggttacctgtggtaaagcagataacaacatgcaagggaataagatgtatgtacaccctgagagtcctaacacaggaagtcactggatgagacaagaaatatcatttggcaaattgaagctcaccaataacaaaggggcaaacaataataatacacagatgattgtcttgcagtctctccacaaatatcaacctagacttcatatagttgaggtaaccgaggatggtgttgaagaccttaatgagccctcaaagactcaaactttcacattcagcgagacccaatttattgccgtgactgcatatcaaaacacagatatcactcagctcaaaattgatcataatccattcgcaaaaggattcagagataactatgacagtatgtacaccgctagtgaaaatgaccgattgacccctagtcccactgatagtccacggtcccatcagatcgtccccggaggtcggtatggagtccaaaacttcttcccagaaccattcgttaacacacttcctcaggctcgatattacaacggtgagagaactgtaccccagactaacggcctgctgtccccccagcagtcagaagaggtggcaaatccaccacagcggtggcttgtcactcccgtacagcaacccgtgactaacaagctcgatatcggttcatatgagagcgagtacacttcatccacactgctcccatatggaataaagagcctgcccctgcaaacctctcatgccctggggtactacccagacccaacttttccagccatggccggttggggcggacggggagcctatcaaaggaaaatggctgcagggcttccttggacaagcaggatgagccccccagtttttcccgaggatcagcttgccaaagagaaagtgaaggaagagatatcttcttcctggattgaaacacctcctagtatcaaatccctggattctagtgacagcggggtttataattcagcatgtaaacgcaaacgcctgagcccttctactcccagtaatggaaattcccccccaatcaagtgcgaagatatcaacacagaagagtatagcaaagatacaagcaaaggcatgggagcttattacgccttttacacaagtccttag

SEQ ID NO: 33 - DNA, 3659 bp, synthetic construct
  misc_feature 1..3659: note = Description of Artificial Sequence: Synthetic polynucleotide
  source 1..3659: mol_type = other DNA
tgaagcagccaatcatggccgacggccccagatgcaagaggagaaagcaggctaacccacggcgcaagaacgtggtgaactacgacaacgtggtggatgctggatctgagaccgacgaggaggataagctgcacatcgccgaggacgatagcctggctaaccccctggaccaggatacaagccctgcctccatgccaaaccacgagagctccccacacatgagccagggactgctgcctagggaggaggaggaggaggagctgagggagtccgtggtggagcactcttggcacagcggcgagatcctgcaggcctctgtggctggacccgaggagatgaaggaggactacgatgccatgggccctgaggctaccatccagaccacaatcaacaacggaaccgtgaagaacgccaactgtacatccgatttcgaggagtactttgctaagagaaagctcgaggagagggacggacacgccgtgtctatcgaggagtacctgcagagaagcgataccgccatcatctaccctgaggctccagaggagctgagcaggctgggcacacctgaggctaacggacaggaggagaacgatctgccacctggcacccctgacgccttcgctcagctgctgacatgcccatactgtgaccgcggatacaagaggctgacctccctgaaggagcacatcaagtacagacacgagaagaacgaggagaacttctcttgccctctgtgcagctacacctttgcttaccgcacacagctggagaggcacatggtgacccacaagccaggcacagatcagcaccagatgctgacccagggcgctggaaaccggaagttcaagtgcacagagtgtggaaaggcctttaagtacaagcaccacctgaaggagcacctgcgcatccacagcggcgagaagccctacgagtgccctaactgtaagaagaggttctctcacagcggcagctactctagccacatctcctctaagaagtgcatcggcctgatctccgtgaacggacgcatgaggaacaacatcaagaccggcagctccccaaactccgtgtctagctcccccaccaactctgctatcacacagctgcgcaacaagctggagaacggcaagccactgtctatgagcgagcagaccggactgctgaagatcaagacagagcccctggacttcaacgattacaaggtgctgatggccacccacggcttctccggatctagccccttcatgaacggaggactgggagccacatctccactgggagtgcacccttccgctcagtctccaatgcagcacctgggagtgggaatggaggctccactgctgggcttccctaccatgaacagcaacctgtccgaggtgcagaaggtgctgcagatcgtggacaacaccgtgtctcggcagaagatggattgcaagacagaggacatcagcaagctgaagggctaccacatgaaggacccctgtagccagccagaggagcagggagtgacatccccaaacatcccaccagtgggactgccagtggtgtctcacaacggagccaccaagagcatcatcgactacacactggagaaggtgaacgaggccaaggcttgcctgcagtccctgaccacagactctaggagacagatcagcaacatcaagaaggagaagctgcgcaccctgatcgacctggtgacagacgataagatgatcgagaaccactccatctctaccccattctcctgccagttttgtaaggagtctttccccggccctatcccactgcaccagcacgagagatacctgtgcaagatgaacgaggagatcaaggccgtgctgcagcctcacgagaacatcgtgccaaacaaggccggcgtgtttgtggacaacaaggctctgctgctgtcctctgtgctgagcgagaagggactgacctcccccatcaacccttacaaggaccacatgagcgtgctgaaggcctactacgctatgaacatggagcctaacagcgatgagctgctgaagatctccatcgctgtgggcctgccacaggagttcgtgaaggagtggtttgagcagagaaaggtgtaccagtacagcaactccagatctcccagcctggagcggacatctaagccactggcccccaacagcaaccctaccacaaaggattccctgctgcctcggtctccagtgaagcccatggactccatcacctccccctctatcgctgagctgcacaactctgtgacaagctgtgaccctccactgagactgaccaagagctcccacttcacaaacatcaaggccgtggacaagctggatcacagccggtccaacacccccagccctctgaacctgtctagcacatcctctaagaactctcacagctcctcttacacccctaacagctttagctccgaggagctgcaggctgagccactggacctgtccctgcctaagcagatgagagagccaaagggcatcatcgccaccaagaacaagaccaaggctacatccatcaacctggatcacaacagcgtgagcagctcctctgagaacagcgacgagccactgaacctgacattcatcaagaaggagttttctaacagcaacaacctggataacaagagcaacaaccccgtgttcggaatgaaccctttttccgccaagcctctgtacacaccactgccacctcagagcgccttcccaccagctacctttatgcctccagtgcagacatccatcccaggactgaggccatacccaggactggaccagatgtctttcctgcctcacatggcttacacctacccaacaggcgccgctacctttgccgatatgcagcagcggcgcaagtaccagcgcaagcagggcttccagggagacctgctggatggcgctcaggactacatgagcggactggacgatatgacagactccgattcttgcctgtccaggaagaagatcaagaagaccgagtctggcatgtacgcctgcgacctgtgcgataagacatttcagaagagctcctctctgctgagacacaagtacgagcacaccggaaagcggccccaccagtgccagatctgtaagaaggccttcaagcacaagcaccacctgatcgagcactcccgcctgcactctggcgagaagccttaccagtgcgacaagtgtggcaagaggttcagccactccggatcttacagccagcacatgaaccacaggtacagctactgtaagagagaggctgaggagagggaggctgctgagagagaggctagggagaagggacacctggagccaaccgagctgctgatgaacagggcctacctgcagtccatcacaccacagggatactccgattctgaggagagggagagcatgccaagggacggagagtccgagaaggagcacgagaaagaaggagaggagggctacggaaagctgaggagacgggacggcgatgaagaagaagaagaggaggaggaggagagcgagaacaagtccatggacaccgatcctgagacaatcagggatgaggaggagaccggcgaccacagcatggacgatagctccgaggacggaaagatggagacaaagtccgaccacgaggaggataacatggaggacggaatggagcgcaggagacggtag

SEQ ID NO: 34 - DNA, 6341 bp, synthetic construct
  misc_feature 1..6341: note = Description of Artificial Sequence: Synthetic polynucleotide
  source 1..6341: mol_type = other DNA
aagcttatactcgagctctagattgggaacccgggtctctcgaattcaccgccatgggatctagcaaaagcaaacccaaagacccaaaagcccgagttttggcagaggcttcagatcccagggagagaatcccacctggaaacagtggagaagagacaataggagaggccttcgaatggctaaacagaacagtagaggagataaacagagaggcggtaaaccacctaccaagggagctaattttccagggttggcaaagggcttgggaatactggcatgatgaacaagggatgtcacaaagctatgtaaaatacagatacttgtgtttaatacaaaaggctttatttatgcattgcaagaaaggctgtagatgtctaggggaaggacacggggcagggggatggagaccaggacctcctcctcctccccctccaggactagcataagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 35 - DNA, 6365 bp, synthetic construct
  misc_feature 1..6365: note = Description of Artificial Sequence: Synthetic polynucleotide
  source 1..6365: mol_type = other DNA
aagcttatactcgagctctagattgggaacccgggtctctcgaattcaccgccatgggatctagcaaaagcaaacccaaagacccaaaagcccgagttttggcagaggctgattataaagacgatgatgacaaatcagatcccagggagagaatcccacctggaaacagtggagaagagacaataggagaggccttcgaatggctaaacagaacagtagaggagataaacagagaggcggtaaaccacctaccaagggagctaattttccagggttggcaaagggcttgggaatactggcatgatgaacaagggatgtcacaaagctatgtaaaatacagatacttgtgtttaatacaaaaggctttatttatgcattgcaagaaaggctgtagatgtctaggggaaggacacggggcagggggatggagaccaggacctcctcctcctccccctccaggactagcataagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 36 - DNA, 7586 bp, synthetic construct
  misc_feature 1..7586: note = Description of Artificial Sequence: Synthetic polynucleotide
  source 1..7586: mol_type = other DNA
ggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattaagcttatactcgagctctagattgggaacccgggtctctcgaattcgagatctccaccatgcacagacctagacgtcgtggaactcgtccacctccactggcactgctcgctgctctcctcctggctgcacgtggtgctgatgcagaggtgaagctgcaggagagcggccccagcctggtgcagcccagccagaccctgagcctgacctgcagcgtgagcggcttcagcctgatcagcgacagcgtgcactgggtgagacagccccccggcaagggcctggagtggatgggcggcatctgggccgacggcagcaccgactacaacagcgccctgaagagcagactgagcatcagcagagacaccagcaagagccagggcttcctgaagatgaacagcctgcagaccgacgacaccgccatctacttctgcaccagcaacagagagagctactacttcgactactggggccagggcgtgatggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaagagctgcaactggaggagagctgtgcggaggcgcaggacggggagctggacgggctgtggacgaccatcaccatcttcatcacactcttcctgctaagcgtgtgctacagtgccaccgtcaccttcttcaaggtgaagtggatcttctcctcagtggtggacctgaagcagaccatcgtccccgactacaggaacatgataaggcagggggcctgagc

SEQ ID NO: 37 - DNA, 6683 bp, synthetic construct
  misc_feature 1..6683: note = Description of Artificial Sequence: Synthetic polynucleotide
  source 1..6683: mol_type = other DNA
ggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattaagcttatactcgagctctagattgggaacccgggtctctcgaattcgagatctccaccatgcacagacctagacgtcgtggaactcgtccacctccactggcactgctcgctgctctcctcctggctgcacgtggtgctgatgcagacatccagatgacccagagccccgccagcctgagcgccagcctgggcgacaaggtgaccatcacctgccaggccagccagaacatcgacaagtacatcgcctggtaccagcagaagcccggcaaggcccccagacagctgatccactacaccagcaccctggtgagcggcacccccagcagattcagcggcagcggcagcggcagagactacagcttcagcatcagcagcgtggagagcgaggacatcgccagctactactgcctgcagtacgacaccctgtacaccttcggcgccggcaccaagctggagctgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagc

SEQ ID NO: 38 - DNA, 5949 bp, synthetic construct
  misc_feature 1..5949: note = Description of Artificial Sequence: Synthetic polynucleotide
  source 1..5949: mol_type = other DNA
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcattcctgcaggtcgacatcgatcttaagcagtacttctagaatgcctgggggtccaggagtcctccaagctctgcctgccaccatcttcctcctcttcctgctgtctgctgtctacctgggccctgggtgccaggccctgtggatgcacaaggtcccagcatcattgatggtgagcctgggggaagacgcccacttccaatgcccgcacaatagcagcaacaacgccaacgtcacctggtggcgcgtcctccatggcaactacacgtggccccctgagttcttgggcccgggcgaggaccccaatggtacgctgatcatccagaatgtgaacaagagccatgggggcatatacgtgtgccgggtccaggagggcaacgagtcataccagcagtcctgcggcacctacctccgcgtgcgccagccgccccccaggcccttcctggacatgggggagggcaccaagaaccgaatcatcacagccgaggggatcatcctcctgttctgcgcggtggtgcctgggacgctgctgctgttcaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtgaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcgccaccatggccaggctggcgttgtctcctgtgcccagccactggatggtggcgttgctgctgctgctctcagctgagccagtaccagcagccagatcggaggaccggtaccggaatcccaaaggtagtgcttgttcgcggatctggcagagcccacgtttcatagccaggaaacggggcttcacggtgaaaatgcactgctacatgaacagcgcctccggcaatgtgagctggctctggaagcaggagatggacgagaatccccagcagctgaagctggaaaagggccgcatggaagagtcccagaacgaatctctcgccaccctcaccatccaaggcatccggtttgaggacaatggcatctacttctgtcagcagaagtgcaacaacacctcggaggtctaccagggctgcggcacagagctgcgagtcatgggattcagcaccttggcacagctgaagcagaggaacacgctgaaggatggtatcatcatgatccagacgctgctgatcatcctcttcatcatcgtgcctatcttcctgctgctggacaaggatgacagcaaggctggcatggaggaagatcacacctacgagggcctggacattgaccagacagccacctatgaggacatagtgacgctgcggacaggggaagtgaagtggtctgtaggtgagcacccaggccaggagtgactcgagagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 39 - DNA, 4595 bp, synthetic construct
  misc_feature 1..4595: note = Description of Artificial Sequence: Synthetic polynucleotide
  source 1..4595: mol_type = other DNA
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcattcctgcaggtcgacatcgatcttaagcagtacttctagaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcttcgaaggtaccagcacagtggactcgagagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 40 - DNA, 9117 bp, synthetic construct
  misc_feature 1..9117: note = Description of Artificial Sequence: Synthetic polynucleotide
  source 1..9117: mol_type = other DNA
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaaggatcttccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggcggccgcagtgaacatgtcgagcaggtacggctctcactagggccgccgccagtgcttgaggtcgtcctggtacactagcgcgaagagcaaccccagaaacgagtcccgcctgacccacgagtccatcaccaacagcccgtcgtcgtgccccggcagcggctacccccacaagacgaccatcaccagccgctcgacgtgcgacggcaggagctacaacaccgcctagaacttcaagtggaactacggcaagaagacgaacagccggtactatatctgcaacaccgacaacatcaacatgaggtcgaacacggggtcctacaacggcaggaggaacttcagctacgggaagtcgagctacgccaagtggtcccacagcgggagcttgaagtggagccgcgcccagaacatcaacggcagcaggaacttcttctaccacgcgaggacctgcatcggaagcccgtaccgcctgaacttcttcagcacgacgaagtacaccagccccatcgccgacttcgtgacgtgcggcatccagtcccaccagtgctcccacccggtcccgtgcccgtcgaacggccaccacgtctacttgaagtcccagtcgaacggcatccaccgtagcgggagcgggagcggcctgtgcgacttgaacaccggcaaatgcagcggcaggtcgagctggtcctacccgtggtggggccacttgtcgaggagcgggaacgagtggtacccaggtcccaagaggaggtgcagaggtcggacgaagtcgtccgacttcaatcatcgaggcctaggcaatccctttattaacatcttgacaggaagtcagagaaacagcttcccccttccccggggacgaccactcgatctcaggggtgatgaccctattccactataggccgcagcaaatcacagcggaagtgggtccggcgacagactaccaagtccgacttcacttcaatgaagtccggtgtttgacccactccttgggacgagaagcgatctagggttcggtcccaggtacccgttccaggagtatccggatttggtgggtcgtcctgagtagaaccctcccataacaccccgctgaccggtaggacgcggtcctcggtcatgcagaacaggggctattattccatgacatcccggacctcgcctcccgttgtcacaaccaaagtacctctgacgagctttaagtcgaagtatccttacaagggaccctcaaggttcggtcataccgaccgactagtacaggaaaccaacaggaccgttcagacccattttcgcacttccttgacctataaccaacctctcctctcccgactttcctagcggtggctcgtcgaccgttaatccaggcccccctctaccatgcgaccacagatgcgaacgtatgtacgagagcttcaagagagattttgggaaccgcttccccaacaatagttaaaattcaactcattaaagccgcaaaactattcgtcaatgtcgctacttaacacagaggacttttcacttctgcacccacaatgaacgtgatgtacgacggagtccgagtggcagcaactgaagatgctatacgttcgcaccaactatgaacacttccgaaacgttatgctagtagacccagtgtaacaaacttcgcggaaataacaatcactcaaactcgaatggctttgactgaaggacagagtaggtcactcgcggccacaatcctctcagtcccacatgcatgttagataacggcccgtacgacgggagtcgaaatggcgtgacatgggtaaatggcctaactatggcggttaccacaaccaggtgctcgtgttgtagctgcttgtaagccatcactgaacaaccgagttctggccgttgtcatttcctttccttacctttgtaagccgccgggacgaaccactactagtagagccaaaccacaactaatttgaacgaggtgtcgtccaccaatctttctcgatgggagtcaaacggactttgaaggtctggacgcccgtctcgcatcagaagagaccccggtcctatgtcaggtcgcgcacccagtccccgccctatgggcagttgccctcctatcggaagccgaggtcgacctccaccttttttgagcggccctggccccttgggtcgatggactcgggaaccccccatacgtttcctaggttccttcgccggtccacgacgcccatggtcacggggcggcgatattccacagggctccctcgacggccgggaggacagccaacctaggactcggggaccgagacccatctttttggacacaacaactagtcgcggcccgggggcagggagtagcctcccgtacccaagtcacggtcactcgtatagcggtgttggaccgagatgttatgggtagaattcgttggatccttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgtcgagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacgcgtctctagaggtcgccaccatggttcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaaagcttcgaccccatccatcgataaaataaaagattttatttagtctccagaaaaaggggggaatgaaagaccccacctgtaggtttggcaagctagctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagtgtcgagcatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc

SEQ ID NO: 41 - DNA, 6981 bp, synthetic construct
  misc_feature 1..6981: note = Description of Artificial Sequence: Synthetic polynucleotide
  source 1..6981: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttaacgaattcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 42 - DNA, 8638 bp, synthetic construct
  misc_feature 1..8638: note = Description of Artificial Sequence: Synthetic polynucleotide
  source 1..8638: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttaacgaattcatgggtattgtagagccaggttgtggcgatatgctcactggcactgaacccatgccctccgatgagggacgggggcccggcgctgatcaacaacacaggtttttctacccagagccaggggctcaggatccaaccgacaggagggccggcagctccctcgggacaccttatagcggcggggcactggtacccgcagcacctggccgcttccttggatcctttgcataccccccaagggctcaggtagctgggttccccggtcccggcgagttttttccacctccagctggagccgaaggctatcctcccgttgacgggtatcccgcccctgacccacgcgctggactgtatcctggccccagagaagactacgctctgcccgcaggtctggaagtttcaggcaaactgagggtagctctttctaaccacctgctgtggagcaagtttaatcaacaccaaaccgagatgatcatcaccaagcagggccgccgaatgtttccattcctttcctttactgttgccggtcttgagccaacaagtcactaccgaatgttcgtcgatgttgtgctcgtggaccaacaccattggcggtatcaatccggtaaatgggtacagtgcggtaaagctgagggcagcatgcccggcaatagattgtacgtacaccctgactctcctaacaccggcgctcactggatgagacaggaagtcagtttcggtaagctcaaactcactaacaataaaggcgcttcaaacaatgtgacccagatgatcgtattgcaaagccttcacaagtatcaaccacgcttgcatatcgtagaagtcaacgacggtgagcctgaggcagcatgtagtgcaagtaacacccacgtcttcacttttcaggagacacaattcatcgctgtaactgcttatcaaaacgccgaaattactcaacttaaaattgataacaaccccttcgccaagggttttagagagaacttcgagagcatgtatgcaagcgtagacaccagcgtaccatctccccccggacctaattgccagctgctcggtggcgatcctttcagccctctcctctccaaccaatatccagttccttcacgcttttacccagacttgccaggacaaccaaaggacatgatcagccagccatactggcttggaactcccagggaacattcctatgaagctgaatttcgagcagtctccatgaaaccaacactgttgccctccgctccaggccctacagtaccttattatcggggacaagacgtactggctcctggcgcaggatggccagtcgccccacaataccctcccaagatgagtcctgctgggtggtttaggcctatgaggaccttgcccatggaccctggcttgggatctagcgaagagcagggttcctcacccagtttgtggcctgaagtaacttcacttcagcctgaaccatcagacagcggcctgggtgaaggcgacactaaacgacgccggatatcaccttatcccagtagtggggactctagctcaccagcaggggccccttcccccttcgacaaagagactgaaggacagttctacaattatttccctaacggatccggagctactaacttcagcctgctgaagcaggctggagacgtggaggagaaccctggacccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 43 - protein, 9 aa, Human betaherpesvirus 5
NLVPMVATV

SEQ ID NO: 44 - DNA, 27 bp, Human betaherpesvirus 5
  source 1..27: mol_type = unassigned DNA
aacctggtacccatggtcgctaccgtg

SEQ ID NO: 45 - DNA, 1809 bp, synthetic construct
  misc_feature 1..1809: note = Description of Artificial Sequence: Synthetic polynucleotide
  source 1..1809: mol_type = other DNA
tctagagccaccatggaaaagaaccccctggccgctcccctgctgatcctgtggtttcacctggactgcgtgtccatcctgaacgtggaacagagcccccagagcctgcatgtgcaggaaggcgacagcaccaacttcacctgtagcttccccagcagcaacttctacgccctgcactggtacagatgggagacagccaagagccccgaggccctgttcgtgatgaccctgaacggcgacgagaagaagaagggccggatcagcgccaccctgaacaccaaagagggctacagctacctgtacatcaagggcagccagcccgaggactccgccacatacctgtgcgccagaaacaccggcaaccagttctacttcggcaccggcaccagcctgaccgtgatccccaacatccagaaccccgaccccgccgtgtaccagctgaaggaccctagaagccaggacagcaccctgtgcctgttcaccgacttcgacagccagatcaacgtgcccaagaccatggaatccggcaccttcatcaccgataagtgcgtgctggacatgaaggccatggacagcaagagcaacggcgccattgcctggtccaaccagaccagcttcacatgccaggacatcttcaaagagacaaacgccacctaccccagctccgacgtgccctgtgatgccacactgaccgagaagtccttcgagacagacatgaacctgaacttccagaacctgagcgtgatgggcctgagaatcctgctgctgaaggtggccggcttcaacctgctgatgacactgcggctgtggtcctctggcagcggcgccacaaatttcagcctgctgaaacaggccggcgacgtggaagagaaccctggccctatggtcatcggcctgctgtgttgtgccgccctgtctctgctgtgggccggacctgtgaatgccggcgtgacccagacccccaagttccaggtgctgaaaaccggccagagcatgaccctgcagtgcgcccaggacatgaatcacgagtacatgagctggtatcggcaggaccccggcatgggcctgcggctgattcactattctgtgggagccggcatcaccgaccagggcgaagtgcccaatggctacaacgtgtccagaagcaccaccgaggacttcccactgagactgctgtctgccgcccctagccagacctccgtgtacttctgcgccagcagctttcagaccggcgccagctacggctacacatttggctctggcacccggctgacagtgctggaagatctgagaaacgtgaccccccccaaggtgtccctgttcgagcctagcaaggccgagatcgccaacaagcagaaagccaccctcgtgtgcctggccagaggcttcttccccgaccacgtggaactgtcttggtgggtcaacggcaaagaggtgcactccggcgtgtgcaccgatccccaggcctacaaagagagcaactactcctactgcctgtccagcagactgcgggtgtccgccaccttctggcacaacccccggaaccacttcaggtgccaggtgcagtttcacggcctgagcgaagaggacaagtggcccgagggcagccctaagcccgtgacacagaatatcagcgccgaagcctggggcagagccgactgtggaatcacctctgccagctaccaccagggcgtgctgagcgccacaatcctgtacgagatcctgctgggcaaggccaccctgtacgccgtgctggtgtctggcctggtgctgatggccatggtcaagaagaaaaacagctgaggatcc

SEQ ID NO: 46 - DNA, 1608 bp, synthetic construct
  misc_feature 1..1608: note = Description of Artificial Sequence: Synthetic polynucleotide
  source 1..1608: mol_type = other DNA
atgggcattgtagagccgggctgcggcgacatgctcactggtacagaacccatgccaggcagtgacgagggccgagcgcccggtgctgatcctcaacataggtatttctacccggaaccgggagcacaagatgctgacgaacgccgcggcggaggttccctcggctctccctaccccggcggtgccctggtcccagctcctccctcccggtttctgggagcatatgcttatcccccacgaccccaggctgccgggtttcctggggctggcgaaagttttcctccccccgcagacgctgaggggtatcagcccggggagggatatgcagcaccggatccaagggccggattgtatccgggcccccgcgaagactacgcactccccgcaggacttgaggtatcaggcaaacttagagttgctctgaataatcatctcctgtggtctaagtttaaccaacaccagaccgagatgattataacgaaacagggtagacgaatgttcccgttcctctcttttacagttgccggattggagccgacatctcactatcgaatgttcgtggatgttgtgctggttgaccaacatcattggcgctatcaaagcggaaagtgggtccaatgcggtaaagcagagggctctatgccagggaatagactgtatgtccaccccgactccccgaacactggtgctcattggatgcgacaggaggtatcattcggcaaactgaaactgactaataacaaaggcgcgagcaataatgtgactcaaatgatagtattgcagagtcttcataaataccaaccaagattgcatatagttgaggtaaacgacggcgaacctgaagcagcatgtaacgcctccaacacacacatatttacctttcaagaaacccagttcatcgccgtcactgcataccagaatgcagagattacacaactcaagatagataacaatccttttgccaagggatttcgcgagaattttgagagtatgtacacctctgtggacacgagtataccctccccgcccggtccgaattgccaattccttgggggagatcactacagcccgcttcttccgaaccaataccctgtccctagtcgattctacccggaccttcccggacaggccaaagatgttgttccccaagcttactggctgggtgctccccgggaccacagctatgaggcagaattccgggccgtctctatgaaacctgcttttcttccgagcgctccgggccccaccatgtcatactatagaggtcaagaggtacttgccccgggggccgggtggcccgtcgcacctcaataccctccaaagatgggccctgccagctggtttcgaccaatgcgaacattgccaatggaacctgggcccggcggatccgaaggacgcggcccagaagatcagggccctccactcgtttggaccgaaatagcacctatccgacccgaaagttctgattctggattgggggagggggattccaagagacggcgagtttccccgtacccctctagtggcgatagctcatccccggcaggtgctccttcaccatttgataaagaggcagaaggacagttttacaactattttccaaattag

SEQ ID NO: 47 - DNA, 10006 bp, synthetic construct
  misc_feature 1..10006: note = Description of Artificial Sequence: Synthetic polynucleotide
  source 1..10006: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatggggatcgttgagcctggatgcggggacatgctgaccgggacagagcccatgccgggaagtgatgaaggcagagctcctggagcagatccgcagcatagatatttctaccctgaaccgggcgcccaggatgcggacgaacggcgcggcgggggatctcttgggtccccgtaccctggtggcgccttggtacctgccccccccagtaggttccttggggcgtacgcatacccccctcgaccacaagcagcaggctttccaggcgctggagagagctttccaccgcccgcagacgctgaaggctaccagcctggcgaagggtatgcagcgcccgatccgcgggctggcctctacccgggtcctagagaagattatgcgcttcccgcgggcctggaagtttctggtaagctcagggtcgctcttaacaaccacctgttgtggtccaagtttaatcagcatcagacagaaatgataattaccaaacaaggtcggcgaatgttcccctttctctctttcacggttgccggattggagccaacatcacattatcgaatgtttgtggacgtcgtactcgttgatcagcaccactggcgatatcaatctggcaaatgggtccaatgtggcaaggcggagggcagcatgccaggaaatcgactctacgtgcatccggatagtccgaacactggcgcgcactggatgcgacaagaagtaagttttgggaaactgaaactcactaacaataagggggcctctaacaatgtcactcaaatgatcgtattgcagagtcttcataaatatcagcccaggcttcatatagtcgaagtgaatgatggagagccagaggccgcctgcaacgcaagtaatacacacatttttaccttccaagagacccagtttatagcggtcaccgcataccaaaacgccgagattacgcaacttaagattgacaataatcctttcgcaaaaggtttccgggaaaatttcgagagcatgtacacgtccgtagatacgtctataccatctccgcctggtcctaattgtcaatttctcggaggagaccactacagcccacttctgcctaatcaatacccggtacctagtcggttctatcccgacctgccagggcaagctaaagatgttgtgccgcaagcatattggctcggggctccaagagatcactcctatgaagcggagtttcgagcggtgtctatgaagcccgccttcttgccctcagcgcccggacctacgatgtcttactataggggacaggaagttctcgcgccgggtgccggttggcccgtggcgccgcagtatcctccgaagatgggtcccgcctcctggttcagaccgatgaggacactcccaatggaaccggggccgggaggctctgaaggacgggggccggaagaccaagggccccctctcgtctggaccgaaatcgctccgataagacctgaatcatcagactccggtttgggcgagggggacagcaagcgacggcgggtatctccatacccgagcagcggcgactccagttcccccgcaggagctcctagcccatttgacaaggaggctgaggggcaattttacaactacttccccaatagaagaagaagaggatccggagctactaacttcagcctgctgaagcaggctggagacgtggaggagaaccctggacccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 48 - DNA, 10518 bp, synthetic construct
  misc_feature 1..10518: note = Description of Artificial Sequence: Synthetic polynucleotide
  source 1..10518: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagtaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagatgagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcgccaccatggggatcgttgagcctggatgcggggacatgctgaccgggacagagcccatgccgggaagtgatgaaggcagagctcctggagcagatccgcagcatagatatttctaccctgaaccgggcgcccaggatgcggacgaacggcgcggcgggggatctcttgggtccccgtaccctggtggcgccttggtacctgccccccccagtaggttccttggggcgtacgcatacccccctcgaccacaagcagcaggctttccaggcgctggagagagctttccaccgcccgcagacgctgaaggctaccagcctggcgaagggtatgcagcgcccgatccgcgggctggcctctacccgggtcctagagaagattatgcgcttcccgcgggcctggaagtttctggtaagctcagggtcgctcttaacaaccacctgttgtggtccaagtttaatcagcatcagacagaaatgataattaccaaacaaggtcggcgaatgttcccctttctctctttcacggttgccggattggagccaacatcacattatcgaatgtttgtggacgtcgtactcgttgatcagcaccactggcgatatcaatctggcaaatgggtccaatgtggcaaggcggagggcagcatgccaggaaatcgactctacgtgcatccggatagtccgaacactggcgcgcactggatgcgacaagaagtaagttttgggaaactgaaactcactaacaataagggggcctctaacaatgtcactcaaatgatcgtattgcagagtcttcataaatatcagcccaggcttcatatagtcgaagtgaatgatggagagccagaggccgcctgcaacgcaagtaatacacacatttttaccttccaagagacccagtttatagcggtcaccgcataccaaaacgccgagattacgcaacttaagattgacaataatcctttcgcaaaaggtttccgggaaaatttcgagagcatgtacacgtccgtagatacgtctataccatctccgcctggtcctaattgtcaatttctcggaggagaccactacagcccacttctgcctaatcaatacccggtacctagtcggttctatcccgacctgccagggcaagctaaagatgttgtgccgcaagcatattggctcggggctccaagagatcactcctatgaagcggagtttcgagcggtgtctatgaagcccgccttcttgccctcagcgcccggacctacgatgtcttactataggggacaggaagttctcgcgccgggtgccggttggcccgtggcgccgcagtatcctccgaagatgggtcccgcctcctggttcagaccgatgaggacactcccaatggaaccggggccgggaggctctgaaggacgggggccggaagaccaagggccccctctcgtctggaccgaaatcgctccgataagacctgaatcatcagactccggtttgggcgagggggacagcaagcgacggcgggtatctccatacccgagcagcggcgactccagttcccccgcaggagctcctagcccatttgacaaggaggctgaggggcaattttacaactacttccccaatcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 49 - DNA, 2061 bp, synthetic construct
  misc_feature 1..2061: note = Description of Artificial Sequence: Synthetic polynucleotide
  source 1..2061: mol_type = other DNA
atgcagctgggggaacaacttttggtaagtagtgtaaatctccccggcgcgcacttctacccactcgaaagcgcgcgcggcggatcaggaggtagtgcgggtcacttgccgtcagcagcaccctcaccacaaaaattggatctggataaagcctccaagaagttttcagggagtctttcctgtgaggcggttagtggcgaacccgccgcagctagcgcaggagcgccggcagccatgctgagcgatactgatgctggggatgcgttcgctagcgcggcggctgtggcgaaacccggtccaccagatggtaggaaaggctcaccatgcggcgaggaggaattgccctcagcggccgccgccgccgccgcggcagctgctgccgctgctgccacggcaaggtattctatggatagcctctccagtgagcggtactacttgcagtcccccggaccccaaggttctgagttggcagctccgtgcagcctcttcccttaccaagccgcggctggggcgccacatggtcctgtatatccggctccgaacggtgccagatacccctatgggtccatgctgccacctggcggcttccccgccgcggtttgcccaccgggacgggctcagtttggccctggcgcaggtgcgggttccggagccggaggcagttctggaggggggggcggacctggtacgtatcagtactcacaaggggcgccgctctatggtccatacccgggggctgcggctgctggtagctgtggtggtcttggtggcctcggggttcccgggagcggctttcgggcacacgtctacctctgcaatcgccccctgtggttgaaattccaccgacaccaaacggaaatgatcataacgaagcagggccgacggatgtttcctttcctgagttttaatataaacgggctcaacccgactgcgcattataacgtcttcgttgaggtcgttcttgctgacccaaaccactggcgcttccaagggggtaagtgggtcacttgcggaaaggccgataacaacatgcaagggaataaaatgtacgttcacccggaatcccctaacacaggttcccactggatgcgacaagaaattagtttcgggaagctcaaactgaccaataacaaaggggctaacaataacaatacccagatgattgtccttcagtcactccacaaataccagcctcggctgcacatagttgaagtcacagaagatggggttgaggacttgaatgaaccgtccaaaactcaaacctttactttctccgagacgcaatttatcgctgttactgcataccaaaatacagacataactcagctgaaaattgaccacaatccttttgcaaaagggtttcgagacaactacgatagctctcaccagattgtcccggggggccggtacggcgtgcagagtttctttccggaaccgttcgtcaacacacttcctcaagcgcgctattataatggggaaagaacagttccccaaacaaatggactgctgtccccacaacagtcagaagaggttgccaatcctccacagcgctggctggtaacgccggtccaacagccaggaacaaataagttggacattagcagttacgaaagcgagtatacaagcagtactctgctcccgtacggaataaaaagtctcccattgcagacttcccatgccctcggttactaccctgacccaacattccctgcaatggccgggtggggtggtcgcggtagctatcaacgaaaaatggcggcgggcttgccatggacttcccgaacctcacccactgtcttttcagaagaccaactgtccaaagaaaaggttaaggaggagatagggtcaagttggatagagacccctccgtcaataaagtctctggactcaaatgacagcggtgtctacactagcgcatgcaagaggcgacgactgtctccctctaactcttcaaatgaaaactcaccttcaattaagtgcgaagacatcaatgcggaggaatattctaaagacacgtcaaaaggcatggggggttattacgccttctataccacgccgtag

SEQ ID NO: 50 - DNA, 3645 bp, synthetic construct
  misc_feature 1..3645: note = Description of Artificial Sequence: Synthetic polynucleotide
  source 1..3645: mol_type = other DNA
atgaagcagcctatcatggccgacggcccaaggtgcaagaggagaaagcaggctaaccctcggcgcaagaacgtggtgaactacgacaacgtggtggataccggaagcgagacagacgaggaggataagctgcacatcgccgaggacgatggcatcgctaacccactggatcaggagacctcccccgcttctgtgcctaaccacgagagctccccacacgtgtctcaggccctgctgcctagggaggaggaggaggacgagatcagagagggcggagtggagcacccttggcacaacaacgagatcctgcaggcctccgtggatggcccagaggagatgaaggaggactacgatacaatgggacccgaggccaccatccagacagctatcaacaacggcaccgtgaagaacgccaactgtacatccgatttcgaggagtactttgctaagaggaagctggaggagagggacggacacgccgtgtccatcgaggagtacctgcagcggtctgataccgccatcatctacccagaggctcctgaggagctgagcaggctgggcacaccagaggctaacggacaggaggagaacgatctgccacctggaaccccagacgccttcgctcagctgctgacatgcccttactgtgacaggggctacaagagactgacctccctgaaggagcacatcaagtacaggcacgagaagaacgaggagaacttcagctgccctctgtgctcctacacctttgcctacaggacacagctggagagacacatggtgacccacaagccaggaacagaccagcaccagatgctgacccagggcgctggaaacagaaagttcaagtgcacagagtgtggcaaggcctttaagtacaagcaccacctgaaggagcacctgagaatccacagcggagagaagccatacgagtgccccaactgtaagaagagattctctcacagcggctcctactctagccacatctcctctaagaagtgcatcggcctgatctccgtgaacggaaggatgagaaacaacatcaagaccggcagctccccaaacagcgtgtctagctcccccaccaactccgctatcacacagctgagaaacaagctggagaacggcaagccactgtctatgagcgagcagaccggactgctgaagatcaagacagagcccctggacttcaacgattacaaggtgctgatggccacccacggcttcagcggaacatccccctttatgaacggaggactgggagccaccagcccactgggagtgcacccttctgctcagagcccaatgcagcacctgggagtgggaatggaggctccactgctgggctttcctaccatgaactccaacctgtctgaggtgcagaaggtgctgcagatcgtggataacacagtgtcccggcagaagatggactgcaaggccgaggagatctctaagctgaagggataccacatgaaggacccttgttctcagccagaggagcagggcgtgaccagcccaaacatcccaccagtgggactgccagtggtgtcccacaacggagccaccaagtctatcatcgactacacactggagaaggtgaacgaggccaaggcttgcctgcagtctctgaccacagacagcaggagacagatctccaacatcaagaaggagaagctgcggaccctgatcgacctggtgacagacgataagatgatcgagaaccacaacatcagcacccctttctcctgccagttttgtaaggagagcttcccaggacccatccctctgcaccagcacgagagatacctgtgcaagatgaacgaggagatcaaggctgtgctgcagccccacgagaacatcgtgcctaacaaggccggagtgtttgtggacaacaaggctctgctgctgtctagcgtgctgtctgagaagggcatgacaagcccaatcaacccctacaaggaccacatgagcgtgctgaaggcctactacgctatgaacatggagcccaacagcgatgagctgctgaagatctccatcgccgtgggcctgcctcaggagttcgtgaaggagtggtttgagcagaggaaggtgtaccagtactccaactctcggagcccctccctggagaggagctctaagcctctggctccaaactccaaccctccaaccaaggattctctgctgcccagaagccctgtgaagccaatggacagcatcacctctcctagcatcgccgagctgcacaactccgtgacaaactgtgacccccctctgcggctgaccaagccttcccacttcacaaacatcaagccagtggagaagctggatcactcccgctctaacaccccatctcccctgaacctgagctccacatctagcaagaactcccactcctctagctacacccccaactctttttcctctgaggagctgcaggccgagccactggacctgtctctgcccaagcagatgaaggagcctaagagcatcatcgccaccaagaacaagacaaaggctagctccatctccctggatcacaacagcgtgagcagctcctctgagaacagcgacgagcccctgaacctgacattcatcaagaaggagtttagcaactccaacaacctggacaacaagtccaccaaccccgtgttcagcatgaacccattttccgccaagcccctgtacacagctctgccaccccagtctgccttccctccagctacctttatgccccctgtgcagacaagcatccctggcctgagaccttacccaggactggaccagatgtccttcctgcctcacatggcttacacctacccaacaggcgccgctacctttgccgatatgcagcagcggcgcaagtaccagcggaagcagggattccagggagagctgctggacggagctcaggattacatgagcggactggacgatatgacagactctgatagctgcctgagccgcaagaagatcaagaagaccgagtccggaatgtacgcctgcgacctgtgcgataagacatttcagaagagctcctctctgctgcggcacaagtacgagcacaccggcaagcgcccacaccagtgccagatctgtaagaaggccttcaagcacaagcaccacctgatcgagcactctcggctgcacagcggcgagaagccctaccagtgcgacaagtgtggcaagaggttttcccactctggaagctactcccagcacatgaaccacagatacagctactgtaagagggaggctgaggagagggaggctgctgagcgggaggctcgcgagaagggacacctggagccaaccgagctgctgatgaaccgcgcctacctgcagtccatcacacctcagggctactctgatagcgaggagagggagtctatgccaagagacggcgagagcgagaaggagcacgagaaagagggagaggacggctacggaaagctgggcaggcaggacggagatgaggagttcgaagaggaggaggaggagtccgagaacaagtctatggacaccgatcccgagacaatcagagatgaggaggagaccggcgaccactctatggacgatagctccgaggatggaaagatggagacaaagagcgaccacgaggaggataacatggaggacggcatgtag

SEQ ID NO: 51 - DNA, 7496 bp, synthetic construct
  misc_feature 1..7496: note = Description of Artificial Sequence: Synthetic polynucleotide
  source 1..7496: mol_type = other DNA
ggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattaagcttatactcgagctctagattgggaacccgggtctctcgaattcgaggtctccaccatgcacagacctagacgtcgtggaactcgtccacctccactggcactgctcgctgctctcctcctggctgcacgtggtgctgatgcaaacctggtacccatggtcgctaccgtgggatgcggaggtagtgggggaggtggctccgggggagggggatccatacagcggactccaaagatccaggtgtatagtcgccaccccgcagagaatggaaagtccaacttccttaactgttatgtgagcggatttcacccttcagacatagaggtagaccttctgaaaaacggagagagaatagagaaggtagagcatagtgatctttctttttccaaggactggagcttctacctcctgtactataccgagtttacacccactgagaaggatgagtacgcatgccgcgtgaaccacgtaaccctttctcaaccaaagattgtaaaatgggacagagacatgggtggcggcggtagcggcgggggcggaagcggcggaggaggctccggtgggggtggttcagggtcccattctatgagatatttttttacaagcgtgagccggcctgggaggggggagccccggtttatcgctgtggggtacgtggatgatactcaattcgtgcggttcgactctgacgccgcctcacagcgcatggagcctagggcaccctggattgaacaggagggcccggaatactgggacggcgaaacccgcaaggtaaaagcgcacagccagacgcatagggtggatctgggcaccctgcggggctgttataaccagtcagaggctggctcacacactgtacagcggatgtatggttgtgacgtcggttccgattggagatttcttcgcggctaccaccaatacgcctacgatggcaaggactacatcgcactgaaggaagatttgagatcttggaccgcggcggatatggcagcacagactaccaaacacaagtgggaagctgctcatgtggcagaacagctccgcgcctatctggagggcacttgcgtcgagtggttgcggagatacctggagaatgggaaagaaacactgcagcggaccgatgcacctaaaacacatatgacgcaccacgctgtgtccgaccacgaggcaacactccggtgttgggcactcagcttctaccctgctgaaataacgctgacctggcagcgcgacggcgaagatcagacgcaggacacagagcttgttgaaactcgccctgctggcgacggcaccttccaaaaatgggcagcagtagtcgtcccctccggacaggaacagcggtatacttgccacgtgcagcatgaaggtctccccaagcccctcactttgagatgggagccgtcttctcaacccacgatccctattgttggcattatcgctggtctggtgctttttggagccgtgattactggagcagtggtggcagctgtgatgtggagacgcaagtcttctgataggaagggcggatcttattcccaggctgcgtccagtgacagcgcccaggggtcagatgtcagcctgaccgcatgcaaggtgtgagc

SEQ ID NO: 52 - DNA, 11060 bp, synthetic construct
  misc_feature 1..11060: note = Description of Artificial Sequence: Synthetic polynucleotide
  source 1..11060: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagatctagagccaccatggaaaagaaccccctggccgctcccctgctgatcctgtggtttcacctggactgcgtgtccatcctgaacgtggaacagagcccccagagcctgcatgtgcaggaaggcgacagcaccaacttcacctgtagcttccccagcagcaacttctacgccctgcactggtacagatgggagacagccaagagccccgaggccctgttcgtgatgaccctgaacggcgacgagaagaagaagggccggatcagcgccaccctgaacaccaaagagggctacagctacctgtacatcaagggcagccagcccgaggactccgccacatacctgtgcgccagaaacaccggcaaccagttctacttcggcaccggcaccagcctgaccgtgatccccaacatccagaaccccgaccccgccgtgtaccagctgaaggaccctagaagccaggacagcaccctgtgcctgttcaccgacttcgacagccagatcaacgtgcccaagaccatggaatccggcaccttcatcaccgataagtgcgtgctggacatgaaggccatggacagcaagagcaacggcgccattgcctggtccaaccagaccagcttcacatgccaggacatcttcaaagagacaaacgccacctaccccagctccgacgtgccctgtgatgccacactgaccgagaagtccttcgagacagacatgaacctgaacttccagaacctgagcgtgatgggcctgagaatcctgctgctgaaggtggccggcttcaacctgctgatgacactgcggctgtggtcctctggcagcggcgccacaaatttcagcctgctgaaacaggccggcgacgtggaagagaaccctggccctatggtcatcggcctgctgtgttgtgccgccctgtctctgctgtgggccggacctgtgaatgccggcgtgacccagacccccaagttccaggtgctgaaaaccggccagagcatgaccctgcagtgcgcccaggacatgaatcacgagtacatgagctggtatcggcaggaccccggcatgggcctgcggctgattcactattctgtgggagccggcatcaccgaccagggcgaagtgcccaatggctacaacgtgtccagaagcaccaccgaggacttcccactgagactgctgtctgccgcccctagccagacctccgtgtacttctgcgccagcagctttcagaccggcgccagctacggctacacatttggctctggcacccggctgacagtgctggaagatctgagaaacgtgaccccccccaaggtgtccctgttcgagcctagcaaggccgagatcgccaacaagcagaaagccaccctcgtgtgcctggccagaggcttcttccccgaccacgtggaactgtcttggtgggtcaacggcaaagaggtgcactccggcgtgtgcaccgatccccaggcctacaaagagagcaactactcctactgcctgtccagcagactgcgggtgtccgccaccttctggcacaacccccggaaccacttcaggtgccaggtgcagtttcacggcctgagcgaagaggacaagtggcccgagggcagccctaagcccgtgacacagaatatcagcgccgaagcctggggcagagccgactgtggaatcacctctgccagctaccaccagggcgtgctgagcgccacaatcctgtacgagatcctgctgggcaaggccaccctgtacgccgtgctggtgtctggcctggtgctgatggccatggtcaagaagaaaaacagctgaggatccgcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgtaccaaagatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 53 - protein, 12 aa, synthetic construct
  REGION 1..12: note = Description of Artificial Sequence: Synthetic peptide
QLQPFPQPELPY

SEQ ID NO: 54 - DNA, 36 bp, synthetic construct
  misc_feature 1..36: note = Description of Artificial Sequence: Synthetic oligonucleotide
  source 1..36: mol_type = other DNA
cagctgcagcccttcccccagcccgagctgccctac

SEQ ID NO: 55 - protein, 12 aa, synthetic construct
  REGION 1..12: note = Description of Artificial Sequence: Synthetic peptide
PQPELPYPQPQL

SEQ ID NO: 56 - DNA, 36 bp, synthetic construct
  misc_feature 1..36: note = Description of Artificial Sequence: Synthetic oligonucleotide
  source 1..36: mol_type = other DNA
ccgcaacccgagttgccctacccccagccacaactg

SEQ ID NO: 57 - DNA, 9847 bp, synthetic construct
  misc_feature 1..9847: note = Description of Artificial Sequence: Synthetic polynucleotide
  source 1..9847: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagaattcggatccaccatgaactattctccaggcttagtatctctgatactcttactgcttggaagaacccgtggagattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaggaaaccacttctttccacttggagaaaggctcagttcaagtgtcagactcagcggtgtacttctgtgctctgagtgatcactattcttctggttctgcaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggctgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 58 - DNA, 10202 bp, synthetic construct
  misc_feature 1..10202: note = Description of Artificial Sequence: Synthetic polynucleotide
  source 1..10202: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagaattcggatccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactggggggtccgtctccactctgacgatccagcgcacacagcaggaggactcggccgtgtatctctgtgccagcagcacagcggtcctagcgggagggccgcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgtaccaaagatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 59 - DNA, 9814 bp, synthetic construct
  misc_feature 1..9814: note = Description of Artificial Sequence: Synthetic polynucleotide
  source 1..9814: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcggatccaccatgaactattctccaggcttagtatctctgatactcttactgcttggaagaacccgtggagattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaggaaaccacttctttccacttggagaaaggctcagttcaagtgtcagactcagcggtgtacttctgtgctctgagtgagtctggagccaatagtaagctgacatttggaaaaggaataactctgagtgttagaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggctgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 60 - DNA, 10196 bp, synthetic construct
  misc_feature 1..10196: note = Description of Artificial Sequence: Synthetic polynucleotide
  source 1..10196: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagaattcggatccaccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactggggggtccgtctccactctgacgatccagcgcacacagcaggaggactcggccgtgtatctctgtgccagcagcttacgggcgtgggagacccagtacttcgggccaggcacgcggctcctggtgcttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgtaccaaagatggatagatccggaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 61 - DNA, 1290 bp, synthetic construct
  misc_feature 1..1290: note = Description of Artificial Sequence: Synthetic polynucleotide
  source 1..1290: mol_type = other DNA
atgcctaatccccgacccgccaagcccatggctccttcccttgctctcggaccttcccccggtgtgttgccctcttggaaaaccgcaccaaaagggtcagaactgctggggaccagaggcagcggtggacctttccagggacgagaccttagatctggcgcacacacttcttccagcttgaatccactccccccatcacagcttcaactgccaaccgtgcctctcgtgatggttgctccttcaggtgccagacttggccctagcccccacttgcaggcattgttgcaggaccgccctcacttcatgcaccaactttcaacagtagacgcccacgcacaaaccccagttcttcaggtacgccccctggacaaccccgcaatgattagtctccccccaccctccgctgctacaggcgtgtttagtctcaaagccaggccaggacttccaccaggcatcaatgtcgcttccctggagtgggtctctcgcgagcctgctctgctctgtacattcccacgctccggtacccctcggaaggattctaacctccttgcagccccccagggttcataccccttgttggcaaacggtgtgtgcaaatggcccggttgcgaaaaagtttttgaggagcctgaagaattccttaaacactgtcaggcagatcacttgttggacgaaaagggtaaagctcagtgcctgctccaaagagaagtagtccaaagtcttgaacagcaactcgaactcgaaaaagagaaattgggggcaatgcaagcccaccttgctgggaagatggctctggcaaaggctccatccgtggcatctatggataagtccagctgttgcatcgtggctacaagcacacaagggtccgtcttgcccgcttggtcagcaccccgagaagcacctgatggcgggcttttcgcagtccgaagacacctttgggggtctcacggaaattcctctttcccagagttctttcataacatggactactttaagtatcataacatgagacctccctttacatacgcaacccttatacgatgggcaattctcgaagctcctgagagacagcgcactctcaacgaaatctatcattggttcactcggatgttcgcttacttccgcaaccaccctgccacctggaagaacgcaatacgacataatctctcactgcacaagtgctttgtaagagtggaatcagagaagggagcagtgtggaccgtggacgagtttgagttccgaaaaaaaaggtcccaacgccccaataagtgctcaaacccttgcccctag

SEQ ID NO: 62 - protein, 498 aa, Lymphocytic choriomeningitis mammarenavirus
MGQIVTMFEALPHIIDEVINIVIIVLIIITSIKAVYNFATCGILALVSFLFLAGRSCGMYGLNGPDIYKGVYQFKSVEFDMSHLNLTMPNACSVNNSHHYISMGSSGLEPTFTNDSILNHNFCNLTSALNKKSFDHTLMSIVSSLHLSIRGNSNYKAVSCDFNNGITIQYNLSSSDPQSAMSQCRTFRGRVLDMFRTAFGGKYMRSGWGWTGSDGKTTWCSQTSYQYLIIQNRTWENHCRYAGPFGMSRILFAQEKTKFLTRRLSGTFTWTLSDSSGVENPGGYCLTKWMILAAELKCFGNTAVAKCNVNHDEEFCDMLRLIDYNKAALSKFKQDVESALHVFKTTLNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 63 - DNA, 6707 bp, synthetic construct
  misc_feature 1..6707: note = Description of Artificial Sequence: Synthetic polynucleotide
  source 1..6707: mol_type = other DNA
aagcttatactcgagctctagattgggaacccgggtctctcgaattcatgatcctaaacaaagctctgatgctgggggcccttgccctgaccaccgtgatgagcccctgtggaggtgaagacattgtggctgaccacgtcgcctcttatggtgtaaacttgtaccagtcttacggtccctctggccagtacacccatgaatttgatggagatgagcagttctacgtggacctggggaggaaggagactgtctggtgtttgcctgttctcagacaatttagatttgacccgcaatttgcactgacaaacatcgctgtcctaaaacataacttgaacagtctgattaaatgctccaactctaccgctgctaccaatgaggttcctgaggtcacagtgttttccaagtctcccgtgacactgggtcagcccaacatcctcatctgtcttgtggacaacatctttcctcctgtggtcaacatcacatggctgagcaatgggcactcagtcacagaaggtgtttctgagaccagcttcctctccaagagtgatcattccttcttcaagatcagttacctcaccctcctcccttctgctgaggagagttatgactgcaaggtggagcactggggcctggacaagcctcttctgaaacactgggagcctgagattccagcccctatgtcagagctcacagagactgtggtctgcgccctgggattgtctgtgggcctcgtgggcattgtggtgggcactgtcttcatcatccgaggcctgcgttcagttggtgcttccagacaccaagggcccttgtgagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 64 - DNA, 6809 bp, synthetic construct
  misc_feature 1..6809: note = Description of Artificial Sequence: Synthetic polynucleotide
  source 1..6809: mol_type = other DNA
aagcttatactcgagctctagattgggaacccgggtctctcgaattcatgtcttggaaaaaggctttgcgaatccccggaggccttcgggcagcaactgtgaccttgatgctgtcgatgctgagcaccccagtggctgagggccagctgcagcccttcccccagcccgagctgccctacggatgtggtggaagtggaggtggtggatctggtggaggtggatcaagagactctcccgaggatttcgtgtaccagtttaagggcatgtgctacttcaccaacgggacagagcgcgtgcgtcttgtgagcagaagcatctataaccgagaagagatcgtgcgcttcgacagcgacgtgggggagttccgggcggtgacgctgctggggctgcctgccgccgagtactggaacagccagaaggacatcctggagaggaaacgggcggcggtggacagggtgtgcagacacaactaccagttggagctccgcacgaccttgcagcggcgagtggagcccacagtgaccatctccccatccaggacagaggccctcaaccaccacaacctgctggtctgctcggtgacagatttctatccagcccagatcaaagtccggtggtttcggaatgaccaggaggagacagctggcgttgtgtccaccccccttattaggaatggtgactggaccttccagatcctggtgatgctggaaatgactccccagcgtggagacgtctacacctgccacgtggagcaccccagcctccagagccccatcaccgtggagtggcgggctcaatctgaatctgcccagagcaagatgctgagtggcattggaggcttcgtgctggggctgatcttcctcgggctgggccttatcatccatcacaggagtcagaaagggctcctgcactgagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 65 - DNA, 6809 bp, synthetic construct
  misc_feature 1..6809: note = Description of Artificial Sequence: Synthetic polynucleotide
  source 1..6809: mol_type = other DNA
aagcttatactcgagctctagattgggaacccgggtctctcgaattcatgtcttggaaaaaggctttgcgaatccccggaggccttcgggcagcaactgtgaccttgatgctgtcgatgctgagcaccccagtggctgagggcccgcaacccgagttgccctacccccagccacaactgggatgtggtggaagtggaggtggtggatctggtggaggtggatcaagagactctcccgaggatttcgtgtaccagtttaagggcatgtgctacttcaccaacgggacagagcgcgtgcgtcttgtgagcagaagcatctataaccgagaagagatcgtgcgcttcgacagcgacgtgggggagttccgggcggtgacgctgctggggctgcctgccgccgagtactggaacagccagaaggacatcctggagaggaaacgggcggcggtggacagggtgtgcagacacaactaccagttggagctccgcacgaccttgcagcggcgagtggagcccacagtgaccatctccccatccaggacagaggccctcaaccaccacaacctgctggtctgctcggtgacagatttctatccagcccagatcaaagtccggtggtttcggaatgaccaggaggagacagctggcgttgtgtccaccccccttattaggaatggtgactggaccttccagatcctggtgatgctggaaatgactccccagcgtggagacgtctacacctgccacgtggagcaccccagcctccagagccccatcaccgtggagtggcgggctcaatctgaatctgcccagagcaagatgctgagtggcattggaggcttcgtgctggggctgatcttcctcgggctgggccttatcatccatcacaggagtcagaaagggctcctgcactgagcggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaatt

SEQ ID NO: 66 - DNA, 8502 bp, synthetic construct
  misc_feature 1..8502: note = Description of Artificial Sequence: Synthetic polynucleotide
  source 1..8502: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcggatccggagctactaacttcagcctgctgaagcaggctggagacgtggaggagaaccctggacccatgggagcaggggcaaccggtagggcgatggatggtccgcggcttttgcttcttttgttgctgggggtgagcttgggcggcgctaaagaagcatgtcccaccggactgtacacgcatagtggcgagtgttgtaaagcatgtaaccttggggagggcgttgctcagccatgcggggccaatcaaacagtttgcgagccttgtttggactcagtcacgttctcagatgttgtatctgccaccgaaccatgtaaaccctgcacggaatgtgtaggtcttcaaagcatgtctgcaccttgtgtcgaagcggacgacgctgtttgtcgatgtgcttatggttactatcaggacgaaacgaccgggcggtgtgaagcctgtcgggtatgcgaagccggctctggtcttgtgttttcctgccaagacaaacagaacactgtctgcgaggaatgcccagatggtacctatagcgatgaggccaaccatgttgatccctgcctcccctgtactgtttgcgaagataccgaaagacagcttagagaatgtacccgatgggccgatgccgagtgtgaagagattcccggtcgctggataacgcgcagcacccccccagaggggagtgattcaacagcgccaagcacccaggaaccggaagctcccccggagcaagaccttattgcgtcaacggttgcgggagttgtgaccactgttatggggtctagccaacctgtagtgaccaggggaacgaccgataatctcataccggtatattgctcaatacttgctgccgtggtcgttggcctggtggcgtacattgcctttaagagatagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 67 - DNA, 9031 bp, synthetic construct
  misc_feature 1..9031: note = Description of Artificial Sequence: Synthetic polynucleotide
  source 1..9031: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagtaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagatgagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatgggagcaggggcaaccggtagggcgatggatggtccgcggcttttgcttcttttgttgctgggggtgagcttgggcggcgctaaagaagcatgtcccaccggactgtacacgcatagtggcgagtgttgtaaagcatgtaaccttggggagggcgttgctcagccatgcggggccaatcaaacagtttgcgagccttgtttggactcagtcacgttctcagatgttgtatctgccaccgaaccatgtaaaccctgcacggaatgtgtaggtcttcaaagcatgtctgcaccttgtgtcgaagcggacgacgctgtttgtcgatgtgcttatggttactatcaggacgaaacgaccgggcggtgtgaagcctgtcgggtatgcgaagccggctctggtcttgtgttttcctgccaagacaaacagaacactgtctgcgaggaatgcccagatggtacctatagcgatgaggccaaccatgttgatccctgcctcccctgtactgtttgcgaagataccgaaagacagcttagagaatgtacccgatgggccgatgccgagtgtgaagagattcccggtcgctggataacgcgcagcacccccccagaggggagtgattcaacagcgccaagcacccaggaaccggaagctcccccggagcaagaccttattgcgtcaacggttgcgggagttgtgaccactgttatggggtctagccaacctgtagtgaccaggggaacgaccgataatctcataccggtatattgctcaatacttgctgccgtggtcgttggcctggtggcgtacattgcctttaagagatagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 68 - DNA, 7583 bp, synthetic construct
  misc_feature 1..7583: note = Description of Artificial Sequence: Synthetic polynucleotide
  source 1..7583: mol_type = other DNA
ggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattaagcttatactcgagctctagattgggaacccgggtctctcgaattcgagatctccaccatgcacagacctagacgtcgtggaactcgtccacctccactggcactgctcgctgctctcctcctggctgcacgtggtgctgatgcagaggtgcagctgcagcaggacggcgccgagctgggcaagcccggcaccagcgtgaagctgagctgcaaggtgagcgactacaacatcagaagaacctacatgcactgggtgaaccagagacccggcaagggcctggagtggatcggcagaatcgaccccgccaacggcaacaccatctacggcgagaagttcaagagcaaggccaccctgaccgccgacaccagcagcaacaccgcctacatgcagctgagccagctgaagagcgacgacaccgccatctacttctgcgccatcggcgtgcagtacctggactactggggccagggcgtgatggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaagagctgcaactggaggagagctgtgcggaggcgcaggacggggagctggacgggctgtggacgaccatcaccatcttcatcacactcttcctgctaagcgtgtgctacagtgccaccgtcaccttcttcaaggtgaagtggatcttctcctcagtggtggacctgaagcagaccatcgtccccgactacaggaacatgataaggcagggggcctgagc

SEQ ID NO: 69 - DNA, 6683 bp, synthetic construct
  misc_feature 1..6683: note = Description of Artificial Sequence: Synthetic polynucleotide
  source 1..6683: mol_type = other DNA
ggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctaccggtagggcccctctcttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggcgcgcctcatactcctgcaggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaggtaccaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccggatccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatggatctatagatctctcgtgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagtcgcgaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattaagcttatactcgagctctagattgggaacccgggtctctcgaattcgagatctccaccatgcacagacctagacgtcgtggaactcgtccacctccactggcactgctcgctgctctcctcctggctgcacgtggtgctgatgcagacaccgtgctgacccagagccccgccctggccgtgagccccggcgagagagtgaccatcagctgcagagccaccgagagcgtgagcaccctgatccactggttccagcagagacccggccagcagcccaagctgctgatctacctgaccagccacctggacagcggcgtgcccgccagattcagcggcagcggcagcggcaccgacttcaccctgaccatcgaccccgtggaggccgacgacaccgccacctactactgccagcagacctggaacgacccctggaccttcggcggcggcaccaagctggagctgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagc

SEQ ID NO: 70 - protein, 5 aa, synthetic construct
  REGION 1..5: note = Description of Artificial Sequence: Synthetic peptide
GGGGS

SEQ ID NO: 71 - protein, 4 aa, Sindbis virus
SLKQ

SEQ ID NO: 72 - protein, 4 aa, synthetic construct
  REGION 1..4: note = Description of Artificial Sequence: Synthetic peptide
AAAA

SEQ ID NO: 73 - protein, 5 aa, synthetic construct
  REGION 1..5: note = Description of Artificial Sequence: Synthetic peptide
GSGGS

SEQ ID NO: 74 - protein, 4 aa, synthetic construct
  REGION 1..4: note = Description of Artificial Sequence: Synthetic peptide
GGGS

SEQ ID NO: 75 - protein, 4 aa, synthetic construct
  REGION 1..4: note = Description of Artificial Sequence: Synthetic peptide
GGSG

SEQ ID NO: 76 - protein, 5 aa, synthetic construct
  REGION 1..5: note = Description of Artificial Sequence: Synthetic peptide
GGSGG

SEQ ID NO: 77 - protein, 5 aa, synthetic construct
  REGION 1..5: note = Description of Artificial Sequence: Synthetic peptide
GSGSG

SEQ ID NO: 78 - protein, 5 aa, synthetic construct
  REGION 1..5: note = Description of Artificial Sequence: Synthetic peptide
GSGGG

SEQ ID NO: 79 - protein, 5 aa, synthetic construct
  REGION 1..5: note = Description of Artificial Sequence: Synthetic peptide
GGGSG

SEQ ID NO: 80 - protein, 5 aa, synthetic construct
  REGION 1..5: note = Description of Artificial Sequence: Synthetic peptide
GSSSG

SEQ ID NO: 81 - protein, 15 aa, synthetic construct
  REGION 1..15: note = Description of Artificial Sequence: Synthetic peptide
GCGASGGGGSGGGGS

SEQ ID NO: 82 - protein, 10 aa, synthetic construct
  REGION 1..10: note = Description of Artificial Sequence: Synthetic peptide
GGGGSGGGGS

SEQ ID NO: 83 - protein, 15 aa, synthetic construct
  REGION 1..15: note = Description of Artificial Sequence: Synthetic peptide
GGGASGGGGSGGGGS

SEQ ID NO: 84 - protein, 15 aa, synthetic construct
  REGION 1..15: note = Description of Artificial Sequence: Synthetic peptide
GGGGSGGGGSGGGGS

SEQ ID NO: 85 - protein, 10 aa, synthetic construct
  REGION 1..10: note = Description of Artificial Sequence: Synthetic peptide
GGGASGGGGS

SEQ ID NO: 86 - protein, 20 aa, synthetic construct
  REGION 1..20: note = Description of Artificial Sequence: Synthetic peptide
GGGGSGGGGSGGGGSGGGGS

SEQ ID NO: 87 - protein, 6 aa, synthetic construct
  REGION 1..6: note = Description of Artificial Sequence: Synthetic 6xHis tag
HHHHHH

SEQ ID NO: 88 - protein, 8 aa, Human immunodeficiency virus 1
KARVLAEA

SEQ ID NO: 89 - DNA, 12 bp, synthetic construct
  misc_feature 1..12: note = Description of Artificial Sequence: Synthetic oligonucleotide
  source 1..12: mol_type = other DNA
gggggtggttcc

SEQ ID NO: 90 - DNA, 12 bp, synthetic construct
  misc_feature 1..12: note = Description of Artificial Sequence: Synthetic oligonucleotide
  source 1..12: mol_type = other DNA
ggtgggtctggg

SEQ ID NO: 91 - DNA, 15 bp, synthetic construct
  misc_feature 1..15: note = Description of Artificial Sequence: Synthetic oligonucleotide
  source 1..15: mol_type = other DNA
gggtccgggggctcc

SEQ ID NO: 92 - DNA, 15 bp, synthetic construct
  misc_feature 1..15: note = Description of Artificial Sequence: Synthetic oligonucleotide
  source 1..15: mol_type = other DNA
ggtgggagcggtggt

SEQ ID NO: 93 - DNA, 15 bp, synthetic construct
  misc_feature 1..15: note = Description of Artificial Sequence: Synthetic oligonucleotide
  source 1..15: mol_type = other DNA
ggcagcggaagcgga

SEQ ID NO: 94 - DNA, 15 bp, synthetic construct
  misc_feature 1..15: note = Description of Artificial Sequence: Synthetic oligonucleotide
  source 1..15: mol_type = other DNA
gggagtgggggaggt

SEQ ID NO: 95 - DNA, 15 bp, synthetic construct
  misc_feature 1..15: note = Description of Artificial Sequence: Synthetic oligonucleotide
  source 1..15: mol_type = other DNA
ggtgggggaagtgga

SEQ ID NO: 96 - DNA, 15 bp, synthetic construct
  misc_feature 1..15: note = Description of Artificial Sequence: Synthetic oligonucleotide
  source 1..15: mol_type = other DNA
ggcagctcatctggt

SEQ ID NO: 97 - DNA, 45 bp, synthetic construct
  misc_feature 1..45: note = Description of Artificial Sequence: Synthetic oligonucleotide
  source 1..45: mol_type = other DNA
ggatgtggtgcatctggagggggaggctctggggggggtggatct

SEQ ID NO: 98 - DNA, 45 bp, synthetic construct
  misc_feature 1..45: note = Description of Artificial Sequence: Synthetic oligonucleotide
  source 1..45: mol_type = other DNA
gggtgtggtgctagtgggggtggcggatcaggtggaggcgggagc

SEQ ID NO: 99 - DNA, 30 bp, synthetic construct
  misc_feature 1..30: note = Description of Artificial Sequence: Synthetic oligonucleotide
  source 1..30: mol_type = other DNA
gggggcggaggatctgggggagggggatca

SEQ ID NO: 100 - DNA, 42 bp, synthetic construct
  misc_feature 1..42: note = Description of Artificial Sequence: Synthetic oligonucleotide
  source 1..42: mol_type = other DNA
ggggggggcgcttcaggcggaggtggaagtggtggaggaggt

SEQ ID NO: 101 - DNA, 45 bp, synthetic construct
  misc_feature 1..45: note = Description of Artificial Sequence: Synthetic oligonucleotide
  source 1..45: mol_type = other DNA
ggagggggaggttctggcggcgggggatcaggaggcggtgggagc

SEQ ID NO: 102 - DNA, 30 bp, synthetic construct
  misc_feature 1..30: note = Description of Artificial Sequence: Synthetic oligonucleotide
  source 1..30: mol_type = other DNA
ggtgggggggcgtcaggtggaggcggaagt

SEQ ID NO: 103 - DNA, 45 bp, synthetic construct
  misc_feature 1..45: note = Description of Artificial Sequence: Synthetic oligonucleotide
  source 1..45: mol_type = other DNA
ggcggcggaggttctggtgggggtggcagtggaggaggaggcagc

SEQ ID NO: 104 - DNA, 60 bp, synthetic construct
  misc_feature 1..60: note = Description of Artificial Sequence: Synthetic oligonucleotide
  source 1..60: mol_type = other DNA
ggaggtggaggtagtggcggtggtgggtcagggggaggcgggtccggtggcggtgggagt

SEQ ID NO: 105 - DNA, 12 bp, synthetic construct
  misc_feature 1..12: note = Description of Artificial Sequence: Synthetic oligonucleotide
  source 1..12: mol_type = other DNA
ggtggcggtagt

SEQ ID NO: 106 - DNA, 12 bp, synthetic construct
  misc_feature 1..12: note = Description of Artificial Sequence: Synthetic oligonucleotide
  source 1..12: mol_type = other DNA
gggggatctggt

SEQ ID NO: 107 - DNA, 15 bp, synthetic construct
  misc_feature 1..15: note = Description of Artificial Sequence: Synthetic oligonucleotide
  source 1..15: mol_type = other DNA
ggcagtggcggtagc

SEQ ID NO: 108 - DNA, 15 bp, synthetic construct
  misc_feature 1..15: note = Description of Artificial Sequence: Synthetic oligonucleotide
  source 1..15: mol_type = other DNA
ggagggagtggaggg

SEQ ID NO: 109 - DNA, 15 bp, synthetic construct
  misc_feature 1..15: note = Description of Artificial Sequence: Synthetic oligonucleotide
  source 1..15: mol_type = other DNA
gggtctggctcaggc

SEQ ID NO: 110 - DNA, 15 bp, synthetic construct
  misc_feature 1..15: note = Description of Artificial Sequence: Synthetic oligonucleotide
  source 1..15: mol_type = other DNA
ggttctggcggaggt

SEQ ID NO: 111 - DNA, 15 bp, synthetic construct
  misc_feature 1..15: note = Description of Artificial Sequence: Synthetic oligonucleotide
  source 1..15: mol_type = other DNA
ggtggtgggagtgga

SEQ ID NO: 112 - DNA, 15 bp, synthetic construct
  misc_feature 1..15: note = Description of Artificial Sequence: Synthetic oligonucleotide
  source 1..15: mol_type = other DNA
ggctcaagcagtgga

SEQ ID NO: 113 - DNA, 45 bp, synthetic construct
  misc_feature 1..45: note = Description of Artificial Sequence: Synthetic oligonucleotide
  source 1..45: mol_type = other DNA
ggctgtggggctagtgggggaggtggtagtggtggtggcggttcc

SEQ ID NO: 114 - DNA, 45 bp, synthetic construct
  misc_feature 1..45: note = Description of Artificial Sequence: Synthetic oligonucleotide
  source 1..45: mol_type = other DNA
ggatgtggggcctcaggtgggggtggcagcggtggtggagggtca

SEQ ID NO: 115 - DNA, 30 bp, synthetic construct
  misc_feature 1..30: note = Description of Artificial Sequence: Synthetic oligonucleotide
  source 1..30: mol_type = other DNA
ggtggcgggggctctggtggaggaggatct

SEQ ID NO: 116 - DNA, 42 bp, synthetic construct
  misc_feature 1..42: note = Description of Artificial Sequence: Synthetic oligonucleotide
  source 1..42: mol_type = other DNA
ggaggcggcgcttctgggggcgggggtagtgggggtggaggt

SEQ ID NO: 117 - DNA, 45 bp, synthetic construct
  misc_feature 1..45: note = Description of Artificial Sequence: Synthetic oligonucleotide
  source 1..45: mol_type = other DNA
ggtggaggtggaagtggaggagggggatcaggcggaggcgggagc

SEQ ID NO: 118 - DNA, 30 bp, synthetic construct
  misc_feature 1..30: note = Description of Artificial Sequence: Synthetic oligonucleotide
  source 1..30: mol_type = other DNA
ggagggggagcctctggcggtggaggatca

SEQ ID NO: 119 - DNA, 45 bp, synthetic construct
  misc_feature 1..45: note = Description of Artificial Sequence: Synthetic oligonucleotide
  source 1..45: mol_type = other DNA
gggggaggaggcagtggaggtgggggaagtggtggaggggggtct

SEQ ID NO: 120 - DNA, 60 bp, synthetic construct
  misc_feature 1..60: note = Description of Artificial Sequence: Synthetic oligonucleotide
  source 1..60: mol_type = other DNA
gggggtggaggatcaggaggcggtggttctgggggaggtggatccggcgggggtggtagt

SEQ ID NO: 121 - protein, 11 aa, Cytomegalovirus sp.
CAVGYGNQFYF

SEQ ID NO: 122 - protein, 11 aa, Cytomegalovirus sp.
CILDNNNDMRF

SEQ ID NO: 123 - DNA, 15 bp, synthetic construct
  source 1..15: mol_type = other DNA
tcccatataagaaac

(19) United States
(12) Patent Application Publication
(10) Pub. No.: US 2026/0226500 A1
(43) Pub. Date: Aug. 6, 2026

(54) PSEUDOTYPED VIRAL PARTICLES FOR TARGETING TCR-EXPRESSING CELLS

(71) Applicant: Regeneron Pharmaceuticals, Inc., Tarrytown, NY (US)

(72) Inventors: Christos KYRATSOUS, Irvington, NY (US); Andrew J. MURPHY, Croton-on-Hudson, NY (US); Mathieu DESCLAUX, Brooklyn, NY (US); Augustine CHOY, Dobbs Ferry, NY (US)

(73) Assignee: Regeneron Pharmaceuticals, Inc., Tarrytown, NY (US)

(21) Appl. No.: 18/871,786

(22) PCT Filed: Jun. 7, 2023
(86) PCT No.: PCT/US2023/068048

§ 371 Date: Dec. 4, 2024

Related U.S. Application Data
(60) Provisional application No. 63/349,795, filed on Jun. 7, 2022.
(60) Provisional application No. 63/470,286, filed on Jun. 1, 2023.

(51) Int. Cl. C12N 15/86; A61K 39/00; A61K 39/12; A61P 35/00; A61P 37/04; C07K 14/005; C07K 14/705; C07K 16/28; C12N 15/10

(52) U.S. Cl. CPC C12N 15/86; A61K 39/12; A61P 35/00; A61P 37/04; C07K 14/005; C07K 14/70539; C07K 16/2809; C12N 15/1086; A61K 2039/5256; C12N 2740/13043; C12N 2740/13052; C12N 2740/15043; C12N 2740/15052; C12N 2760/10022; C12N 2760/10034

(57) ABSTRACT

Described herein are pseudotyped viral particles for specific targeting of TCR-expressing cells and the use of such particles for treatment of various diseases.

### CROSS-REFERENCE TO RELATED APPLICATIONS

This application claims priority to U.S. Provisional Application No. 63/349,795, filed Jun. 7, 2022, and U.S. Provisional Application No. 63/470,286, filed Jun. 1, 2023, the disclosure of each of which is herein incorporated by reference in its entirety.

### SEQUENCE LISTING

The instant application contains a Sequence Listing which has been submitted electronically in XML file format and is hereby incorporated by reference in its entirety. Said XML copy, created on Jun. 6, 2023, is named 250298_000493_SL.xml and is 507,278 bytes in size.

### TECHNICAL FIELD

Described herein are pseudotyped viral particles that target a T cell receptor (TCR), e.g., a specific TCR, and the use of such particles for the genetic modification of TCR-expressing cells, e.g., in the treatment of various diseases.

### BACKGROUND

Lymphocytes, such as T cells, play important roles in adaptive anti-infection, antitumor, autoimmune, and transplant rejection responses. Generally, a T cell mediated immune response involves close contact, e.g., an immunological synapse, between a T cell and an antigen presenting cell (APC).

T cell receptors are heterodimeric structures composed of two types of chains (an α (alpha) and β (beta) chain, or a γ (gamma) and δ (delta) chain). The a chain is encoded by the nucleic acid sequence located within the α locus (on human or mouse chromosome 14), which also encompasses the entire δ locus that encodes the δ chain, and the β chain is encoded by the nucleic acid sequence located within the β locus (on mouse chromosome 6 or human chromosome 7). The majority of T cells have an αβ TCR; while a minority of T cells bear a γδ TCR. T cell receptor a and β polypeptides (and similarly γ and δ polypeptides) are linked to each other via a disulfide bond. Each of the two polypeptides that make up the TCR contains an extracellular domain comprising constant and variable regions, a transmembrane domain, and a cytoplasmic tail (the transmembrane domain and the cytoplasmic tail also being a part of the constant region).

The variable region of each TCR comprises a unique and characteristic structure, i.e., an idiotope or idiotype, that determines the specificity of the TCR. Generally, a TCR will bind to a pMHC complex only if it comprises an idiotype that recognizes the particular peptide being presented in the context of MHC, e.g., the unique conformation of a particular pMHC complex.

Immunotherapeutic approaches to treating diseases work to regulate T cell activity in vivo, e.g., to enhance anti-infection and antitumor responses, or downregulate autoimmune and transplant rejection responses, etc. However, such methods typically lack specificity since many immunotherapies target signaling by the TCR complex by binding CD3 and/or the pairing of costimulatory molecules, etc. Such approaches often result in undesirable side effects, e.g., a hyperactive immune response or generalized immune suppression. Accordingly, therapies that take advantage of the uniquely specific interaction between a TCR and pMHC complex may provide the ability to specifically modulate the activity of specific T cells in vivo, and provide new treatments based on T cell modulation.

### SUMMARY

The disclosure herein addresses this need by providing a recombinant viral particle pseudotyped with T cell receptor (TCR)-binding molecule, which may specifically bind a TCR in accordance with the specificity of the TCR, e.g., the TCR-binding molecule binds a TCR idiotype, e.g., the TCR-binding molecule specifically binds an antigen-specific TCR. The viral particles described herein are capable of selectively targeting and modulating the activity of cell subpopulations carrying antigen-specific TCRs making said viral particles useful for treatment of various inflammatory, autoimmune, and infectious disorders as well as cancers.

In one aspect, described herein is a recombinant viral particle that is capable of binding to a TCR, the recombinant viral particle comprising on its surface (e.g., in a lipid envelope or capsid) (i) a TCR-binding molecule, and optionally (ii) a fusogen. In some embodiments, the TCR-binding molecule specifically binds a TCR idiotype, e.g., the TCR-binding molecule binds a TCR in accordance with the specificity of the TCR. In some embodiments, the TCR-binding molecule that binds a TCR in accordance with the specificity of the TCR (e.g., specifically binds a TCR idiotype) comprises one or more polypeptides, e.g., an anti-TCR antibody (or portion thereof), a peptide-major histocompatibility complex (pMHC), etc.

In some embodiments, the TCR-binding molecule comprises an anti-TCR antibody (e.g., a non-human anti-TCR antibody, a human anti-TCR antibody, etc.) or portion thereof. In some embodiments, the TCR-binding molecule comprises an anti-TCR antibody that specifically binds an antigen-specific TCR, e.g., a TCR idiotype. In some embodiments, the TCR-binding molecule comprises an anti-TCR antibody comprising one or more variable domains that specifically bind a TCR idiotype. In some embodiments, the TCR-binding molecule comprises a single-chain-Fv (scFv). In some embodiments, the scFv specifically binds an antigen-specific TCR, e.g., a TCR idiotype.

In another embodiment, the TCR-binding molecule comprises a pMHC complex, i.e., a peptide (p) presented in the context of a major histocompatibility complex (MHC) molecule, e.g., a peptide (p) associated with(in) a peptide binding groove of an MHC molecule. In some embodiments, the TCR-binding molecule comprises a pMHC complex that specifically binds (i.e., is specifically recognized by) an antigen-specific TCR, e.g., a TCR idiotype.

In some embodiments, described herein is a recombinant viral particle that is capable of binding to an antigen-specific TCR, the recombinant viral particle comprising on its surface (e.g., in a lipid envelope or capsid) (i) a peptide (p) presented in the context of a major histocompatibility complex (MHC) molecule, i.e., a pMHC complex, and optionally (ii) a fusogen, wherein the pMHC complex comprises the peptide associated with(in) the peptide binding groove of a class I or class II MHC, and wherein the pMHC complex binds the antigen-specific TCR. In some embodiments, the pMHC complex specifically binds the antigen-specific TCR. In some embodiments, the pMHC complex is the antigen to which the targeted TCR is specific.

In some embodiments, the recombinant viral particle described herein is derived from an enveloped virus. In some embodiments, the recombinant viral particle described herein is derived from a non-enveloped virus.

In some embodiments, the pMHC complex and/or the fusogen comprises a transmembrane domain embedded in the lipid envelope of an enveloped virus.

In some embodiments, the TCR-binding molecule, e.g., an anti-TCR antibody (or portion thereof) or a peptide-major histocompatibility complex (pMHC), is expressed on the outer surface of a virus capsid of a non-enveloped virus. The TCR-binding molecule may be attached to the outer surface of the virus capsid by a linker, such as, e.g., a SpyTag:SpyCatcher system, SpyTag002:SpyCatcher002 system, SpyTag:KTag system, isopeptag:pilin C system, or SnoopTag:SnoopCatcher system.

In some embodiments, the tropism of a viral particle described herein is determined, in part or in whole, by the TCR-binding molecule, e.g., pMHC complex. In some embodiments, the MHC-binding molecule displayed by a viral particle described herein binds to, e.g., in a specific manner, an antigen-specific TCR. In some embodiments, a viral particle is capable of binding a TCR, e.g., an antigen-specific TCR, or a cell expressing the TCR, only when a TCR-binding molecule, e.g., a pMHC complex, is displayed on the surface of the viral particle.

In some embodiments, a viral particle as described herein comprises at least one, e.g., a plurality of, capsomer(s), e.g., a capsid. In some embodiments, the capsomer determines the reference wild-type virus from which a viral particle as described herein is derived, e.g., in some embodiments, a viral particle as described herein comprises at least one capsomer, e.g., a plurality of capsomers, e.g., a capsid, of a reference wild-type virus. In some embodiments, a viral particle as described herein comprises an adeno-associated virus (AAV) capsid, and thus, may be considered an AAV particle and/or derived from a wild-type AAV. In some embodiments, a viral particle as described herein comprises a retroviral capsomer, e.g., capsomer, and thus, may be considered a retroviral particle and/or derived from a wild-type retrovirus. In some embodiments, a viral particle as described herein comprises a lentiviral capsomer and/or capsid, and thus, may be considered a lentiviral particle and/or derived from a wild-type lentivirus. In some embodiments, one or more of proteins typically present in the lipid envelope of a reference wild-type enveloped virus, from which a recombinant viral particle described herein is derived, are absent or mutated from the recombinant viral particle. For example, in some embodiments, a viral particle described herein (a) lacks one or more of the proteins typically present in the lipid envelope of its reference wild-type virus and/or (b) comprises a protein present in the lipid envelope of its reference wild-type virus, wherein the protein is mutated. In some embodiments, a viral particle described herein is not capable of binding to any of the cells targeted by its reference wild-type virus in the absence of the TCR-binding molecule. In some embodiments, a viral particle described herein lacks any, e.g., all, of the proteins which are typically present in the lipid envelope of its reference wild-type virus. In this specific embodiment, a viral particle described herein comprises none of the proteins normally present in the lipid envelope of its reference wild-type virus.

In some embodiments, the viral particle described herein is derived from a virus of the family Retroviridae. In some embodiments, the viral particle described herein is a retroviral particle. In some embodiments, the viral particle described herein is a lentiviral particle. In some embodiments, the lentiviral particle does not contain gp120 surface envelope protein and/or gp41 transmembrane envelope protein. In some embodiments, the lentiviral particle contains a mutant gp120 surface envelope protein and/or a mutant gp41 transmembrane envelope protein such that the lentiviral particle is not capable of binding to a cell, e.g., T cell, in the absence of the TCR-binding molecule. In some embodiments, the lentiviral particle contains a wild-type gp120 surface envelope protein and/or a wild-type gp41 transmembrane envelope protein.

In some embodiments, the viral particle described herein is derived from a virus of the family Parvoviridae. In some embodiments, the viral particle described herein is an adeno-associated virus (AAV), e.g., an AAV serotype selected from the group consisting of AAV1, AAV2, AAV3, AAV4, AAV5, AAV6, AAV7, AAV8 and AAV9, a hybrid of some of those serotypes (e.g., AAV-DJ), or a genetically modified variant thereof.

In some embodiments, the viral particle is replication deficient.

In some embodiments, the viral particle further comprises a cytotoxic agent. In one specific embodiment, the cytotoxic agent is a toxin, a radioactive isotope (e.g., a radioconjugate) or a suicide gene. Non-limiting examples of toxins include, e.g., enzymatically active toxins of bacterial, fungal, plant, or animal origin, or fragments, mutants or derivatives thereof (e.g., diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes). Non-limiting examples of suicide genes include, e.g., thymidine kinase, cytosine deaminase, purine nucleoside phosphorylase, nitroreductase, β-galactosidase, hepatic cytochrome P450-2B1, linamarase, horseradish peroxidase, and carboxypeptidase.

In some embodiments, the viral particle described herein binds, e.g., in a specific manner, an antigen-specific TCR on a surface of a cell, e.g., a T cell. In some embodiments, a viral particle is capable of binding a TCR-expressing cell, e.g., a T cell, only when a TCR-binding molecule, e.g., a pMHC complex, is displayed on its surface.

In some embodiments, the viral particle described herein binds, e.g., in a specific manner, an antigen-specific TCR on a surface of a cell, e.g., a T cell, and infects the cell, e.g., T cell, with a nucleotide of interest. In some embodiments, a viral particle is capable of infecting a TCR-expressing cell, e.g., a T cell, only when a TCR-binding molecule, e.g., a pMHC complex, is displayed on its surface. In some embodiments, the viral particle is capable of binding and infecting a TCR-expressing cell, e.g., a T cell, only when a TCR-binding molecule, e.g., a pMHC complex, is displayed on its surface.

Accordingly, in some embodiments, the viral particle comprises a nucleotide sequence of interest, e.g., a nucleotide sequence encoding a gene desired to be expressed in a TCR-expressing cell targeted by the viral particle, which nucleotide sequence of interest may be DNA or RNA. In some embodiments, a viral particle described herein comprises one or more transfer vectors comprising the nucleotide sequence of interest. In some embodiments, the nucleotide sequence of interest is an RNA molecule transcribed by a transfer vector. In some embodiments a viral particle described herein comprises RNA comprising the nucleotide sequence of interest. In some embodiments, a viral particle as described herein comprises one or more transfer vectors, or one or more RNA molecule encoded by the transfer vector, wherein said transfer vector or RNA molecule comprises the nucleotide sequence of interest, and optionally, further comprises a viral element. In one specific embodiment, upon infection of the TCR-expressing cell with the viral particle, the nucleotide sequence of interest becomes integrated into the cell genome. In some embodiments, the at least one viral element is a retroviral element. In another specific embodiment, the at least one viral element is a lentiviral element. In one specific embodiment, the at least one viral element is a Psi (ψ) packaging signal. In one specific embodiment, in addition to a Psi (ψ) packaging signal, the viral element further comprises a 5′ Long Terminal Repeat (LTR) and/or a 3′ LTR, or a derivative or mutant thereof. In one specific embodiment, the at least one viral element is selected from the group consisting of a 5′ Long Terminal Repeat (LTR), a Psi (ψ) packaging signal, a Rev Response Element (RRE), a promoter that drives expression of the nucleotide sequence of interest, a Central Polypurine Tract (cPPT), a Woodchuck hepatitis virus post-transcriptional regulatory element (WPRE), a Unique 3′ (U3), a Repeat (R) region, a Unique 5′ (U5), a 3′ LTR, a 3′LTR with the U3 element deleted (e.g., to make the lentivirus non-replicative), a Trans-activating response element (TAR), and any combination thereof.

In some embodiments, a viral particle described herein is designed such that a nucleotide of interest is integrated into a genome of a target TCR-expressing cell upon infection of the TCR-expressing cell by the viral particle. Such integration may be mediated by viral enzymes carried by the viral particle. In some embodiments, a viral particle as described herein further comprises an enzyme, e.g., an integrase, or a nucleic acid encoding same, such that a nucleotide of interest is integrated into a genome of a target TCR-expressing cell upon infection of the TCR-expressing cell by the viral particle.

In some embodiments, a viral particle described herein is designed such that a nucleotide of interest remains episomal to a genome of a target TCR-expressing cell upon infection of the TCR-expressing cell by the viral particle. In some embodiments, a viral particle described herein lacks an enzyme that mediates integration of a nucleotide of interest, e.g., an integrase, or a nucleic acid encoding same.

In some embodiments, the viral particle described herein comprises components, e.g., capsomers, glycoproteins, etc., from a virus selected from the group consisting of Human Immunodeficiency Virus (HIV), Bovine Immunodeficiency Virus (BIV), Feline Immunodeficiency Virus (FIV), Simian Immunodeficiency Virus (SIV), Equine Infectious Anemia Virus (EIAV), Murine Stem Cell Virus (MSCV), or Murine Leukemia Virus (MLV). In some embodiments, a viral particle described herein comprises an HIV capsomer, a plurality of HIV capsomers, and/or an HIV capsid, e.g., is a HIV viral particle and/or is derived from HIV.

In some embodiments, a viral particle as described herein displays, in addition to a TCR-binding molecule, a fusogen. In some embodiments, the fusogen is a protein; e.g., a viral protein (e.g., a vesiculovirus protein [e.g., vesicular stomatitis virus G glycoprotein (VSVG)], an alphavirus protein [e.g., a Sindbis virus glycoprotein], an orthomyxovirus protein [e.g., an influenza HA protein], a paramyxovirus protein [e.g., a Nipah virus F protein or a measles virus F protein]), a retrovirus protein, a lentivirus protein, or a fragment, mutant or derivative thereof. In one specific embodiment, the fusogen is heterologous to the reference wild-type virus from which the particle is derived. In some embodiments, the fusogen is a mutated protein which does not bind its natural ligand. In some embodiments, the fusogen comprises a sequence set forth as SEQ ID NO: 5.

In some embodiments, the TCR-binding molecule, e.g., pMHC complex, and the fusogen are comprised within a fusion protein.

In one specific embodiment, the peptide and MHC (or portion thereof, e.g., MHC peptide binding groove) are separated by a linker sequence (e.g., a linker sequence comprising one or more G₄S repeats (SEQ ID NO: 70)).

In some embodiments, the MHC comprises (i) a class I MHC polypeptide or a fragment (e.g., the peptide binding groove), mutant or derivative thereof, and optionally, (ii) a β2 microglobulin polypeptide or a fragment, mutant or derivative thereof. In some embodiments, the MHC comprises α1 and α2 domains of a class I MHC polypeptide, or a fragment, mutant or derivative thereof. In some embodiments, the MHC comprises α1, α2, and α3 domains of a class I MHC polypeptide, or a fragment, mutant or derivative thereof. In some embodiments, MHC comprises the class I MHC polypeptide (or portion thereof) and β2 microglobulin. In some embodiments, the class I MHC (or portion thereof) and β2 microglobulin polypeptide are linked, e.g., by a peptide linker. In some embodiments, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H-2L, H-2Q, H-2M, and H-2T.

In some embodiments, the MHC comprises a class II MHC polypeptide or a fragment, mutant or derivative thereof. In some embodiments, the MHC comprises α and β polypeptides of a class II MHC complex, or respective fragments, mutants or derivatives thereof. In some embodiments, the MHC comprises α1 and β1 domains of α and β polypeptides, respectively, of a class II MHC complex or a fragment, mutant or derivative thereof. In some embodiments, the α and β polypeptides (or fragments thereof, e.g., the α1 and β1 domains) are linked by a peptide linker. In one specific embodiment, the MHC comprises α and β polypeptides (or fragments thereof, e.g., the α1 and β1 domains) of a human class II MHC complex selected from the group consisting of HLA-DP, HLA-DR, HLA-DQ, HLA-DM and HLA-DO. In another specific embodiment, the MHC comprises α and β polypeptides (or fragments thereof, e.g., the α1 and β1 domains) of a murine H-2A or H-2E class II MHC complex.

In some embodiments, the nucleotide sequence of interest encodes a protein, a peptide, an RNAi molecule, an antisense oligonucleotide, a CRISPR/Cas9 system or derivatives thereof, or a miRNA. In some embodiments, the nucleotide sequence of interest encodes a molecule toxic to the TCR-expressing cell. In some embodiments, the nucleotide sequence of interest encodes a molecule that modulates an activity of the TCR-expressing cell (e.g., a molecule that inhibits an activity of the TCR-expressing cell or a molecule that enhances an activity of the TCR-expressing cell). Non-limiting examples of activities of TCR-expressing cells which can be modulated include, e.g., cell proliferation, cell homing, cell trafficking, production of memory T cells, cytotoxicity towards a target cell, and cytokine production. In another embodiment, the nucleotide sequence of interest encodes a reporter molecule or a selectable marker. In one specific embodiment, the nucleotide sequence of interest is under the control of a promoter selected from the group consisting of a viral promoter homologous to a reference wild-type virus from which the viral particle is derived, a viral promoter heterologous to a reference wild-type virus from which the viral particle is derived, a bacterial promoter, a mammalian promoter, an avian promoter, a fish promoter, an insect promoter, and any combination thereof. In one specific embodiment, the nucleotide sequence of interest is under the control of a human promoter. In one specific embodiment, the nucleotide sequence of interest is under the control of a non-human promoter. In one specific embodiment, the nucleotide sequence of interest is under the control of a promoter that is specific to a T cell (e.g., TCR α-chain promoter, TCR β-chain promoter, TCR β-chain V-region gene promoter, CD2 promoter, CD3 promoter, CD4 promoter, CD5 promoter, CD6 promoter, CD7 promoter, CD8 promoter, CD28 promoter, CD278 promoter, FOXP3 promoter, TIM3 promoter, Granzyme A promoter, Granzyme B promoter, activation-induced cell death (AICD) promoter, TIM promoter, p56lck promoter, NKG5 promoter, or variants thereof).

In some embodiments, the TCR-binding molecule comprises a pMHC complex, wherein the peptide is selected from the group consisting of a self-peptide associated with an autoimmune disorder, a viral peptide, a tumor associated peptide, a bacterial peptide, a fungal peptide, a protozoan parasite peptide, a helminth parasite peptide, and an ectoparasite peptide. In some embodiments, the peptide is associated with an autoimmune or an inflammatory disorder. In one specific embodiment, the peptide comprises a gliadin or a fragment thereof (e.g., (i) α-gliadin fragment corresponding to amino acids 57-73, (ii) γ-gliadin fragment corresponding to amino acids 139-153 and/or (iii) ω-gliadin fragment corresponding to amino acids 102-118 [see, e.g., Camarca et al., Endocrine, Metabolic & Immune Disorders—Drug Targets, 12:207-219 (2012); Camarca et al., J. Immunol., 182(7): 4158-4166 (2009)). In one specific embodiment, the viral peptide comprises gp33 protein or a fragment thereof.

In some embodiments, the TCR targeted by a viral particle disclosed herein is immobilized on a surface. In some embodiments, the TCR targeted by a viral particle disclosed herein is present on a surface of a cell (e.g., a lymphocyte such as, e.g., a T-cell [e.g., a CD4+ T-cell or a CD8+ T-cell]).

In a related aspect, described herein is a method of producing a viral particle that is capable of binding to a TCR, said method comprising culturing a packaging cell in conditions sufficient for the production of a plurality of viral particles, wherein the packaging cell comprises one or more plasmids comprising (i) one or more viral elements involved in assembly of the viral particle, (ii) a nucleotide sequence encoding a TCR-binding molecule and, optionally, (iii) a nucleotide sequence encoding a fusogen.

In some embodiments, the nucleotide sequence (ii) encodes an anti-TCR antibody, or portion thereof. In some embodiments, the nucleotide sequence (ii) encodes a pMHC complex.

In some embodiments, provided herein is a method of producing a viral particle that is capable of binding to an antigen-specific TCR, said method comprising culturing a packaging cell in conditions sufficient for the production of a plurality of viral particles, wherein the packaging cell comprises one or more plasmids comprising (i) one or more viral elements involved in the assembly of the viral particle, (ii) a nucleotide sequence encoding a peptide, (iii) one or more nucleotide sequence(s) encoding a major histocompatibility complex (MHC) molecule (or portion thereof), and optionally (iv) a nucleotide sequence encoding a fusogen, wherein the viral particles present the peptide in the context of the MHC, e.g., the peptide associated with(in) a peptide binding groove of a class I or class II MHC.

In some embodiments of any of the above described methods, the method further comprises collecting the viral particles. In some embodiments, the collecting step comprises one or more of the following steps: clearing cell debris, treating the supernatant containing viral particles with DNase I and MgCl₂, concentrating viral particles, and purifying the viral particles.

In embodiments of any of the above described methods, the packaging cell further comprises a nucleotide sequence of interest, e.g., one or more transfer vectors or a RNA molecule(s), e.g., which may be encoded by the one or more transfer vector, wherein said transfer vector or RNA molecule(s) comprises the nucleotide sequence of interest (and optionally one or more viral elements), and wherein the viral particles comprise said transfer vector(s) or RNA molecule(s). In some embodiments, the (optional) one viral element associated with a nucleotide of interest is a retroviral element. In some embodiments, the (optional) one viral element associated with a nucleotide of interest is a lentiviral element. In some embodiments, the (optional) one viral element associated with a nucleotide of interest is a Psi (ψ) packaging signal. In some embodiments, in addition to a Psi (ψ) packaging signal, the viral element further comprises a 5′ Long Terminal Repeat (LTR) and/or a 3′ LTR, or a derivative or mutant thereof. In some embodiments, the (optional) one viral element associated with a nucleotide of interest is selected from the group consisting of a 5′ Long Terminal Repeat (LTR), a Psi (ψ) packaging signal, a Rev Response Element (RRE), a promoter that drives expression of the nucleotide sequence of interest, a Central Polypurine Tract (cPPT), a Woodchuck hepatitis virus post-transcriptional regulatory element (WPRE), a Unique 3′ (U3), a Repeat (R) region, a Unique 5′ (U5), a 3′ LTR, a 3′LTR with the U3 element deleted (e.g., to make the lentivirus non-replicative), a Trans-activating response element (TAR), and any combination thereof.

In some embodiments of any of the above methods, the one or more plasmids comprises (a) GAG, (b) POL, (c) TAT and/or (d) REV lentiviral or retroviral elements, each of which may be individually considered a viral element involved in the assembly of the viral particle. In some embodiments, the one or more plasmids comprises GAG and POL retroviral and/or lentiviral elements. In some embodiments, the one or more plasmids comprises GAG, POL and TAT retroviral and/or lentiviral elements.

In some embodiments of any of the above-described methods, the plasmid(s) present in the packaging cell do not comprise a functional lentiviral or functional retroviral ENV gene, e.g., the plasmid(s) lack any lentiviral or retroviral ENV gene or comprise a mutant ENV gene that encodes a non-functional protein, e.g., a protein that does not bind its natural ligand. In another embodiment, the one or more plasmids comprise a mutant lentiviral ENV gene, which does not produce gp120 surface envelope protein or gp41 transmembrane envelope or which encodes a mutant gp120 surface envelope protein and/or a mutant gp41 transmembrane envelope protein and wherein the resulting viral particle is not capable of binding to a target cell in the absence of the TCR-binding molecule.

In some embodiments of any of the above methods, the one or more viral elements encodes Human Immunodeficiency Virus (HIV) component(s), Bovine Immunodeficiency Virus (BIV) component(s), Feline Immunodeficiency Virus (FIV) component(s), Simian Immunodeficiency Virus (SIV) component(s), Equine Infectious Anemia Virus (EIAV) component(s), Murine Stem Cell Virus (MSCV) component(s), or Murine Leukemia Virus (MLV) component(s). In some embodiments, the one or more viral elements encode HIV components, e.g., an HIV capsomer, a plurality of HIV capsomers, an HIV capsid, etc.

In some embodiments of any of the above methods, the fusogen is a protein; e.g., a viral protein (e.g., a vesiculovirus protein [e.g., vesicular stomatitis virus G glycoprotein (VSVG)], an alphavirus protein [e.g., a Sindbis virus glycoprotein], an orthomyxovirus protein [e.g., an influenza HA protein], a paramyxovirus protein [e.g., a Nipah virus F protein or a measles virus F protein]), a retrovirus protein, a lentivirus protein, or a fragment, mutant or derivative thereof. In some embodiments, the fusogen is a mutated protein which does not bind its natural ligand. In some embodiments, a method as described above comprises a nucleic acid sequence encoding a fusogen having a sequence set forth as SEQ ID NO: 5. In one specific embodiment, the nucleic acid sequence encoding a fusogen is SEQ ID NO: 6. In one specific embodiment, the fusogen is a vesicular stomatitis virus G glycoprotein (VSVG) or a fragment, mutant or derivative thereof. In one specific embodiment, at least one chain of the MHC and the fusogen are comprised within a fusion protein. In one specific embodiment, the pMHC complex and the fusogen are comprised within a fusion protein.

In one specific embodiment, the MHC and the peptide are separated by a linker sequence (e.g., a linker sequence comprising one or more G₄S repeats (SEQ ID NO: 70)).

In some embodiments, the MHC comprises (i) a class I MHC polypeptide or a fragment, mutant or derivative thereof, and, optionally, (ii) a β32 microglobulin polypeptide or a fragment, mutant or derivative thereof. In some embodiments, the MHC comprises α1 and α2 domains of a class I MHC polypeptide, or a fragment, mutant or derivative thereof. In some embodiments, the MHC comprises α1, α2, and α3 domains of a class I MHC polypeptide, or a fragment, mutant or derivative thereof. In some embodiments, MHC comprises the class I MHC polypeptide (or portion thereof) and β2 microglobulin. In some embodiments, the class I MHC and β2 microglobulin polypeptide are linked, e.g., by a peptide linker. In some embodiments, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H-2L, H-2Q, H-2M, and H-2T.

In some embodiments, the MHC comprises a class II MHC polypeptide or a fragment, mutant or derivative thereof. In some embodiments, the MHC comprises α and β polypeptides of a class II MHC complex or respective fragments, mutants or derivatives thereof. In some embodiments, the MHC comprises α1 and β1 domains of α and β polypeptides, respectively, of a class II MHC complex or a fragment, mutant or derivative thereof. In one specific embodiment, the α and β polypeptides (or fragments thereof, e.g., the α1 and β1 domains) are linked by a peptide linker. In one specific embodiment, the MHC comprises α and β polypeptides (or fragments thereof, e.g., the α1 and β1 domains) of a human class II MHC complex selected from the group consisting of HLA-DP, HLA-DR, HLA-DQ, HLA-DM and HLA-DO. In another specific embodiment, the MHC comprises α and β polypeptides (or fragments thereof, e.g., the α1 and β1 domains) of a murine H-2A or H-2E class II MHC complex.

In some embodiments of any of the above methods, the nucleotide sequence of interest encodes a protein, a peptide, an RNAi molecule, an antisense oligonucleotide, a CRISPR/Cas9 system or derivatives thereof, or a miRNA. In some embodiments, the nucleotide sequence of interest encodes a molecule toxic to the TCR-expressing cell. In some embodiments, the nucleotide sequence of interest encodes a molecule that modulates an activity of the TCR-expressing cell (e.g., a molecule that inhibits an activity of the TCR-expressing cell or a molecule that enhances an activity of the TCR-expressing cell). Non-limiting examples of activities of TCR-expressing cells which can be modulated include, e.g., cell homing, cell trafficking, production of memory T cells, cytotoxicity towards a target cell, and cytokine production. In another embodiment, the nucleotide sequence of interest encodes a reporter molecule or a selectable marker. In one specific embodiment, the nucleotide sequence of interest is under the control of a promoter selected from the group consisting of a viral promoter homologous to the at least one viral element, a viral promoter heterologous to the at least one viral element, a bacterial promoter, a mammalian promoter, an avian promoter, a fish promoter, an insect promoter, and any combination thereof. In one specific embodiment, the nucleotide sequence of interest is under the control of a human promoter. In one specific embodiment, the nucleotide sequence of interest is under the control of a non-human promoter. In one specific embodiment, the nucleotide sequence of interest is under the control of a promoter that is specific to a T cell (e.g., TCR α-chain promoter, TCR β-chain promoter, TCR β-chain V-region gene promoter, CD2 promoter, CD3 promoter, CD4 promoter, CD5 promoter, CD6 promoter, CD7 promoter, CD8 promoter, CD28 promoter, CD278 promoter, FOXP3 promoter, TIM3 promoter, Granzyme A promoter, Granzyme B promoter, activation-induced cell death (AICD) promoter, TIM promoter, p56lck promoter, NKG5 promoter, and variants thereof).

In some embodiments of any of the above described methods, the peptide is selected from the group consisting of a self-peptide associated with an autoimmune disorder, a viral peptide, a tumor associated peptide, a bacterial peptide, a fungal peptide, a protozoan parasite peptide, a helminth parasite peptide, and an ectoparasite peptide. In some embodiments, the peptide is associated with an autoimmune or an inflammatory disorder. In one specific embodiment, the peptide comprises a gliadin or a fragment thereof (e.g., (i) α-gliadin fragment corresponding to amino acids 57-73 or (ii) γ-gliadin fragment corresponding to amino acids 139-153 or (iii) ω-gliadin fragment corresponding to amino acids 102-118). In one specific embodiment, the viral peptide comprises gp33 protein or a fragment thereof.

In some embodiments of any of the above methods, the packaging cell does not comprise any MHCs other than the MHC of the viral particle.

In a related aspect, disclosed herein are viral particles made by any of the above methods.

Also described herein are pharmaceutical compositions comprising the viral particles described herein and a pharmaceutically acceptable carrier or excipient. In addition, described herein are pharmaceutical dosage forms comprising the viral particle described herein.

In another aspect, disclosed herein are methods for delivering a nucleotide sequence of interest to a cell comprising a TCR, e.g., expressing the TCR, e.g., on its cell surface, the method comprising contacting the cell with a viral particle described herein, wherein the viral particle comprises a molecule that binds the TCR, e.g., a TCR-binding molecule. In some embodiments, the TCR expressed by the cell is antigen specific, and the TCR-binding molecule specifically binds the idiotype of the TCR, e.g., the TCR-binding molecule is an antigen to which the antigen-specific T cell specifically binds. In some embodiments, the delivery of the nucleotide sequence of interest results in modulating an activity or survival of the TCR-expressing cell.

In a further aspect, described herein is a method for modulating an activity or survival of a cell comprising a TCR, comprising contacting the cell with a viral particle described herein, wherein the viral particle comprises a TCR-binding molecule that binds the TCR, e.g., specifically binds the TCR.

In some embodiments of any of the above methods, the cell is a lymphocyte such as, e.g., a T-cell (e.g., a CD4+ T-cell or a CD8+ T-cell).

In some embodiments of any of the above methods, said contacting is ex vivo.

In some embodiments of the above methods, said contacting is in vivo in a subject (e.g., human).

In some embodiments of any of the above methods, the cell is a mammalian cell (e.g., a human cell).

In a related aspect, described herein are modified cells comprising the viral particles, or one or more portions thereof (e.g., capsomer, nucleotide of interest, viral element, pMHC complex, a combination thereof, etc.) described herein. In some embodiments, the cell is a lymphocyte such as, e.g., a T-cell (e.g., a CD4+ T-cell or a CD8+ T-cell). In some embodiments, the cell is a mammalian cell (e.g., a human cell).

In another aspect, described herein is a method of treating a disorder in a subject in need thereof comprising administering to said subject an effective amount of a viral particle described herein, wherein the viral particle comprising a TCR-binding molecule as described herein binds to an antigen-specific TCR and wherein the antigen recognized by the TCR is associated with the disorder. In some embodiments, the antigen is a pMHC complex and the TCR-binding molecule comprises the pMHC complex, or a portion thereof.

In some embodiments, the disorder an inflammatory or an autoimmune disorder and the administration results in a downregulation of an inflammatory or autoimmune response. In one specific embodiment, the disorder is celiac disease or gluten sensitivity. In one specific embodiment, the antigen comprises a gliadin or a fragment thereof (e.g., (i) α-gliadin fragment corresponding to amino acids 57-73 or (ii) γ-gliadin fragment corresponding to amino acids 139-153 or (iii) ω-gliadin fragment corresponding to amino acids 102-118), which may be presented in the context of a class II MHC. Accordingly, in some embodiments, a viral particle comprises a TCR-binding molecule that comprises a gliadin peptide presented in the context of MHC, e.g., the TCR-binding molecule comprises a peptide selected from the group consisting of amino acids 57-73 of α-gliadin, a portion thereof, amino acids 139-153 of 7-gliadin fragment, a portion thereof, amino acids 102-118 of ω-gliadin fragment, and a portion thereof, wherein the peptide is associated with(in) a peptide binding groove of a class II MHC. In some embodiments, the disorder is a tumor and the administration results in an upregulation of an anti-tumor immune response. In another embodiment, the disorder is an infection caused by an infectious agent and the administration results in an upregulation of an immune response against the infectious agent. In some embodiments, the infectious agent is selected from the group consisting of a virus, a bacterium, a fungus, a protozoa, a parasite, a helminth, and an ectoparasite. In some embodiments, a viral particle described herein comprises a TCR-binding molecule that comprises a viral peptide associated with(in) a peptide binding groove of a class I MHC. In some embodiments, the infectious agent is lymphocytic choriomeningitis virus (LCMV) and the peptide is gp33, which may be presented in the context of MHC, e.g., MHC I. In some embodiments, the subject is a mammal (e.g., human).

In another aspect, described herein is a packaging cell for producing the viral particles described herein comprising one or more plasmids comprising (i) one or more viral elements involved in the assembly of the viral particle, (ii) a nucleotide sequence encoding a TCR-binding molecule and optionally (iii) a nucleotide sequence encoding a fusogen. In some embodiments, the nucleotide sequence (ii) encodes an anti-TCR antibody or portion thereof. In some embodiments, described herein is a packaging cell for producing the viral particles described herein comprising one or more plasmids comprising (i) one or more viral elements involved in the assembly of the viral particle, (ii) a nucleotide sequence encoding an antigen-derived peptide, (iii) one or more nucleotide sequence(s) encoding a major histocompatibility complex (MHC) molecule, optionally and (iv) a nucleotide sequence encoding a fusogen. In some embodiments, the packaging cell further comprises a nucleotide sequence of interest, e.g., comprised in a transfer vector or as an RNA encoded by the transfer vector. In some embodiments, the packaging cell does not comprise any MHCs other than the MHC of the viral particle.

In another aspect, described herein is a library comprising a plurality of viral particles of described herein.

In some embodiments, the viral particles within the library differ in the sequence of the peptides (p) presented in the context of a major histocompatibility complex (MHC) molecule (pMHC complex) on the surface of the viral particles.

In some embodiments, the library comprises at least 10², at least 10³, at least 10⁴, at least 10⁵, at least 10⁶, at least 10⁷, at least 10⁸, at least 10⁹, or at least 10¹⁰ unique viral particles.

In some embodiments, each different peptide (p) sequence within the library is generated based on mass-spectrometry and/or computational analysis and/or validated TCR epitopes.

In some embodiments, each viral particle within the library comprises a polynucleotide encoding a reporter protein. In some embodiments, each viral particle within the library comprises a reporter protein. In some embodiments, the reporter protein is fused to a viral particle protein. In some embodiments, the viral particle is a lentiviral particle and the viral particle protein is VPR. In some embodiments, the reporter protein is a fluorescent protein.

In some embodiments, each viral particle within the library comprises a polynucleotide encoding a pMHC complex and optionally a universal primer binding sequence. In some embodiments, each viral particle within the library comprises a polynucleotide encoding a pMHC complex and a universal primer binding sequence, wherein the pMHC complex is the same as the pMHC complex which is present on the surface of said viral particle.

In some embodiments, each viral particle within the library is a lentiviral particle which comprises a fusogen, wherein said fusogen comprises a Sindbis virus glycoprotein or a fragment, mutant or derivative thereof. In one specific embodiment, the fusogen is a mutated Sindbis virus glycoprotein which does not bind its natural ligand. In one specific embodiment, the fusogen comprises the sequence set forth as SEQ ID NO: 5.

In another aspect, described herein is a method for identifying an antigen-specific T-cell receptor (TCR), comprising: a) contacting a sample comprising TCR-expressing cells, wherein said sample has been isolated from a subject who has been previously exposed to an antigen, with a library described herein, wherein the library comprises a plurality of viral particles comprising peptides (p) derived from said antigen, wherein said peptides are presented in the context of pMHC complexes on the surface of the viral particles, and wherein the viral particles in the library comprise a reporter protein or a polynucleotide encoding the reporter protein, b) identifying one or more cells comprising the reporter protein, and c) determining the sequence(s) of TCR(s) expressed by the one or more cells identified in step (b).

In some embodiments, the TCR is identified using a single cell RNA sequencing.

In some embodiments, TCR-expressing cells are selected from CD8+ T cells, CD4+ T cells, or pan CD3+ T cells.

In some embodiments, the sample is isolated peripheral blood mononuclear cells (PBMCs).

In some embodiments, prior to step (a) the TCR-expressing cells in the sample are expanded and/or activated in the presence of the same plurality of antigen-derived peptides (p) as the peptides present in the library.

In some embodiments, the method further comprises determining the activation state of the one or more cells identified in step (b).

In some embodiments, the activation state of the one or more cells identified in step (b) is determining the expression level of one or more genes selected from IFNγ, granzyme B, 4-1BB, CD28, CD25, CD69, OX40, and CD40L.

In some embodiments, each of viral particles in the library comprises a polynucleotide encoding a pMHC complex and a universal primer binding sequence, wherein the pMHC complex is the same as the pMHC complex which is present on the surface of said viral particle.

In some embodiments, the method further comprises identifying the cognate peptide(s) recognized by the identified TCR(s), by determining the sequence of the peptide(s) contained within the pMHC-encoding polynucleotide(s) in the one or more cells identified in step (b).

In some embodiments, the peptide sequence is determined by sequencing using a primer that binds the universal primer binding sequence.

In another aspect, described herein is a method for identifying an antigenic peptide recognized by one or more antigen-specific TCR-expressing cells, comprising: a) contacting a sample comprising TCR-expressing cells, wherein said sample has been isolated from a subject who has been previously exposed to an antigen, with a library described herein, wherein the library comprises a plurality of viral particles comprising peptides (p) derived from said antigen, wherein said peptides are presented in the context of pMHC complexes on the surface of the viral particles, and wherein the viral particles in the library comprise (i) a reporter protein or a polynucleotide encoding the reporter protein and (ii) a polynucleotide encoding pMHC complex and a universal primer binding sequence, wherein the pMHC complex is the same as the pMHC complex which is present on the surface of the viral particle, b) identifying one or more cells comprising the reporter protein, and c) identifying the peptide(s) encoded by the viral particle(s) which had infected the one or more cells identified in step (b).

In some embodiments, TCR-expressing cells are selected from CD8+ T cells, CD4+ T cells, or pan CD3+ T cells.

In some embodiments, the sample is isolated peripheral blood mononuclear cells (PBMCs).

In some embodiments, prior to step (a) the TCR-expressing cells in the sample are expanded in the presence of the same plurality of antigen-derived peptides (p) as the peptides present in the library.

In some embodiments, identifying the peptide(s) in step (c) comprises determining the sequence of the peptide(s) contained within the pMHC-encoding polynucleotide expressed by the one or more cells identified in step (b).

In some embodiments, the peptide sequence is determined by sequencing using a primer that binds the universal primer binding sequence.

In some embodiments, the method further comprises identifying the antigenic peptide recognized by the largest number of TCR-expressing cells in samples isolated from a plurality of subjects who have been previously exposed to the antigen.

These and other aspects described herein will be apparent to those of ordinary skill in the art in the following description, claims and drawings.

### BRIEF DESCRIPTION OF THE DRAWINGS

FIG. 1A is a schematic representation of an exemplary enveloped lentiviral particle of the invention that is pseudotyped with a peptide-loaded MHC class I or class II complex (pMHC-I or pMHC-II respectively), displays a fusogen, and comprises a nucleotide of interest within a capsid, and its use for peptide-specific genetic modification of CD4+ or CD8+ T cells expressing specific TCR with the nucleotide of interest. FIG. 1B shows therapeutic applications of two exemplary enveloped lentiviral particles pseudotyped with a peptide-loaded MHC class I or class II complex, displaying a fusogen, and comprising a nucleotide encoding a nucleotide of interest.

FIG. 2A represents a schematic of an exemplary lentiviral particle pseudotyped with a mouse class I MHC molecule (H-2D^{b}) bound to the lymphocytic choriomeningitis virus (LCMV) gp33 peptide and comprising enhanced green fluorescent protein (EGFP) as a nucleotide of interest. The schematics of the transfer vector, packaging plasmid, and envelope plasmids (one encoding the pMHC complex and another encoding a mutant Sindbis (SINmu) fusogen) used to generate the lentiviral particle are depicted on the left-hand side. FIG. 2B shows a schematic representation of the lentiviral particle pseudotyped with mouse H-2D^{b} bound to the LCMV gp33 peptide and its interaction with a CD8+ T cell expressing a P14 TCR.

FIG. 3 demonstrates a general protocol for LV-H-2D^{b}/gp33-EGFP lentiviral particles production, purification and transduction of cells expressing or not expressing P14, a gp33-specific T cell receptor (TCR).

FIG. 4 provides immunofluorescence microscopy images evaluating EGFP expression upon transduction of P14 TCR-expressing cells (J.RT3-T3.5/mCD28/mCD8αβ/P14) or control cells without P14 TCR (J.RT3-T3.5/mCD28/mCD8αβ) with lentiviral particles pseudotyped with the H-2D^{b}/gp33 complex (LV-H-2D^{b}/gp33-EGFP), lentiviral particles displaying only SINmu fusogen (LV-SINmu-EGFP), or control solution without any lentiviral particles (“untransduced”).

FIG. 5 shows fluorescence-activated cell sorting (FACS) analysis evaluating EGFP expression upon transduction of P14 TCR-expressing cells (J.RT3-T3.5/mCD28/mCD8αβ/P14) or control cells without P14 TCR (J.RT3-T3.5/mCD28/mCD8αβ) with lentiviral particles pseudotyped with the H-2D^{b}/gp33 complex (LV-H-2D^{b}/gp33-EGFP), lentiviral particles displaying only SINmu fusogen (LV-SINmu-EGFP), or solution without any lentiviral particles (“untransduced”).

FIGS. 6A-6B represent a schematic of a lentiviral particle (FIG. 6A) pseudotyped with a mouse class I MHC molecule (H-2D^{b}) bound to the LCMV gp33 peptide or (FIG. 6B) pseudotyped with a mouse class I MHC molecule (H-2K^{b}) bound to an ovalbumin (OVA) peptide, each comprising human FOXP3 and the EGFP reporter linked via a self-cleaving P2A peptide as a nucleotide of interest. The schematics of the transfer vector, packaging plasmid, and envelope plasmids (one encoding the pMHC complex and another encoding a mutant Sindbis (SINmu) fusogen) used to generate the lentiviral particle are depicted on the left-hand side.

FIG. 7A illustrates an in vitro model for testing modulation of Jurkat-derived T-cell repression. In this model, engineered J.RT3-T3.5 cells expressing either the gp33-restricted P14 TCR or the OVA-restricted OT-1 TCR are infected with lentiviral particles (LV) carrying a human FOXP3-P2A-EGFP cassette as nucleotide of interest, and pseudotyped either with MHC class I (H-2D^{b}) molecule displaying gp33 peptide or with MHC class I (H-2K^{b}) molecule displaying ovalbumin (OVA) peptide. Additional control groups include untransduced cells (Mock) and pan-tropic lentiviral particles displaying the envelope glycoprotein of the Vesicular Stomatitis Virus (LV-VSV-hFOXP3). Mock and transduced cells are peptide stimulated and the T-cell phenotype, e.g., activation or suppressive (Treg-like) phenotype, of the cells is measured. The expected T-cell phenotype for each test group is depicted in the right-hand column. FIG. 7B shows FACS analysis evaluating EGFP expression upon transduction of OT1 TCR expressing cells (J.RT3-T3.5/AP1-Luc/hCD28/hCD8αβ/OT1) or control cells without OT1 TCR (J.RT3-T3.5/AP1-Luc/hCD28/hCD8αβ) with lentiviral particles pseudotyped with the H-2K^{b}/OVA complex (LV-H-2K^{b}/OVA-EGFP). FIGS. 7C-7D provide additional data showing the pan-tropic lentiviral particle LV-VSV-hFOXP3 mediate hFOXP3 expression and modulates T cell phenotype in Jurkat/NFAT-Luc cells. FIGS. 7E-7G provide additional data showing that ectopic expression of Foxp3 by the pan-tropic lentiviral particle LV-VSV-hFOXP3 induces a Treg-like phenotype in Jurkat/NFAT-Luc cells. FIGS. 7H-7I provide additional data showing ectopic expression of FOXP3 with H-2K^{b}-OVA single chain induces a specific TL-8 increase in J.RT3 cells displaying the OT1 TCR.

FIG. 8 shows fluorescence-activated cell sorting (FACS) analysis evaluating LNGFR expression upon transduction of mixed cultures of wild-type primary T cells (isolated from C57Bl/6 mouse splenocytes) and OT1 TCR-expressing primary T cells (isolated from transgenic OT-1 mouse splenocytes) with lentiviral particles carrying the cDNA sequence of LNGFR as nucleotide of interest, and pseudotyped respectively with an anti-mouse CD8 antibody (LV-αmCD8-LNGFR), an anti-mouse CD4 antibody (LV-αmCD4-LNGFR), the H-2D^{b}/gp33 complex (LV-H-2D^{b}/gp33-LNGFR), the H-2K^{b}/OVA complex (LV-H-2K^{b}/OVA-LNGFR) or solution without any lentiviral particles (“untransduced”).

FIG. 9 illustrates an ex vivo functional assay for testing modulation of mouse primary CD8+ cytotoxic T cells phenotype. Naïve CD8+ T-cells are isolated from splenocytes of transgenic P14 mice and OT-1 mice and then transduced with either LV-H-2D^{b}/gp33-mTBX21-P2A-EGFP or LV-H-2K^{b}/OVA-mTBX21-P2A-EGFP lentiviral particles. Additional controls include transduction without the lentiviral particles (mock) or with lentiviral particles targeting all CD8+ T cells by displaying and anti-mouse CD8 monoclonal antibody on their surface (LV-αmCD8-mTBX21-P2A-EGFP). Mock and transduced cells are then stimulated to activate T cells and the T-cell phenotype, e.g., differentiation into cytotoxic effector T cells, is measured. The expected T-cell phenotype for each test group is depicted in the right-hand column.

FIG. 10 illustrates an ex vivo model for testing modulation of mouse primary CD8+ cytotoxic T-cells phenotype. In this model, memory CD8+ T cells are generated by immunizing wild type C57Bl/6 mice with gp33 or ovalbumin (OVA) peptide. The population of splenocytes from immunized mice, which comprises memory CD8+ T cells expressing gp33-restricted TCR or OVA-restricted TCR are either not infected (mock) or infected with lentiviral particles (LV) comprising LV-αmCD8-mTBX21-P2A-EGFP, LV-H-2D^{b}/gp33-mTBX21-P2A-EGFP or LV-H-2K^{b}/OVA-mTBX21-P2A-EGFP. Mock and transduced cells are then stimulated to activate T cells and the T-cell phenotype, e.g., differentiation into cytotoxic effector T cells, of the cells measured. The expected T-cell phenotype for each test group is depicted in the right-hand column.

FIG. 11 represents a schematic of a lentiviral particle pseudotyped with a human class I MHC molecule (HLA-A2) bound to the human cytomegalovirus (CMV) pp65 peptide and comprising enhanced green fluorescent protein (EGFP) as a nucleotide of interest. The schematics of the transfer vector, packaging plasmid, and envelope plasmids (one encoding the pMHC complex and another encoding a mutant Sindbis (SINmu) fusogen) used to generate the lentiviral particle are depicted on the left-hand side.

FIG. 12A shows fluorescence-activated cell sorting (FACS) analysis evaluating EGFP expression upon transduction of pp65 TCR expressing cells (J.RT3-T3.5/AP1-Luc/hCD28/hCD8αβ/pp65) or control cells without pp65 TCR (J.RT3-T3.5/AP1-Luc/hCD28/hCD8αβ) with lentiviral particles pseudotyped with the HLA-A2/pp65 complex (LV-HLA-A2/pp65-EGFP), pan-tropic lentiviral particles displaying the envelope glycoprotein of the Vesicular Stomatitis Virus (VSV), or solution without any lentiviral particles (“untransduced”). FIG. 12B shows additional FACS analysis evaluating EGFP expression upon transduction of pp65 TCR expressing cells (J.RT3-T3.5/AP1-Luc/hCD28/hCD8αβ/pp65) or control cells without pp65 TCR (J.RT3-T3.5/AP1-Luc/hCD28/hCD8αβ) with lentiviral particles pseudotyped with the HLA-A2/pp65 complex (LV-HLA-A2/pp65-EGFP).

FIG. 13A illustrates in vitro functional studies of the capacity of lentiviral particles pseudotyped with HLA-A2/pp65 and carrying the human transcription factor TBX21 as a nucleotide of interest to trigger an enhanced cytotoxic-like phenotype in Jurkat-derived T cells displaying the pp65-restricted TCR. In this model, parental J.RT3-T3.5/AP1-Luc/hCD28/hCD8αβ cells and engineered J.RT3-T3.5/AP1-Luc/hCD28/hCD8αβ/pp65 cells are transduced either with the LV-HiLA-A2/pp65-hTBX21-P2A-EGFP or the pantropic LV-VSV-A2/pp65 as a control. Additional control groups include untransduced cells (Mock) and pan-tropic lentiviral particles displaying the envelope glycoprotein of the Vesicular Stomatitis Virus (LV-VSV-hTBX21). After lentiviral transductions, J.RT3-T3.5/AP1-Luc/hCD8ab/hCD28 and J.RT3-T3.5/AP1-Luc/hCD8ab/hCD28/pp65 cells are then stimulated with treatment with phytohemagglutinin (PHA) and phorbol 12-myristate 13-acetate (PMA), treatment with soluble anti-CD3/anti-CD28 monoclonal antibodies, treatment with beads coupled with anti-CD3/anti-CD8 monoclonal antibodies, or treatment with mouse antigen-presenting cells (APC) previously pulsed with gp33 and OVA peptides. The expected T-cell phenotype for each test group is depicted in the right-hand column. FIGS. 13B-13D provide additional data showing that ectopic expression of T-bet by the lentiviral particles LV-VSV-hTBX21 induces a decrease in NFAT activity but an increase in IFNγ in Jurkat/NFAT-Luc cells.

FIG. 14A represents a schematic of a lentiviral particle displaying a human class II MHC molecule (HLA-DQ2.5) bound to the α1-gliadin peptide and comprising enhanced green fluorescent protein (EGFP) as a nucleotide of interest (LV-HLA-DQ2.5/α1-gliadin-EGFP). The schematics of the transfer vector, packaging plasmid, and envelope plasmids (two encoding the pMHC complex and another encoding a mutant Sindbis (SINmu) fusogen) used to generate the lentiviral particle are depicted on the left-hand side. FIG. 14B shows a schematic representation of the lentiviral particle pseudotyped with HLA-DQ2.5 bound to the α1-gliadin peptide and its interaction with a CD4+ T cell expressing a α1-gliadin TCR.

FIGS. 15A-15C show fluorescence-activated cell sorting (FACS) analysis evaluating EGFP expression upon transduction of α1-gliadin restricted TCR expressing cells (J.RT3-T3.5/AP1-Luc/hCD28/hCD8αβ/α1-gliadin TCR) or control cells expressing α2-gliadin restricted TCR (JRT3/AP1-luc/hCD28/hCD8αβ/α2-gliadin TCR), or control cells without TCR (J.RT3-T3.5/AP1-Luc/hCD28/hCD8αβ) with lentiviral particles pseudotyped with HLA-DQ2.5/α1-gliadin complex (LV-HLA-DQ2/α1-gliadin-EGFP), or control lentiviral particles expressing only SINmu fusogen (LV-SINmu-EGFP), or control lentiviral particles displaying only VSV (LV-VSV-EGFP), or solution without any lentiviral particles (“untransduced”).

FIG. 16A illustrates an ex vivo model for testing modulation of T-cell activation. In this model, CD4⁺ T cells are isolated from human peripheral blood mononuclear cells (PBMCs) of patients reactive for α1- and α2-gliadin, and are infected by lentiviral particles (LV) comprising the human transcription factor hFOXP3 and the EGFP reporter as nucleotide of interest and pseudotyped with the MHC class II (HLA-DQ2.5) displaying the α1-gliadin or α2-gliadin peptide. Additional control groups include untransduced cells (Mock) and pan-tropic lentiviral particles displaying the envelope glycoprotein of the Vesicular Stomatitis Virus (LV-VSV-hFOXP3-P2A-GFP) and effect of activation of the T cells prior to transduction is also tested. Mock and transduced cells are then peptide stimulated and the T cell phenotype, e.g., activation or suppressive phenotype, of the cells is measured. The expected T cell phenotype for each test group is depicted in the right-hand column. FIG. 16B provides additional data showing the pan-tropic lentiviral particle LV-VSV-hFOXP3 mediate hFOXP3 expression and modulates T cell phenotype in CD4⁺ enriched human PBMCs. FIG. 16C provides additional data showing ectopic expression of FOXP3 by the pan-tropic lentiviral particle LV-VSV-hFOXP3 partially induces a Treg-like phenotype in human CD4 T cells.

FIG. 17 illustrates ex vivo models for testing the ability of lentiviral particles presenting either the gp33 peptide in the context of mouse MHC I (H2-D^{b}) molecules or the OVA peptide in the context of mouse MHC I (H-2K^{b}) molecule, and carrying the mouse transcription factor FOXP3 (mFOXP3) as a nucleotide of interest to trigger a suppressive phenotype in mouse naïve CD8+ T cells. Naïve CD8+ T cells are first isolated from splenocytes of transgenic P14 and OT-1 mice and are then transduced with lentiviral particles displaying gp33 peptide in the groove of the MHC I (H-2D^{b}) molecule or control OVA peptide in the groove of the MHC I (H-2K^{b}) molecule on its envelope surface and comprising a mFOXP3-P2A-EGFP cassette as a nucleotide of interest. Additional control groups include untransduced cells (Mock) and pan-CD8+ T cells targeting lentiviral particles displaying an anti-mouse CD8 monoclonal antibody on their surface (LV-αmCD8-mFOXP3-P2A-GFP). Mock or transduced T cells are then re-stimulated with treatment with phytohemagglutinin (PHA) and phorbol 12-myristate 13-acetate (PMA), treatment with soluble anti-CD3/anti-CD28 monoclonal antibodies, treatment with beads coupled with anti-CD3/anti-CD8 monoclonal antibodies, or treatment with mouse antigen-presenting cells (APC) pulsed with gp33 and OVA peptides. The expected T-cell phenotype for each test group is depicted in the right-hand column.

FIG. 18 illustrates the identification of TCRs that are specific for the CMV pp65 peptide. In this model memory human CD8+ T cells are first isolated from patients that are seropositive for CMV. The isolated CD8⁺ T cells are then transduced with lentiviral particles displaying the pp65 peptide in the groove of the MHC I (HLA-A2) molecule on its surface and delivering the coding sequence of the human telomerase reverse transcriptase (hTERT-P2A-EGFP cassette) as nucleotide of interest. A lentiviral particle displaying the pp65 peptide in the groove of the MHC I (HLA-A2) molecule on its surface and encoding EGFP are used as control. After 3 to 4 weeks in culture only cells efficiently transduced and expressing hTERT-P2A-EGFP are surviving and proliferating in comparison to cells transduced only with the EGFP encoding lentiviral particle. EGFP+ cells are then sorted and pp65-restricted TCRs can be sequenced.

FIG. 19 illustrates an in vivo tumor model for testing enhancement of gp33 specific CD8+ T cell immunity by transduction with LV-H-2D^{b}/gp33 pseudotyped vectors. In this model, B16F10 and B16F10/gp33 melanoma cell lines are injected subcutaneously in C57Bl/6 mice to generate tumors. The mice that developed tumors are then injected intratumorally with either LV-H-2D^{b}/gp33-mTBX21 or LV-H-2K^{b}/OVA-mTBX21, and tumor growth is analyzed after different time points post-treatment. The expected phenotype for each test group is depicted in the right-hand column.

FIG. 20 illustrates an overview of the platform used to identify virus specific CD8 T cells and obtain matched TCR/antigen data.

FIG. 21 illustrates modified lentiviruses specifically infecting antigen specific CD8 T cells.

FIG. 22 illustrates experiments conducted for sorting and sequencing lentivirus-infected cells.

FIGS. 23A-23B illustrate that sequencing of GFP+ cells shows expected ratio of cells.

FIGS. 24A-24C illustrate the production of BK Virus (BKV) lentiviral library.

### DETAILED DESCRIPTION

The present invention provides recombinant viral particles pseudotyped with T cell receptor (TCR)-binding molecules. The viral particles described herein are capable of selectively targeting and modulating the activity of cell subpopulations carrying antigen-specific TCRs making said viral particles useful for treatment of various inflammatory, autoimmune and infectious disorders as well as cancers.

### Definitions

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “antigen” refers to any agent (e.g., protein, peptide, polysaccharide, glycoprotein, glycolipid, nucleic acid, portions thereof, or combinations thereof) that, when introduced into a host, animal or human, having an immune system (directly or upon expression as in, e.g., DNA vaccines), is recognized by the immune system of the host and is capable of eliciting an immune response.

As described herein, the T-cell receptor (TCR) recognizes a peptide presented in the context of a major histocompatibility complex (MHC) molecule. The peptide-MHC (pMHC) complex is recognized by TCR, with the peptide (antigenic determinant) and the TCR idiotype providing the specificity of the interaction. Accordingly, the term “antigen” encompasses peptides presented in the context of MHC molecules. The peptide displayed on an MHC molecule may also be referred to as an “epitope” or an “antigenic determinant”. The terms “peptide,” “antigenic determinant” and “epitope” as used herein encompass not only those presented naturally by antigen-presenting cells (APCs) but may be any desired peptide so long as it is recognized by an immune cell, e.g., when presented appropriately to the cells of an immune system. For example, a peptide having an artificially prepared amino acid sequence may also be used as the epitope.

The terms “Major Histocompatibility Complex”, “MHC” and “MHC molecule” encompass naturally occurring MHC molecules as well as individual chains of MHC molecules (e.g., MHC class I α (heavy) chain, β2-microglobulin, MHC class II α chain, MHC class II β chain), individual subunits of such chains of MHC molecules (e.g., α1, α2, and/or α3 subunits of MHC class I α chain, α1 and/or α2 subunits of MHC class II α chain, β1 and/or β2 subunits of MHC class II β chain) as well as fragments, mutants and various derivatives thereof (including fusion proteins), wherein such fragments, mutants and derivatives retain the ability to display an antigenic peptide for recognition by a TCR, e.g., an antigen-specific TCR. An MHC class I molecule comprises a peptide binding groove formed by the α1 and α2 domains of the heavy α chain that can stow a peptide of around 8-10 amino acids. Despite the fact that both classes of MHC bind a core of about 9 amino acids within peptides, the open-ended nature of MHC class II peptide-binding groove (the α1 domain of a class II MHC a polypeptide in association with the 31 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide-binding groove, changing which 9-mer sits directly within the groove at any given time. Conventional identifications of particular MHC variants are used herein. For example, HLA-B 17 refers to a human leucocyte antigen from the B gene group (hence a class I type MHC) gene position (known as a gene locus) number 17; gene HLA-DR11, refers to a human leucocyte antigen coded by a gene from the DR region (hence a class II type MHC) locus number 11.

The term “pseudotyped” in connection with viral particles described herein refers to viral particles comprising in their lipid envelope or capsid molecules, e.g., proteins, glycoproteins, etc., which are mutated and/or heterologous compared to molecules typically found on the surface of the virus from which the particles are derived, and which may affect, contribute to, direct, redirect and/or completely change the tropism of the viral particle in comparison to a reference wild-type virus from which the viral particle is derived. In some embodiments, a viral particle is pseudotyped such that it recognizes, binds and/or infects a target (ligand or cell) that is different to that of a reference wild-type virus from which the viral particle is derived. In some embodiments, a viral particle is pseudotyped such that it does not recognize, bind, and/or infect a target (ligand or cell) of the reference wild-type virus from which the viral particle is derived. Pseudotyping according to the methods described herein leads to the ability of the viral particles to target cells comprising a TCR, e.g., an antigen-specific TCR.

“Retargeting” or “redirecting” may include a scenario in which the wildtype vector targets several cells within a tissue and/or several organs within an organism, which general targeting of the tissue or organs is reduced to abolished by insertion of the heterologous epitope, and which retargeting to more a specific cell in the tissue or a specific organ in the organism is achieved with the targeting ligand that binds a marker expressed by the specific cell. Such retargeting or redirecting may also include a scenario in which the wildtype vector targets a tissue, which targeting of the tissue is reduced to abolished by insertion of the heterologous epitope, and which retargeting to a completely different tissue is achieved with the targeting ligand.

The terms “viral element” and “viral component” are used herein to refer to viral genes (e.g., genes encoding polymerase or structural proteins) or other elements of the viral genome (e.g., packaging signals, regulatory elements, LTRs, ITRs, etc.).

The term “fusogen” or “fusogenic molecule” is used herein to refer to any molecule that can trigger membrane fusion when present on the surface of a virus particle. A fusogen can be, for example, a protein (e.g., a viral glycoprotein) or a fragment, mutant or derivative thereof.

The term “T cell” is used herein in its broadest sense to refer to all types of immune cells expressing CD3, including T-helper cells (CD4+ cells), cytotoxic T-cells (CD8+ cells), T-regulatory cells (Treg), and NK-T cells.

Terms “antibody” and “antibodies” refer to monoclonal antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “specifically binds,” “binds in a specific manner,” “antigen-specific” or the like, indicates that the molecules involved in the specific binding are able to form a complex with each other that is relatively stable under physiological conditions, and are unable to form stable complexes non-specifically with other molecules outside the specified binding pair. Accordingly, a TCR-binding molecule that binds in a specific manner to an antigen-specific TCR indicates not only that the TCR-binding molecule forms a stable complex with the antigen-specific TCR, but also the antigen-specific TCR forms a stable complex with a distinct antigen. Accordingly, a TCR-binding molecule that binds in a specific manner to an antigen-specific TCR may be considered the antigen to which the TCR is specific, e.g., the TCR-binding molecule (i) does not target the constant domain(s) of a TCR or other components of a TCR complex (e.g., CD3) at all, (ii) targets the constant domain(s) of a TCR or other components of a TCR complex (e.g., CD3) in addition to targeting the variable domain(s) and/or idiotype of a TCR, or (iii) solely targets the variable domain(s) and/or idiotype of a TCR. Specific binding can be characterized by an equilibrium dissociation constant (K_{D}) of about 3000 nM or less (i.e., a smaller K_{D} denotes a tighter binding), about 2000 nM or less, about 1000 nM or less; about 500 nM or less; about 300 nM or less; about 200 nM or less; about 100 nM or less; about 50 nM or less; about 1 nM or less; or about 0.5 nM or less. Methods for determining whether two molecules specifically bind are well known in the art and include, for example, equilibrium dialysis, surface plasmon resonance, and the like.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

An “individual” or “subject” or “animal” refers to humans, veterinary animals (e.g., cats, dogs, cows, horses, sheep, pigs, etc.) and experimental animal models of diseases (e.g., mice, rats).

In a preferred embodiment, the subject is a human.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid” and “nucleotide” encompass both DNA and RNA unless specified otherwise.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

In accordance with the disclosure herein, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

### Viral Particles

In one aspect, described herein is a recombinant viral particle that is capable of binding to a T cell receptor (TCR), the recombinant viral particle comprising on its surface (e.g., in a lipid envelope or capsid) (i) a TCR-binding molecule, and optionally (ii) a fusogen. The viral particle may be derived from an enveloped virus or a non-enveloped virus.

In some embodiments, the TCR-binding molecule comprises a TCR-specific antibody, or portion thereof.

In another embodiment, the TCR-binding molecule comprises a peptide presented in the context of a major histocompatibility complex (MHC) molecule, e.g., is an antigenic determinant associated with(in) a peptide binding groove of an MHC. In some embodiments, a recombinant viral particle described herein is capable of binding to an antigen-specific T-cell receptor (TCR), the recombinant viral particle comprising a lipid envelope comprising (i) a peptide (p) presented in the context of a major histocompatibility complex (MHC) molecule, i.e., a pMHC complex, and (ii) a fusogen, wherein the antigen-specific TCR specifically binds the pMHC complex. In one specific embodiment, the pMHC complex and/or the fusogen comprises a transmembrane domain embedded in the lipid envelope of an enveloped virus.

In some embodiments, the TCR-binding molecule, e.g., an anti-TCR antibody (or portion thereof) or a peptide-major histocompatibility complex (pMHC), is expressed on the outer surface of a virus capsid of a non-enveloped virus. A virus capsid comprises one or more capsid proteins. The term “capsid protein” includes a protein that is part of the capsid of a virus.

In some embodiments, the viral particle described herein is capable of binding to the antigen-specific TCR on a surface of a cell.

In some embodiments, the viral particle is capable of binding and/or infecting the TCR-expressing cell only in the presence of the pMHC complex on its surface.

In some embodiments, one or more of the proteins present in the lipid envelope of a reference wild-type virus corresponding to the recombinant viral particle are absent or mutated so that said recombinant viral particle is not capable of binding to any of the target cells of the reference wild-type virus in the absence of the TCR-binding molecule. In one specific embodiment, all of the proteins which are normally present in the lipid envelope of a reference wild-type virus are absent in a viral particle as disclosed herein.

In some embodiments, the viral particle is replication deficient.

In some embodiments, the viral particle further comprises a cytotoxic agent. In one specific embodiment, the cytotoxic agent is a toxin or a radioactive isotope (e.g., a radioconjugate) or a suicide gene. Non-limiting examples of toxins include, e.g., enzymatically active toxins of bacterial, fungal, plant, or animal origin, or fragments, mutants or derivatives thereof (e.g., diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. Non-limiting examples of suicide genes include, e.g., thymidine kinase, cytosine deaminase, purine nucleoside phosphorylase, nitroreductase, β-galactosidase, hepatic cytochrome β450-2P1, linamarase, horseradish peroxidase, and carboxypeptidase.

In some embodiments, the viral particle further comprises a nucleotide sequence of interest, e.g., one or more transfer vectors comprising the nucleotide sequence of interest and/or an RNA molecule(s), which may be encoded by the transfer vector, wherein said transfer vector or RNA molecule may optionally further comprise at least one viral element. In one specific embodiment, upon infection of the TCR-expressing cell with the viral particle, the nucleotide sequence of interest becomes integrated into the cell genome. In another specific embodiment, upon infection of the TCR-expressing cell with the viral particle, the nucleotide sequence of interest does not become integrated into the cell genome.

The viral particles described herein can be derived from any enveloped or non-enveloped virus. Non-limiting examples of enveloped viruses from which the viral particles described herein can be derived include, e.g., retroviruses (e.g., rous sarcoma virus, human and bovine T-cell leukaemia virus (HTLV and BLV)), lentiviruses (e.g., human and simian immunodeficiency viruses (HIV and SIV), Mason-Pfizer monkey virus), foamy viruses (e.g., Human Foamy Virus (HFV)), herpes viruses (herpes simplex virus (HSV), varicella-zoster virus, VZVEBV, HCMV, HHV), hantaviruses, pox viruses (e.g., vertebrate and avian poxviruses, vaccinia viruses), orthomyxoviruses (e.g., influenza A, influenza B, influenza C viruses), paramyxoviruses (e.g., parainfluenza virus, respiratory syncytial virus, Sendai virus, mumps virus, measles and measles-like viruses), rhabdoviruses (e.g., vesicular stomatitis virus, rubella virus, rabies virus), coronaviruses (e.g., SARS, MERS), flaviviruses (e.g., Marburg virus, Reston virus, Ebola virus), alphaviruses (e.g., Sindbis virus), bunyaviruses, arenaviruses (e.g., LCMV, GTOV, JUNV, LASV, LUJV, MACV, SABV, WWAV), iridoviruses, and hepadnaviruses.

Non-limiting examples of non-enveloped viruses from which the viral particles described herein can be derived include, viruses from the families Picornaviridae, Reoviridae, Caliciviridae, Adenoviridae and Parvoviridae, such as calicivirus, picornavirus, astrovirus, adenovirus, adeno-associated virus. reovirus, polyomavirus, papillomavirus, parvovirus (e.g., adeno-associated virus (AAV)), and type E Hepatitis virus.

In some embodiments, the viral particle is derived from a lentivirus or a retrovirus. Compared to other gene transfer systems, lentiviral and retroviral vectors offer a wide range of advantages, including their ability to transduce a variety of cell types, to stably integrate transferred genetic material into the genome of the targeted host cell, and to express the transduced gene at significant levels. Vectors derived from the gamma-retroviruses, for example, the murine leukemia virus (MLV), have been used in clinical gene therapy trials (Ross et al., Hum. Gen Ther. 7:1781-1790, 1996).

In one specific embodiment, the at least one viral element associated with a nucleotide of interest is a retroviral element. In another specific embodiment, the at least one viral element associated with a nucleotide of interest is a lentiviral element. In one specific embodiment, the at least one viral element associated with a nucleotide of interest is a Psi (ψ) packaging signal. In one specific embodiment, in addition to a Psi (ψ) packaging signal, the viral element further comprises a 5′ Long Terminal Repeat (LTR) and/or a 3′ LTR, or a derivative or mutant thereof. In one specific embodiment, the at least one viral element associated with a nucleotide of interest is selected from the group consisting of a 5′ Long Terminal Repeat (LTR), a Psi (ψ) packaging signal, a Rev Response Element (RRE), a promoter that drives expression of the nucleotide sequence of interest, a Central Polypurine Tract (cPPT), a Woodchuck hepatitis virus post-transcriptional regulatory element (WPRE), a Unique 3′ (U3), a Repeat (R) region, a Unique 5′ (U5), a 3′ LTR, a 3′LTR with the U3 element deleted (e.g., to make the lentivirus non-replicative), a Trans-activating response element (TAR), and any combination thereof.

In some embodiments, the viral particle described herein is derived from a virus of the family Retroviridae. In one specific embodiment, the viral particle described herein is a retroviral particle. In another specific embodiment, the viral particle described herein is a lentiviral particle. In one specific embodiment, the lentiviral particle does not contain gp120 surface envelope protein and/or gp41 transmembrane envelope protein. In another specific embodiment, the lentiviral particle contains a mutant gp120 surface envelope protein and/or a mutant gp41 transmembrane envelope protein and wherein said lentiviral particle is not capable of binding to the cell in the absence of the TCR. In some embodiments, the lentiviral particle contains a wild-type gp120 surface envelope protein and/or a wild-type gp41 transmembrane envelope protein. In other embodiments, the lentivirual particle contains a wild type gp120 surface envelope protein and/or mutant gp41 transmembrane envelope protein.

In some embodiments, the viral particle described herein comprises components from a virus selected from the group consisting of Human Immunodeficiency Virus (e.g., HIV-1 or HIV-2), Bovine Immunodeficiency Virus (BIV), Feline Immunodeficiency Virus (FIV), Simian Immunodeficiency Virus (SIV), Equine Infectious Anemia Virus (EIAV), Murine Stem Cell Virus (MSCV), Murine Leukemia Virus (MLV), Avian leukosis virus (ALV), Feline leukemia virus (FLV), Bovine leukemia virus (BLV), Human T-lymphotropic virus (HTLV), feline sarcoma virus, avian reticuloendotheliosis virus, caprine arthritis encephalitis virus (CAEV), and Visna-Maedi virus (VMV).

In some embodiments, the viral particles described herein are replication deficient and only contain an incomplete genome of the virus from which they are derived. For example, in some embodiments, the lentiviral and retroviral particles do not comprise the genetic information of the gag, env, or pol genes (which may be involved in the assembly of the viral particle), which is a known minimal requirement for successful replication of a lentivirus or retrovirus. In these cases, the minimal set of viral proteins needed to assemble the vector particle are provided in trans by means of a packaging cell line. In one specific embodiment, for lentiviral particles derived from HIV-1, env, tat, vif, vpu and nef genes are lacking and are not provided in trans or are made inactive by the use of frame shift mutation(s).

In some embodiments, the RNA molecule incorporated into the lentiviral or retroviral particles comprises the psi packaging signal and LTRs. In some embodiments, the RNA molecule incorporated into the lentiviral or retroviral particles comprises a nucleotide sequence of interest. To achieve expression of such nucleotide sequence of interest in the TCR-expressing cell, such sequence is usually placed under the control of a suitable promoter, for example, the CMV promoter.

In some embodiments of lentiviral and retroviral particles, RNA molecule together with the gag and pol encoded proteins, provided in trans by the packaging cell line, are then assembled into the vector particles, which then infect their target TCR-expressing cells, reverse-transcribe the RNA molecule that may comprise a nucleotide sequence of interest under the control of a promoter, and either integrate said genetic information into the genome of the target cells or remain episomal (if one or more of the components required for integration are disrupted). If the genetic information for the gag and pol encoded proteins is not present on the transduced RNA molecule, the vector particles are replication deficient, i.e., no new generation of said vector particles will thus be generated by the transduced cell, thus ensuring safety in clinical applications.

In some embodiments, the viral particle is derived from an adeno-associated virus (AAV). In some embodiments, a recombinant viral particle described herein is derived from an adeno-associated virus (AAV), e.g., an AAV serotype selected from the group consisting of AAV1, AAV2, AAV3, AAV4, AAV5, AAV6, AAV7, AAV8 and AAV9, a hybrid of some of those serotypes (e.g. AAV-DJ), or a genetically modified variant thereof.

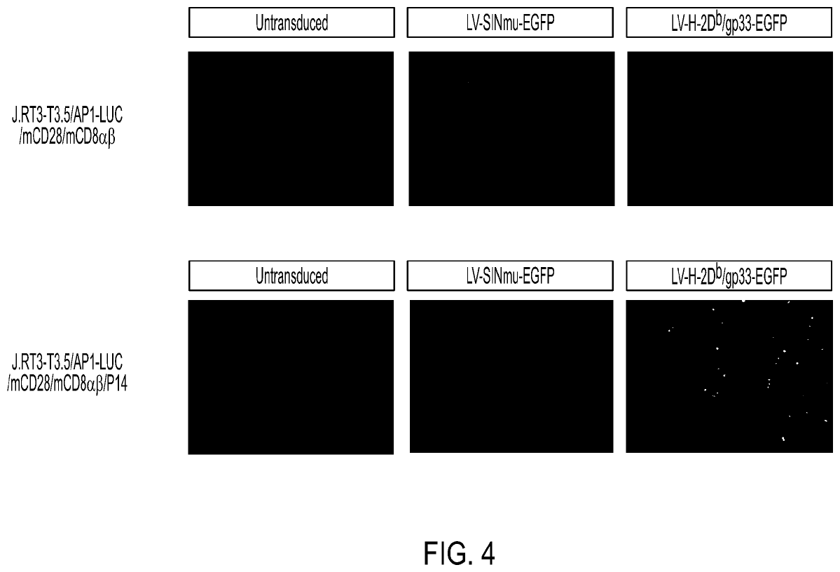

Adeno-associated viruses comprise three capsid proteins generally referred to as VP1, VP2 and/or VP3, and each are encoded by a single cap gene. For AAV, the three AAV capsid proteins are produced in an overlapping fashion from the cap open reading frame (ORF) via alternative mRNA splicing and/or alternative translational start codon usage, although all three proteins use a common stop codon. Warrington et al. (2004) J. Virol. 78:6595, incorporated herein by reference in its entirety. VP1 of AAV2 is generally translated from an ATG start codon (amino acid M1) on a 2.4-kb mRNA, while VP2 and VP3 of AAV2 arise from a smaller 2.3-kb mRNA, using a weaker ACG start codon for VP2 production (amino acid T138) and readthrough translation to the next available ATG codon (amino acid M203) for the production of the most abundant capsid protein, VP3. Warrington, supra; Rutledge et al. (1998) J. Virol. 72:309-19, incorporated herein by reference in its entirety. The amino acid sequences of capsid proteins of adeno-associated viruses are well-known in the art and generally conserved, particularly upon the dependoparvoviruses. See, Rutledge et al., supra. For example, Rutledge et al. (1998), supra, provides at FIG. 4B amino acid sequence alignments for VP1, VP2, and VP3 capsid proteins of AAV2, AAV3, AAV4 and AAV6, wherein the start sites for each of the VP1, VP2, and VP3 capsid proteins are indicated by arrows and the variable domains are boxed. Accordingly, although amino acid positions provided herein may be provided in relation to the VP1 capsid protein of the AAV, a skilled artisan would be able to respectively and readily determine the position of that same amino acid within the VP2 and/or VP3 capsid protein of the AAV, and the corresponding position of amino acids among different serotypes. Additionally, a skilled artisan would be able to swap domains between capsid proteins of a different AAV serotypes for the formation of a “chimeric capsid protein.”

Domain swapping between two AAV capsid protein constructs for the generation of a “chimeric AAV capsid protein” has been described, see, e.g., Shen et al. (2007) Mol. Therapy 15(11):1955-1962, incorporated herein in its entirety by reference. A “chimeric AAV capsid protein” includes an AAV capsid protein that comprises amino acid sequences, e.g., domains, from two or more different AAV serotypes and that is capable of forming and/or forms an AAV-like viral capsid/viral particle. A chimeric AAV capsid protein is encoded by a chimeric AAV capsid gene, e.g., a nucleotide comprising a plurality, e.g., at least two, nucleic acid sequences, each of which plurality is identical to a portion of a capsid gene encoding a capsid protein of distinct AAV serotypes, and which plurality together encodes a functional chimeric AAV capsid protein. Reference to a chimeric capsid protein in relation to a specific AAV serotype indicates that the capsid protein comprises one or more domains from a capsid protein of that serotype and one or more domains from a capsid protein of a different serotype. For example, an AAV2 chimeric capsid protein includes a capsid protein comprising one or more domains of an AAV2 VP1, VP2, and/or VP3 capsid protein and one or more domains of a VP1, VP2, and/or VP3 capsid protein of a different AAV.

A “mosaic capsid” comprises at least two sets of VP1, VP2, and/or VP3 proteins, each set of which is encoded by a different cap gene.

In some embodiments, a mosaic capsid described herein comprises recombinant VP1, VP2, and/or VP3 proteins encoded by a cap gene genetically modified with an insertion of a nucleic acid sequence encoding a heterologous epitope, and further comprises VP1, VP2, and/or VP3 proteins encoded by a reference cap gene, e.g., a wildtype reference cap gene encoding the wildtype VP1, VP2, and/or VP3 proteins of the same AAV serotype as the recombinant VP1, VP2, and/or VP3 proteins, a control reference cap gene encoding VP1, VP2, and/or VP3 proteins identical to the recombinant VP1, VP2, and VP3 proteins but for the absence of the heterologous epitope, a mutated wildtype reference cap gene encoding substantially wildtype VP1, VP2, and/or VP3 proteins of the same AAV serotype as the recombinant VP1, VP2, and/or VP3 proteins but for a mutation (e.g., insertion, substitution, deletion), which mutation preferably reduces the tropism of the wildtype VP1, VP2, and VP3 proteins. In some embodiments, the reference capsid protein is a chimeric reference protein comprising at least one domain of VP1, VP2, and/or VP3 proteins of the same AAV serotype as the recombinant VP1, VP2, and/or VP3 proteins. In some embodiments, the reference cap gene encodes a chimeric VP1, VP2, and/or VP3 protein.

The term “recombinant capsid protein” includes a capsid protein that has at least one mutation in comparison to the corresponding capsid protein of the wild-type virus, which may be a reference and/or control virus for comparative study. A recombinant capsid protein includes a capsid protein that comprises a heterologous epitope, which may be inserted into and/or displayed by the capsid protein. “Heterologous” in this context means heterologous as compared to the virus, from which the capsid protein is derived. The inserted amino acids can simply be inserted between two given amino acids of the capsid protein. An insertion of amino acids can also go along with a deletion of given amino acids of the capsid protein at the site of insertion, e.g. 1 or more capsid protein amino acids are substituted by 5 or more heterologous amino acids).

AAVs comprise symmetrical nucleic acid sequences in the genome, known as “Inverted terminal repeat” or “ITR”, for efficient replication. ITR sequences are located at each end of the AAV DNA genome. The ITRs serve as the origins of replication for viral DNA synthesis and are essential cis components for generating AAV integrating vectors.

Generally, a recombinant viral particle as described herein comprises a heterologous epitope inserted into and/or displayed by a capsid protein such that the heterologous epitope reduces and/or abolishes the natural tropism of the capsid protein or capsid comprising same. In some embodiments, the heterologous epitope is inserted into a region of the capsid protein involved with the natural tropism of the wildtype reference capsid protein, e.g., a region of the capsid protein involved with cell receptor. In some embodiments, the heterologous epitope is inserted into and/or displayed by a knob domain of an Ad fiber protein. In some embodiments, the heterologous epitope is inserted into and/or displayed by the HI loop of an Ad fiber protein. In some embodiments, the heterologous epitope is inserted after an amino acid position selected from the group consisting of G453 of AAV2 capsid protein VP1, N587 of AAV2 capsid protein VP1, Q585 of AAV6 capsid protein VP1, G453 of AAV9 capsid protein VP1, and A589 of AAV9 capsid protein VP1. In some embodiments, the heterologous epitope is inserted and/or displayed between amino acids N587 and R588 of an AAV2 VP1 capsid. Additional suitable insertion sites identified by using AAV2 are well known in the art (Wu et al. (2000) J. Virol. 74:8635-8647) and include I-1, I-34, I-138, I-139, I-161, I-261, I-266, I-381, I-447, I-448, I-459, I-471, I-520, I-534, 1-570, I-573, I-584, I-587, I-588, I-591, I-657, I-664, I-713 and I-716. A recombinant virus capsid protein as described herein may be an AAV2 capsid protein comprising a heterologous epitope inserted into a position selected from the group consisting of I-1, I-34, I-138, I-139, I-161, I-261, 1-266, I-381, I-447, I-448, I-459, I-471, I-520, I-534, I-570, I-573, I-584, I-587, I-588, I-591, I-657, I-664, I-713, I-716, and a combination thereof. Additional suitable insertion sites identified by using additional AAV serotypes are well-known and include I-587 (AAV1), 1-589 (AAV1), I-585 (AAV3), 1-585 (AAV4), and I-585 (AAV5). In some embodiments, a recombinant virus capsid protein as described herein may be an AAV2 capsid protein comprising a heterologous epitope inserted into a position selected from the group consisting of I-587 (AAV1), I-589 (AAV1), I-585 (AAV3), 1-585 (AAV4), I-585 (AAV5), and a combination thereof.

The used nomenclature I-### herein refers to the insertion site with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and mutations thereof) may be easily identified for the VP2 and VP3 capsid proteins encoding by the capsid gene by performing a sequence alignment of the VP1, VP2 and VP3 proteins encoding by the reference AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Additionally, due to the high conservation of at least large stretches and the large member of closely related family member, the corresponding insertion sites for AAV other than the enumerated AAV can be identified by performing an amino acid alignment or by comparison of the capsid structures. See, e.g., Rutledge et al. (1998) J. Virol. 72:309-19 and U.S. Pat. No. 9,624,274 for exemplary alignments of different AAV capsid proteins, each of which reference is incorporated herein by reference in its entirety.

In some embodiments of the recombinant viral particle described herein, the recombinant viral particle comprises an AAV2 capsid protein VP1 with a heterologous epitope is inserted at an 1587 site, wherein the heterologous epitope does not comprise an Arg-Gly-Asp (RGD) motif, an NGR motif, or c-myc. In some compositions of the recombinant viral particle described herein, the recombinant viral particle comprises a VP1 capsid protein with a heterologous epitope is inserted between T448 and N449, wherein the heterologous epitope does not comprise c-myc. In some compositions of the recombinant viral particle described herein, the recombinant viral particle comprises a VP1 capsid protein with a heterologous epitope is inserted at an 1-447 site, wherein the heterologous epitope does not comprise L14 or HA.

In some embodiments of the recombinant viral particle described herein, the recombinant viral particle comprises a VP1 capsid protein with a heterologous epitope is inserted at an 1587 site, wherein the heterologous epitope comprises an Arg-Gly-Asp (RGD) motif, an NGR motif, or c-myc. In some embodiments of the recombinant viral particle described herein, the viral capsid is a VP1 capsid, the heterologous epitope comprises c-myc, and the heterologous epitope is inserted between T448 and N449, or between N587 and R588. In some embodiments of the recombinant viral particle described herein, the recombinant viral particle comprises a VP1 capsid protein with a heterologous epitope inserted at an I-447 site, wherein the heterologous epitope comprises L14 or HA. In some embodiments of the recombinant viral particle described herein, the recombinant viral particle comprises a VP1 capsid protein with a heterologous epitope inserted between T448 and N449, wherein the heterologous epitope comprises c-myc. U.S. Pat. No. 9,624,274 describes I-453 of an AAV capsid protein as a suitable insertion site for a heterologous epitope.

In some embodiments, insertion (display) of the heterologous epitope abolishes the natural tropism of the viral vector, e.g., transduction of a cell naturally permissive to infection by wildtype reference viral vectors and/or a target cell is undetectable in the absence of an appropriate binding molecule. In some embodiments, insertion (display) of the heterologous epitope reduces the natural tropism of the viral vector, e.g., compared to transduction of a cell naturally permissive to infection by wildtype reference viral vectors. In some embodiments, the insertion (display) of the heterologous epitope reduces the natural tropism of the viral vector by at least 5%. In some embodiments, the insertion (display) of the heterologous epitope reduces the natural tropism of the viral vector by at least 5%. In some embodiments, the insertion (display) of the heterologous epitope reduces the natural tropism of the viral vector by at least 10%. In some embodiments, the insertion (display) of the heterologous epitope reduces the natural tropism of the viral vector by at least 20%. In some embodiments, the insertion (display) of the heterologous epitope reduces the natural tropism of the viral vector by at least 30%. In some embodiments, the insertion (display) of the heterologous epitope reduces the natural tropism of the viral vector by at least 40%. In some embodiments, the insertion (display) of the heterologous epitope reduces the natural tropism of the viral vector by at least 50%. In some embodiments, the insertion (display) of the heterologous epitope reduces the natural tropism of the viral vector by at least 60%. In some embodiments, the insertion (display) of the heterologous epitope reduces the natural tropism of the viral vector by at least 70%. In some embodiments, the insertion (display) of the heterologous epitope reduces the natural tropism of the viral vector by at least 80%. In some embodiments, the insertion (display) of the heterologous epitope reduces the natural tropism of the viral vector by at least 90%. In some embodiments, the insertion (display) of the heterologous epitope reduces the natural tropism of the viral vector by at least 95%. In some embodiments, the insertion (display) of the heterologous epitope reduces the natural tropism of the viral vector by at least 90%. In these embodiments, wherein the insertion (display) of the heterologous epitope does not abolish the natural tropism of the recombinant viral capsids, the natural tropism of such recombinant viral capsids may be abolished by a second and different mutation. For example, in one embodiment, a recombinant viral capsid protein as described herein may be derived from an AAV9 capsid gene, comprise a heterologous epitope, and may further comprise a mutation, e.g., a W503A mutation. Other non-limiting examples of a second mutation include, e.g., Y445F and V473D for AAV1 or AAV6 capsid.

This detargeting of the virus from its natural host cell is important especially if systemic versus local or loco-regional administration of the viral vectors is intended, as uptake of the viral vectors by the natural host cells limits the effective dose of the viral vectors. In case of AAV2 and AAV6 HSPG is reported to be the primary receptor for viral uptake in a large number of cells, especially liver cells. For AAV2 HSPG-binding activity is dependent on a group of 5 basic amino acids, R484, R487, R585, R588 and K532 (Kern et al., (2003) J Virol. 77(20):11072-81). Recently it was reported that the lysine-to-glutamate amino acid substitution K531E leads to the suppression of AAV6's ability to bind heparin or HSPG ((Wu et al., 2006) J. of Virology 80(22):11393-11397). Accordingly, preferred point mutations are those that reduce the transducing activity of the viral vector for a given target cell mediated by the natural receptor by at least 50%, preferably at least 80%, especially at least 95%, in case of HSPG as primary receptor the binding of the viral vectors to HSPG.

Consequently, further mutations preferred for HSPG-binding viral vectors are those mutations that deplete or replace a basic amino acid such as R, K or H, preferably R or K which is involved in HSPG binding of the respective virus, by a non-basic amino acid such as A, D, G, Q, S and T, preferably A or an amino acid that is present at the corresponding position of a different but highly conserved AAV serotype lacking such basic amino acid at this position. Consequently, preferred amino acid substitutions are R484A, R487A, R487G, K532A, K532D, R585A, R585S, R585Q, R585A or R588T, especially R585A and/or R588A for AAV2, and K531A or K531E for AAV6. One especially preferred embodiment of the invention are such capsid protein mutants of AAV2 that additionally contain the two point mutations R585A and R588A as these two point mutations are sufficient to ablate HSPG binding activity to a large extent. These point mutations enable an efficient detargeting from HSPG-expressing cells which—for targeting purposes—increases specificity of the respective mutant virus for its new target cell.

A recombinant viral particle of the present disclosure can include a capsid protein or viral capsid comprising a heterologous epitope, which enables the retargeting of the viral vector, e.g., via a TCR-binding molecule.

In some embodiments, a recombinant viral capsid as described herein comprises the amino acid sequence EQKLISEEDL flanked by and/or operably linked to at least 5 contiguous amino acids of an AAV VP1 capsid protein. In some embodiments, a recombinant vital capsid as described herein comprises the amino acid sequence EQKLISEEDL flanked by and/or operably linked to at least 5 contiguous amino acids of an AAV2 VP1 capsid protein. In some embodiments, a recombinant viral capsid as described herein comprises EQKLISEEDL inserted between N587 and R588 of an AAV2 VP1 capsid protein.

In some embodiments, the heterologous epitope comprises an affinity tag and one or more linkers. In some embodiments, the heterologous epitope comprises an affinity tag flanked by a linker, e.g., the heterologous epitope comprises from N-terminus to C-terminus a first linker, an affinity tag, and a second linker. In some embodiments, the first and second linkers are each independently at least one amino acid in length. In some embodiments, the first and second linkers are identical.

Generally, a heterologous epitope as described herein, e.g., an affinity tag by itself or in combination with one or more linkers, is between about 5 amino acids to about 35 amino acids in length. In some embodiments, the heterologous epitope (by itself or in combination with one or more linkers) is at least 5 amino acids in length. In some embodiments, the heterologous epitope (by itself or in combination with one or more linkers) is 6 amino acids in length. In some embodiments, the heterologous epitope (by itself or in combination with one or more linkers) is 7 amino acids in length. In some embodiments, the heterologous epitope (by itself or in combination with one or more linkers) is 8 amino acids in length. In some embodiments, the heterologous epitope (by itself or in combination with one or more linkers) is 9 amino acids in length. In some embodiments, the heterologous epitope (by itself or in combination with one or more linkers) is 10 amino acids in length. In some embodiments, the heterologous epitope (by itself or in combination with one or more linkers) is 11 amino acids in length. In some embodiments, the heterologous epitope (by itself or in combination with one or more linkers) is 12 amino acids in length. In some embodiments, the heterologous epitope (by itself or in combination with one or more linkers) is 13 amino acids in length. In some embodiments, the heterologous epitope (by itself or in combination with one or more linkers) is 14 amino acids in length. In some embodiments, the heterologous epitope (by itself or in combination with one or more linkers) is 15 amino acids in length. In some embodiments, the heterologous epitope (by itself or in combination with one or more linkers) is 16 amino acids in length. In some embodiments, the heterologous epitope (by itself or in combination with one or more linkers) is 17 amino acids in length. In some embodiments, the heterologous epitope (by itself or in combination with one or more linkers) is 18 amino acids in length. In some embodiments, the heterologous epitope (by itself or in combination with one or more linkers) is 19 amino acids in length. In some embodiments, the heterologous epitope (by itself or in combination with one or more linkers) is 20 amino acids in length. In some embodiments, the heterologous epitope (by itself or in combination with one or more linkers) is 21 amino acids in length. In some embodiments, the heterologous epitope (by itself or in combination with one or more linkers) is 22 amino acids in length. In some embodiments, the heterologous epitope (by itself or in combination with one or more linkers) is 23 amino acids in length. In some embodiments, the heterologous epitope (by itself or in combination with one or more linkers) is 24 amino acids in length. In some embodiments, the heterologous epitope (by itself or in combination with one or more linkers) is 25 amino acids in length. In some embodiments, the heterologous epitope (by itself or in combination with one or more linkers) is 26 amino acids in length. In some embodiments, the heterologous epitope (by itself or in combination with one or more linkers) is 27 amino acids in length. In some embodiments, the heterologous epitope (by itself or in combination with one or more linkers) is 28 amino acids in length. In some embodiments, the heterologous epitope (by itself or in combination with one or more linkers) is 29 amino acids in length. In some embodiments, the heterologous epitope (by itself or in combination with one or more linkers) is 30 amino acids in length. In some embodiments, the heterologous epitope (by itself or in combination with one or more linkers) is 31 amino acids in length. In some embodiments, the heterologous epitope (by itself or in combination with one or more linkers) is 32 amino acids in length. In some embodiments, the heterologous epitope (by itself or in combination with one or more linkers) is 33 amino acids in length. In some embodiments, the heterologous epitope (by itself or in combination with one or more linkers) is 34 amino acids in length. In some embodiments, the heterologous epitope (by itself or in combination with one or more linkers) is 35 amino acids in length.

In various embodiments, a viral vector as described herein has reduced to abolished transduction capabilities in the absence of a binding molecule, specifically a TCR-binding molecule.

In a related aspect, described herein is a method of producing a viral particle that is capable of binding to a TCR, said method comprising culturing a packaging cell in conditions sufficient for the production of a plurality of viral particles, wherein the packaging cell comprises one or more plasmids comprising (i) one or more viral elements involved in the assembly of the viral particle, (ii) a nucleotide sequence encoding a TCR-binding molecule and, optionally, (iii) a nucleotide sequence encoding a fusogen. In some embodiments, the nucleotide sequence (ii) encodes a TCR-specific antibody comprising the TCR-binding molecule.

In some embodiments, described herein is a method of producing a viral particle that is capable of binding to an antigen-specific TCR, said method comprising culturing a packaging cell in conditions sufficient for the production of a plurality of viral particles, wherein the packaging cell comprises one or more plasmids comprising (i) one or more viral elements involved in the assembly of the viral particle, (ii) a nucleotide sequence encoding a peptide, e.g., an antigenic determinant, (iii) one or more nucleotide sequence(s) encoding a major histocompatibility complex (MHC) molecule, and optionally (iv) a nucleotide sequence encoding a fusogen, wherein the viral particles present the antigen-derived peptide in the context of the MHC.

In some embodiments of any of the above methods, the method further comprises collecting the viral particles. In one specific embodiment, the collecting step comprises one or more of the following steps: clearing cell debris, treating the supernatant containing viral particles with DNase I and MgCl₂, concentrating viral particles, and purifying the viral particles.

In some embodiments of any of the above methods, the packaging cell further comprises one or more transfer vectors or a RNA molecule(s) encoded by the transfer vector, wherein said transfer vector or RNA molecule comprises at least one viral element and a nucleotide sequence of interest and, wherein the viral particles comprise said transfer vector(s) or RNA molecule(s). In one specific embodiment, the at least one viral element is a retroviral element. In another specific embodiment, the at least one viral element is a lentiviral element. In one specific embodiment, the at least one viral element is a Psi (ψ) packaging signal. In one specific embodiment, in addition to a Psi (ψ) packaging signal, the viral element further comprises a 5′ Long Terminal Repeat (LTR) and/or a 3′ LTR, or a derivative or mutant thereof. In one specific embodiment, the at least one viral element is selected from the group consisting of a 5′ Long Terminal Repeat (LTR), a Psi (ψ) packaging signal, a Rev Response Element (RRE), a promoter that drives expression of the nucleotide sequence of interest, a Central Polypurine Tract (cPPT), a Woodchuck hepatitis virus post-transcriptional regulatory element (WPRE), a Unique 3′ (U3), a Repeat (R) region, a Unique 5′ (U5), a 3′ LTR, a 3′LTR with the U3 element deleted (e.g., to make the lentivirus non-replicative), a Trans-activating response element (TAR), and any combination thereof.

In some embodiments of any of the above methods, the one or more plasmids comprises (a) GAG, (b) POL, and (c) TAT and/or REV lentiviral or retroviral elements, each of which may be considered involved with the assembly of the viral particle.

In some embodiments of any of the above methods, the plasmids present in the packaging cell do not comprise a lentiviral or retroviral ENV gene or comprise a mutant non-functional ENV gene. In another embodiment, the one or more plasmids comprise a mutant lentiviral ENV gene, which does not produce gp120 surface envelope protein or gp41 transmembrane envelope or which encodes a mutant gp120 surface envelope protein and/or a mutant gp41 transmembrane envelope protein and wherein the resulting viral particle is not capable of binding to a target cell in the absence of the TCR-binding molecule.

In some embodiments of any of the above methods, the one or more viral elements encodes Human Immunodeficiency Virus (HIV) component(s), Bovine Immunodeficiency Virus (BIV) component(s), Feline Immunodeficiency Virus (FIV) component(s), Simian Immunodeficiency Virus (SIV) component(s), Equine Infectious Anemia Virus (EIAV) component(s), Murine Stem Cell Virus (MSCV) component(s), Murine Leukemia Virus (MLV) component(s), Avian leukosis virus (ALV) component(s), Feline leukemia virus (FLV) component(s), Bovine leukemia virus (BLV) component(s), Human T-lymphotropic virus (HTLV) component(s), feline sarcoma virus component(s), avian reticuloendotheliosis virus component(s), caprine arthritis encephalitis virus (CAEV) component(s), and/or Visna-Maedi virus (VMV) component(s).

In conjunction with the viral particles described herein, described herein are methods for detecting and/or isolating cells, which are bearing an antigen-specific TCR targeted by the TCR-binding molecule located in the envelope of the viral particle. In some embodiments, such method uses viral particles comprising a selectable marker (e.g., neomycin resistance) and/or a reporter (e.g., GFP or eGFP) as a nucleotide sequence of interest allowing target cells to be selected using, e.g., selection compound exposure or fluorescent activated cell sorting (FACS). The TCR-encoding cDNA expressed in the selected cells can be cloned and sequenced.

In the methods described herein, plasmids/vectors used for viral particle production can be introduced into the packaging cells using methods well known in the art such as, e.g., electroporation (using for example Multiporator (Eppendorf), Genepulser (BioRad), MaxCyte Transfection Systems (Maxcyte)), PEI (Polysciences Inc. Warrington, Eppelheim), Ca2+-mediated transfection or via liposomes (for example: “Lipofectamine” (Invitrogen)), non-liposomal compounds (for example: “Fugene” (Roche) or nucleofection (Lonza)) into cells.

In some embodiment, the viral particles described herein are derived from murine leukaemia virus (MLV). Retroviral vectors encoding MLV are widely available to those skilled in the art, such as PINCO (Grignani et al., 1998) or the pBabe vector series (Morgenstern and Land, 1990).

In some embodiments, the viral particles described herein are derived from a lentivirus such as human immunodeficiency virus (HIV). Suitable vectors encoding HIV and other useful viruses can be readily identified and/or prepared by the skilled person.

In some embodiments described herein, for generating lentiviral vectors, 2-4 basic components, usually provided on separate plasmids, are used: (i) sequences encoding molecules involved in assembly of the lentiviral particle (e.g., a psi-negative gag/pol gene) provided on an packaging plasmid, (ii) sequences encoding a TCR-binding molecule to replace the viral env protein (or as a fusion with the viral env protein) provided on an envelope expression plasmid, optionally (iii) a nucleic acid of interest, e.g., provided on a transfer vector together, optionally with one or more viral elements needed for packaging (e.g., psi packaging signal and optionally LTR), or provided as an RNA encoded by the vector, and, optionally, (iv) a sequence encoding a fusogen provided on a fusogen encoding plasmid.

In some embodiments, the transfer vector and its corresponding RNA preferably comprises an inactivated or self-inactivating 3′ LTR. The 3′ LTR may be made self-inactivating by any method known in the art. In the preferred embodiment, the U3 element of the 3′ LTR contains a deletion of its enhancer sequence, preferably the TATA box, Spl and NF-kappa B sites. As a result of the self-inactivating 3′ LTR, the provirus that is integrated into the host cell genome will comprise an inactivated 5′ LTR.

In some embodiments the transfer vector preferably comprises at least one RNA Polymerase II or III promoter. The RNA Polymerase II or III promoter is operably linked to the nucleotide sequence of interest and can also be linked to a termination sequence. RNA polymerase II and III promoters are well known to one of skill in the art. A suitable range of RNA polymerase III promoters can be found, for example, in Paule and White. Nucleic Acids Research., Vol 28, pp 1283-1298 (2000); Ohkawa and Taira Human Gene Therapy, Vol. 11, pp 577-585 (2000); Meissner et al. Nucleic Acids Research, Vol. 29, pp 1672-1682 (2001). Non-limiting examples of useful promoters include, e.g., cytomegalovirus (CMV)-promoter, the spleen focus forming virus (SFFV)-promoter, the elongation factor 1 alpha (EF 1a)-promoter (the 1.2 kb EFla-promoter or the 0.2 kb EFla-promoter), the chimeric EF 1 a/IF4-promoter, and the phospho-glycerate kinase (PGK)-promoter. An internal enhancer may also be present in the viral construct to increase expression of the gene of interest. For example, the CMV enhancer (Karasuyama et al. 1989. J. Exp. Med. 169:13) may be used. In some embodiments, the CMV enhancer can be used in combination with the chicken β-actin promoter. One of skill in the art will be able to select the appropriate enhancer based on the desired expression pattern.

In addition, in some embodiments, the transfer vector and the corresponding RNA can contain more than one nucleotide sequence of interest.

The constructs described herein may also contain additional genetic elements. The types of elements that may be included in the constructs are not limited in any way and will be chosen by the skilled practitioner to achieve a particular result. For example, a signal that facilitates nuclear entry of the RNA corresponding to the transfer vector in the target cell may be included. An example of such a signal is the HIV-1 flap signal.

In addition, transfer vector may contain one or more genetic elements designed to enhance expression of the gene of interest. For example, a woodchuck hepatitis virus responsive element (WRE) may be placed into the construct (Zufferey et al. 1999. J. Virol. 74:3668-3681; Deglon et al. 2000. Hum. Gene Ther. 11:179-190).

A chicken β-globin insulator may also be included in the transfer vector. This element has been shown to reduce the chance of silencing of the integrated provirus in the target cell due to methylation and heterochromatinization effects. In addition, the insulator may shield the internal enhancer, promoter and exogenous gene from positive or negative positional effects from surrounding DNA at the integration site on the chromosome.

In one specific embodiment, the transfer vector comprises: a cytomegalovirus (CMV) enhancer/promoter sequence; the R and U5 sequences from the HIV 5′ LTR; the HIV-1 flap signal; an internal enhancer; an internal promoter; a nucleotide sequence of interest; the woodchuck hepatitis virus responsive element; a tRNA amber suppressor sequence; a U3 element with a deletion of its enhancer sequence; the chicken β-globin insulator; and the R and U5 sequences of the 3′ HIV LTR.

### Packaging Cells and Production of Viral Particles

Also disclosed herein is a packaging cell for producing the viral particles described herein comprising one or more plasmids comprising (i) one or more viral elements involved in assembly of the viral particle, (ii) a nucleotide sequence encoding a TCR-binding molecule and optionally (iii) a nucleotide sequence encoding a fusogen. In some embodiments, the nucleotide sequence (ii) encodes a TCR-specific antibody comprising the TCR-binding molecule. In some embodiments, a packaging cell for producing the viral particles described herein comprises one or more plasmids comprising (i) one or more viral elements involved in assembly of the viral particle, (ii) a nucleotide sequence encoding a peptide (e.g., antigenic determinant), (iii) one or more nucleotide sequence(s) encoding a major histocompatibility complex (MHC) molecule, and optionally (iv) a nucleotide sequence encoding a fusogen. In some embodiments, the packaging cell further comprises one or more transfer vectors, wherein each transfer vector comprises at least one viral element and a nucleotide sequence of interest. In some embodiments, the packaging cell does not comprise any MHCs other than the MHC of the viral particle.

Disclosed herein is a method of producing a viral particle that is capable of binding to a TCR, said method comprising culturing a packaging cell in conditions sufficient for the production of a plurality of viral particles, wherein the packaging cell comprises one or more plasmids comprising (i) one or more viral elements involved in assembly of the viral particle, (ii) a nucleotide sequence encoding a TCR-binding molecule and, optionally, (iii) a nucleotide sequence encoding a fusogen. In some embodiments, the nucleotide sequence (ii) encodes a TCR-specific antibody comprising the TCR-binding molecule. In some embodiments, a method of producing a viral particle that is capable of binding to an antigen-specific TCR is described, said method comprising culturing a packaging cell in conditions sufficient for the production of a plurality of viral particles, wherein the packaging cell comprises one or more plasmids comprising (i) one or more viral elements involved in assembly of the viral particle, (ii) a nucleotide sequence encoding a peptide, e.g., an antigenic determinant, (iii) one or more nucleotide sequence(s) encoding a major histocompatibility complex (MHC) molecule, and optionally (iv) a nucleotide sequence encoding a fusogen, wherein the viral particles present the peptide in the context of the MHC.

In some embodiments of any of the above methods, the method further comprises collecting the viral particles. In one specific embodiment, the collecting step comprises one or more of the following steps: clearing cell debris, treating the supernatant containing viral particles with DNase I and MgCl₂, concentrating viral particles, and purifying the viral particles.

In some embodiments of any of the above methods, the packaging cell further comprises a nucleotide of interest, e.g., one or more transfer vectors or a RNA molecule(s) encoded by the transfer vector comprising the nucleotide of interest, wherein said transfer vector or RNA molecule may optionally further comprise at least one viral element. In one specific embodiment, the at least one viral element is a retroviral element. In another specific embodiment, the at least one viral element is a lentiviral element. In one specific embodiment, the at least one viral element is a Psi (ψ) packaging signal. In one specific embodiment, in addition to a Psi (ψ) packaging signal, the viral element further comprises a 5′ Long Terminal Repeat (LTR) and/or a 3′ LTR, or a derivative or mutant thereof. In one specific embodiment, the at least one viral element is selected from the group consisting of a 5′ Long Terminal Repeat (LTR), a Psi (ψ) packaging signal, a Rev Response Element (RRE), a promoter that drives expression of the nucleotide sequence of interest, a Central Polypurine Tract (cPPT), a Woodchuck hepatitis virus post-transcriptional regulatory element (WPRE), a Unique 3′ (U3), a Repeat (R) region, a Unique 5′ (U5), a 3′ LTR, a 3′LTR with the U3 element deleted (e.g., to make the lentivirus non-replicative), a Trans-activating response element (TAR), and any combination thereof.

In some embodiments of any of the above methods, the one or more plasmids comprises (a) GAG, (b) POL, and (c) TAT and/or REV lentiviral or retroviral elements.

In some embodiments of any of the above methods, the plasmids present in the packaging cell do not comprise a lentiviral or retroviral ENV gene or comprise a mutant non-functional ENV gene. In another embodiment, the one or more plasmids comprise a mutant lentiviral ENV gene, which does not produce gp120 surface envelope protein or gp41 transmembrane envelope or which encodes a mutant gp120 surface envelope protein and/or a mutant gp41 transmembrane envelope protein and wherein the resulting viral particle is not capable of binding to a target cell in the absence of the TCR-binding molecule.

In some embodiments, a viral particle as described herein comprises a mosaic capsid, e.g., a capsid comprising capsid proteins genetically modified as described herein (in the absence or presence of a covalent bond with a targeting ligand) in a certain ratio with reference capsid proteins. Mosaic capsid and methods of making such mosaic viral particles may be find in, for example, WO2020242984, the content of which is incorporated herein by reference in its entirety, and in the Examples sections below. An exemplary method for making such a mosaic viral particle comprises
a) expressing a nucleic acid encoding the recombinant capsid protein and a nucleotide encoding a reference capsid protein at a ratio (wt/wt) of 1:1 and 10:1 under suitable conditions, and
b) isolating the expressed capsid protein of step a).

Generally speaking, a mosaic capsid formed according to the method will be considered to have a modified capsid protein:reference capsid protein ratio similar to the ratio (wt:wt) of nucleic acids encoding same used to produce the mosaic capsid. Accordingly, in some embodiments, a composition described herein comprises, or a method described herein combines, a recombinant viral capsid protein and a reference capsid protein (or combination of reference capsid proteins) at a ratio that ranges from 1:1 to 1:15. In some embodiments, the ratio is 1:2. In some embodiments, the ratio is 1:3. In some embodiments, the ratio is 1:4. In some embodiments, the ratio is 1:5. In some embodiments, the ratio is 1:6. In some embodiments, the ratio is 1:7. In some embodiments, the ratio is 1:8. In some embodiments, the ratio is 1:9. In some embodiments, the ratio is 1:10. In some embodiments, the ratio is 1:11. In some embodiments, the ratio is 1:12. In some embodiments, the ratio is 1:13. In some embodiments, the ratio is 1:14. In some embodiments, the ratio is 1:15.

In some embodiments, a viral particle as described herein comprises a non-primate animal AAV. In some AAV capsid protein embodiments of the invention, the non-primate animal AAV is a non-primate AAV listed in Table 2 of WO2020242984, the content of which is incorporated herein by reference in its entirety. In some embodiments, the non-primate AAV is an avian AAV (AAAV), a sea lion AAV or a bearded dragon AAV. In some embodiments, the non-primate animal AAV is an AAAV, and optionally an amino acid sequence of an AAAV capsid protein comprises a modification is at position 1444 or 1580 of a VP1 capsid protein of AAAV. In some embodiments, the non-primate animal AAV is a squamate AAV, e.g., a bearded dragon AAV, and optionally an amino acid sequence of a bearded dragon AAV comprises a modification is at position 1573 or 1436 of a VP1 capsid protein of a bearded dragon AAV. In some embodiments, the non-primate animal AAV is a mammalian AAV, e.g., a sea lion AAV, and optionally an amino acid sequence of a seal lion AAV comprises a modification at position selected from the group consisting of 1429, 1430, 1431, 1432, 1433, 1434, 1436, 1437, and A565 of a VP1 capsid protein of a sea lion AAV.

Further embodiments of the present invention includes a method for altering the tropism of a virus, the method comprising the steps of: (a) inserting a nucleic acid encoding a heterologous epitope into a nucleic acid sequence encoding an viral capsid protein to form a nucleotide sequence encoding a genetically modified capsid protein comprising the heterologous epitope and/or (b) culturing a packaging cell in conditions sufficient for the production of viral vectors, wherein the packaging cell comprises the nucleotide sequence. A further embodiment of the present invention is a method for displaying a heterologous epitope on the surface of a capsid protein, the method comprising the steps of: a) expressing the nucleic acid according to this invention under suitable conditions, and b) isolating the expressed capsid protein of step a).

Packaging cells useful for production of the viral particles described herein include, e.g., animal cells permissive for the virus, or cells modified so as to be permissive for the virus; or the packaging cell construct, for example, with the use of a transformation agent such as calcium phosphate. Non-limiting examples of packaging cell lines useful for producing viral particles described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raj i (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

L929 cells, the FLY viral packaging cell system outlined in Cosset et al (1995) J. Virol 69,7430-7436, NS0 (murine myeloma) cells, human amniocytic cells (e.g., CAP, CAP-T), yeast cells (including, but not limited to, S. cerevisiae, Pichia pastoris), plant cells (including, but not limited to, Tobacco NTl, BY-2), insect cells (including but not limited to SF9, S2, SF21, Tni (e.g. High 5)) or bacterial cells (including, but not limited to, E. coli).

For additional packaging cells and systems, packaging techniques and vectors for packaging the nucleic acid genome into the pseudotyped viral particle see, for example, Polo, et al, Proc Natl Acad Sci USA, (1999) 96:4598-4603. Methods of packaging include using packaging cells that permanently express the viral components, or by transiently transfecting cells with plasmids.

### Nucleotide Sequences of Interest

In some embodiments, the viral particles described herein comprise one or more transfer vectors or a RNA molecule(s) encoded by the transfer vector, wherein said transfer vector or RNA molecule comprises at least one viral element and a nucleotide sequence of interest. In one specific embodiment, upon infection of the TCR-expressing cell with the viral particle, the nucleotide sequence of interest becomes integrated into the cell genome. In another specific embodiment, upon infection of the TCR-expressing cell with the viral particle, the nucleotide sequence of interest does not become integrated into the cell.

In some embodiments of any of the above methods, the nucleotide sequence of interest encodes a protein, a peptide, an RNAi molecule, an antisense oligonucleotide, a CRISPR/Cas9 system or derivatives thereof, or a miRNA. In some embodiments, the nucleotide sequence of interest encodes a molecule toxic to the TCR-expressing cell. In some embodiments, the nucleotide sequence of interest encodes a molecule that modulates an activity of the TCR-expressing cell (e.g., a molecule that inhibits an activity of the TCR-expressing cell or a molecule that enhances an activity of the TCR-expressing cell). In another embodiment, the nucleotide sequence of interest encodes a reporter molecule or a selectable marker (e.g., the neomycin resistance gene or other antibiotic resistance genes, fluorescent reporter molecules such as the green fluorescent protein (GFP) or derivatives thereof such as, e.g., enhanced GFP (EGFP), blue fluorescent protein derivatives (e.g. EBFP, EBFP2, Azurite, or mKala-mal), cyan fluorescent protein derivatives (e.g. ECFP, Cerulean, or CyPet), yellow fluorescent protein derivatives (e.g. YFP, Citrine, Venus, or YPet), as well as oxidative enzymes such as, e.g., firefly luciferase or antibody detectable cell markers, allowing, inter alia, fluorescence activated cells sorting (FACS) of the transduced cells). Other non-limiting examples of nucleotide sequences of interest which can be used in the viral particles described herein include, e.g., genes encoding apoptotic factors, genes encoding cytotoxic molecules, genes encoding anti-apoptotic factors, genes encoding immune-stimulatory molecules, TNF-a gene, p53 gene, interferon genes, “suicide genes” (i.e., the genes which cause a cell to kill itself through apoptosis; non-limiting examples of suicide genes include, e.g., herpes simplex virus thymidine kinase (HSV-TK), which converts ganciclovir (GCV) into cytotoxic compounds, Escherichia coli cytosine deaminase, which allows the formation of a cytotoxic chemotherapeutic agent from a non-toxic precursor, Varicella-zoster virus thymidine kinase, deoxycytidine kinase, purine nucleoside phosphorylase, nitroreductase, β-galactosidase, hepatic cytochrome P450-2P1, linamarase, horseradish peroxidase, and carboxypeptidase).

RNAi or microRNA molecules encoded by the nucleotide sequences of interest can be used, e.g., to down-regulate (or upregulate—for miRNA) expression of certain molecules in a cell. RNAi molecules (including, e.g., siRNA and shRNA) and microRNA molecules are known in the art. See, e.g., Shen, L. et al. 2004. Nat Biotech 22(12): 1546-1553; Zhou, H. et al. 2006. Biochemical and Biophysical Research Communications 347:200-207; Song, X-T., et al. 2006. PLoS Medicine 3(1):el 1; Kobayashi, T. and A. Yoshimura. 2005. TRENDS in Immunology 26(4):177-179; Taganov, K., et al. 2007. Immunity 26:133-137; Dahlberg, J. E. and E. Lund. 2007. Sci. STKE 387:pe25.

In one specific embodiment, the nucleotide sequence of interest is under the control of a promoter selected from the group consisting of a viral promoter homologous to the at least one viral element, a viral promoter heterologous to the at least one viral element, a bacterial promoter, a mammalian promoter, an avian promoter, a fish promoter, an insect promoter, and any combination thereof. In one specific embodiment, the nucleotide sequence of interest is under the control of a human promoter. In one specific embodiment, the nucleotide sequence of interest is under the control of a non-human promoter. In one specific embodiment, the nucleotide sequence of interest is under the control of a promoter that is specific to a T cell (e.g., TCR α-chain promoter, TCR β-chain promoter, TCR β-chain V-region gene promoter, CD2 promoter, CD3 promoter, CD4 promoter, CD5 promoter, CD6 promoter, CD7 promoter, CD8 promoter, CD28 promoter, CD278 promoter, FOXP3 promoter, TIM3 promoter, Granzyme A promoter, Granzyme B promoter, activation-induced cell death (AICD) promoter, TIM promoter, p56lck promoter, NKG5 promoter, and variants thereof, see, e.g., Anderson et al., Proc. Natl. Acad. Sci. USA (1988) 85:3551-3554; Winoto and Baltimore, EMBO J. (1989) 8:729-33; Haddad et al., European J. Immunol. 23:625-629; Halle et al., Mol. And Cell. Biol. (1997) 17:4220-4229).

The nucleotide sequence of interest can activate or inhibit a target TCR-expressing cell (e.g., T cell). In some embodiments, the target T cell is a CD4+ T cell such as, e.g., a helper T cell (e.g., a Th1, Th2, or Th17 cell) or a CD4+/CD25+/FOXP3+ regulatory T (Treg) cell. In some cases, the target T cell is a CD8+ T cell such as, e.g., a cytotoxic T cell. In some cases, the target T cell is a memory T cell, which can be a CD4+ T cell or a CD8+ T cell, where memory T cells are generally CD45RO+. In some cases, the target T cell is an NK-T cell.

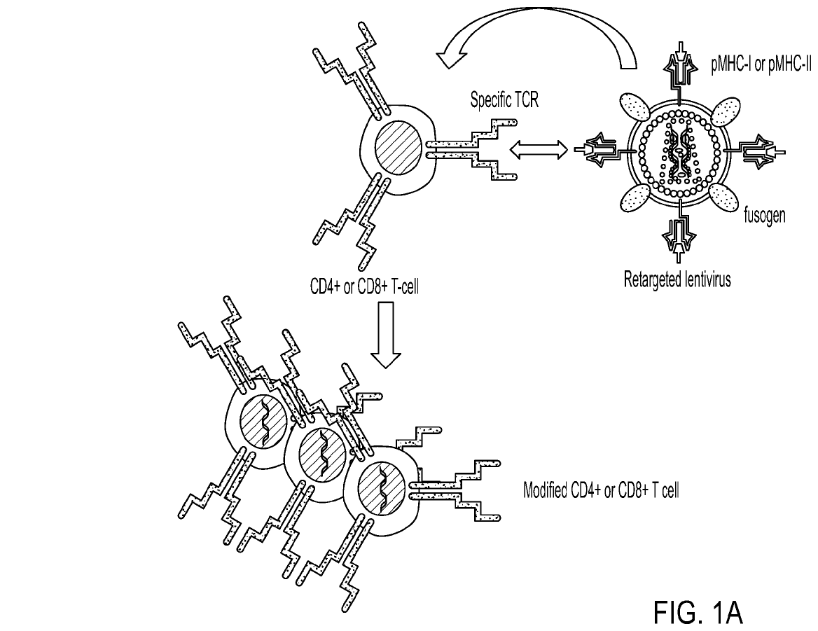

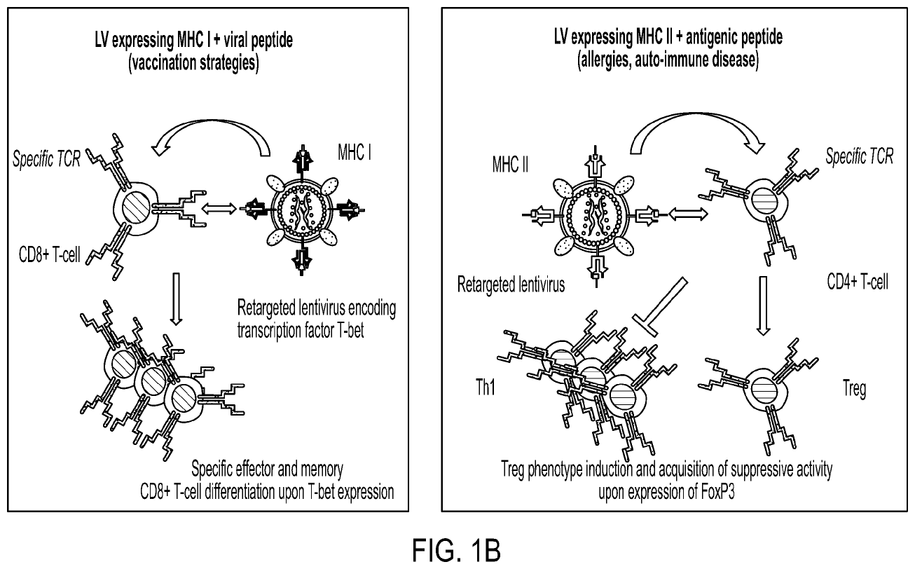

Non-limiting examples of molecules which can be encoded by the nucleotide sequence of interest and can be useful for modulating an activity of TCR-expressing cells include, e.g., T-bet, ZEB2, IL35, NFATc1, IL-15, IL-2, IL-7, IL-21, FOXP3, GATA-1, IKZF2, IKZF4, IRF4, SATB1, LEF1, Eomes, ZbTb25, BLIMP1, and chimeric antigen receptors (CARs). FIG. 1B shows two exemplary enveloped lentiviral particles of the invention carrying a nucleotide sequence encoding a transcription factor T-bet or FOXP3.

In some embodiments, the expression of the nucleotide sequence of interest enhances T cell homing and trafficking. For example, in some embodiments, the expression of nucleotide sequence of interest increases extravasation of the target T cell to a treatment site. Increased extravasation can increase the number of T cells at a treatment site.

In some embodiments, the expression of the nucleotide sequence of interest increases the expression by a target T cell of one or more proteins that mediate or regulate lymphocyte trafficking by the target T cell. For example, in some embodiments, the expression of the nucleotide sequence of interest increases the level of one or more adhesion molecules and/or chemokine receptor molecules in the target T cell. Examples of adhesion molecules include adhesion molecules produced by CD8 T cells, where examples of such adhesion molecules include, but are not limited to, CD44, LFA-1, and VLA-4. Examples of chemokine receptors include chemokine receptors produced by CD8 T cells, where examples of such chemokine receptors include, but are not limited to, CCR5, CCR7 and CXCR3.

In some embodiments, the expression of the nucleotide sequence of interest results in the generation of memory T cells capable of rapid cytotoxic responses against a previously experienced epitope.

In some embodiments, the expression of the nucleotide sequence of interest increases proliferation of a target T cell.

In some embodiments, the expression of the nucleotide sequence of interest increases cytotoxic activity of a T cell toward a target cell.

In some cases, the expression of the nucleotide sequence of interest increases cytokine production by a target T cell. Non-limiting examples of such cytokines include cytokines produced by Th1 cells, e.g., IL-2, IFN-γ, and TNF-α; cytokines produced by Th1 7 cells, e.g., IL-17, IL-21, and IL-22; cytokines produced by Treg cells, e.g., TGF-β, IL-35, and IL-10.

In some embodiments, the expression of the nucleotide sequence of interest inhibits cytokine production by a target T cell. Non-limiting examples of such cytokines include cytokines produced by Th2 cells, e.g., IL-4, IL-5, IL-6, IL-10, and IL-13.

### Fusogens

In some embodiments described herein, the viral particles comprise a fusogen. Many different protein and non-protein fusogens can be used. In some embodiments, the fusogen is a protein. In one specific embodiment, the fusogen is a viral protein. Non-limiting examples of useful viral fusogens include, e.g., vesiculovirus fusogens (e.g., vesicular stomatitis virus G glycoprotein (VSVG)), alphavirus fusogens (e.g., a Sindbis virus glycoprotein), orthomyxovirus fusogens (e.g., influenza HA protein), paramyxovirus fusogens (e.g., a Nipah virus F protein or a measles virus F protein), retrovirus fusogens, lentivirus fusogens, as well as fusogens from Dengue virus (DV), Lassa fever virus, tick-borne encephalitis virus, Dengue virus, Hepatitis B virus, Rabies virus, Semliki Forest virus, Ross River virus, Aura virus, Borna disease virus, Hantaan virus, SARS-CoV virus, and various fragments, mutants and derivatives thereof. Other exemplary fusogenic molecules and related methods are described, for example, in U.S. Pat. Appl. Pub. 2005/0238626 and 2007/0020238.

In one specific embodiment, the fusogen is heterologous to the virus from which the particle is derived. In one specific embodiment, the fusogen is a mutated protein which does not bind its natural ligand. In one specific embodiment, at least one chain of the MHC and the fusogen are comprised within a fusion protein.

There are two recognized classes of viral fusogens and both can be used as targeting molecules (D. S. Dimitrov, Nature Rev. Microbio. 2, 109 (2004)). The class I fusogens trigger membrane fusion using helical coiled-coil structures, whereas the class II fusogens trigger fusion with 13 barrels. In some embodiments, class I fusogens are used. In other embodiments, class II fusogens are used. In still other embodiments, both class I and class II fusogens are used. See, e.g., Skehel and Wiley, Annu. Rev. Biochem. 69, 531-569 (2000); Smit, J. et al. J. Virol. 73, 8476-8484 (1999), Morizono et al. J. Virol. 75, 8016-8020 (2005), Mukhopadhyay et al. (2005) Rev. Microbiol. 3, 13-22.

In some specific embodiments, a form of hemagglutinin (HA) from influenza A/fowl plague virus/Rostock/34 (FPV), a class I fusogen, is used (Hatziioannou et al., J. Virol. 72, 5313 (1998)). In some specific embodiments, a form of FPV HA is used (Lin et al., Hum. Gene. Ther. 12, 323 (2001)). HA-mediated fusion is generally considered to be independent of receptor binding (Lavillette et al., Cosset, Curr. Opin. Biotech. 12, 461 (2001)).

In other embodiments, the Sindbis virus glycoprotein (a class II fusogen) from the alphavirus family is used (Wang et al., J. Virol. 66, 4992 (1992); Mukhopadhyay et al., Nature Rev. Microbio. 3, 13 (2005), Morizono et al., Nature Med. 11, 346 (2005)).

In some embodiments, mutant fusogens are used which maintain their fusogenic ability but have a decreased or eliminated binding ability or specificity. Functional properties of mutant fusogens can be tested, e.g., in cell culture or by determining their ability to stimulate an immune response without causing undesired side effects in vivo.

To select most effective and non-toxic combinations of TCR-binding molecule (e.g., pMHC complex) and fusogens (either wild-type or mutant), viral particles bearing these molecules can be tested for their selectivity and/or their ability to facilitate penetration of the target cell membrane. Viral particles that display wild-type fusogens can be used as controls for examining titer effects in mutants. For example, cells can be transduced by the viral particles using a standard infection assay. After a specified time, for example 48 hours post-transduction, cells can be collected and the percentage of transduced cells can be determined by, for example, monitoring reporter gene expression (e.g., using FACS analysis). The selectivity can be scored by calculating the percentage of cells infected by the viral particles. Similarly, the effect of mutations on viral titer can be quantified by dividing the percentage of cells infected by viral particles comprising a mutant targeting molecule by the percentage of cells infected by virus comprising the corresponding wild type targeting molecule. The titers of viral particles can be determined, e.g., by limited dilution of the stock solution and transduction of cells expressing the TCR of interest. A preferred mutant will give the best combination of selectivity and infectious titer.

To investigate whether fusogen-mediated cell penetration is dependent upon pH, and select fusogens with the desired pH dependence, NH₄Cl or other compound that alters pH can be added at the infection step (NH₄Cl will neutralize the acidic compartments of endosomes). In the case of NH₄Cl, the disappearance of cells expressing the reporter will indicate that penetration of viruses is low pH-dependent. In addition, to confirm that the activity is pH-dependent, lysosomotropic agents, such as ammonium chloride, chloroquine, concanamycin, bafilomycin Al, monensin, nigericin, etc., may be added into the incubation buffer. These agents can elevate the pH within the endosomal compartments (Drose and Altendorf, J. Exp. Biol. 200, 1-8, 1997). The inhibitory effect of these agents will reveal the role of pH for viral fusion and entry. The different entry kinetics between viruses displaying different fusogenic molecules may be compared and the most suitable selected for a particular application.

PCR-based viral particle entry assays may be utilized to measure kinetics of viral DNA synthesis as an indication of the kinetics of viral particle entry. For example, viral particles comprising a particular TCR-binding molecule and fusogen can be incubated with target cells, unbound viruses can be then removed and aliquots of the cells can be analyzed by extracting DNA and performing semi-quantitative PCR (e.g., using LTR-specific primers for lentiviral or retroviral particles). The appearance of LTR-specific DNA products will indicate the success of viral particle entry and uncoating.

The fusogen preferably exhibits fast enough kinetics that the viral particle contents can empty into the cytosol before the degradation of the viral particle. In addition, the fusogen can be modified to reduce or eliminate any binding activity and thus reduce or eliminate any non-specific binding. That is, by reducing the binding ability of the fusogen, binding of the viral particles to the target TCR-expressing cell is determined predominantly or entirely by the TCR-binding molecule, allowing for high target specificity and reducing undesired side-effects.

The measles virus (MeV), a prototype morbillivirus of the genus Paramyxoviridae, utilizes two envelope glycoproteins (the fusion protein (F) and the hemagglutinin protein (H)) to gain entry into the target cell. Protein F is a type I transmembrane protein, while protein H is a type II transmembrane domain, i.e., its amino-terminus is exposed directly to the cytoplasmic region. Both proteins thus comprise a transmembrane and a cytoplasmic region. One known function of the F protein is mediating the fusion of viral membranes with the cellular membranes of the host cell. Functions attributed to the H protein include recognizing the receptor on the target membrane and supporting F protein in its membrane fusion function. The direct and highly efficient membrane fusion at the cellular surface membrane is a particular property of measles virus and the morbilliviruses, thus distinguishing themselves from many other enveloped viruses that become endocytosed and will only fuse upon pH drop upon endocytosis. Both proteins are organized on the viral surface in a regular array of tightly packed spikes, H tetramers, and F trimers (Russell et al., Virology 199:160-168, 1994).

In certain embodiments, the fusogenic molecule is a Sindbis virus envelope protein (SIN). The SINdbis virus transfers its RNA into the cell by low pH mediated membrane fusion. SIN contains five structural proteins, E1, E2, E3, 6K and capsid. E2 contains the receptor binding sequence that allows the wild-type SIN to bind, while E1 is known to contain the properties necessary for membrane fusion (Konoochik et al., Virology Journal 2011, 8:304). E1, E2, and E3 are encoded by a polyprotein, the amino acid sequence of which is provided, e.g., by Accession No. VHWVB, VHWVB2, and β03316: the nucleic acid sequence is provided, e.g., by Accession No. SVU90536 and V01403 (see also Rice & Strauss, Proc. Nat'l Acad. Sci USA 78:2062-2066 (1981); and Strauss et al., Virology 133:92-110 (1984)).

In certain embodiments, the Sindbis virus envelope protein is mutated (SINmu). In certain embodiments, the mutation reduces the natural tropism of the Sindbis virus. In certain embodiments, a SINmu comprising SIN proteins E1, E2, and E3, wherein at least one of E1, E2, or E3 is mutated as compared to a wild-type sequence. For example, one or more of the E1, E2, or E3 proteins can be mutated at one or more amino acid positions. In addition, combinations of mutations in E1, E2, and E3 are encompassed by fusogen as described herein, e.g., mutations in E1 and E2, or in E2 and E3, or E3 and E1, or E1, E2, and E3. In certain embodiments, at least E2 is mutated.

In certain embodiments, the SINmu comprises the following envelope protein mutations in comparison to wild-type Sindbis virus envelope proteins: (i) deletion of E3 amino acids 61-64; (ii) E2 KE159-160AA; and (iii) E2 SLKQ68-71AAAA (“SLKQ” and “AAAA” disclosed as SEQ ID NOS 71-72, respectively) (see SEQ ID NO: 5 and coding sequence SEQ ID NO: 6). In a further embodiment, the SINmu additionally comprises the envelope protein mutation E1 AK226-227SG. Examples of SINmu may be found in, for example, in U.S. Pat. No. 9,163,248; WO2011011584; Cronin et. al., Curr Gene Ther. 2005 August; 5(4): 387-398.

Other Togaviridae family envelopes, e.g., from the Alphavirus genus, e.g., Semliki Forest Virus, Ross River Virus, and equine encephalitis virus, can also be used to pseudotype the vectors described herein. The envelope protein sequences for such Alphaviruses are known in the art.

In certain embodiments, the fusogen is a vesicular stomatitis virus (VSV) envelope protein. In certain embodiments, the fusogen is the G protein of VSV (VSV-G; Burns et al., Proc. Natl. Acad. Sci. U.S.A. 1993, vol. 90, no. 17, p. 1833-7) or a fragment, mutant, derivative or homolog thereof. VSV-G interacts with a phospholipid component of the cell (e.g., T cell) membrane to mediate viral entry by membrane fusion (Mastromarino et al., J Gen Virol. 1998, vol. 68, no. 9, p. 2359-69; Marsh et al., Adv Virus Res. 1989, vol. 107, no. 36, p. 107-51. Examples of VSV-G may be found in, for example, WO2008058752.

### MHCs Useful in Viral Particles and their Interaction with Antigenic Determinants

Disclosed herein are recombinant viral particles that are capable of binding to an antigen-specific T-cell receptor (TCR), wherein said particles comprise a on its surface (e.g., lipid envelope or capsid) comprising (i) a peptide, e.g., an antigenic determinant, presented in the context of a major histocompatibility complex (MHC) molecule, and, optionally, (ii) a fusogen.

MHC molecules are generally classified into two categories: class I and class II MHCs. An MHC class I molecule is an integral membrane protein comprising a glycoprotein heavy chain, also referred to herein as the α chain, which has three extracellular domains (i.e., α1, α2 and α3) and two intracellular domains (i.e., a transmembrane domain (TM) and a cytoplasmic domain (CYT)). The heavy chain is noncovalently associated with a soluble subunit called β2-microglobulin (β2m or B2M). An MHC class II protein is a heterodimeric integral membrane protein comprising one α chain and one β chain in noncovalent association. The α chain has two extracellular domains (α1 and α2), and two intracellular domains (a TM domain and a CYT domain). The β chain contains two extracellular domains (β1 and β2), and two intracellular domains (a TM domain and CYT domain).

The domain organization of class I and class II MHCs forms the antigenic determinant binding site, or peptide binding groove. A peptide binding groove refers to a portion of an MHC protein that forms a cavity in which a peptide, e.g., antigenic determinant, can bind. The conformation of a peptide binding groove is capable of being altered upon binding of a peptide to enable proper alignment of amino acid residues important for TCR binding to the peptide-MHC (pMHC) complex.

The MHCs described herein include fragments of MHC chains that are sufficient to form a peptide binding groove. For example, a peptide binding groove of a class I protein can comprise portions of the α1 and α2 domains of the heavy chain capable of forming two β-pleated sheets and two a helices. Inclusion of a portion of the β2-microglobulin chain stabilizes the complex. While for most versions of MHC Class II molecules, interaction of the α and β chains can occur in the absence of a peptide, the two chain complex of MHC Class I is unstable until the binding groove is filled with a peptide. A peptide binding groove of a class II protein can comprise portions of the α1 and β1 domains capable of forming two β-pleated sheets and two a helices. A first portion of the α1 domain forms a first β-pleated sheet and a second portion of the α1 domain forms a first a helix. A first portion of the β1 domain forms a second β-pleated sheet and a second portion of the β1 domain forms a second a helix. The X-ray crystallographic structure of class II protein with a peptide engaged in the binding groove of the protein shows that one or both ends of the engaged peptide can project beyond the MHC protein (Brown et al., pp. 33-39, 1993, Nature, Vol. 364). Thus, the ends of the α1 and β1 α helices of class II form an open cavity such that the ends of the peptide bound to the binding groove are not buried in the cavity. Moreover, the X-ray crystallographic structure of class II proteins shows that the N-terminal end of the MHC β chain apparently projects from the side of the MHC protein in an unstructured manner since the first 4 amino acid residues of the β chain could not be assigned by X-ray crystallography.

Many human and other mammalian MHC molecules are well known in the art and any MHC Class I or Class II molecules may be part of a TCR-binding molecule as described herein.

MHC molecules useful in the viral particles described herein include naturally occurring full-length MHC molecules as well as individual chains of MHC molecules (e.g., MHC class I a (heavy) chain, β2-microglobulin, MHC class II α chain, and MHC class II β chain), individual subunits of such chains of MHCs (e.g., α1, α2 and/or α3 subunits of MHC class I α chain, α1 and/or α2 subunits of MHC class II α chain, 31 and/or β2 subunits of MHC class II β chain) as well as fragments, mutants and various derivatives thereof, wherein such fragments, mutants and derivatives retain the ability to display an antigenic determinant for recognition by an antigen-specific TCR. In one specific embodiment, the MHC comprises a transmembrane domain embedded in the lipid envelope of the viral particle.

Naturally-occurring MHC molecules are encoded by a cluster of genes on human chromosome 6 or mouse chromosome 17. Said MHCs, referred to as H-2 in mice and HLA (Human Leucocyte Antigen) in humans, are classified as either class I molecules or class II molecules. MHC class I molecules specifically bind CD8 molecules expressed on cytotoxic T lymphocytes (CD8+ T cells), whereas MHC class II molecules specifically bind CD4 molecules expressed on helper T lymphocytes (CD4+ T cells). MHCs include, but are not limited to, HLA specificities such as A (e.g. A1-A74), B (e.g., B 1-β77), C (e.g., C1-C11), D (e.g., D1-D26), E, G, DR (e.g., DR1-DR8), DQ (e.g., DQ1-DQ9) and DP (e.g. DP1-DP6). More preferably, HLA specificities include A1, A2, A3, A11, A23, A24, A28, A30, A33, B7, B8, B35, B44, B53, B60, B62, DR1, DR2, DR3, DR4, DR7, DR8, and DR-11.

The MHCs useful in the viral particles described herein may be from any mammalian or avian species, for example, primates (e.g., humans), rodents, rabbits, equines, bovines, canines, felines, pigs, etc.

Naturally occurring MHC class I molecules bind peptides derived from proteolytically degraded proteins, especially endogenously synthesized proteins, by a cell. Small peptides obtained accordingly are transported into the endoplasmic reticulum where they associate with nascent MHC class I molecules before being routed through the Golgi apparatus and displayed on the cell surface for recognition by cytotoxic T lymphocytes.

Naturally occurring MHC class I molecules consist of an α (heavy) chain associated with β2-microglobulin. The heavy chain consists of subunits α1-α3. The β2-microglobulin protein and 3 subunit of the heavy chain are associated. In certain embodiments, β2-microglobulin and α3 subunit are covalently bound. In certain embodiments, β2-microglobulin and α3 subunit are noncovalently bound. The α1 and α2 subunits of the heavy chain fold to form a groove for a peptide, e.g., antigenic determinant, to be displayed and recognized by TCR.

Class I molecules bind peptides of about 8-9 amino acids in length. All humans have between three and six different class I molecules, which can each bind many different types of peptides.

In some embodiments, the MHC contained in the vector particles comprises (i) a class I MHC polypeptide or a fragment, mutant or derivative thereof, and, optionally, (ii) a β2 microglobulin polypeptide or a fragment, mutant or derivative thereof. In one specific embodiment, the class I MHC polypeptide is linked to the β2 microglobulin polypeptide by a peptide linker.

In one specific embodiment, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H-2L, H2-IA, H2-IB, H2-IJ, H2-IE, and H2-IC.

In some embodiments, the viral particle comprises one or more MHC class I a heavy chains. In some embodiments, the MHC class I a heavy chain is fully human. In some embodiments, the MHC class I a heavy chain is humanized. Humanized MHC class I a heavy chains are described, e.g., in U.S. Pat. Pub. Nos. 2013/0111617, 2013/0185819 and 2014/0245467.

In some embodiments, the MHC class I a heavy chain comprises a human extracellular domain (human α1, α2, and/or α3 domains) and a cytoplasmic domain of another species. In some embodiments, the class I a heavy chain polypeptide is HLA-A, HLA-B, HLA-C, HLA-E, HLA-F, HLA-G, HLA-K, or HLA-L. In some embodiments, the HLA-A sequence can be an HLA-A*0201 sequence. In various aspects, the peptide-MHC can include all the domains of an MHC class I heavy chain.

In some embodiments, the viral particle comprises a β2-microglobulin. In some embodiments, the β2-microglobulin is fully human. In some embodiments, the β2-microglobulin is humanized. Humanized β2-microglobulin polypeptides are described, e.g., in U.S. Pat. Pub. Nos. 2013/0111617 and 2013/0185819.

In some embodiments, the MHC class I molecule comprises a mutation in a β2-microglobulin (β2m or B2M) polypeptide and in the Heavy Chain sequence so as to affect a disulfide bond between the B2M and the Heavy Chain. In some cases, the Heavy Chain is an HLA and wherein the disulfide bond links one of the following pairs of residues: B2M residue 12, HLA residue 236; B2M residue 12, HLA residue 237; B2M residue 8, HLA residue 234; B2M residue 10, HLA residue 235; B2M residue 24, HLA residue 236; B2M residue 28, HLA residue 232; B2M residue 98, HLA residue 192; B2M residue 99, HLA residue 234; B2M residue 3, HLA residue 120; B2M residue 31, HLA residue 96; B2M residue 53, HLA residue 35; B2M residue 60, HLA residue 96; B2M residue 60, HLA residue 122; B2M residue 63, HLA residue 27; B2M residue Arg3, HLA residue Glyl20; B2M residue His31, HLA residue Gln96; B2M residue Asp53, HLA residue Arg35; B2M residue Trp60, HLA residue G1n96; B2M residue Trp60, HLA residue Asp122; B2M residue Tyr63, HLA residue Tyr27; B2M residue Lys6, HLA residue Glu232; B2M residue Gln8, HLA residue Arg234; B2M residue TyrlO, HLA residue Pro235; B2M residue Serl 1, HLA residue Gln242; B2M residue Asn24, HLA residue Ala236; B2M residue Ser28, HLA residue Glu232; B2M residue Asp98, HLA residue His 192; and B2M residue Met99, HLA residue Arg234, first linker position Gly 2, Heavy Chain (HLA) position Tyr 84; Light Chain (B2M) position Arg 12, HLA Ala236; and/or B2M residue Argl2, HLA residue Gly237. See, e.g., Int. Pat. Appl. Pub. WO2015/195531.

In some embodiments, the antigenic determinant amino acid sequence can be that of a peptide which can be presented by an MHC class I molecule. In certain embodiments, the sequence can comprise from about 8 to about 15 contiguous amino acids. In certain embodiments, a peptide sequence can be that of a protein fragment, wherein the protein is a derived from, e.g., a portion of, an infectious agent or a cellular protein, such as, for example, a protein expressed by a cancer cell, and wherein the peptide can be bound to the MHC class I heavy chain.

In some embodiments, at least one chain of the MHC and the peptide are comprised within a fusion protein. In one specific embodiment, the MHC and the peptide are separated by a linker sequence. For example, the single chain molecule can comprise, from amino to carboxy terminal, an antigenic determinant, a β2-microglobulin sequence, and a class I a (heavy) chain sequence. Alternatively, the single chain molecule can comprise, from amino to carboxy terminal, an antigenic determinant, a class I α (heavy) chain sequence, and a β2-microglobulin sequence. The single-chain molecule can further comprise a signal peptide sequence at the amino terminal. In certain embodiments, there can be a linker sequence between the peptide sequence and the β2-microglobulin sequence. In certain embodiments, there can be a linker sequence between the β2-microglobulin sequence and the class I α (heavy) chain sequence. A single-chain molecule can further comprise a signal peptide sequence at the amino terminal, as well as first linker sequence extending between the peptide sequence and the β2-microglobulin sequence, and/or a second linker sequence extending between the β2-microglobulin sequence and the class I heavy chain sequence. In certain embodiments, the β2-microglobulin and the class I α (heavy) chain sequences can be human, murine, or porcine.

In some embodiments, a single-chain molecule can comprise a first flexible linker between the peptide ligand segment and the β2-microglobulin segment. For example, linkers can extend from and connect the carboxy terminal of the peptide ligand segment to the amino terminal of the β2-microglobulin segment. Preferably, the linkers are structured to allow the linked peptide ligand to fold into the binding groove resulting in a functional MHC-antigen peptide. In some embodiments, this linker can comprise at least about 10 amino acids, up to about 15 amino acids. In some embodiments, a single-chain molecule can comprise a second flexible linker inserted between the β2-microglobulin and heavy chain segments. For example, linkers can extend from and connect the carboxy terminal of the β2-microglobulin segment to the amino terminal of the heavy chain segment. In certain embodiments, the β2-microglobulin and the heavy chain can fold into the binding groove resulting in a molecule which can function in promoting T cell expansion.

Suitable linkers used in the MHCs can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Non-limiting examples of linkers include, e.g., glycine polymers (G)n, glycine-serine polymers (including, for example, (GS)n, (GSGGS (SEQ ID NO: 73)) and (GGGS (SEQ ID NO: 74)) (TABLE 4), where n is an integer of at least one), glycine-alanine polymers, alanine-serine polymers, and other flexible linkers known in the art (see, e.g., Chichili et al, Protein Science, 22:153-167 (2013)). Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 1 1173-142 (1992)). Exemplary linkers can comprise amino acid sequences including, but not limited to, GGSG (SEQ ID NO: 75), GGSGG (SEQ ID NO: 76), GSGSG (SEQ ID NO: 77), GSGGG (SEQ ID NO: 78), GGGSG (SEQ ID NO: 79), GSSSG (SEQ ID NO: 80), GCGASGGGGSGGGGS (SEQ ID NO: 81), GGGGSGGGGS (SEQ ID NO: 82), GGGASGGGGSGGGGS (SEQ ID NO: 83), GGGGSGGGGSGGGGS (SEQ ID NO: 84), GGGASGGGGS (SEQ ID NO: 85), or GGGGSGGGGSGGGGSGGGGS (SEQ ID NO: 86) (TABLE 4), and the like. In some embodiments, a linker polypeptide includes a cysteine residue that can form a disulfide bond with a cysteine residue present in a second polypeptide.

In certain embodiments, the single-chain molecule can comprise a peptide covalently attached to an MHC class I α (heavy) chain via a disulfide bridge (i.e., a disulfide bond between two cystines). In certain embodiments, the disulfide bond comprises a first cysteine, comprised by a linker extending from the carboxy terminal of an antigen peptide, and a second cysteine comprised by an MHC class I heavy chain (e.g., an MHC class I α (heavy) chain which has a non-covalent binding site for the antigen peptide). In certain embodiments, the second cysteine can be a mutation (addition or substitution) in the MHC class I α (heavy) chain. In certain embodiments, the single-chain molecule can comprise one contiguous polypeptide chain as well as a disulfide bridge. In certain embodiments, the single-chain molecule can comprise two contiguous polypeptide chains which are attached via the disulfide bridge as the only covalent linkage. In some embodiments, the linking sequences can comprise at least one amino acid in addition to the cysteine, including one or more glycines, one or more, alanines, and/or one or more serines.

In certain embodiments, the disulfide bridge can link an antigen peptide in the class I groove of the pMHC complex if the pMHC complex comprises a first cysteine in a Gly-Ser linker extending between the C-terminus of the peptide and the β2-microglobulin, and a second cysteine in a proximal heavy chain position.

In some embodiments, the β2-microglobulin sequence can comprise a full-length β2-microglobulin sequence. In certain embodiments, the β2-microglobulin sequence lacks the leader peptide sequence. As such, in some configurations, the β2-microglobulin sequence can comprise about 99 amino acids, and can be a mouse β2-microglobulin sequence (e.g., Genebank X01838). In some other configurations, the β2-microglobulin sequence can comprise about 99 amino acids, and can be a human β2-microglobulin sequence (e.g., Genebank AF072097.1).

In some embodiments, the pMHC complex sequence can be that as disclosed in U.S. Pat. Nos. 4,478,82; 6,011,146; 8,518,697; 8,895,020; 8,992,937; WO 96/04314; Mottez et al. J. Exp. Med. 181: 493-502, 1995; Madden et al. Cell 70: 1035-1048, 1992; Matsumura et al., Science 257: 927-934, 1992; Mage et al., Proc. Natl. Acad. Sci. USA 89: 10658-10662, 1992; Toshitani et al, Proc. Nat'l Acad. Sci. 93: 236-240, 1996; Chung et al, J. Immunol. 163:3699-3708, 1999; Uger and Barber, J. Immunol. 160: 1598-1605, 1998; Uger et al., J. Immunol. 162, pp. 6024-6028, 1999; White et al., J. Immunol. 162: 2671-2676, 1999; Yu et al., J. Immunol. 168:3145-3149, 2002; Truscott et al., J. Immunol. 178: 6280-6289, 2007.

In some embodiments, the MHC comprises a class II MHC polypeptide or a fragment, mutant or derivative thereof. In one specific embodiment, the MHC comprises α and βpolypeptides of a class II MHC complex or a fragment, mutant or derivative thereof. In one specific embodiment, the α and β polypeptides are linked by a peptide linker. In one specific embodiment, the MHC comprises α and β polypeptides of a human class II MHC complex selected from the group consisting of HLA-DP, HLA-DR, HLA-DQ, HLA-DM and HLA-DO. In another specific embodiment, the MHC comprises α and β polypeptides of a murine H-2A or H-2E class II MHC complex.

Naturally occurring MHC class II molecules consist of two polypeptide chains, α and β. The chains may come from the DP, DQ, or DR gene groups. There are about 40 known different human MHC class II molecules. All have the same basic structure, but vary subtly in their molecular structure. MHC class II molecules bind peptides of 13-18 amino acids in length.

In some embodiments, the viral particle comprises one or more MHC class II α chains. In some embodiments, the MHC class II α chain is fully human. In some embodiments, the MHC class II α chain is humanized. Humanized MHC class II α chains are described, e.g., in U.S. Pat. Nos. 8,847,005 and 9,043,996 and U.S. Pat. Pub. No. 2014/0245467. In some embodiments, the humanized MHC class II α chain polypeptide comprises a human extracellular domain and a cytoplasmic domain of another species. In some embodiments, the class II α chain is HLA-DMA, HLA-DOA, HLA-DPA, ILA-DQA or HLA-DRA. In some embodiments, the class II α chain polypeptide is humanized HLA-DMA, HLA-DOA, HLA-DPA, HLA-DQA and/or HLA-DRA.

In some embodiments, the viral particle comprises one or more MHC class II β chains. In some embodiments, the MHC class II § chain is fully human. In some embodiments, the MHC class II β chain polypeptide is humanized. Humanized MHC class II β chain polypeptides are described, e.g., in U.S. Pat. Nos. 8,847,005 and 9,043,996 and U.S. Pat. Pub. No. 2014/0245467.

In some embodiments, the humanized MHC class II β chain comprises a human extracellular domain and a cytoplasmic domain of another species. In some embodiments, the class II β chain is HLA-DMB, HLA-DOB, HLA-DPB, HLA-DQB or HLA-DRB. In some embodiments, the class II β chain is humanized HLA-DMB, HLA-DOB, HLA-DPB, HLA-DQB and/or HLA-DRB.

### Antigens and Antigen-Derived Peptides

An antigenic determinant comprised in the pseudotyped viral particles described herein can comprise any peptide that is capable of binding to an MHC protein in a manner such that the pMHC complex can bind to a TCR, preferably in a specific manner. In certain embodiments, such binding induces a T cell response.

Examples include peptides produced by hydrolysis and most typically, synthetically produced peptides, including randomly generated peptides, specifically designed peptides, and peptides where at least some of the amino acid positions are conserved among several peptides and the remaining positions are random.

In nature, peptides that are produced by hydrolysis undergo hydrolysis prior to binding of the antigen to an MHC protein. Class I MHC typically present peptides derived from proteins actively synthesized in the cytoplasm of the cell. In contrast, class II MHC typically present peptides derived either from exogenous proteins that enter a cell's endocytic pathway or from proteins synthesized in the ER. Intracellular trafficking permits a peptide to become associated with an MHC protein.

The binding of a peptide to an MHC peptide binding groove can control the spatial arrangement of MHC and/or peptide amino acid residues recognized by a TCR. Such spatial control is due in part to hydrogen bonds formed between a peptide and an MHC protein. Based on the knowledge on how peptides bind to various MHCs, the major MHC anchor amino acids and the surface exposed amino acids that are varied among different peptides can be determined. Preferably, the length of an MHC-binding peptide is from about 5 to about 40 amino acid residues, more preferably from about 6 to about 30 amino acid residues, and even more preferably from about 8 to about 20 amino acid residues, and even more preferably between about 9 and 11 amino acid residues, including any size peptide between 5 and 40 amino acids in length, in whole integer increments (i.e., 5, 6, 7, 8, 9 . . . 40). While naturally MHC Class II-bound peptides vary from about 9-40 amino acids, in nearly all cases the peptide can be truncated to an about 9-11 amino acid core without loss of MHC binding activity or T cell recognition.

Peptides include peptides comprising at least a portion, e.g., an antigenic determinant, of a protein selected from a group consisting of a self-protein associated with an autoimmune disorder, proteins of infectious agents, and tumor associated proteins.

Non-limiting examples of self proteins associated with an autoimmune disorder include, e.g., gliadin (celiac disease; e.g., (i) α-gliadin fragment corresponding to amino acids 57-73 or (ii) 7-gliadin fragment corresponding to amino acids 139-153 or (iii) w-gliadin fragment corresponding to amino acids 102-118), GAD 65, IA-2 and insulin B chain (for type 1-diabetes), glatiramer acetate (GA) (for multiple sclerosis), achetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)).

In certain embodiments, the antigen comprises a peptide (e.g., antigenic determinant of a protein) that is the target of an autoreactive T cell involved in celiac disease. For example, the peptide can be derived from, comprises a portion of gluten peptides, such as α-gliadin, γ-gliadin, and/or glutenins. In certain embodiments, the epitope is derived from α-gliadin (33-mer (57-89) and its truncated forms, 25-mer (64-89), 18-mer (71-89), 17-mer (57-73), 13-mer (57-68), and glia-20); from γ-gliadin (DQ2-γ-I, DQ2-γ-II, DQ2-γ-III, DQ2-γ-IV, and DQ2-γ-V, 14-mer-1 (105-118) and 14-mer-2 (173-186)); glutenin (Glt-19-39 and glt-156 (42-56)); and/or glu-5. In certain embodiments, the antigen-derived peptide can include α-gliadin (57-73), γ-gliadin (139-153), and/or ω-gliadin (102-118). See, e.g., Camarca et al., Endocrine, Metabolic & Immune Disorders—Drug Targets, 12:207-219 (2012); Camarca et al., J. Immunol., 182(7): 4158-4166 (2009).

In certain embodiments, the peptide comprises an antigenic determinant of a protein that is the target of an autoreactive T cell involved in psoriasis, e.g., BV3 and/or BV13S1.

In certain embodiments, the peptide comprises an antigenic determinant of a protein that is the target of an autoreactive T cell involved in multiple sclerosis, e.g., BV5S2, BV6S5, and/or BV13SI.

In certain embodiments, the peptide comprises an antigenic determinant of a protein that is the target of an autoreactive T cell involved in rheumatoid arthritis, e.g., BV3, BV14, and/or BV17.

Non-limiting examples of viral proteins from which peptides may be derived to be used in the viral particles described herein include LCMV gp33, CMV pp65, HIV gag, EBV BMLF1 as well as antigens derived from influenza virus (e.g., surface glycoproteins hemagluttinin (HA) and neuramimidase (NA)); immunodeficiency virus (e.g., a human immunodeficiency virus antigens (HIV) such as gp120, gp160, p18 antigen Gag p17/p24, Tat, Pol, Nef, and Env); herpesvirus (e.g., a glycoprotein from herpes simplex virus (HSV), Marek's Disease Virus, cytomegalovirus (CMV), or Epstein-Barr virus); hepatitis virus (e.g., Hepatitis B surface antigen (HBsAg)); papilloma virus; rous associated virus (e.g., RAV-1 env); infectious bronchitis virus (e.g., matrix and/or preplomer); flavivirus (e.g., a Japanese encephalitis virus (JEV) antigen, a Yellow Fever antigen, or a Dengue virus antigen); Morbillivirus (e.g., a canine distemper virus antigen, a measles antigen, or rinderpest antigen such as HA or F); rabies (e.g., rabies glycoprotein G); parvovirus (e.g., a canine parvovirus antigen); poxvirus (e.g., an ectromelia antigen, a canary poxvirus antigen, or a fowl poxvirus antigen); chicken pox virus (varicella zoster antigen); infectious bursal disease virus (e.g., VP2, VP3, or VP4); Hantaan virus, and mumps virus.

Non-limiting examples of bacterial proteins which may be a source of bacterial peptides, e.g., antigenic determinants, that can be used in the viral particles described herein include lipopolysaccharides isolated from gram-negative bacterial cell walls and staphylococcus-specific, streptococcus-specific, pneumococcus-specific (e.g., PspA; see PCT Publication No. WO 92/14488), Neisseria gonorrhea-specific, Borrelia-specific (e.g., OspA, OspB, OspC of Borrelia associated with Lyme disease such as Borrelia burgdorferi, Borrelia afzelli, and Borrelia garinii [see, e.g., U.S. Pat. No. 5,523,089; PCT Publication Nos. WO 90/04411, WO 91/09870, WO 93/04175, WO 96/06165, WO93/08306; PCT/US92/08697; Bergstrom et al., Mol. Microbiol., 1999; 3: 479486; Johnson et al., Infect. and Immun. 1992; 60: 1845-1853; Johnson et al., Vaccine 1995; 13: 1086-1094; The Sixth International Conference on Lyme Borreliosis: Progress on the Development of Lyme Disease Vaccine, Vaccine 1995; 13: 133-135]), and pseudomonas-specific proteins or peptides. Additional non-limiting examples of bacterial antigens include, e.g., antigens from Neisseria gonorrhea, Mycobacterium tuberculosis, Haemophilus vaginalis, Group B Streptococcus sp., Microplasma hominis, Hemophilus ducreyi, Granuloma inguinale, Lymphopathia venereum, Ireponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Escherichia coli, Actinobacillus equuli, Salmonella abortus ovis, Salmonella abortus equi, Pseudomonas aeruginosa, Corynebacterium equi, Corynebacterium pyogenes, and Actinobaccilus seminis.

Non-limiting examples of malaria-specific proteins from which antigenic determinants may be isolated include circumsporozoite (CS) protein, Thrombospondin Related Adhesion (Anonymous) protein (TRAP), also called Sporozoite Surface Protein 2 (SSP2), LSA I, hsp70, SALSA, STARP, Hep17, MSA, RAP-1, RAP-2.

Non-limiting examples of fungal proteins from which antigenic determinants may be isolated include those isolated from candida (e.g., MP65 from Candida albicans), trichophyton, and ptyrosporum.

Non-limiting examples of tumor-associated proteins from which antigenic determinants may be isolated include, e.g., adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, MEl, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAPI, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a, Kras, NY-ESO1, MAGE-A3, HPV E2, HPV E6, HPV E7, WT-1 antigen (in lymphoma and other solid tumors), ErbB receptors, Melan A [MARTI], gp 100, tyrosinase, TRP-1/gp 75, and TRP-2 (in melanoma); MAGE-1 and MAGE-3 (in bladder, head and neck, and non-small cell carcinoma); HPV EG and E7 proteins (in cervical cancer); Mucin [MUC-1](in breast, pancreas, colon, and prostate cancers); prostate-specific antigen [PSA](in prostate cancer); carcinoembryonic antigen [CEA](in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-1/LAGE-2, NA-88, GnTV, TRP2-INT2. In some embodiments, the protein is a neo-antigen. In some embodiments, the protein is a tumor specific antigen.

In some embodiments, the MHC pseudotyped viral particles described herein are exposed to libraries of synthetically produced peptides to identify the antigenic determinants recognized by a specific T cell. Such peptide libraries include, e.g., peptide libraries produced by PCR (including by introducing random mutations into various positions of a template peptide). A peptide library can include up to 20⁹ or 2×10¹¹ members, or as few as a few hundred to a few thousand members, depending on the knowledge of the peptide binding characteristics of a given MHC. Since 4-5 amino acids are generally involved in MHC binding and can not directly contact the TCR, prior knowledge of the nature of these amino acids means that only about 5-7 amino acids in the peptide need vary, so that libraries of 10⁶ to 10⁹ members are typically sufficient. In addition, in some cases, T cell recognition is dominated by only a few amino acids in the core of the peptide, and in these cases, libraries with only a few hundred to a few thousand members may be sufficient to identify functional peptide-MHC complexes.

Extensive knowledge regarding the binding of peptides to MHC complexes is available to a person of ordinary skill in the art, so that for a given MHC complex, one can design MHC-groove binding peptides that vary in less than all of the available positions. For example, the MHCBN is a comprehensive database of Major Histocompatibility Complex (MHC) binding and non-binding peptides compiled from published literature and existing databases. The latest version of the database has 19,777 entries including 17,129 MHC binders and 2648 MHC non-binders for more than 400 MHCs. The database has sequence and structure data of (a) source proteins of peptides and (b) MHCs. MHCBN has a number of web tools that include: (i) mapping of peptide on query sequence; (ii) search on any field; (iii) creation of data sets; and (iv) online data submission (Bioinformatics 2003 Mar. 22; 19(5):665-6).

In one specific embodiment, a library of candidate peptides is produced by genetically engineering the library using polymerase chain reaction (PCR) or any other suitable technique to construct a DNA fragment encoding the peptide. With PCR techniques, by using oligonucleotides that are randomly mutated within particular triplet codons, the resultant fragment pool encodes all possible combination of codons at these positions. Preferably, certain of the amino acid positions are maintained constant, which are the conserved amino acids that are required for binding to the MHC peptide binding groove and which do not contact the T cell receptor. See, e.g., U.S. Pat. Appl. Pub. 2004/0110253.

In this screening method, the target TCR is a TCR for which it is desired to identify the peptide epitope recognized by the receptor. In one embodiment, the target TCR is from a patient with a T cell-mediated disease, such as an autoimmune disease, infection or cancer. See, e.g., Rossjohn and Koning, Mucosal Immunology, 9(3):583-586 (2016); Qiao et al., J Immunol., 187:3064-3071 (2011); Broughton et al., Immunity, 37:611-621 (2012); Qiao et al., International Immunology, 26 (1):13-19. In one embodiment, the TCR includes an α-chain and a β-chain.

Attaching the peptide to the MHC Class I or MHC Class II molecule via a flexible linker has the advantage of assuring that the peptide will occupy and stay associated with the MHC during biosynthesis, transport and display. However, there may be situations in which this linker interferes with peptide binding to the MHC or with TCR recognition of the complex. As an alternate approach, in some embodiments, the MHC and the peptide are expressed separately.

In some embodiments, a library of MHC-peptide pseudotyped viral particles may be constructed as described herein. In some embodiments, such library is capable of screening against a population of antigen-specific cells (e.g., B cells or T cells) to identify antigen-specific cells or antigen-specific molecules (e.g., TCRs, CARs, antibodies, or peptides).

### TCR-Binding Antibodies

In certain embodiments, the TCR-binding molecule of the viral particles comprises an anti-TCR antibody, or portion thereof.

TCR-specific antibodies described herein include, e.g., antibodies or antigen binding fragments thereof, which specifically bind TCR α chains, antibodies which specifically bind TCR β chains, antibodies which specifically bind TCR γ chains, and antibodies which specifically bind TCR δ chains. In some embodiments, the antibodies or portions thereof described herein recognize an idiotype of an α/β TCR or a γ/δ TCR (i.e., recognizes only TCR specific for one particular antigen of interest). In some embodiments, the antibodies described herein recognize α/β TCR, generally. In some embodiments, the antibodies described herein specifically bind the extracellular domain of TCR, e.g., the extracellular constant domain of the α chain of TCR.

Assays to determine the binding specificity of an anti-TCR antibody or antigen binding fragment thereof include but are not limited to ELISA, Western blot, surface plasmon resonance (e.g., BIAcore) and radioimmunoassay. Any method known in the art for assessing protein-protein binding specificity may be used to identify antibodies or antigen binding fragments of the invention that exhibit a Kd of 0.001-50 nM, or 0.001-25 nM, or 0.001-10 nM, or 0.001-5 nM. In certain embodiments, the isolated VH or VL domains of the anti-TCR antibodies of the invention exhibit a Kd of no greater than 5 nM, no greater than 10 nM, no greater than 25 nM, no greater than 50 nM, e.g., as determined by BIAcore assay.

In certain embodiments of the invention, the anti-TCR antibodies are monoclonal antibodies, synthetic antibodies, recombinantly produced antibodies, human antibodies, chimeric antibodies, camelized antibodies, single-chain Fvs (scFv), single chain antibodies, Fab fragments, F(ab′) fragments, disulfide-linked Fvs (sdFv), intrabodies, or epitope-binding fragments of any of the above. In certain embodiments, the anti-TCR antibodies of the invention, or antigen fragments thereof, are Ig-DARTS or covalent diabodies such as those disclosed in U.S. Pat. Appl. Pub. 2007/0004909.

The anti-TCR antibodies of the invention, or antigen binding fragments thereof, may be humanized by any method known in the art for modifying proteins for therapeutic use in humans. In addition to methods commonly known in the art for combining heterologous CDR sequences with human framework and/or constant domains, the term “humanization” also includes methods of protein and/or antibody resurfacing such as those disclosed, e.g., in U.S. Pat. Nos. 5,770,196; 5,776,866; 5,821,123; and 5,896,619.

The anti-TCR antibodies of the invention, or antigen binding fragments thereof may derived from any species (e.g., rabbit, mouse, rat, donkey, cow, camel, llama, sheep, goat, horse, primate), but are preferably derived from human immunoglobulin molecules that can be of any type (e.g., IgG, IgE, IgM, IgD, IgA and IgY), or class (e.g., IgG1, IgG2, IgG3, IgG4, IgA1, and IgA2) or subclass. The anti-TCR antibodies of the invention, or antigen binding fragments thereof, can be produced by any method known in the art, for example, chemical synthesis or recombinant techniques.

The invention encompasses anti-TCR antibodies comprising one or more amino acid modifications which, e.g., alter antibody binding or effector functions. See, e.g., U.S. Pat. Appl. Pub. Nos. U.S. 2005/0037000 and U.S. 2005/0064514; U.S. Pat. Nos. 5,624,821 and 5,648,260; European Pat. No. EP0307434; Int. Pat. Appl. Pub. Nos. WO 04/029207, WO 04/029092, WO 04/028564, WO 99/58572, WO 99/51642, WO 98/23289, WO 89/07142, WO 88/07089, U.S. Pat. Nos. 5,843,597 and 5,642,821). In some embodiments, mutation of the amino acids of a protein creates an equivalent, or even an improved, second-generation molecule. For example, certain amino acids may be substituted for other amino acids in a protein structure without detectable loss of affinity or avidity.

In some embodiments, such fusion proteins comprise linker sequences. In some embodiments, the anti-TCR antibodies may be conjugated to a cytotoxic agent or a therapeutic agent that modifies a given biological response. For example, the cytotoxic agent may be a toxin (e.g., abrin, ricin A, pseudomonas exotoxin (i.e., PE-40), diphtheria toxin, ricin, gelonin), pokeweed antiviral protein, a tumor necrosis factor (TNF), an interferon (e.g., IFN-α, IFN-β), nerve growth factor (NGF), platelet derived growth factor (PDGF), tissue plasminogen activator (TPA), an apoptotic agent (e.g., TNF-α, TNF-β, AIM I, AIM II, Fas ligand, VEGF), a thrombotic agent, an anti-angiogenic agent (e.g., angiostatin or endostatin), or a biological response modifier such as, for example, a lymphokine (e.g., IL-1, IL-2, IL-6, GM-CSF, M-CSF), a growth factor (e.g., growth hormone (GH)), a protease, or a ribonuclease. Alternatively (or in addition) ant-TCR antibodies can be fused to a reporter to facilitate a functional analysis or to a tag sequence(s) to facilitate antibody purification. Non-limiting examples of useful peptide tags include, e.g., a His-tag (e.g., His₆ (SEQ ID NO: 87)) (Gentz et al., 1989 Proc. Natl. Acad. Sci. USA, 86:821-824), a hemagglutinin (HA) tag (Wilson et al., 1984 Cell, 37:767) and the “flag” tag (Knappik et al., 1994 Biotechniques, 17(4):754-761).

Modified antibodies or fragments thereof can be produced, e.g., by random mutagenesis by error-prone PCR, random nucleotide insertion or other methods prior to recombination, DNA shuffling, etc.

The present invention also encompasses anti-TCR antibodies conjugated to a diagnostic or therapeutic agent. For example, an antibody may be conjugated to a cytotoxin (e.g., a cytostatic or cytocidal agent such as, e.g., paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof), an antimetabolite (e.g., methotrexate, 6-mercaptopurine, 6-thioguanine, cytarabine, 5-fluorouracil decarbazine), an alkylating agent (e.g., mechlorethamine, thioepa chlorambucil, melphalan, carmustine (BSNU) and lomustine (CCNU), cyclothosphamide, busulfan, dibromomannitol, streptozotocin, mitomycin C, or cisdichlorodiamine platinum (II) (DDP) cisplatin), an anthracycline (e.g., daunorubicin (formerly daunomycin) or doxorubicin), an antibiotic (e.g., dactinomycin (formerly actinomycin), bleomycin, mithramycin, or anthramycin), or an anti-mitotic agent (e.g., vincristine or vinblastine), a radioactive element (e.g., alpha-emitters, gamma-emitters, etc. such as, e.g., bismuth (²¹³Bi), carbon (¹⁴C), chromium (⁵¹Cr), cobalt (⁵⁷Co), fluorine (⁸F) gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb) yttrium (⁹⁰Y), zinc (⁶⁵Zn), including macrocyclic chelators useful for conjugating radiometal ions such as, e.g., 1,4,7,10-tetraazacyclododecane-N,N′,N″,N″-tetraacetic acid (DOTA) which can be attached to the antibody via a linker molecule. Examples of linker molecules are provided, for example, in Denardo et al., 1998, Clin Cancer Res. 4:2483-90; Peterson et al., 1999, Bioconjug. Chem. 10:553; and Zimmerman et al., 1999, Nucl. Med. Biol. 26:943-50.

Techniques for conjugating therapeutic moieties to antibodies are well known in the art. See, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al., Immunol. Rev., 62:119-58, 1982. Detection can be facilitated by coupling the antibody to a detectable substance. Examples of detectable substances include various enzymes (e.g., horseradish peroxidase, alkaline phosphatase, 0-galactosidase, or acetylcholinesterase), prosthetic groups (e.g., avidin/biotin and streptavidin/biotin), fluorescent materials (e.g., umbelliferone, fluorescein, fluorescein isothiocyanate, rhodamine, dichlorotriazinylamine fluorescein, dansyl chloride, or phycoerythrin), luminescent materials (e.g., luminal, luciferase, luciferin, and aequorin), radioactive materials (e.g., bismuth (²¹³Bi), carbon (¹⁴C), chromium (⁵¹Cr), cobalt (⁵⁷Co), fluorine (⁸F) gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I) lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb) yttrium (⁹⁰Y), zinc (⁶⁵Zn)), positron emitting metals, and nonradioactive paramagnetic metal ions. The detectable substance may be coupled or conjugated either directly to the antibody or indirectly, e.g., through a linker.

Anti-TCR antibodies of the invention can be produced by any method known in the art for the synthesis of antibodies, including chemical synthesis and recombinant expression techniques.

For example, for production of polyclonal antibodies, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including, e.g., hybridoma, recombinant, and phage display technologies, or a combination thereof. See, e.g., Harlow et al., Antibodies: A Laboratory Manual, (Cold Spring Harbor Laboratory Press, 2nd ed. 1988); Hammerling, et al., in: Monoclonal Antibodies and T cell Hybridomas 563 681 (Elsevier, N.Y., 1981). Methods for producing and screening for specific antibodies using hybridoma technology are routine and well known in the art. Briefly, mice can be immunized with a TCR antigen and once an immune response is detected, e.g., antibodies specific for a TCR antigen (e.g., the extracellular constant domain of the TCR α chain) are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Antibody fragments which recognize specific TCR antigen (e.g., the extracellular constant domain of the TCR α chain) may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

Further, the antibodies of the present invention can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include, e.g., those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; Int. Pat. Appl. Pub. Nos. PCT/GB91/01134, WO 90/02809, WO 91/10737, WO 92/01047, WO 92/18619, WO 93/1 1236, WO 95/15982, WO 95/20401, and WO97/13844; U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108. As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in Int. Pat. Appl. Pub. No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and Int. Pat. App. Pub. Nos. WO 98/46645, WO 98/50433, WO 98/24893, WO98/16654, WO 96/34096, WO 96/33735, and WO 91/10741.

Single domain antibodies, for example, antibodies lacking the light chains, can be produced by methods well-known in the art. See, e.g., Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and Int. Pat. App. Pub. Nos. WO 94/04678, WO 94/25591, and WO 01/44301.

### Cytotoxic Molecules

In some embodiments, the viral particles described herein further comprise a cytotoxic agent. In one specific embodiment, the cytotoxic agent is a toxin or a radioactive isotope (e.g., a radioconjugate) or a suicide gene. Non-limiting examples of toxins which can be used in the viral particles described herein include, e.g., enzymatically active toxins of bacterial, fungal, plant, or animal origin, or fragments, mutants or derivatives thereof. Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. Non-limiting examples of suicide genes include, e.g., thymidine kinase, cytosine deaminase, purine nucleoside phosphorylase, nitroreductase, β-galactosidase, hepatic cytochrome P450-2P1, linamarase, horseradish peroxidase, and carboxypeptidase.

### Binding Pairs

A recombinant viral particle of the present disclosure may be attached to a TCR-binding molecule (e.g., anti-TCR antibody or portions, or a pMHC complex) via a linker moiety. The linker moeity may be a specific binding pair or protein:protein binding pair. For example, the viral particle described herein may comprise a first member of the specific binding pair that is inserted into/displayed by a recombinant viral capsid protein, and further comprises a second member of the specific binding pair that specifically forms a covalent bond with the first member of the specific binding pair and wherein the second member is fused to a TCR-binding molecule.

“Specific binding pair,” “protein:protein binding pair” and the like includes two proteins (e.g., a first member (e.g., a first polypeptide) and a second cognate member (e.g., a second polypeptide)) that interact to form a covalent isopeptide bond under conditions that enable or facilitate isopeptide bond formation, wherein the term “cognate” refers to components that function together, i.e. to react together to form an isopeptide bond. Thus, two proteins that react together efficiently to form an isopeptide bond under conditions that enable or facilitate isopeptide bond formation can also be referred to as being a “complementary” pair of peptide linkers. Specific binding pairs capable of interacting to form a covalent isopeptide bond are reviewed in Veggiani et al. (2014) Trends Biotechnol. 32:506, and include peptide:peptide binding pairs such as SpyTag:SpyCatcher; SpyTag002:SpyCatcher002; SpyTag:KTag; isopeptag:pilin C, SnoopTag:SnoopCatcher, etc. Generally, a peptide tag refers to member of a protein:protein binding pair, which is generally less than 30 amino acids in length, and which forms a covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e. between two peptide/protein molecules, e.g. between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SpyTag:SpyCatcher system is described in U.S. Pat. No. 9,547,003 Zakeri et al. (2012) PNAS 109:E690-E697, and WO2019006046, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK which forms an amide bond to its cognate protein “SpyCatcher”. (Zakeri (2012), supra). An additional specific binding pair derived from CnaB2 domain is SpyTag:KTag, which forms an isopeptide bond in the presence of SpyLigase. (Fierer (2014) PNAS 111:E1176-1181) SpyLigase was engineered by excising the p strand from SpyCatcher that contains a reactive lysine, resulting in KTag, 10-residue peptide tag having the amino acid sequence ATHIKFSKRD. The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK, and binds SpyCatcher002.

The SnoopTag:SnoopCatcher system is described in Veggiani (2016) PNAS 113:1202-07. The D4 Ig-like domain of RrgA, an adhesion from Streptococcus pneumoniae, was split to form SnoopTag (residues 734-745) and SnoopCatcher (residues 749-860). Incubation of SnoopTag and SnoopCatcher results in a spontaneous isopeptide bond that is specific between the complementary proteins. Veggiani (2016)), supra.

The isopeptag:pilin-C specific binding pair was derived from the major pilin protein Spy0128 from Streptococcus pyogenes. (Zakeir and Howarth (2010) J. Am. Chem. Soc. 132:4526-27). Isopeptag has the amino acid sequence TDKDMTITFTNKKDAE, and binds pilin-C (residues 18-299 of Spy0128). Incubation of SnoopTag and SnoopCatcher results in a spontaneous isopeptide bond that is specific between the complementary proteins. Zakeir and Howarth (2010), supra.

In some embodiments, the specific binding pair is a SpyTag:SpyCatcher binding pair, wherein the first member is SpyTag, and wherein the second cognate member is SpyCatcher. In some embodiments, the specific binding pair is SpyTag:KTag, wherein the first member is SpyTag and wherein the second cognate member is KTag. In some embodiments, the specific binding pair is SpyTag:KTag, wherein the first member is KTag and wherein the second cognate member is SpyTag. In some embodiments, the specific binding pair is isopeptag:pilin-C, wherein the first member is isopeptag, and wherein the second cognate member is pilin-C, or a portion thereof. In some embodiments, the specific binding pair is SnoopTag:SnoopCatcher, and the first member is SnoopTag, and the second cognate member is SnoopCatcher.

### Treatment Methods

In another aspect, disclosed herein is a method for delivering a nucleotide sequence of interest to a cell comprising a TCR comprising contacting the cell with a viral particle described herein, wherein the viral particle comprises the TCR-binding molecule. In some embodiments, the delivery of the nucleotide sequence of interest results in modulating an activity or survival of the TCR-expressing cell.

In a further aspect, disclosed herein is a method for modulating an activity or survival of a cell comprising a TCR, comprising contacting the cell with a viral particle described herein, wherein the viral particle comprises the TCR-binding molecule.

In some embodiments of any of the above methods, the cell is a lymphocyte such as, e.g., a T-cell (e.g., a CD4+ T-cell or a CD8+ T-cell).

In some embodiments of any of the above methods, said contacting is ex vivo.

In some embodiments of the above methods, said contacting is in vivo in a subject (e.g., human).

In some embodiments of any of the above methods, the cell is a mammalian cell (e.g., a human cell).

In a related aspect, disclosed herein are modified cells comprising the viral particles, or one or more portions thereof (e.g., capsomer, nucleotide of interest, viral element, a combination of same, etc.) described herein. In some embodiments, the cell is a lymphocyte such as, e.g., a T-cell (e.g., a CD4+ T-cell or a CD8+ T-cell). In some embodiments, the cell is a mammalian cell (e.g., a human cell).

In some embodiments, e.g., where the target T cell is a CD8+ T cell, the viral particle described herein comprises a class I MHC polypeptide. In some embodiments, e.g., where the target T cell is a CD4+ T cell, the viral particle described herein comprises class II MHC polypeptides.

Where the viral particle described herein includes a nucleotide sequence of interest which encodes an immunomodulatory polypeptide that is an activating polypeptide, transduction of the T cell with the viral particle activates the epitope-specific T cell. In some instances, the epitope-specific T cell is a T cell that is specific for an epitope present on a cancer cell, and contacting the epitope-specific T cell with the viral particle increases cytotoxic activity of the T cell toward the cancer cell. In some embodiments, the epitope-specific T cell is a T cell that is specific for an epitope present on a cancer cell, and contacting the epitope-specific T cell with the viral particle increases the number of the epitope-specific T cells.

In some embodiments, the epitope-specific T cell is a T cell that is specific for an epitope present on a virus-infected cell, and contacting the epitope-specific T cell with the viral particle increases cytotoxic activity of the T cell toward the virus-infected cell. In some instances, the epitope-specific T cell is a T cell that is specific for an epitope present on a virus-infected cell, and contacting the epitope-specific T cell with the viral particle increases the number of the epitope-specific T cells.

Where the viral particle described herein includes an immunomodulatory polypeptide that is an inhibiting polypeptide, contacting the T cell with the viral particle inhibits the epitope-specific T cell. In some instances, the epitope-specific T cell is a self-reactive T cell that is specific for an epitope present in a self antigen, and the contacting reduces the number of the self-reactive T cells.

The interaction of a T cell with the viral particles described herein can result in, e.g., activation, induction of anergy, or death of a T cell that occurs when the TCR of the T cell is bound by a TCR-binding molecule (e.g., MHC-peptide complex). “Activation of a T cell” refers to induction of signal transduction pathways in the T cell resulting in production of cellular products (e.g., interleukin-2) by that T cell. “Anergy” refers to the diminished reactivity by a T cell to an antigen. Activation and anergy can be measured by, for example, measuring the amount of IL-2 produced by a T cell after an pMHC complex has bound to the TcR. Anergic cells will have decreased IL-2 production when compared with stimulated T cells. Another method for measuring the diminished activity of anergic T cells includes measuring intracellular and/or extracellular calcium mobilization by a T cell upon engagement of its TCR's. “T cell death” refers to the permanent cessation of substantially all functions of the T cell.

T-cell phenotypes may be evaluated using well-known methods, e.g., T cell activation may be determined, e.g., by measuring changes in the level of expression of cytokines and/or T cell activation markers, and/or the induction of antigen-specific proliferating cells. Techniques known to those of skill in the art, including, but not limited to, immunoprecipitation followed by Western blot analysis, ELISAs, flow cytometry, Northern blot analysis, and RT-PCR can be used to measure the expression cytokines and T cell activation markers. Cytokine release may be measured by measuring secretion of cytokines including but not limited to Interleukin-2 (IL-2), Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, IFN-γ, TFN-γ, IGF-I, and IGF-II (see, e.g., Isaacs et al., 2001, Rheumatology, 40: 724-738; Soubrane et al., 1993, Blood, 81(1): 15-19).

T cell modulation may also be evaluated, e.g., after infection with a viral particle as described herein, by measuring, e.g., proliferation by, e.g., ³H-thymidine incorporation, trypan blue cell counts, and fluorescence activated cell sorting (FACS).

The anti-tumor responses of T cells after infection with viral particles described herein may be determined in xenograft tumor models. Tumors may be established using any human cancer cell line expressing the tumor associated antigen presented by the viral particles. In order to establish xenograft tumor models, about 5×10⁶ viable cells, may be injected, e.g., s.c., into nude athymic mice using for example Matrigel (Becton Dickinson). The endpoint of the xenograft tumor models can be determined based on the size of the tumors, weight of animals, survival time and histochemical and histopathological examination of the cancer, using methods known to one skilled in the art.

The anergic state or death of T cells after infection with viral particles described herein, e.g., which may be useful for treatment of inflammatory and autoimmune disorders, can be tested in vitro or in vivo by, e.g., ⁵¹Cr-release assays. The ability to mediate the depletion of peripheral blood T cells can be assessed by, e.g., measuring T cell counts using flow cytometry analysis.

Non-limiting examples of useful animal models for analyzing the effect of infection of T cells by viral particles described herein on inflammatory diseases include adjuvant-induced arthritis rat models, collagen-induced arthritis rat and mouse models and antigen-induced arthritis rat, rabbit and hamster models (see, e.g., Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993); Trenthorn et al., 1977, J. Exp. Med. 146:857; Courtenay et al., 1980, Nature 283:665; Cathcart et at, 1986, Lab. Invest. 54:26; Holmdahl, R., 1999, Curr. Biol. 15:R528-530). Other useful animal models of inflammatory diseases include animal models of inflammatory bowel disease, ulcerative cholitis and Crohn's disease induced, e.g., by sulfated polysaccharides (e.g., amylopectin, carrageen, amylopectin sulfate, dextran sulfate) or chemical irritants (e.g., trinitrobenzenesulphonic acid (TNBS) or acetic acid). See, e.g., Kim et al., 1992, Scand. J. Gastroentrol. 27:529-537; Strober, 1985, Dig. Dis. Sci. 30(12 Suppl):3S-10S). Additional useful models are animal models for asthma such as, e.g., adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (see, e.g., Cohn et al., 1997, J. Exp. Med. 1861737-1747). Useful animal models of studying the effect of the viral particles of the invention on multiple sclerosis (MS) include an experimental allergic encephalomyelitis (EAE) model (see, e.g., Zamvil et al, 1990, Ann. Rev, Immunol. 8:579). Animal models which can be used for analyzing the effect of the viral particles of the invention on autoimmune disorders such as type 1 diabetes, thyroid autoimmunity, systemic lupus eruthematosus, and glomerulonephritis have been also developed (see, e.g., Bluestone et al., 2004, PNAS 101:14622-14626; Flanders et al., 1999, Autoimmunity 29:235-246; Krogh et al., 1999, Biochimie 81:511-515; Foster, 1999, Semin. Nephrol. 19:12-24).

Efficacy of the viral particles disclosed herein to downregulate immune responses in treating an autoimmune disorder may be evaluated, e.g., by detecting their ability to reduce one or more symptoms of the autoimmune disorder, to reduce mean absolute lymphocyte counts, to decrease T cell activation, to decrease T cell proliferation, to reduce cytokine production, or to modulate one or more particular cytokine profiles (e.g., Interleukin-2 (IL-2). Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, IFN-γ, TFN-γ, IGF-I, and IGF-II) (see, e.g., Isaacs et al., 2001, Rheumatology, 40: 724-738; Soubrane et al., 1993, Blood, 81(1): 15-19).

Efficacy of the viral particles for use in treating diabetes may be evaluated, e.g. by the ability of the viral particles to reduce one or more symptoms of diabetes, to preserve the C-peptide response to MMTT, to reduce the level HA1 or HA1c, to reduce the daily requirement for insulin, or to decrease T cell activation in pancreatic islet tissue. Efficacy in treating arthritis may be assessed through tender and swollen joint counts, determination of a global scores for pain and disease activity, ESRICRP, determination of progression of structural joint damage (e.g., by quantitative scoring of X-rays of hands, wrists, and feet (Sharp method)), determination of changes in functional status (e.g., evaluated using the Health Assessment Questionnaire (HAQ)), or determination of quality of life changes (assessed, e.g., using SF-36).

In a related aspect, disclosed herein is a method of treating a disorder in a subject in need thereof comprising administering to said subject an effective amount of the viral particle described herein, wherein the viral particle binds to an antigen-specific TCR and wherein the antigen is associated with the disorder. In some embodiments, the disorder an inflammatory or an autoimmune disorder and the administration results in a downregulation of an inflammatory or autoimmune response. In one specific embodiment, the disorder is celiac disease or gluten sensitivity. In one specific embodiment, the antigen comprises a gliadin or a fragment thereof (e.g., (i) α-gliadin fragment corresponding to amino acids 57-73 or (ii) γ-gliadin fragment corresponding to amino acids 139-153 or (iii) ω-gliadin fragment corresponding to amino acids 102-118). In one specific embodiment, the viral particle presents a peptide derived from the antigen in the context of a class II MHC. In some embodiments, the disorder is a tumor and the administration results in an upregulation of an anti-tumor immune response. In another embodiment, the disorder is an infection caused by an infectious agent and the administration results in an upregulation of an immune response against the infectious agent. In one specific embodiment, the infectious agent is selected from the group consisting of a virus, a bacterium, a fungus, a protozoa, a parasite, a helminth, and an ectoparasite. In one specific embodiment, the infectious agent is lymphocytic choriomeningitis virus (LCMV) and the antigen is gp33 protein. In one specific embodiment, the viral particle presents a peptide derived from the antigen in the context of a class I MHC. In some embodiments, the subject is a mammal (e.g., human).

Non-limiting examples of cancers treatable by the methods described herein include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all typies including but not limited to EBV-associated Hodkin's and non-Hodgkin's lymphoma, all forms of post-transplant lymphomas including post-transplant lymphoproliferative disorder (PTLD), uterine cancer, renal cell carcinoma, hepatoma, hepatoblastoma, Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Non-limiting examples of the inflammatory and autoimmune diseases include, e.g., inflammatory bowel disease (IBD), ulcerative colitis (UC), Crohn's disease, diabetes (e.g., diabetes mellitus type 1), multiple sclerosis, arthritis (e.g., rheumatoid arthritis), Graves' disease, lupus erythematosus, ankylosing spondylitis, psoriasis, Behcet's disease, autistic enterocolitis, Guillain-Barre Syndrome, myasthenia gravis, pemphigus vulgaris, acute disseminated encephalomyelitis (ADEM), transverse myelitis autoimmune cardiomyopathy, Celiac disease, dermatomyositis, Wegener's granulomatosis, allergy, asthma, contact dermatitis, atherosclerosis (or any other inflammatory condition affecting the heart or vascular system), autoimmune uveitis, as well as other autoimmune skin conditions, autoimmune kidney, lung, or liver conditions, autoimmune neuropathies, asthma, allergy, celiac disease, systemic lupus erythematosis (SLE), scleroderma, sarcoidosis, thyroiditis, multiple sclerosis, spondylitis, periarteritis, eczema, atopic dermatitis, myasthenia gravis, insulin-dependent diabetes mellitus, Crohn's disease, Guillain-Barre syndrome, Graves' disease, glomerulonephritis, ulcerative colitis, Crohn's disease, sprue, autoimmune arthritis, rheumatoid arthritis, osteoarthritis, juvenile chronic arthritis, psoriatic arthritis, reactive arthritis, spondyloarthropathy, psoriasis, acute or chronic immune disease associated with organ transplantation, an inflammatory disease, skin or organ transplantation rejection, graft-versus-host disease (GVHD), or autoimmune diseases, comprising administering to a subject a pharmaceutical composition described herein (e.g., a pharmaceutic composition comprising a viral particle described herein. Examples of autoimmune diseases include, for example, glomerular nephritis, arthritis, dilated cardiomyopathy-like disease, ulcerous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis.

In another embodiment, the viral particles described herein are used for treating or preventing a transplantation-related condition. In another embodiment, the viral particles described herein are used for treating or preventing graft-versus-host disease. In another embodiment, the viral particles described herein are used for treating or preventing a post-transplant lymphoproliferative disorder.

Thus, in yet another embodiment, the viral particles described herein are used for treating or preventing an infection. The infections include, without limitation, infections caused by viruses, bacteria, fungi, protozoa, parasites, helminths, and ectoparasites. The infectious agent can be, without limitation, a virus, a bacterium, a fungus, a protozoa, a parasite, a helminth, and an ectoparasite.

In one specific embodiment, the infectious agent is lymphocytic choriomeningitis virus (LCMV) and the antigen is gp33 protein.

It is contemplated that when used to treat various diseases, the compositions and methods can be combined with other therapeutic agents suitable for the same or similar diseases. Also, two or more embodiments described herein may be also ω-administered to generate additive or synergistic effects. When ω-administered with a second therapeutic agent, the embodiment described herein and the second therapeutic agent may be simultaneously or sequentially (in any order). Suitable therapeutically effective dosages for each agent may be lowered due to the additive action or synergy.

As a non-limiting example, the methods described herein can be combined with other therapies that block inflammation (e.g., via blockage of IL1, INFα/β, IL6, TNF, IL13, IL23, etc.).

In some embodiments, the compositions and methods disclosed herein are useful to enhance the efficacy of vaccines directed to tumors or infections. Thus, the compositions and methods described herein can be administered to a subject either simultaneously with or before (e.g., 1-30 days before) a reagent (including but not limited to small molecules, antibodies, or cellular reagents) that acts to elicit an immune response (e.g., to treat cancer or an infection) is administered to the subject.

The compositions and methods described herein can be also administered in combination with an anti-tumor antibody or an antibody directed at a pathogenic antigen or allergen.

The compositions and methods described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The inhibitory treatments described herein can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods described herein can be combined with additional immunotherapies and therapies. For example, when used for treating cancer, NKT cells described herein can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

For treatment of infections, combined therapy described herein can encompass co-administering compositions and methods described herein with an antibiotic, an anti-fungal drug, an anti-viral drug, an anti-parasitic drug, an anti-protozoal drug, or a combination thereof.

Non-limiting examples of useful antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. See also Physician's Desk Reference, 59.sup.th edition, (2005), Thomson P D R, Montvale N.J.; Gennaro et al., Eds. Remington's The Science and Practice of Pharmacy, 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Non-limiting examples of useful anti-fungal agents include imidazoles (such as griseofulvin, miconazole, terbinafine, fluconazole, ketoconazole, voriconazole, and itraconizole); polyenes (such as amphotericin B and nystatin); Flucytosines; and candicidin or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Non-limiting examples of useful anti-viral drugs include interferon alpha, beta or gamma, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Non-limiting examples of useful anti-parasitic agents include chloroquine, mefloquine, quinine, primaquine, atovaquone, sulfasoxine, and pyrimethamine or any salts or variants thereof. See also Physician's Desk Reference, 59th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

Non-limiting examples of useful anti-protozoal drugs include metronidazole, diloxanide, iodoquinol, trimethoprim, sufamethoxazole, pentamidine, clindamycin, primaquine, pyrimethamine, and sulfadiazine or any salts or variants thereof. See also Physician's Desk Reference, 59th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

### Pharmaceutical Compositions, Dosage Forms and Administration

Also disclosed herein are pharmaceutical compositions comprising the viral particles described herein and a pharmaceutically acceptable carrier and/or excipient. In addition, disclosed herein are pharmaceutical dosage forms comprising the viral particle described herein.

As discussed herein, the pseudotyped viral particles described herein can be used for various therapeutic applications (in vivo and ex vivo) and as research tools.

Pharmaceutical compositions based on the vector particles disclosed herein can be formulated in any conventional manner using one or more physiologically acceptable carriers and/or excipients. The vector particles may be formulated for administration by, for example, injection, inhalation or insulation (either through the mouth or the nose) or by oral, buccal, parenteral or rectal administration, or by administration directly to a tumor.

The pharmaceutical compositions can be formulated for a variety of modes of administration, including systemic, topical or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. ationd oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long-acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the vector particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered viral particles described herein on a daily or weekly basis for a time period or on a monthly, bi-yearly or yearly basis depending on need or exposure to a pathogenic organism or to a condition in the subject (e.g. cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the viral particles and any other reagent containers in close confinement for commercial sale. Such containers may include injection or blow-molded plastic containers into which the desired vials are retained. Optionally, one or more additional active agents such as, e.g., anti-inflammatory agents, anti-viral agents, anti-fungal or anti-bacterial agents or anti-tumor agents may be needed for compositions described.

Dose ranges and frequency of administration can vary depending on the nature of the viral particles and the medical condition as well as parameters of a specific patient and the route of administration used. In some embodiments, viral particle compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the viral particle compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×10⁸ pfu to about 1×10¹⁰ pfu, while an adult human subject can receive a dose from about 1×10¹⁰ pfu to about 1×10¹² pfu.

Compositions disclosed herein may be administered by any means known in the art. For example, compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Any method known to one skilled in the art maybe used for large scale production of viral particles, packaging cells and vector constructs described herein. For example, master and working seed stocks may be prepared under GMP conditions in qualified primary CEFs or by other methods. Packaging cells may be plated on large surface area flasks, grown to near confluence and viral particles purified. Cells may be harvested, and viral particles released into the culture media isolated and purified, or intracellular viral particles released by mechanical disruption (cell debris can be removed by large-pore depth filtration and host cell DNA digested with endonuclease). Virus particles may be subsequently purified and concentrated by tangential-flow filtration, followed by diafiltration. The resulting concentrated bulk maybe formulated by dilution with a buffer containing stabilizers, filled into vials, and lyophilized. Compositions and formulations may be stored for later use. For use, lyophilized viral particles may be reconstituted by addition of diluent.

Certain additional agents used in the combination therapies can be formulated and administered by any means known in the art.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. For example, adjuvants can stimulate Th1 or Th2 immunity. Compositions and methods as disclosed herein can also include adjuvant therapy.

### Library and Screening Methods

In one aspect, the present disclosure provides a library comprising a plurality of viral particles of described herein.

In some embodiments, the viral particles within the library differ in the sequence of the peptides (p) presented in the context of a major histocompatibility complex (MHC) molecule (pMHC complex) on the surface of the viral particles.

In some embodiments, the library comprises at least 10², at least 10³, at least 10⁴, at least 10⁵, at least 10⁶, at least 10⁷, at least 10⁸, at least 10⁹, or at least 10¹⁰ unique viral particles. In some embodiments, the library comprises about 10², about 10³, about 10⁴, about 10⁵, about 10⁶, about 10⁷, about 10⁸, about 10⁹, or about 10¹⁰ unique viral particles.

In some embodiments, each different peptide (p) sequence within the library is generated based on mass-spectrometry, computational analysis, Edman degradation using a protein sequenator, and/or validated TCR epitopes (e.g., epitopes discovered by enzyme-linked immunosorbent spot (ELISPOT), dextramer/tetramer stain, or other methods of detection of T cell activation).

In some embodiments, each viral particle within the library comprises a polynucleotide encoding a reporter protein. In some embodiments, each viral particle within the library comprises a reporter protein.

In one embodiment, the reporter protein comprises a luciferase. Non-limiting examples of useful luciferase include, e.g., Renilla luciferase, RLuc8 mutant Renilla luciferase, (dCpG)Luciferase, NanoLuc reporter, firefly luciferase, Gaussia luciferase (gLuc), MetLuc, Vibriofischeri lumazine protein, Vibrio harveyi luminaze protein, inoflagellate luciferase, firefly luciferase YY5 mutant, firefly luciferase LGR mutant, firefly luciferase mutant E, and fragments or derivatives thereof.

In one embodiment, the reporter protein comprises a fluorescent protein. Non-limiting examples of useful fluorescent proteins include, e.g., green fluorescent protein (GFP), GFP-like fluorescent proteins, (GFP-like), enhanced green fluorescent protein (EGFP), yellow fluorescent protein (YFP), enhanced yellow fluorescent protein (EYFP), blue fluorescent protein (BFP), enhanced blue fluorescent protein (EBFP), cyan fluorescent protein (CFP), enhanced cyan fluorescent protein (ECFP); red fluorescent protein, superfolder GFP, superfolder YFP, orange fluorescent protein, red fluorescent protein, small ultrared fluorescent protein, FMN-binding fluorescent protein, dsRed, qFP611, Dronpa, TagRFP, KFP, EosFP, IrisFP, Dendra, Kaede, KikGrl, emerald fluorescent protein, Azami Green, mWasabi, TagGFP, TurboGFP, AcGFP, ZsGreen, T-Sapphire, and fragments or derivatives thereof.

In one embodiment, the method comprises adding a reporter protein substrate for obtaining the reporter signal. Non-limiting examples of useful reporter protein substrates for luciferases include, e.g., Luciferin (e.g., d-luciferin), EnduRen, and coelenterazine luciferase substrates.

In some embodiments, the reporter protein is fused to a viral particle protein. In some embodiments, the viral particle is a lentiviral particle, and the viral particle protein is VPR.

In some embodiments, each viral particle within the library comprises a polynucleotide encoding a pMHC complex and optionally a universal primer binding sequence (a barcode).

In some embodiments, each viral particle within the library comprises a polynucleotide encoding a pMHC complex and a universal primer binding sequence, wherein the pMHC complex is the same as the pMHC complex which is present on the surface of the viral particle.

In some embodiments, each viral particle within the library is a lentiviral particle which comprises a fusogen, wherein the fusogen comprises a Sindbis virus glycoprotein or a fragment, mutant or derivative thereof. The fusogen may be a mutated Sindbis virus glycoprotein which does not bind its natural ligand. In one embodiment, the fusogen comprises the sequence set forth as SEQ ID NO: 5.

In some embodiments, libraries include pMHC-encoded (peptide/MHC-encoded) viral (e.g., lentiviral) particles described herein can be used in screening a population of antigen-specific cells (e.g., B cells or T cells). In some embodiments, libraries include pMHC-encoded (peptide/MHC-encoded) viral (e.g., lentiviral) particles described herein can be used in screening a population of T cells. In such libraries, the pMHC displayed on the virus surface will enable T cell interaction (e.g., viral fusion or T cell activation) in a TCR-specific manner. Infected T cells can be collected and sequenced, allowing for the identification of pMHCs that can infect a subset of a T cell population of interest. TCR sequences and its cognate peptide sequences can also be identified. In some embodiments, pMHC viral libraries comprise at least randomized viral vectors containing randomized pMHCs.

In some embodiments, pMHC viral libraries described herein are specific for a unique set of antigens. For example, the antigen may be a viral or bacterial antigen, or a neoantigen for a particular cancer such as those disclosed herein.

The libraries of the disclosure may include viral libraries and cellular libraries. Viral or cell libraries can vary in size from hundreds to thousands, millions, or more unique viral particles or unique cells. A library can comprise a thousand or inure, e.g, at least 1,000; 2,000; 3,000; 4,000; 5,000; 10,000; 50,000; 100,000; 500,000; 600,000; 700,000; 800,000; 900,000; 1,000,000; 2,000,000; 3,000,000; 4,000,000; or more members. In some embodiments, the libraries of the disclosure comprise at least 100,000 unique viral particles or unique cells. In some embodiments, the libraries of the disclosure comprise at least 200,000 unique viral particles or unique cells. In some embodiments, the libraries of the disclosure comprise at least 300,000 unique viral particles or unique cells. In some embodiments, the libraries of the disclosure comprise at least 400,000 unique viral particles or unique cells. In some embodiments, the libraries of the disclosure comprise at least 500,000 unique viral particles or unique cells.

In another aspect, the present disclosure provides a method for identifying an antigen-specific T-cell receptor (TCR), comprising:
a) contacting a sample comprising TCR-expressing cells, wherein the sample has been isolated from a subject who has been previously exposed to an antigen, with a library described herein, wherein the library comprises a plurality of viral particles comprising peptides (p) derived from the antigen, wherein the peptides are presented in the context of pMHC complexes on the surface of the viral particles, and wherein the viral particles in the library comprise a reporter protein or a polynucleotide encoding the reporter protein,
b) identifying one or more cells comprising the reporter protein, and
c) determining the TCR(s) or sequence(s) of TCR(s) expressed by the one or more cells identified in step (b).

In some embodiments, the TCR is identified using a single cell RNA sequencing.

In some embodiments, TCR-expressing cells are selected from CD8+ T cells, CD4+ T cells, or pan CD3+ T cells.

In some embodiments, the sample is isolated peripheral blood mononuclear cells (PBMCs).

In some embodiments, prior to step (a) the TCR-expressing cells in the sample are activated and expanded in the presence of the same plurality of antigen-derived peptides (p) as the peptides present in the library. The expansion may be conducted over the span of 10 days in culture media (e.g., RPMI medium containing human serum, IL-2, IL-15, IL-7, GM-CSF, IL-4, FLT3L, amd/or IFNα).

In some embodiments, the method further comprises determining the activation state of the one or more cells identified in step (b).

In some embodiments, the activation state of the one or more cells identified in step (b) is determining the expression level of one or more genes selected from IFNγ, granzyme B, 4-1BB, CD28, CD25, CD69, OX40, and CD40L.

In some embodiments, each of the viral particles in the library comprises a polynucleotide encoding pMHC complex and a universal primer binding sequence, wherein the pMHC complex is the same as the pMHC complex which is present on the surface of the viral particle.

In some embodiments, the method further comprises identifying the cognate peptide(s) recognized by the identified TCR(s), by determining the sequence of the peptide(s) contained within the pMHC-encoding polynucleotide(s) in the one or more cells identified in step (b).

In some embodiments, the peptide sequence is determined by sequencing using a primer that binds the universal primer binding sequence. In one embodiment, a primer that binds the universal primer binding sequence comprises the nucleotide sequence TCCCATATAAGAAAC (SEQ ID NO: 123) or a variant having at least about 70%, about 75%, about 80%, about 85%, or about 90% sequence identity thereto.

In one aspect, provided herein is a method for identifying an antigenic peptide recognized by one or more antigen-specific TCR-expressing cells, comprising:
a) contacting a sample comprising TCR-expressing cells, wherein the sample has been isolated from a subject who has been previously exposed to an antigen, with a library described herein, wherein the library comprises a plurality of viral particles comprising peptides (p) derived from the antigen, wherein the peptides are presented in the context of pMHC complexes on the surface of the viral particles, and wherein the viral particles in the library comprise (i) a reporter protein or a polynucleotide encoding the reporter protein and (ii) a polynucleotide encoding pMHC complex and a universal primer binding sequence, wherein the pMHC complex is the same as the pMHC complex which is present on the surface of the viral particle,
b) identifying one or more cells comprising the reporter protein, and
c) identifying the peptide(s) encoded by the viral particle(s) which had infected the one or more cells identified in step (b).

In some embodiments, the TCR-expressing cells are selected from CD8+ T cells, CD4+ T cells, or pan CD3+ T cells.

In some embodiments, the sample is isolated peripheral blood mononuclear cells (PBMCs).

In some embodiments, prior to step (a) the TCR-expressing cells in the sample are expanded in the presence of the same plurality of antigen-derived peptides (p) as the peptides present in the library.

In some embodiments, identifying the peptide(s) in step (c) comprises determining the sequence of the peptide(s) contained within the pMHC-encoding polynucleotide expressed by the one or more cells identified in step (b).

In some embodiments, the peptide sequence is determined by sequencing the viral genome using a primer that binds the universal primer binding sequence.

In some embodiments, the method further comprises identifying the antigenic peptide recognized by the largest number of TCR-expressing cells in samples isolated from a plurality of subjects who have been previously exposed to the antigen.

The identification of antigenic peptides can allow for the development of immunotherapeutic reagents designed to specifically target and destroy cells presenting the antigenic peptides. Such reagents may be moieties that bind to the antigenic peptide and/or pMHC complexes and induce a T cell response. Such reagents may be based on, e.g., antibodies, TCRs, and/or CARs. For example, in the case of viral infection, antigenic viral peptides can be identified using the methods described herein which can then be utilized as potential targets for developing immunotherapeutics (such as antibodies (e.g., bispecific antibodies), engineered TCR- or CAR-based cellular therapies) against the viral infection and/or related diseases or disorders.

An antigenic peptide of the disclosure may be present in a complex with an MHC molecule. Preferably, the peptide is bound to the peptide binding groove of the MHC molecule. In some embodiments, the peptide and the MHC molecule form a non-covalent complex. In other embodiments, the peptide and the MHC molecule may be covalently linked, for example, via a linker.

Peptides or pMHC complexes disclosed herein may be fused or conjugated to one or more heterologous molecules. Peptides or pMHC complexes of the disclosed herein may also be in multimeric form. Accordingly, the present disclosure also provides fusion proteins, conjugates, and oligomeric complexes comprising a peptide or a pMHC complex of the disclosure.

In some embodiments, peptides are fused or conjugated to one or more heterologous molecules which includes an MHC molecule (or fragments thereof). Heterologous molecules suitable for genetical fusion and/or chemical conjugation with the peptides or the pMHC complexes of the disclosure include, but are not limited to, peptides, polypeptides, small molecules, polymers, nucleic acids, lipids, sugars, etc. The heterologous molecule(s) may be fused at the N- and/or C-terminus of the peptide and/or another polypeptide chain in the pMHC complex.

An antigenic peptide of the disclosure, or pharmaceutically acceptable salt thereof, or fragment or derivative thereof, may be used to induce an immune response. For this purpose, peptides or pMHC complexes, or other peptide-based molecules (such as a complex, fusion protein, or conjugate comprising a peptide disclosed herein) of the present disclosure may be provided in the form of a vaccine composition. For example, the vaccine composition may be useful for the treatment or prevention of a viral infection and/or virus-induced diseases or disorders. As will be appreciated, vaccines may take several forms (see, e.g., Schlom, J Natl Cancer Inst. 2012; 104(8):599-613; Salgaller, Cancer Res. 1996; 56(20):4749-57 and Marchand, Int J Cancer. 1999; 80(2):219-30). The vaccine composition may include a plurality of peptides or pMHC complexes. Adjuvants may be added to the vaccine composition to augment the immune response. In particular for peptide-containing vaccines compositions of the disclosure, pharmaceutically acceptable adjuvants include, but are not limited to, aluminum salts, Amplivax, AS 15, Aquila's QS21 stimulon, AsA404 (DMXAA), beta-glucan, BCG, CP-870,893, CpG7909, CyaA, dSLIM, GM-CSF, IC30, IC31, Imiquimod, ImuFact EV1P321, IS Patch, ISS, 1018 ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, poly-ICLC, PepTel®, Pam3Cys, PLGA microparticles, resiquimod, SRL172, Virosomes and other Virus-like particles, YF-17D, VEGF trap, R848, and/or vadimezan.

Alternatively, the vaccine composition may take the form of an antigen-presenting cell (APC) displaying the peptide of the disclosure in complex with MHC. Preferably the APC is an immune cell, more preferably a dendritic cell or a B cell. The peptide may be pulsed onto the surface of the cell (Thurner, J Exp Med. 1999; 190(11):1669-78), or nucleic acid encoding for the peptide of the disclosure may be introduced into dendritic cells or B cells (e.g., by electroporation. Van Tendeloo, Blood. 2001; 98(1):49-56).

A polynucleotide encoding one or more antigenic peptides disclosed herein also can be used to make a vaccine. For example, the polynucleotide may be delivered in a viral vector or as an RNA vaccine. RNA vaccines may comprise mRNA and/or self-replicating RNA (also known as RNA replicons). Delivery techniques for RNA vaccines may also encompass, for example, condensation with protamine and encapsulation into liposomes or nanoparticles.

Peptides, pMHC complex, or other peptide-based molecules (such as a complex, fusion protein, or conjugate comprising a peptide disclosed herein) of the present disclosure can be used to identify and/or isolate binding moieties that bind specifically to a peptide, pMHC complex, or other peptide-based molecule of the disclosure. Such binding moieties may be used as immunotherapeutic reagents and may include, e.g., antibodies (or antigen-binding fragments thereof), alternative scaffolds, TCRs, and CARs.

In one aspect, the disclosure provides a peptide binding moiety that binds a peptide of the disclosure. Preferably the peptide binding moiety binds a peptide when the peptide is in complex with MHC. In the latter instance, the peptide binding moiety may bind partially to the MHC, provided that the peptide binding moiety also binds to the peptide. The peptide binding moiety may bind only the peptide, and that binding may be specific. The peptide binding moiety may bind only the pMHC complex and that binding may be specific.

The disclosure also provides a method of identifying a peptide binding moiety that binds a pMHC complex of the disclosure, the method comprising contacting a candidate peptide binding moiety with the pMHC complex and determining whether the candidate peptide binding moiety binds the complex. Methods to determine binding to pMHC complexes include, for example, surface plasmon resonance, or any other biosensor technique, ELISA, flow cytometry, chromatography, microscopy. Alternatively, or in addition, binding may be determined by functional assays in which a biological response is detected upon binding, for example, cytokine release or cell apoptosis.

The candidate peptide binding moiety may be a peptide binding moiety of the type already described, such as an antibody or a TCR.

For example, antibodies and TCRs may be obtained from display libraries in which the pMHC complex of the disclosure is used to pan the library. TCRs can be displayed on the surface of phage particles and yeast particles, for example, and such libraries have been used for the isolation of high affinity variants of TCR derived from T cell clones. TCR phage libraries can be used to isolate TCRs with novel antigen specificity. Such libraries can be constructed with α- and β-chain sequences corresponding to those found in a natural repertoire. However, the random combination of these α- and β-chain sequences, which occurs during library creation, can produce a repertoire of TCRs that may not be naturally occurring.

In some embodiments, the pMHC complex of the disclosure may be used to screen a library of diverse TCRs displayed on the surface of phage particles. The TCRs displayed by said library may not correspond to those contained in a natural repertoire, for example, they may contain α- and β-chain pairing that would not be present in vivo, and/or the TCRs may contain non-natural mutations and/or the TCRs may be in soluble form. Screening may involve panning the phage library with pMHC complexes of the disclosure and subsequently isolating bound phage particles.

For this purpose, pMHC complexes may be attached to a solid support, such as a magnet bead, or column matrix and phage bound pMHC complexes isolated, with a magnet, or by chromatography, respectively. The panning steps may be repeated several times. Isolated phage may be further expanded in E. coli cells. Isolated phage particles may be tested for specific binding to pMHC complexes of the disclosure. Binding can be detected using techniques including, but not limited to, ELISA, or SPR for example using a BiaCore instrument. The DNA sequence of the T cell receptor displayed by pMHC binding phage can be further identified by PCR methods.

Alternatively, antigen binding T cells and TCRs can be isolated from fresh blood obtained from patients or healthy donors. Such a method involves stimulating T cells using autologous dendritic cells (DCs), followed by autologous B cells, and then pulsed with a peptide disclosed herein. Several rounds of stimulation may be carried out, for example three or four rounds. Activated T cells may then be tested for specificity by measuring cytokine release in the presence of T2 cells pulsed with the peptide of the disclosure (for example using an IFNγ ELISpot assay). Activated cells may then be sorted by fluorescence-activated cell sorting (FACS) using labelled antibodies to detect intracellular cytokine production (e.g. IFNγ), or expression of a cell surface marker (such as CD137). Sorted cells may be expanded and further validated, for example, by ELISpot assay and/or cytotoxicity against target cells and/or staining by peptide-MHC tetramer. The TCR chains from validated clones may then be amplified by rapid amplification of cDNA ends (RACE) and sequenced.

A peptide binding moiety disclosed herein can include, for example, without limitation, an antibody, a TCR, or a CAR.

In some embodiments, the peptide binding moiety of the disclosure may be an antibody or antigen-binding fragment thereof. Antibodies or antigen-binding fragments thereof encompass derivatives, functional equivalents, and homologues of antibodies, humanized antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. A humanized antibody may be a modified antibody having the variable regions of a non-human, e.g. murine, antibody, and the constant region of a human antibody. Examples of antibodies are the immunoglobulin isotypes (e.g., IgG, IgE, IgM, IgD and IgA) and their isotypic subclasses; or fragments that comprise an antigen binding domain such as Fab, scFv, Fv, dAb, Fd; and diabodies. Antibodies may be polyclonal or monoclonal. A monoclonal antibody may be referred to herein as “mab”.

In some embodiments, the antibody is a multispecific antibody. In some embodiments, the antibody is a bispecific antibody. The bispecific antibody may comprise a second targeting moiety that targets to the desired cell or tissue, e.g., liver, or to another desired antigen associated with the same or similar disease or disorder (e.g., liver cancer antigen).

It is possible to take an antibody, for example a monoclonal antibody, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers, (ix) “diabodies”, multivalent or multispecific fragments constructed by gene fusion, and (x) VHH or VNAR antibodies, also known as single-domain antibodies or nanobodies (Nb), which may be derived from heavy-chain antibodies from e.g., dromedaries, camels, llamas, alpacas, or sharks.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). Where bispecific antibodies are to be used, these may be conventional bispecific antibodies, which can be manufactured in a variety of ways, e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins”. Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display from libraries. If one arm of the diabody is to be kept constant, for instance, with a specificity directed against an antigen of interest, then a library can be made where the other arm is varied, and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

In some embodiments, the peptide binding moiety may be an antibody-like molecule that has been designed to specifically bind a peptide or peptide-MHC complex of the disclosure. In some embodiments the peptide binding moiety may comprise a TCR-mimic antibody. In some embodiments, such TCR-mimic antibodies can comprise high-affinity soluble antibody molecules endowed with a TCR-like specificity towards tumor or viral epitopes that can target tumor and/or virus-infected cells and mediate their specific killing.

Also encompassed within the present disclosure are binding moieties based on engineered protein scaffolds or “alternative scaffolds”. Alternative scaffolds are derived from stable, soluble, natural protein structures which have been modified to provide a binding site for a target molecule of interest. Examples of alternative scaffolds include, but are not limited to, affibodies, which are based on the Z-domain of staphylococcal protein A that provides a binding interface on two of its α-helices; anticalins, derived from lipocalins, that incorporate binding sites for small ligands at the open end of a β-barrel fold; monobodies, designed to incorporate the fibronectin type III domain (Fn3) of fibronectin or tenascin as a protein scaffold or synthetic FN3 domains (e.g., tencon); nanobodies, and DARPins. Additional alternative scaffolds include Adnectin™, iMab, EETI-II/AGRP, Kunitz domain, thioredoxin peptide aptamer, Affilin, Tetranectin, Fynomer, and Avimer. Alternative scaffolds are typically targeted to bind the same antigenic proteins as antibodies, and are potential therapeutic agents. They may act as inhibitors or antagonists, or as delivery vehicles to target molecules, such as toxins, to a specific tissue in vivo. Short peptides may also be used to bind a target protein. Phylomers are natural structured peptides derived from bacterial genomes. Such peptides represent a diverse array of protein structural folds and can be used to inhibit/disrupt protein-protein interactions in vivo.

Alternative scaffolds are typically single chain polypeptidic frameworks that contain a highly structured core associated with variable domains of high conformational tolerance allowing insertions, deletions, or other substitutions within the variable domains. Libraries introducing diversity to one or more variable domains, and in some cases to the structured core, may be generated using known protocols and the resulting libraries may be screened for binding to the peptide and/or the pMHC complex of the disclosure, and the identified binders may be further characterized for their specificity using known methods. Alternative scaffolds may be derived from Protein A, in particular, the Z-domain thereof (affibodies), ImmE7 (immunity proteins), BPTI/APPI (Kunitz domains), CTLA-4, charybdotoxin (Scorpion toxin), Min-23 (knottins), lipocalins (anticalins), Ras-binding protein AF-6 (PDZ-domains), neokarzinostatin, a fibronectin domain, an ankyrin consensus repeat domain, or thioredoxin.

In some embodiments, the antibodies or alternative scaffolds described herein can be immobilized on viral vectors. Such modified recombinant viral vectors can be useful for the targeted introduction of genetic materials encoded by the viral vectors into cells and/or tissues (e.g., liver cells and/or liver tissues). Various means can be used to mobilize the antibodies or alternative scaffolds to the viral vectors, for example, by using an affinity binding pair, such as c-Myc/anti-Myc antibody, streptavidin/biotin, or via spy-tag/spy-catcher system. Exemplary vectors that may be modified with the antibodies or alternative scaffolds described herein include, but are not limited to, adeno-associated virus (AAV) vectors (e.g., AAV1, AAV2, AAV6, AAV9, or AAV9.PHP), retroviral vectors, lentiviral vectors, and targeted oncolytic viruses (e.g., herpes simplex virus (HSV)).

In some embodiments, the peptide binding moiety may be a TCR. TCRs are described using the International Immunogenetics (IMGT) TCR nomenclature, and the IMGT public database of TCR sequences.

The TCRs of the present disclosure may be in any format. For example, the TCRs may be αβ heterodimers, or αα or ββ homodimers.

α/β heterodimeric TCRs have an α-chain and a β-chain. Broadly, each chain comprises variable, joining and constant region, and the β-chain also usually contains a short diversity region between the variable and joining regions, but this diversity region is often considered as part of the joining region. Each variable region comprises three hypervariable CDRs (Complementarity Determining Regions) embedded in a framework sequence; CDR3 is believed to be the main mediator of antigen recognition. There are several types of α-chain variable (Vα) regions and several types of β-chain variable (Vβ) regions distinguished by their framework, CDR1 and CDR2 sequences, and by a partly defined CDR3 sequence.

The TCRs of the disclosure may not correspond to TCRs as they exist in nature. For example, they may comprise α- and β-chain combinations that are not present in a natural repertoire. Alternatively or additionally, a TCR described herein may be soluble, and/or the α- and/or β-chain constant domain may be truncated relative to the native/naturally occurring TRAC/TRBC sequences such that, for example, the C-terminal transmembrane domain and intracellular regions are not present. Such truncation may result in removal of the cysteine residues from TRAC/TRBC that form the native interchain disulfide bond.

In addition, the TRAC/TRBC domains may contain modifications. For example, the α-chain extracellular sequence may include a modification relative to the native/naturally occurring TRAC whereby amino acid T48 of TRAC, with reference to IMGT numbering, is replaced with C48. Likewise, the β-chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native α- and β-chain extracellular sequences enable the formation of a non-native interchain disulfide bond which stabilizes the refolded soluble TCR, i.e. the TCR formed by refolding extracellular α- and β-chains. This non-native disulfide bond facilitates the display of correctly folded TCRs on phage. In addition, the use of the stable disulfide linked soluble TCR enables more convenient assessment of binding affinity and binding half-life. Alternative positions for the formation of a non-native disulfide include, for example, Thr 45 of exon 1 of TRAC*01 and Ser 77 of exon 1 of TRBC1*01 or TRBC2*01; Tyr 10 of exon 1 of TRAC*01 and Ser 17 of exon 1 of TRBC1*01 or TRBC2*01; Thr 45 of exon 1 of TRAC*01 and Asp 59 of exon 1 of TRBC1*01 or TRBC2*01; and Ser 15 of exon 1 of TRAC*01 and Glu 15 of exon 1 of TRBC1*01 or TRBC2*01. TCRs with a non-native disulfide bond may be full length or may be truncated.

TCRs of the disclosure may be in single chain format. Single chain TCRs include αβTCR polypeptides of the type: Vα-L-Vβ, Vβ-L-Vα, Vα-Cα-L-Vβ, Vα-L-Vβ-Cβ or Vα-Cα-L-Vβ-Cβ, optionally in the reverse orientation, wherein Vα and Vβ are TCR α and β variable regions respectively, Cα and Cβ are TCR α and β constant regions respectively, and L is a linker sequence. Single chain TCRs may contain a non-native disulfide bond. The TCR may be in a soluble form (i.e. having no transmembrane or cytoplasmic domains) or may contain full length α- and β-chains. The TCR may be provided on the surface of a cell, such as a T cell.

TCRs of the disclosure may be engineered to include mutations. Methods for producing mutated high affinity TCR variants such as phage display and site directed mutagenesis. Preferably, mutations to improve affinity are made within the variable regions of α- and/or β-chains. More preferably mutations to improve affinity are made within the CDRs. There may be between 1 and 15 mutations in the α- and or β-chain variable regions.

TCRs of the disclosure may also be labeled with an imaging compound, for example a label that is suitable for diagnostic purposes. Such labelled high affinity TCRs are useful in a method for detecting a TCR ligand selected from CD1-antigen complexes, bacterial superantigens, and MHC-peptide/superantigen complexes, which method comprises contacting the TCR ligand with a high affinity TCR (or a multimeric high affinity TCR complex) which is specific for the TCR ligand; and detecting binding to the TCR ligand. In multimeric high affinity TCR complexes (formed, for example, using biotinylated heterodimers) fluorescent streptavidin can be used to provide a detectable label. A fluorescently labelled multimer is suitable for use in FACS analysis, for example to detect antigen presenting cells carrying the peptide for which the high affinity TCR is specific.

A TCR of the present disclosure (or multivalent complex thereof) may alternatively or additionally be associated with (e.g. covalently or otherwise linked to) a therapeutic agent which may be, for example, a toxic moiety for use in cell killing, or an immunostimulating agent such as an interleukin or a cytokine. A multivalent high affinity TCR complex of the present disclosure may have enhanced binding capability for a TCR ligand compared to a non-multimeric wild-type or high affinity T cell receptor heterodimer. Thus, the multivalent high affinity TCR complexes according to the disclosure are particularly useful for tracking or targeting cells presenting particular antigens in vitro or in vivo, and are also useful as intermediates for the production of further multivalent high affinity TCR complexes having such uses. The high affinity TCR or multivalent high affinity TCR complex may therefore be provided in a pharmaceutically acceptable formulation for use in vivo.

High affinity TCRs of the disclosure may be used in the production of soluble bi-specific reagents. One exemplary embodiment is a reagent which comprises a soluble TCR, fused via a linker to an anti-CD3 specific antibody fragment (see, e.g., US Patent Application US20190016801A1, which is incorporated herein by reference in its entirety).

In a further aspect, the disclosure provides nucleic acid encoding the TCR of the disclosure, a TCR expression vector comprising nucleic acid encoding a TCR of the disclosure, as well as a cell harboring such a vector. The TCR may be encoded either in a single open reading frame or two distinct open reading frames. Also included in the scope of the disclosure is a cell harboring a first expression vector which comprises nucleic acid encoding an α-chain of a TCR of the disclosure, and a second expression vector which comprises nucleic acid encoding a β-chain of a TCR of the disclosure. Alternatively, one vector may encode both an α- and a β-chain of a TCR of the disclosure.

A further aspect of the present disclosure provides a cell displaying on its surface a TCR of the disclosure. The cell may be a T cell, or other immune cell. The T cell may be modified such that it does not correspond to a T cell as it exists in nature. For example, the cell may be transfected with a vector encoding a TCR of the disclosure such that the T cell expresses a further TCR in addition to the native TCR. Additionally or alternatively, the T cell may be modified such that it is not able to present the native TCR. There are a number of methods suitable for the transfection of T cells with DNA or RNA encoding the TCRs of the disclosure. As a non-limiting example, the transfection method may comprise a rapid RNA-based transfection system. T cells expressing the TCRs of the disclosure are suitable for use in adoptive therapy-based treatment of diseases such as cancers. There are a number of suitable methods by which adoptive therapy can be carried out. For example, adoptive cell therapy (ACT) may comprise use of autologous tumor-infiltrating lymphocytes, and may include a lymphodepletion preparative regimen prior to ACT. In some embodiments, viruses, e.g., retroviruses, that encode TCRs may be used for genetic modification of lymphocytes to convert normal lymphocytes into lymphocytes with anti-cancer activity. The adoptive transfer of lymphocytes with anti-cancer activity into patients requiring treatment of, e.g., metastatic melanoma, can mediate tumor regression. In some embodiments, ACT may comprise treatment of patients with cancers expressing viral or alloantigens, treatment of patients with cancers expressing viral antigens, and/or ACT using gene-modified lymphocytes. In some embodiments, ACT methods may include, for example, genetic modification of lymphocytes to introduce new recognition specificities using, e.g., αβTCR(s) and/or chimeric TCR(s); genetic modification of lymphocytes to alter function of T cells using, e.g., co-stimulatory molecules (e.g., CD28, 4-1BB), cytokines (e.g., IL2, 1L15), homing molecules (e.g., CD62L, CCR7), and/or molecules capable of preventing apoptosis (BCL2); modification of host lymphodepletion using, e.g., selective depletion of CD4+ cells or T regulatory cells; blocking of inhibitory signals on reactive lymphocytes using, e.g., antibodies to CTLA4 and/or PD-1; administration of vaccines to stimulate transferred cells using, e.g., recombinant virus encoding antigen(s); administration of alternative cytokines to support cell growth using, e.g., IL15 and/or IL21; stimulation of APCs using, e.g., toll-like receptor agonists; generation of less differentiated lymphocytes using, e.g., alternate culture conditions and growth promoting cytokines in vitro; and, overcoming antigen escape variants using, e.g., natural killer cells.

The TCRs of the disclosure intended for use in adoptive therapy are generally glycosylated when expressed by the transfected T cells. The glycosylation pattern of transfected TCRs may be modified by mutations of the transfected gene.

In some embodiments, the peptide binding moiety may be a chimeric antigen receptor (CAR). CARs are genetically engineered receptors. CARs may be generated that bind the peptides or pMHC complexes of the present disclosure by incorporating an antigen binding domain that specifically binds the peptide or pMHC complex to the extracellular domain of the CAR. CARs may be introduced into and expressed by immune cells, such as T cells, NK cells, or macrophages. CARs can be programmed to both recognize a specific antigen and, when bound to that antigen, activate the immune cell to attack and destroy the cell presenting that antigen. When these antigens exist on tumor cells, an immune cell that expresses the CAR can target and kill the tumor cell.

The general structure of a CAR typically comprises an extracellular domain that binds the antigen (e.g. the peptides or pMHC complexes of the present disclosure), a hinge, a transmembrane domain, and an intercellular domain comprising a signaling domain and optionally one or more co-stimulatory domains.

Extracellular domains of the CAR may contain any polypeptide that specifically binds the desired antigen (e.g. the peptides or pMHC complexes of the present disclosure). For example, the extracellular domain may comprise an antibody fragment such as scFv or VHH. The CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

A hinge domain may be present between the extracellular domain and the transmembrane domain of the CAR, e.g., to provide flexibility to allow effective binding of the extracellular domain to its intended target. The hinge domain may be a polypeptide of about 2 to 100 amino acids in length. The hinge may include or be composed of flexible residues such as Gly and Ser so that the adjacent protein domains are free to move relative to one another. Longer hinges may be used when it is desirable to ensure that two adjacent domains do not sterically interfere with one another. The hinge may be derived from a hinge region or portion of the hinge region of any immunoglobulin. Non-limiting examples of linkers include a part of human CD8α chain, extracellular domain of CD28, an Ig hinge from IgG, IgM, IgA, IgD, or IgE, FeyRllla receptor, or a functional fragment thereof.

Transmembrane domains of the CAR may be derived transmembrane proteins, such as an alpha, beta or zeta chain of a T-cell receptor, CD28, CD3 epsilon, CD2, CD4, CD5, CD8, CD9, CD16, CD18, CD19, CD22, CD27, CD29, CD33, CD37, CD40, CD45, CD49a, CD64, CD80, CD84, CD86, CD96 (Tactile), CD100 (SEMA4D), CD103, CD134, CD154, CD160 (BY55), KIRDS2, OX40, LFA-1 (CD11a, CD18), CD11b, CD11c, CD11d, ICOS (CD278), 4-1 BB (CD137), 4-1 BBL, GITR, BAFFR, HVEM (LIGHTR), SLAMF7, NKp80 (KLRFI), IL2R beta, IL2R gamma, IL7Ra, ITGA1, VLA1, ITGA4, IA4, CD49D, ITGA6, VLA-6, CD49f, ITGAD, ITGAE, ITGAL, LFA-1, ITGAM, ITGAX, ITGB1, ITGB2, LFA-1, ITGB7, TNFR2, DNAM1 (CD226), SLAMF4 (CD244, 2B4), CEACAM1, CRT AM, Ly9 (CD229), PSGL1, SLAMF6 (NTB-A, Ly108), SLAM (SLAMF1, CD150, IPO-3), BLAME (SLAMF8), SELPLG (CD162), LTBR, PAG/Cbp, NKp30, NKp44, NKp46, NKG2D, and NKG2C, or functional fragment thereof.

The intracellular signaling domain of a CAR participates in transducing the signal of effective CAR binding to a target antigen into the interior of the immune effector cell to elicit an effector cell function, e.g., activation, cytokine production, proliferation, and cytotoxic activity, including the release of cytotoxic factors to the CAR-bound target cell, or other cellular responses elicited following antigen binding to the extracellular CAR domain. Non-limiting examples of intracellular signaling domains of the CAR include those derived from CD3ζ, CD3ε, CD3δ, CD3γ, CD5, CD22, CD39, CD79A, CD79B, CD66d, CD226, DAP10, DAP12, Fc epsilon receptor I gamma chain (FCER1G), or FcR β.

Intracellular co-stimulatory domains of the CAR can provide a second signal required for efficient activation and function of T lymphocytes upon binding to antigen. Such co-stimulatory domains may be derived from one or more co-stimulatory molecules, such as, but not limited to, 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, BTLA, GITR, CD226, HVEM, and ZAP70.

The CARs can be generated by standard molecular biology techniques. The extracellular domain that binds the desired antigen may be derived from the antibodies or their antigen binding fragments described herein.

In another aspect, the disclosure further provides cells that comprise peptide a binding moiety (e.g., TCRs and CARs) of the present disclosure. In some embodiments, the host cell is an immune cell. In some embodiments, the immune cell is T cell, NK cell, or a macrophage. The host cell may be autologous or allogeneic with respective to the subject receiving the cell (as treatment).

In some embodiments, TCRs of the present disclosure are provided as TCR-T cells. In some embodiments, CARs of the present disclosure are provided as CAR-T cells. Any methods known in the art for modifying T cells to express a TCR or CAR can be employed to generate the TCR-T or CAR-T cells of the present disclosure.

The cells expressing a peptide binding moiety (e.g., TCRs and CARs) of the present disclosure may also contain one or more additional genes. The additional genes can be used to increase the effector function of the cells expressing the peptide binding moiety (e.g., TCRs and CARs). Non-limiting examples of classes of additional genes include (a) a second targeting moiety, such as antibodies, including fragments thereof and bispecific antibodies (e.g., bispecific T cell engagers (BiTEs)), (b) secretable cytokines (e.g., GM-CSF, IL-7, IL-12, IL-15, IL-18), (c) membrane bound cytokines (e.g., IL-15), (d) chimeric cytokine receptors (e.g., IL-2/IL-7, IL-4/IL-7), (e) constitutive active cytokine receptors (e.g., C7R), (f) dominant negative receptors (DNR; e.g., TGFRII DNR), (g) ligands of co-stimulatory molecules (e.g., CD80, 4-1BBL), (h) nuclear factor of activated T cells (NFATs) (e.g., NFATc1, NFATc2, NFATc3, NFATc4, and NFAT5), or (j) suicide genes (e.g., CD20, truncated EGFR or HER2, inducible caspase 9 molecules). In some embodiments, the cells expressing a peptide binding moiety (e.g., TCRs and CARs) of the present disclosure may express a second targeting moiety that targets to the liver or to another known liver cancer antigen.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Specific and Functional Targeting of Jurkat-Derived T Cells Expressing the P14 and the OT-1 T Cell Receptor (TCR) with Lentiviral Particles Displaying H-2D^{b}-Restricted Gp33 Peptide on its Surface

This example describes generation of lentiviral vectors pseudotyped with MHC class I molecules displaying an antigenic peptide (i.e., epitope) and their use for transducing cells expressing a T cell receptor (TCR) that recognizes this peptide (FIG. 1A). This method can allow for targeting specific T cell subsets to downregulate their function (e.g., in the context of autoimmunity), kill them, activate them, or modify them.

In particular, this example demonstrates the targeting of T cells expressing the P14 TCR with lentiviral particles pseudotyped with a MHC class I molecule (H-2D^{b}) presenting the gp33-41(gp33) peptide in MHC groove. gp33-41—also simply named gp33—(KAVYNFATC, SEQ ID NO: 1 and 2) is a viral peptide corresponding to the residues 33 to 41 of the lymphocytic choriomeningitis virus (LCMV) glycoprotein (LCMVGP, SEQ ID NO: 62, GenBank: AF186080) and displayed by the MHC class I H-2D^{b} (GenBank: M18523.1, U47325.1) molecule in cells infected with LCMV. The P14 TCR specifically recognizes the gp33 peptide when presented by the MHC class I H-2D^{b} molecule.

First, a lentiviral particle comprising the mouse P14 TCR as a nucleotide of interest was generated and used to transduce Jurkat-derived J.RT3-T3.5/AP1-Luc/mCD28/mCD8αβ cells to create a stable immortalized T cell-derived cell line expressing the P14 TCR (J.RT3-T3.5/AP1-Luc/mCD28/mCD8αβ/P14 cell line). The P14 TCR expression cassette (SEQ ID NO: 3) has been synthesized by Integrated DNA Technologies as the association of the P14 TCRα subunit (Vα2-JαTA31, GenBank: X06771.1) linked to the P14 TCRβ subunit (Vβ8.1-Dβ-Jβ2.4-Cβ2, GenBank: X06772.1) via a T2A self-cleaving peptide to allow expression of both subunits from the same gene (de Felipe et al., Trends Biotechnol 24(2):68-75 (2006)). Other methods to generate the J.RT3-T3.5/AP1-Luc/mCD28/mCD8αβ/P14 cell line include nucleofection, electroporation, and transfection of linearized plasmid (using lipofectamine, calcium chloride, calcium phosphate or PEI).

Another lentiviral particle was generated comprising at its surface (i) H-2D^{b} (MHC class I molecule) displaying the gp33 peptide to target the P14 TCR (mROR-gp33-41-β2m-H-2D^{b}, SEQ ID NO: 4) and (ii) a fusogen “SINmu,” having an amino acid sequence set forth as SEQ ID NO: 5 and a nucleotide sequence set forth as SEQ ID NO: 6. SINmu was generated by mutating the Sindbis virus envelope glycoprotein to comprise a deletion of amino acids 61-64 of E3 and the following substitutions: mutations of 68SLKQ71 (“SLKQ” disclosed as SEQ ID NO: 71) into 68AAAA71 (“AAAA” disclosed as SEQ ID NO: 72); mutations of 157KE158 into 157AA158. The mutations resulted in a fusogen which does not bind its natural/cognate receptor but maintains its fusogenic properties (Morizono et al., Nature Medicine, 11(30):346-352 (2005); Yang et al., PNAS 103(31):11479-11484 (2006)).

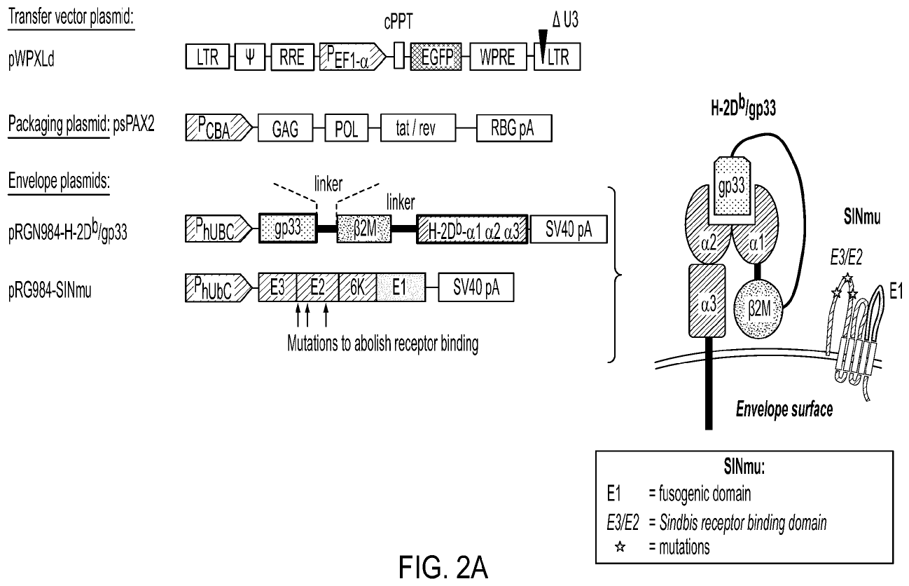

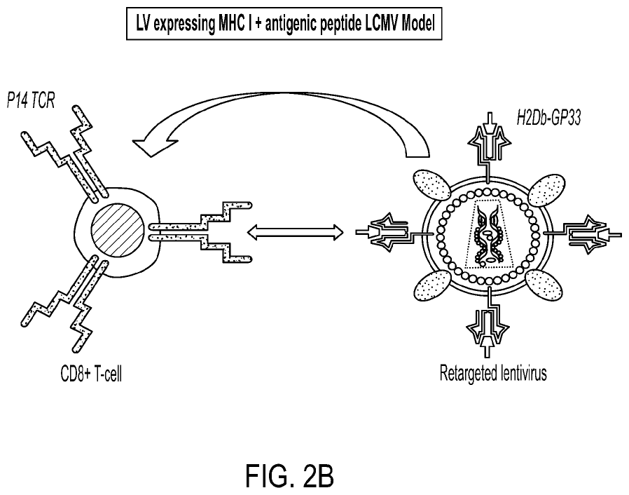

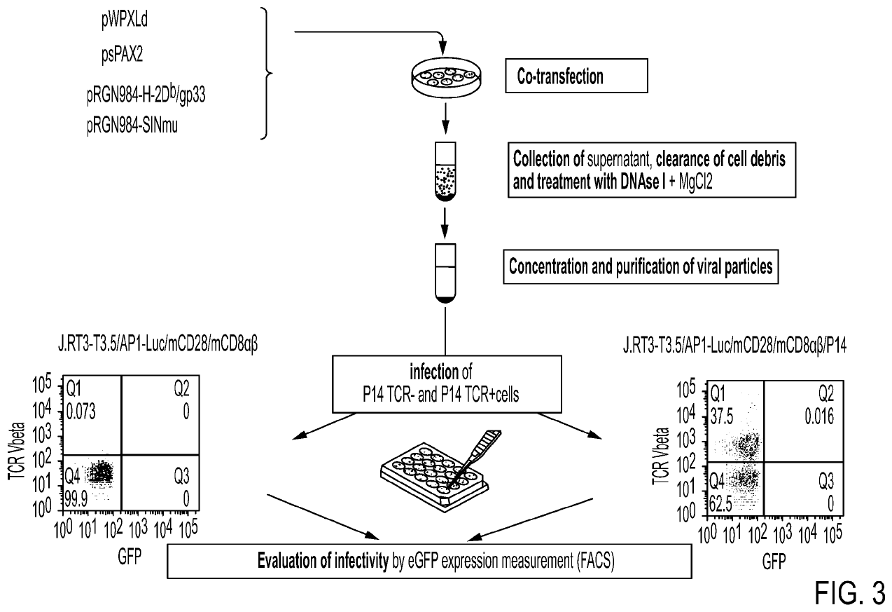

This lentiviral particle displaying H-2D^{b}/gp33 on its surface was also generated to carry an EGFP reporter nucleotide of interest (any other reporter gene can be used, including, but not limited to, LNGFR, Thy1.1, DsRed, RFP, YFP, CFP, mCherry, TdTomato, Luciferase, and Lac-Z). FIG. 2A shows a schematic of the LV-H-2D^{b}/gp33-EGFP lentiviral particle and also outlines the general structure of the plasmids used for particle generation. FIG. 2B depicts the interaction between the LV-H-2D^{b}/gp33-EGFP lentiviral particle and a CD8+ T cell expressing a P14 TCR. FIG. 3 outlines the process of production and purification of LV-H-2D^{b}/gp33-EGFP particles and their use for transducing cells expressing the P14 TCR.

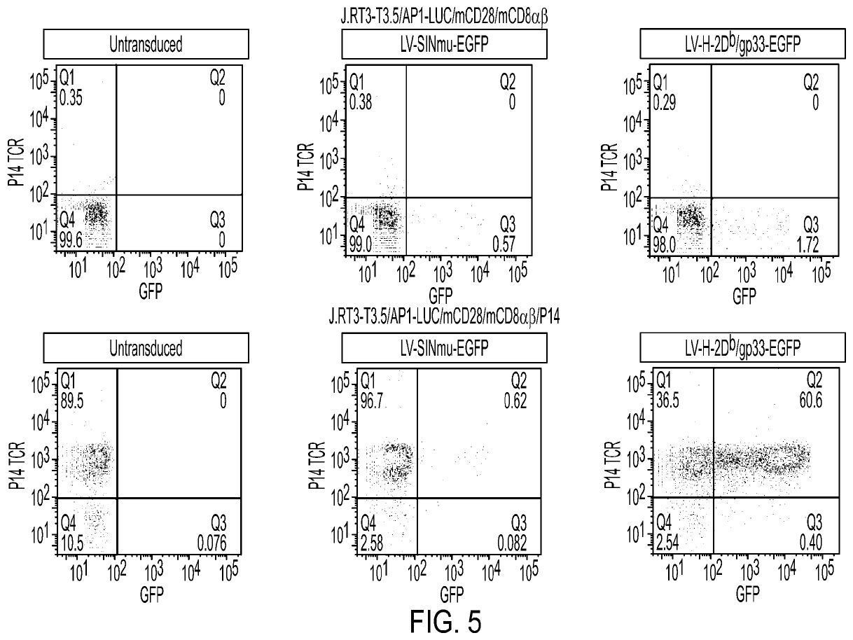

The P14 TCR-expressing cells J.RT3-T3.5/AP1-Luc/mCD28/mCD8αβ/P14 were successfully transduced with the H-2D^{b}/gp33 pseudotyped lentiviral particles LV-H-2D^{b}/gp33-EGFP resulting in the expression of EGFP, while control cells not comprising the TCR (J.RT3-T3.5/AP1-Luc/mCD28/mCD8αβ) did not demonstrate EGFP expression (FIGS. 4 and 5). Other negative controls included transduction without the lentiviral particle or with lentiviral particles comprising the fusogen SINmu on its surface but no H-2D^{b}/gp33 (LV-SINmu-EGFP, FIGS. 4 and 5). These experiments demonstrate the specificity of TCR targeting by the created MHC/epitope pseudotyped lentiviral particles.

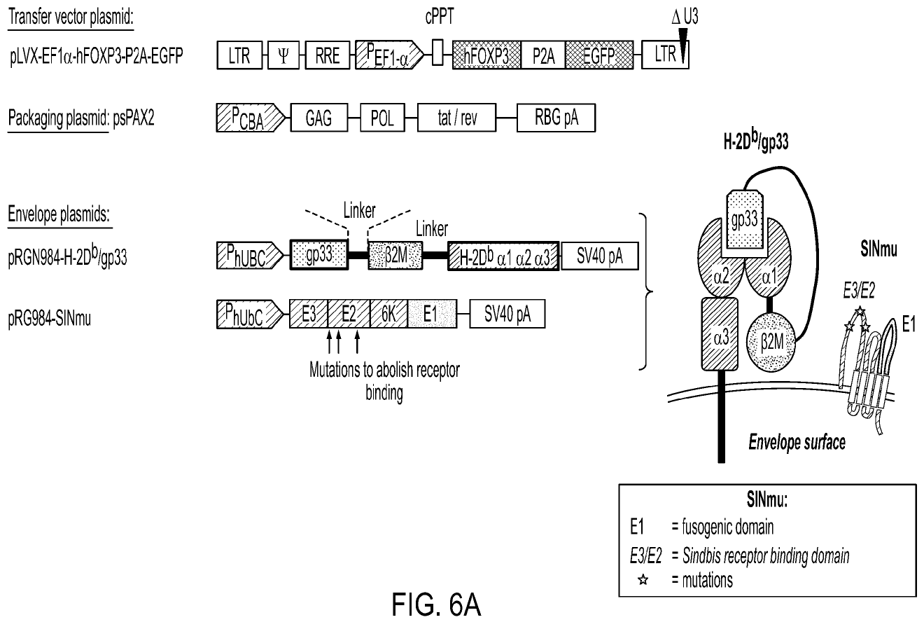

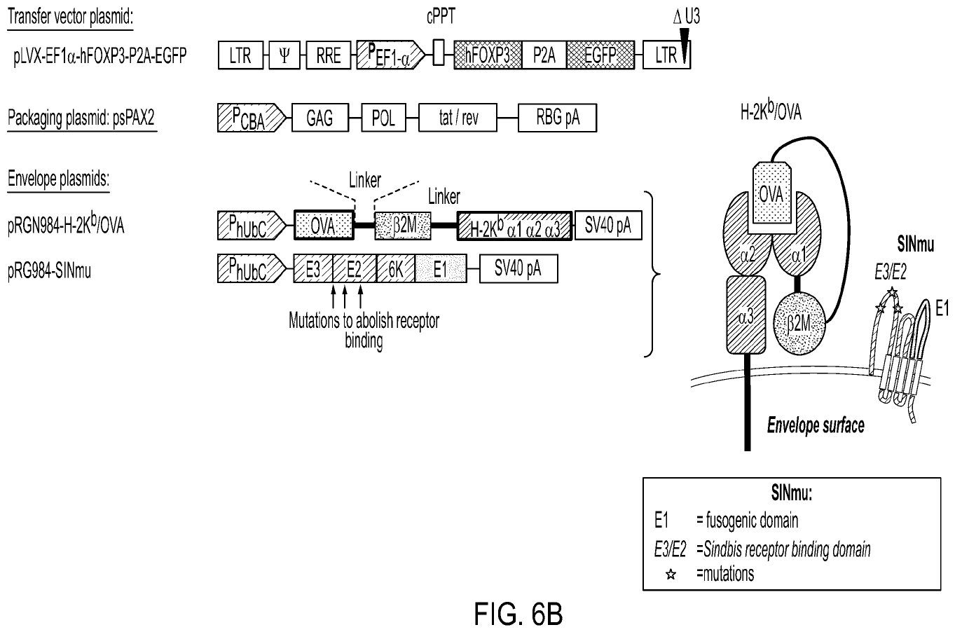

For functional studies, bicistronic lentiviral particles pseudotyped with the H-2D^{b}/gp33 (FIG. 6A) or the H-2K^{b}/OVA (FIG. 6B) complex are used to deliver the transcription factor Forkhead Box β3 (FOXP3) in T cells ex vivo, as well as the reporter protein EGFP to be able to monitor and sort the transduced cells. The effects of such treatment on the T cell phenotype are analyzed. FOXP3 is a transcription factor mostly expressed in CD4+CD25+ regulatory T cells (Tregs) and plays an essential role in the development and maintenance of those cells. Ectopic expression of FOXP3 via pantropic gamma-retroviral vectors or lentiviral vectors has been shown in several studies to convert naïve human primary CD4+ T cells, as well as Jurkat-T cells, into Tregs-like cells (Fontenot et al., Nature Immunology, 4(4):330-336 (2003), Hori et al., Science, 299(5609):1057-1061 (2003), Kim et al., Biochemical and Biophysical Research Communications, 362:44-50 (2007), Allan et al., J. Clin. Invest. 115:3276-3284 (2005), Allan et al., Mol. Ther., 16(1): 194-202 (2008), Himmel et al., Eur. J. Immunol. 41: 306-312 (2011)).

Therefore Treg-like phenotype induced by FOXP3 expression constitutes an ideal readout for assessing the capacity of lentiviral particles displaying peptides in groove of MHC (PIG) to specifically transduce and modulate T cells function. Specifically, the ability of lentiviral particles displaying the H-2-D^{b}/gp33 complex on their surface and comprising the genetic sequence encoding human FOXP3 (hFOXP3, Genbank: AF277993, EF534714, EF534714, DQ010327) as a nucleotide of interest to transfer the said nucleotide of interest and modulate T cell phenotype, in this case a T cell regulatory (Treg) phenotype, in infected cells is evaluated in Jurkat-derived T cells expressing the gp33-specific P14 TCR.

A lentiviral particle was generated comprising at its surface i) H-2K^{b} (MHC class I molecule) displaying the OVA₂₅₇₋₂₆₄ (OVA) peptide (SIINFEKL; SEQ ID NO: 7 and 8) to target the OVA-restricted TCRs (mROR-OVA-β2m-H-2K^{b}, SEQ ID NO: 9) and ii) the fusogen SINmu as described above. This OVA peptide is the MHC class I (K^{b}) restricted peptide epitope corresponding to the residues 257 to 262 of the Ovalbumin protein (GenBank: V00383.1, AH002466.2, V00438, J00895, AY223553, V00382).

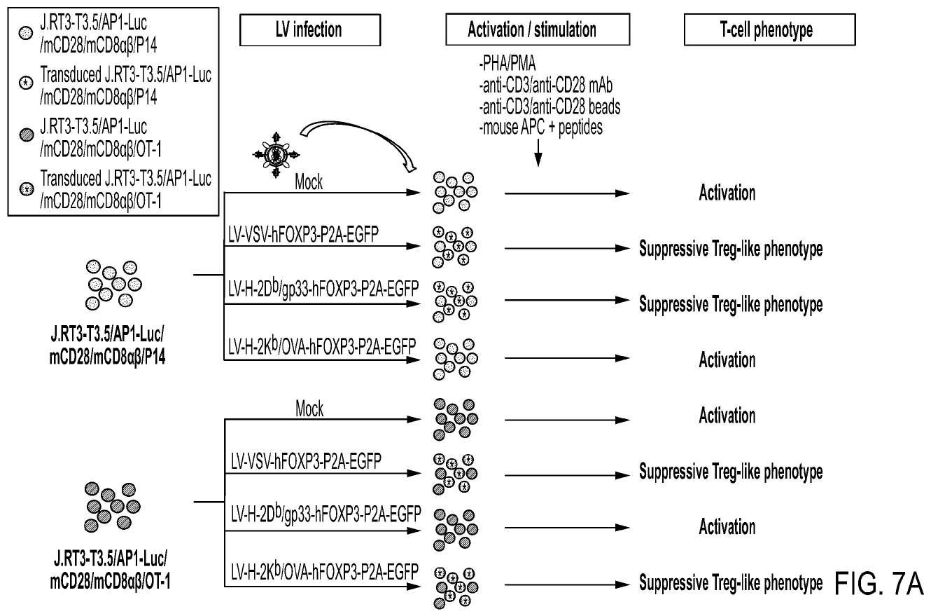

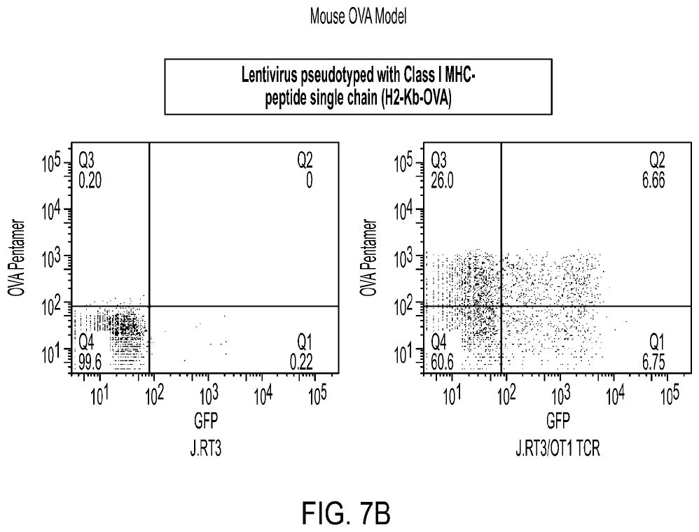

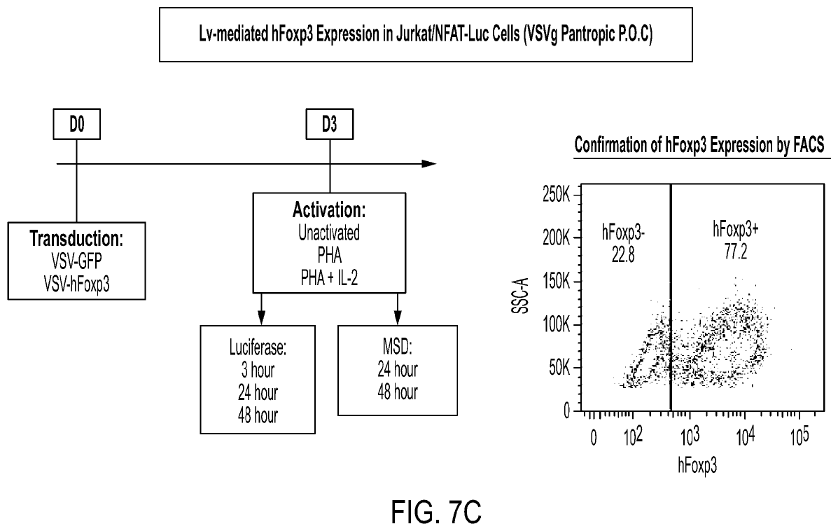

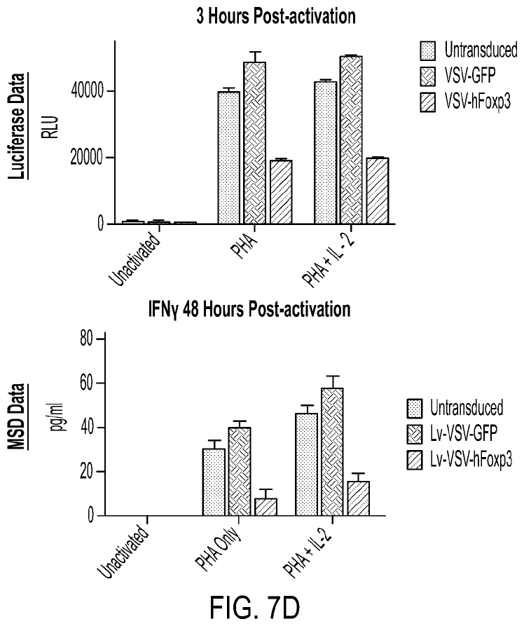

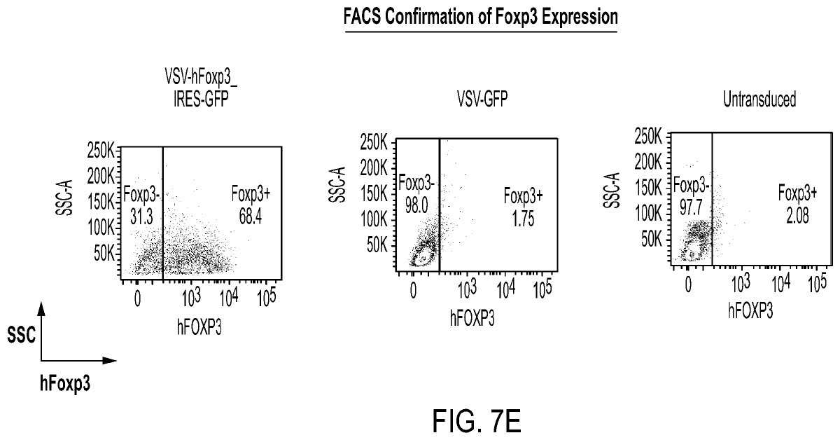

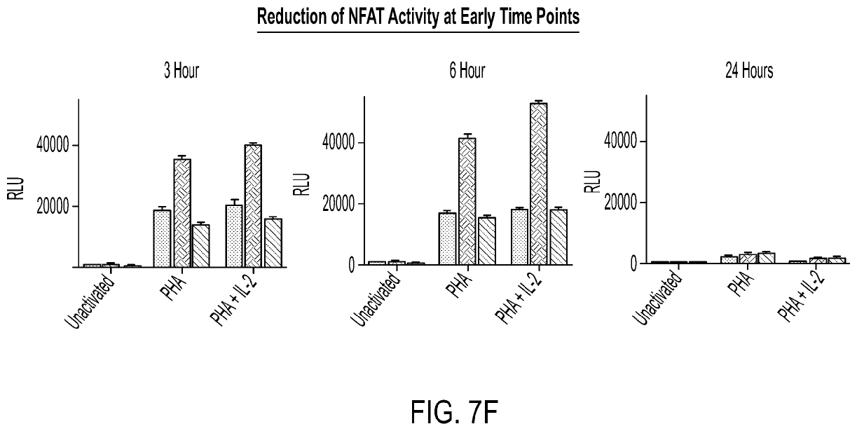

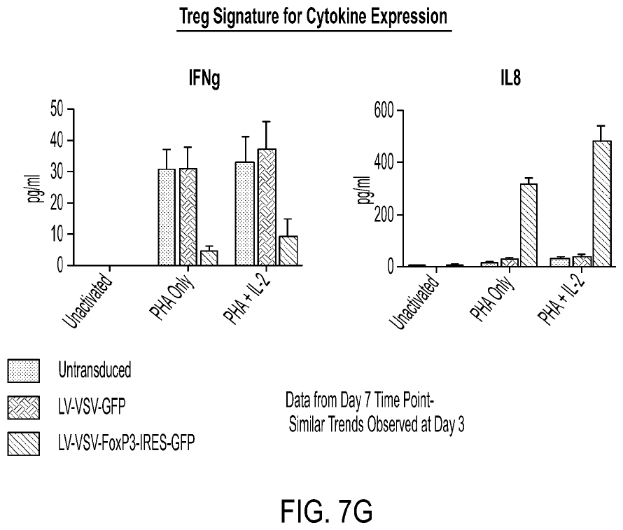

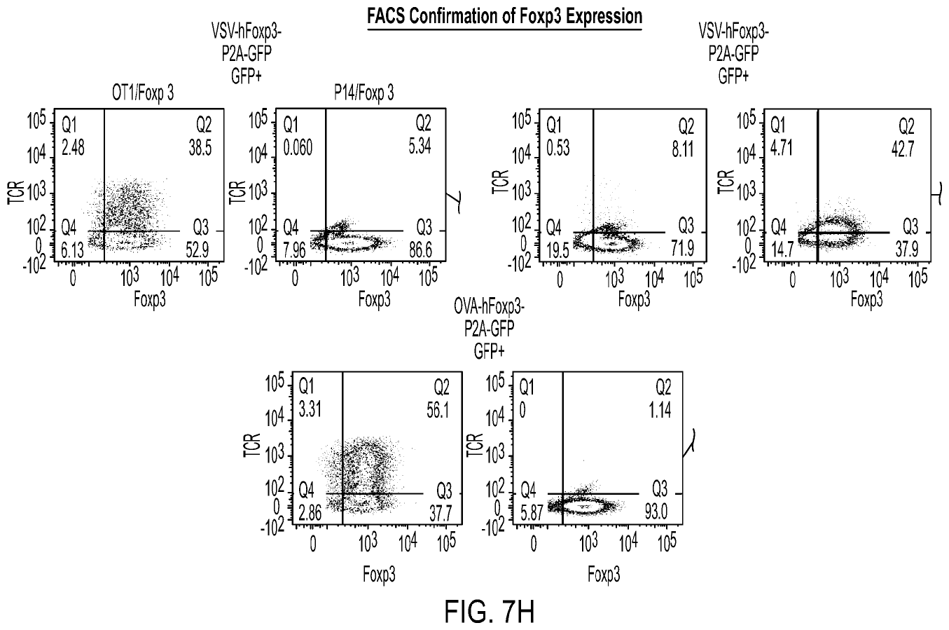

This lentiviral particle displaying H-2K^{b}-OVA on its surface was also generated to carry an EGFP reporter nucleotide of interest (any other reporter gene can be used, including, but not limited to, DsRed, RFP, YFP, CFP, mCherry, TdTomato, Luciferase, and Lac-Z). Other bicistronic lentiviral vector displaying H-2K^{b}-OVA on its surface carries the gene encoding the transcription factor hFOXP3 as a nucleotide of interest (any other transcription factor can be used such as, e.g., IKZF2 (Helios) IKZF4 (EOS), GATA1, IRF4, SATB1, LEF1, etc.) as well as the reporter gene EGFP (any other reporter gene can be used). FIG. 6 shows a schematic of the LV-H2D^{b}/gp33-hFOXP3-P2A-EGFP (FIG. 6A) and LV-H2K^{b}/OVA-hFOXP3-P2A-EGFP (FIG. 6B) lentiviral particles and also outlines the general structure of the plasmids used for particle generation. FACS analysis confirmed successful T cell transduction by the lentiviral particles pseudotyped with the H-2K^{b}/OVA complex carrying an EGFP reporter nucleotide (LV-H-2K^{b}/OVA-EGFP) (FIG. 7B).

Additionally, a lentiviral particle comprising the OVA-restricted OT-1 TCR as a nucleotide of interest and pseudotyped with the pan-tropic envelope VSV-G glycoprotein is also generated and used to transduce J.RT3-T3.5/AP1-Luc/mCD28/mCD8αβ cells to create a stable immortalized Jurkat T cell-derived cell line expressing the OT-1 TCR (J.RT3-T3.5/AP1-Luc/mCD28/mCD8αβ/OT-1). The OT-1 TCR expression cassette (SEQ ID NO: 10) is synthesized by PCR from splenocytes of OT-1 transgenic mice (C57BL/6-Tg(TcraTcrb)1100Mjb/J, Jackson Laboratories, Hogquist et al., Cell, 76:17-27 (1994)), as the association of the OT-1 TCRα subunit (Vα2-Jα26, Addgene #69576, Kaye et al. J. Immunol. 20:2333-2337 (1992)) linked to the OT-1 TCRβ subunit (Vβ35-Dβ32-Jβ2.6, Addgene #69577, Carbone et al. Int. Immunol. 4:861-867 (1992)) via a T2A self-cleaving peptide to allow transcription of both subunits from a same promoter. Non-limiting examples of other methods to generate the J.RT3-T3.5/AP1-Luc/mCD28/mCD8αβ/OT-1 cell line include nucleofection, electroporation, and transfection of linearized plasmid (e.g., using lipofectamine, calcium chloride, calcium phosphate, PEI, or any other suitable transient transfection method).

For functional studies, J.RT3-T3.5/AP1-Luc/mCD28/mCD8αβ/P14 or J.RT3-T3.5/AP1-Luc/mCD28/mCD8αβ/OT-1 cells are transduced in vitro with either LV-H-2D^{b}/gp33-hFOXP3-P2A-EGFP or LV-H-2K^{b}/OVA-hFOXP3-P2A-EGFP lentiviral particles. Other controls include transduction without the lentiviral particles (Mock) or with pantropic lentiviral particles displaying the glycoprotein of the Vesicular Stomatitis Virus (VSV-G). After lentiviral transductions, J.RT3-T3.5/AP1-Luc/mCD28/mCD8α3/P14 or J.RT3-T3.5/AP1-Luc/mCD28/mCD8α3/OT-1 cells are then stimulated with any of the following methods: (i) treatment with phytohemagglutinin (PHA), (ii) treatment with phorbol 12-myristate 13-acetate (PMA), (iii) treatment with both PHA and PMA, (iv) soluble anti-CD3/anti-CD28 monoclonal antibodies, (v) beads coupled with anti-CD3/anti-CD8 antibodies, or (vi) antigen-presenting cells (APC) previously pulsed with gp33 and OVA peptides (FIG. 7A). T-cell phenotype following transduction is evaluated, e.g., by measuring (i) activity of transcription factors involved in cytokines production (e.g., via measurement of luciferase expression driven by AP-1 promoter, activity of other transcription factors such as NF-κB or NFAT involved in cytokine production during T cell activation, etc.), (ii) cytokine (e.g., IL-2, IFNγ, etc.) production, (iii) cell proliferation, (iv) suppressive activity on CD4+CD25+T responder cells (Tres) proliferation in cocultures models as described in Kim et al., Biochemical and Biophysical Research Communications, 362:44-50 (2007).

In a similar set of experiment (FIGS. 7C-7D), Jurkat/NFAT-Luc cells were transduced with the pan-tropic lentiviral particles LV-VSV-GPF or LV-VSV-hFOXP3. Three days post-transduction, cells were stimulated with either PHA or PHA and IL-2. Cells were then evaluated for luciferase expression or cytokine production at indicated time points. FACS analysis confirmed hFOXP3 expression in the transduced cells. Luciferase analysis and IFNγ production revealed that the pan-tropic lentivirus-mediated hFOXP3 expression modulates T cell phenotype in Jurkat/NFAT-Luc cells. It was further shown that ectopic expression of Foxp3 by the pan-tropic lentiviral particle LV-VSV-hFOXP3 induces a Treg-like phenotype in Jurkat/NFAT-Luc cells (FIGS. 7E-7G), while ectopic expression of FOXP3 with H-2K^{b}-OVA single chain induces a specific IL-8 increase in J.RT3 cells displaying the OT1 TCR (FIGS. 7H-7I).

### Material and Methods

### Plasmid for Lentiviral Vector Production:

The production of lentiviral particles displaying the gp33 peptide in the groove of the MHC class I (H-2D^{b}) molecule, or the OVA peptide in the groove of the MHC class I (H-2K^{b}) on its envelope surface and comprising a nucleotide of interest encoding either a reporter protein (EGFP) or a regulating Treg transcription factor FOXP3 as the nucleotide of interest was achieved by co-transfection with the following plasmids:
1. Transfer plasmids comprising a nucleotide sequence of interest:
   pWPXLd (Addgene #12258) that encodes the EGFP reporter protein under control of the ubiquitous EFla promoter. An alternative plasmid also used was pLVX-EF1α-EGFP-IRES-Puro (SEQ ID NO: 11). pLVX-EF1α-EGFP was generated by inserting the EGFP sequence into the commercial pLVX-EF1α-IRES-Puro plasmid (Clontech/Takara). Alternative reporters include, but are not limited to DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-hFOXP3-P2A-EGFP (SEQ ID NO: 12) that comprises a codon-optimized cDNA encoding the canonical isoform of the human transcription factor FOXP3 (sequences synthesized by IDT, SEQ ID NO: 13) under control of the ubiquitous EF1a promoter and an EGFP reporter linked to human FOXP3 via a self-cleaving P2A peptide. pLVX-EF1α-hFOXP3-P2A-EGFP has been generated by inserting the human FOXP3 cDNA sequence into the pLVX-EF1α-GSG-P2A-EGFP (SEQ ID NO: 14) backbone plasmid, itself derived from the commercial pLVX-EF1α-IRES-Puro plasmid (Clonetech/Takara). Alternative transfer vector plasmid contains only human FOXP3 with no reporter, or a different reporter protein, including, but not limited to, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse or human FOXP3 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-hFOXP3-IRES-EGFP (SEQ ID NO: 15) is generated by inserting the sequences encoding human FOXP3 into the pLVX-EF1α-IRES-EGFP plasmid (SEQ ID NO: 16), itself derived from the commercial pLVX-EF1α-IRES-Puro plasmid (Clonetech/Takara) by replacement of the puromycin resistant sequence with the EGFP sequence. Alternative non-limiting example of other transcription factors include human IKZF2 (Helios) IKZF4 (EOS), GATA1, IRF4, SATB1, LEF1 (SEQ ID NO: 17 to 22). Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contains mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. psPAX2 was used as the packaging plasmid (Addgene plasmid #12260). PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2^{nd} generation packaging plasmids, 3^{rd} generation packaging plasmids, such as pRSV-Rev (Addgene plasmid #12253) and pMDLg-pRRE (Addgene plasmid #12251), Dull et al., J Virol, 72(11):8463-8471 (1998)) or 4^{th} generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pRG984-H-2D^{b}/gp33 (SEQ ID NO: 23) and pRG984-H-2K^{b}/OVA (SEQ ID NO: 24) were used as the first envelope plasmid for the production of the LV-H-2D^{b}/gp33-hFOXP3 and LV-H-2K^{b}/OVA-hFOXP3, lentiviral particles respectively. The mROR-gp33-β2m-H-2D^{b} and mROR-OVA-β2m-H-2K^{b} cassettes were subcloned into the expression plasmid pRG984 (SEQ ID NO: 25) under the control of the human Ubiquitin C (hUbC) promoter and the β-globin intron.
4. pRG984-SINmu (SEQ ID NO: 26) was used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of control pantropic lentiviral vector displaying the VSV-G envelope glycoprotein on its surface and expressing comprising a nucleotide of interest encoding either a reporter protein (EGFP), or a regulating Treg transcription factor (FOXP3 or any other T cell transcription factors including, but not limited to, IKZF2 (Helios) IKZF4 (EOS), GATA1, IRF4, SATB1, LEF1) as the nucleotide of interest was achieved by co-transfection with the following plasmids:
1. Transfer plasmids comprising a nucleotide sequence of interest:
   pWPXLd (Addgene #12258) that encodes the EGFP reporter protein under control of the ubiquitous EF1α promoter. An alternative plasmid also used was pLVX-EF1α-EGFP-IRES-Puro (SEQ ID NO: 11). pLVX-EF1α-EGFP was generated by inserting the EGFP sequence into the commercial pLVX-EF1α-IRES-Puro plasmid (Clontech/Takara). Alternative reporters include, but are not limited to DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-hFOXP3-P2A-EGFP (SEQ ID NO: 12) that comprises a codon-optimized cDNA encoding the canonical isoform of the human transcription factor FOXP3 (sequences synthesized by IDT, SEQ ID NO: 13) under control of the ubiquitous EF1α promoter and an EGFP reporter linked to human FOXP3 via a self-cleaving P2A peptide. pLVX-EF1a-hFOXP3-P2A-EGFP has been generated by inserting the human FOXP3 cDNA sequence into the pLVX-EF1a-GSG-P2A-EGFP (SEQ ID NO: 14) backbone plasmid, itself derived from the commercial pLVX-EF1α-IRES-Puro plasmid (Clonetech/Takara). Alternative transfer vector plasmid contains only human FOXP3 with no reporter, or a different reporter protein, including, but not limited to, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse or human FOXP3 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-hFOXP3-IRES-EGFP (SEQ ID NO: 15) is generated by inserting the sequences encoding human FOXP3 into the pLVX-EF1α-IRES-EGFP plasmid (SEQ ID NO: 16), itself derived from the commercial pLVX-EF1α-IRES-Puro plasmid (Clonetech/Takara) by replacement of the puromycin resistant sequence with the EGFP sequence. Alternative non-limiting example of other transcription factors include human IKZF2 (Helios) IKZF4 (EOS), GATA1, IRF4, SATB1, LEF1 (SEQ ID NO: 17 to 22).
Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contains mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. psPAX2 was used as the packaging plasmid (Addgene plasmid #12260). PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2^{nd} generation packaging plasmids, 3^{rd} generation packaging plasmids, such as pRSV-Rev (Addgene plasmid #12253) and pMDLg-pRRE (Addgene plasmid #12251), Dull et al., J Virol, 72(11):8463-8471 (1998)) or 4^{th} generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pMD2.G (Addgene plasmid #12259) as the envelope plasmid displaying the glycoprotein of the Vesicular Stomatitis Virus (VSV).

The production of lentiviral particles LV-VSV-P14 comprising the P14 TCR as the nucleotide of interest was achieved by co-transfection of the following plasmids:
1. pLVX-EF1α-P14 (SEQ ID NO: 27) as the transfer vector plasmid. pLVX-EF1α-P14 has been generated by inserting the P14TCRα-T2A-P14TCRβ cassette (SEQ ID NO: 3) described above into the pLVX-EF1α-IRES-Puro backbone from Clontech/Takara.
2. psPAX2 (Addgene plasmid #12260) was used as the packaging plasmid. PsPAX2 is a second generation packaging plasmid that contains the gag gene coding different structural elements of the lentiviral capsid (i.e., matrix, capsid, and nucelocapsid), and the pol gene coding different necessary enzymes for the viral genome integration (i.e., retrotranscriptase, protease, and integrase). Alternative packaging plasmids include, e.g., other 2nd generation packaging plasmids, 3rd generation packaging plasmids, such as pRSV-Rev (Addgene plasmid #12253) and pMDLg-pRRE (Addgene plasmid #12251), Dull et al., J Virol, 72(11):8463-8471 (1998)) or 4th generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pMD2.G (Addgene plasmid #12259) as the envelope plasmid displaying the glycoprotein of the Vesicular Stomatitis Virus (VSV).

The production of lentiviral particles LV-VSV-OT-1 comprising the OT-1 TCR as the nucleotide of interest was achieved by co-transfection of the following plasmids:
1. pLVX-EF1α-OT-1 (SEQ ID NO: 28) as the transfer vector plasmid. pLVX-EF1α-OT-1 has been generated by inserting the OT-1TCRα-T2A-OT-1TCRβ cassette (SEQ ID NO: 10) described above into the pLVX-EF1α-IRES-Puro backbone from Clontech/Takara.
2. psPAX2 (Addgene plasmid #12260) was used as the packaging plasmid. PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include, e.g., other 2nd generation packaging plasmids, 3rd generation packaging plasmids, such as pRSV-Rev (Addgene plasmid #12253) and pMDLg-pRRE (Addgene plasmid #12251), Dull et al., J Virol, 72(11):8463-8471 (1998)) or 4th generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pMD2.G (Addgene plasmid #12259) as the envelope plasmid displaying the glycoprotein of the Vesicular Stomatitis Virus (VSV).

### Cell Lines

Adherent HEK 293T/17 cells (ATCC) were cultured in DMEM medium (Gibco/Life Technologies) supplemented with 10% fetal bovine serum and 1% penicillin-streptomycin.

The suspension cell line J.RT3-T3.5/AP1-Luc/mCD28/mCD8αβ (an engineered cell line derived from the J.RT3-T3.5, itself derived from the human lymphoma Jurkat cell line), which does not express any TCRs or CD3 on its surface, was used to express the P14 TCR or the OT-1 TCR via lentiviral transduction with LV-VSV-P14TCR and LV-VSV-OT-1TCR lentiviral particles respectively. J.RT3-T3.5/AP1-Luc/mCD28/mCD8αβ, J.RT3-T3.5/AP1-Luc/mCD28/mCD8αβ/P14 and J.RT3-T3.5/AP1-Luc/mCD28/mCD8α/OT-1 were cultured in RPMI 1640 (Gibco/Life Technologies) supplemented with 10% of heat-inactivated Fetal Bovine Serum (Gibco/Life Technologies) and 1% Penicillin/Streptomycin mix (Gibco/Life Technologies).

### Lentiviral Particle Production

Lentiviral particles were produced following standard lipofectamine-mediated co-transfection of HEK 293T cells with the respective transfer vector, packaging and envelope plasmids. The day before transfection cells were washed with phosphate buffered saline solution (PBS) once then detached from vessel with TrypLE™ Express (Life Technologies). After neutralization of TrypLE Express with cell medium containing FBS, cells were centrifuged at 1200 rpm for 5 min at 25 C, then resuspended in complete DMEM medium, counted and seeded in 150 mm cell culture dishes at a density of 10×10⁶ cells/plate. On the day of transfection, the cell culture medium was replaced by fresh Opti-MEM medium (Gibco/Life Technologies) supplemented with 25 nM chloroquine (Sigma-Aldrich). The DNA mix was prepared by mixing 60 ug of total DNA 1.5 mL of Opti-MEM with 60 ul of PLUS™ Reagent (Life Technologies). The specific plasmid ratios for the different lentivirus productions are illustrated in TABLE 2. In parallel 100 μl of Lipofectamine® TLX (Life Technologies) was diluted in 1.5 mL of OptiMEM medium. DNA mix was then added to the lipofectamine mix and the new combined solution was incubated at room temperature for 20 minutes before being added directly to the cells dropwise. The culture medium was changed 6 to 8 h after transfection and the cells were then incubated for 48 h at 37 C in an incubator with 5% CO₂ atmosphere. At day 2 post-transfection, cell media containing the lentiviral particles were centrifuged for 10 min at 3000 rpm to remove the debris, then passed through a 0.45 um pore size filter. The filtered supernatants were then treated with 1 μg/ml DNAse and 1 mM MgCl2 for 15 minutes at 37° C. to remove residual DNA. For concentrating the lentiviral vectors batch, the supernatants were then ultracentrifuged at 27,100 rpm for 90 min. After ultracentrifugation, pellets were resuspended in a suitable volume of PBS (50 to 100 μl) overnight. The resuspended virus was finally processed through a series of short centrifugations (30 sec at 13500 rpm) to clarify the lentiviral solution of remaining debris. The batches of lentiviral particles were titrated by RT-qPCR using a SYBR® technology-based kit from Clontech/Takara then stocked at −80° C. until use for transduction.

Alternative transfections reagents include polyethylenimine (PEI), calcium chloride or calcium phosphate.

### Jurkat T Cells Transduction

On day of transduction, J.RT3-T3.5/AP1-Luc/mCD28/mCD8αβ. J.RT3-T3.5/AP1-Luc/mCD28/mCD8αβ/P14 and J.RT3-T3.5/AP1-Luc/mCD28/mCD8αβ/OT-1 cells were centrifuged for 5 minutes at 1200 RPM and 25° C. After resuspension in fresh complete RPMI medium, cells were counted, seeded in 24-well plates at a density of 100,000 cells/well and mixed with 4 μg/ml polybrene and the suitable amount of virus (standard dose of 25,000 viral copies per cell). Cells were then spinoculated by centrifugating them in presence of the virus at 2000 rpm for 90 min at 30° C. After centrifugation, cells were incubated at 37° C. and 5% CO₂ for 3 days before fixation and staining/FACS analysis.

### Splenocytes and Antigen Presenting Cell Primary Cultures

Spleens of adult C57Bl/6 mouse was first excised and placed in cold PBS (without calcium or magnesium) supplemented with 2% FBS. Tissues were then homogenized using GentleMACS™ Octo Dissociator (Milteniy) to break apart the spleens in GentleMACS™ C Tubes (Milteniy). Dissociated cells were then centrifuged at 500 g, 4° C. for 5 min. After centrifugation the cell pellet was resuspended with gentle vortexing in a suitable volume of ACK lysis buffer (5 ml per spleen, Gibco/Life Technologies) to remove the red blood cells, and incubated for 5 min at room temperature. After incubation the cell suspension was added to 10 ml/spleen of PBS/FBS, and centrifuged at 500 g, 4° C. for 5 min. The cell pellet was then resuspended in PBS/FBS and the cell solution was filtered through a 0.7 μM filter (BD Biosciences). The filtered cell suspension was centrifuged again and the subsequent pellet of individualized cells was resuspended one more time in a suitable volume of PBS/FBS. Total spleen cells were counted, and global antigen-presenting cells (APC) were sorted using anti-MHC class II Microbeads and MACS® sorting technology (Miltenyi Biotec). After elution, the MHC class II-positive cell fraction was collected and resuspended at 1×10⁶ cells/ml in APC cell medium composed of RPMI 1640 (Gibco/ThermoFisher)+1% Horse serum (Gibco/ThermoFisher)+100 U/ml penicillin/streptomycin (Gibco/ThermoFisher)+2 mM L-Glutamin (Gibco/Life technologies) supplemented with 30 ng/ml of IL-4 (Peprotech), 50 ng/ml of GM-CSF (Peprotech) and 1 ng/ml of recombinant mouse TNFα (Peprotech).

Pulsing of APCs with Peptides

APC were then incubated with 10 μg/ml of each peptide (gp33-41 (KAVYNFATC; SEQ ID NO: 1) or OVA₂₅₇₋₂₆₄ peptide (SIINFEKL; SEQ ID NO: 7) overnight. The day after, peptide-pulsed APC were harvested and washed before addition at a ratio 1:1 to the different J.RT3-T3.5 derived cell lines, 48 h after infection with the lentiviral particles. J.RT3-T3.5-derived cell lines were then cocultured with the pulsed APC in J.RT3-T3.5 cell culture medium in duplicate with one half for proliferation assay and one half for luciferase assay+ cytokines production assay.

### Jurkat T Cells Activation Post Transduction

Alternatively, to coculture with peptides-pulsed APCs, J.RT3-T3.5 derived cell lines are activated 48 h after transduction with either phytohemagglutinin (PHA, Sigma, at 5 μg/ml), phorbol 12-myristate 13-acetate (PMA, Sigma, at 2 μg/ml), or a combination of PHA and PMA (5 μg/ml and 2 μg/ml respectively).

Another alternative method to activate J.RT3-T3.5 derived cell lines 48 h after transduction is to use soluble or immobilized anti-CD3 and anti-CD28 monoclonal antibodies (BD Biosciences). Soluble antibodies are added at a concentration of 1 μg/ml. In some experiments plates pre-coated with anti-CD3 antibodies (BD Biosciences) are used in combination with soluble anti-CD28 antibody (BD Biosciences, 1 μg/ml).

Another alternative method to activate J.RT3-T3.5 derived cell lines activating beads coupled with anti-CD3/anti-CD28 antibodies (Dynabeads, ThermoFisher). 48 h after transduction, infected J.RT3-T3.5 derived cells are counted, and anti-CD3/anti-CD28 beads are added to the culture medium at ratio 1:1.

### Cells Staining and FACS Analysis

Fluorescence-activated cell sorting (FACS) was performed on day 3 after the transduction. Transduced cells were counted and seeded equally into a 96-well V bottom plate. Cells were spun at 2000 rpm at 4° C. for 2 min, washed with PBS then spun again. Cells were incubated with Live/Dead® Fixable Near-IR stain (Life Technologies, diluted at 1:10000 in PBS) for 15 min in the dark, washed, and incubated with Fc block (eBioscience, dilution 1:150) in FACS Stain Buffer (BD Biosciences). After being washed twice again with FACS stain buffer, cells were subsequently incubated for 30 min, on ice and in the dark with either of the following antibodies: Alexa Fluor®647-conjugated anti-mouse TCR Vβ8.1,8.2 (Biolegend, 0.25 μg per 10⁶ cells) or Alexa Fluor® 647 Rat IgG2a, κ Isotype Control. Cells were washed one more time with FACS stain buffer, fixed with paraformaldehyde 1%, washed again and finally resuspended in FACS stain buffer. Samples were run for analysis with a BD FACS Canto II analyzer (BD Biosciences).

### Proliferation and Cytokine Production Assays

To measure T cell proliferation, on half of the incubated cells, 1 μCi/well [³H]thymidine (Amersham) is added to assay cultures at 48 h after contact with the peptide-pulsed APC. Following incubation for 12-16 h, cultures are harvested onto Unifiter Plates (Packard Instrument). Microscint 20 scintillation fluid (Packard Instrument) is added to each well, and plates are counted on a Scintillation Counter.

For transcription factor activity analysis and cytokines production assay, the other half of the cells is harvested 5 days after contact with the peptide-pulsed APC, centrifuged for 5 min at 300 g, and both cell pellets and supernatants are collected. Cell pellets are processed for luciferase detection assay to measure AP1 activity, and cytokines production is measured by ELISA from the supernatant using the Human TH1/TH2 10-Plex Tissue Culture Kit from Meso Scale Diagnostics (MSD).

### Example 2: Specific Targeting and Activation of Mouse Primary T Cells Expressing Antigen-Specific T Cell Receptor (TCR) with Lentiviral Particles Displaying Antigenic Peptides in MHC Class I Groove on their Surface

This example describes the ability of lentiviral vectors pseudotyped with MHC class I molecules displaying an antigenic peptide (i.e., epitope) and their use for specifically transducing and modifying the phenotype of mouse primary cells that express an antigen-specific T cell receptor (TCR) that recognizes this peptide.

In particular, this example demonstrates the targeting of mouse primary CD8+ T cells expressing the OT-1 TCR with lentiviral particles pseudotyped with a MHC class I molecule (H-2K^{b}) presenting the OVA₂₅₇₋₂₆₄ peptide (OVA, SIINFEKL, SEQ ID NO: 7 and 8) peptide in MHC groove.

More particularly, the ability of lentiviral particles carrying the sequence encoding the mouse transcription factor TBX21 (mTBX21, SEQ ID NO: 29) as a nucleotide of interest to trigger activation of mouse naïve CD8+ T cells into CD8+T effector T cells and enhance an antiviral response is tested. TBX21, also known as T-bet, is a T cell-specific transcription factor controlling the expression of IFNγ and primarily known for being involved in the development of the Th1 lineage from naïve CD4+ T cells. In CD8+ T cells, TBX21 expression is induced after TCR and IL-12-mediated activation, and is required for the production of IFNγ, Granzyme B and perforin (Lazarevic et al., Nature Reviews Immunology, 13:777-789 (2013)). TBX21 thus plays a well-established role in effector and memory CD8+ T cells differentiation in association with other transcription factors such as eomesodermin (EOMES) and runt-related transcription factor 3 (RUNX3). Moreover, retroviral overexpression of TBX21 in primary CD8+ T cells has been shown to reduce the expression of the T cell exhaustion marker PD1 and to trigger enhanced CD8+ T cells response in chronic infections models (Kao et al., Nat. Immunol. 12(7): 663-671 (2012)) Targeted overexpression of TBX21 in naïve CD8+ T cell with lentiviral particles pseudotyped with MHC class I/peptide complexes thus appears to be a powerful approach to induce an antigen-specific CD8+ effective response.

A first lentiviral particle is generated comprising at its surface (i) H-2K^{b} (MHC class I molecule) displaying the OVA peptide to target the OT1 TCR (mROR-OVA-β2m-H-2K^{b}, SEQ ID NO: 9) and (ii) a fusogen “SINmu,” as previously described.

Another lentiviral particle is generated comprising at its surface (i) H-2D^{b} (MHC Class I molecule) displaying the gp33 peptide to target the P14 TCR (mROR-OVA-β2m-H-2D^{b}, SEQ ID NO: 4) and ii) a fusogen “SINmu” as previously described.

These lentiviral particles are first generated to carry LNGFR as reporter nucleotide of interest (any other reporter gene can be used, including, but not limited to, Thy1.1, EGFP, DsRed, RFP, YFP, CFP, mCherry, TdTomato, Luciferase, and Lac-Z).

In order to demonstrate that lentiviral vectors pseudotyped with a MHC class I presenting an antigenic peptide can target primary T cells expressing a specific TCR, mixed cell cultures containing a ratio 1:1 of primary CD8+ T cells from wild-type C57Bl/6 (WT) and transgenic OT-1 mice (B6.129S6-Rag2^{tm1Fwa} Tg(TcraTcrb)1100Mjb, Taconic) were transduced with either LV-H-2D^{b}/gp33-mTBX21-P2A-LNGFR or LV-H-2K^{b}/OVA-mTBX21-P2A-LNGFR lentiviral particles. Additional controls include transduction without the lentiviral particles (mock) or with lentiviral targeting all CD8+T or all CD4+ T cells by displaying respectively an anti-mouse CD8 monoclonal antibody or an anti-mouse CD4 monoclonal antibody on their surface. This experiment demonstrates the specificity of TCR targeting in mouse primary T cells by the created MHC/epitope pseudotyped lentiviral particles.

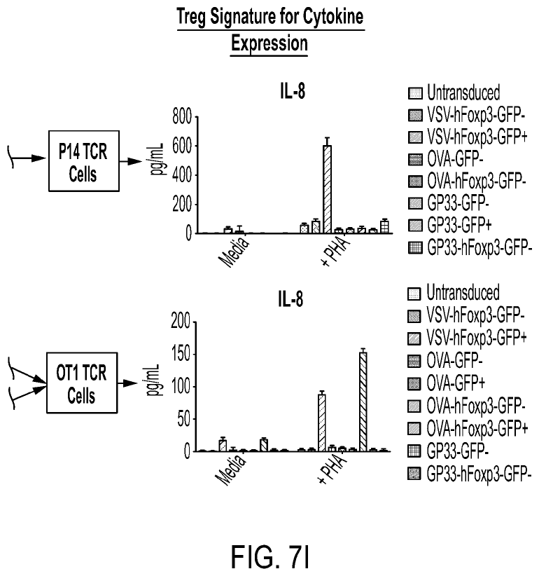

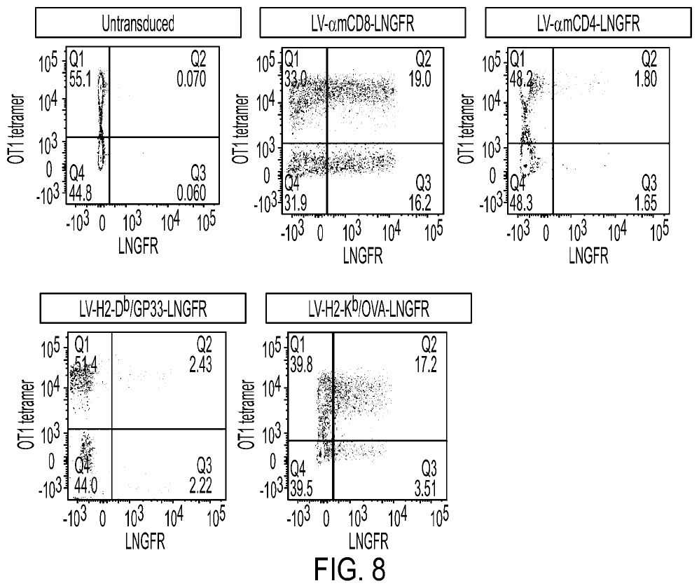

As per shown on FIG. 8, CD8+ primary T cells isolated from WT and OT-1 are equally transduced by the lentiviral vectors pseudotyped with an anti-mouse CD8 antibody on their surface, while lentiviral vectors pseudotyed with an anti-mouse CD4 antibody did not transduce any of the CD8+ T cell populations, as expected. Similarly, mixed T cell cultures transduced with the lentiviral vector pseudotyped with the H-2D^{b}/gp33 complex did not show any LNGFR expression in any of the T cell populations. At the opposite, mixed T cell cultures transduced with the lentiviral vector pseudotyped with H-2K^{b}/OVA complex show a predominant expression of LGNFR reporter in the T cell population expressing the OT-1 TCR compared to the OT-1 TCR-negative T cells.

For functional studies, bicistronic lentiviral particles pseudotyped with the H-2K^{b}/OVA complex or the H-2D^{b}/gp33 or are used to deliver the murine transcription factor TBX21 in T cells ex vivo. The effects of such treatment on the T cell phenotype are analyzed. Specifically, the ability of lentiviral particles displaying the H-2-K^{b}/OVA complex on their surface and comprising the genetic sequence encoding mouse TBX21 (Tbx21, GenBank: AF277994.1, AF27799, AF277992) as a nucleotide of interest to transfer the said nucleotide of interest and modulate T cell phenotype, in this case a cytotoxic phenotype in infected cells is evaluated in primary mouse T cells isolated from transgenic OT-1 mice to expand CD8+ T cells with the OVA-specific OT-1 TCR. Similarly, the ability of lentiviral particles displaying the H-2-K0/OVA complex on their surface and comprising the genetic sequence encoding mouse TBX21 as a nucleotide of interest to transfer the said nucleotide of interest and modulate T cell phenotype, is evaluated in primary mouse T cells isolated from transgenic P14 mice (B6; B10-Rag2^{tm1Fwa} Tg(TcrLCMV)327Sdz, Taconic) to expand CD8+ T cells with the gp33-specific P14 TCR.

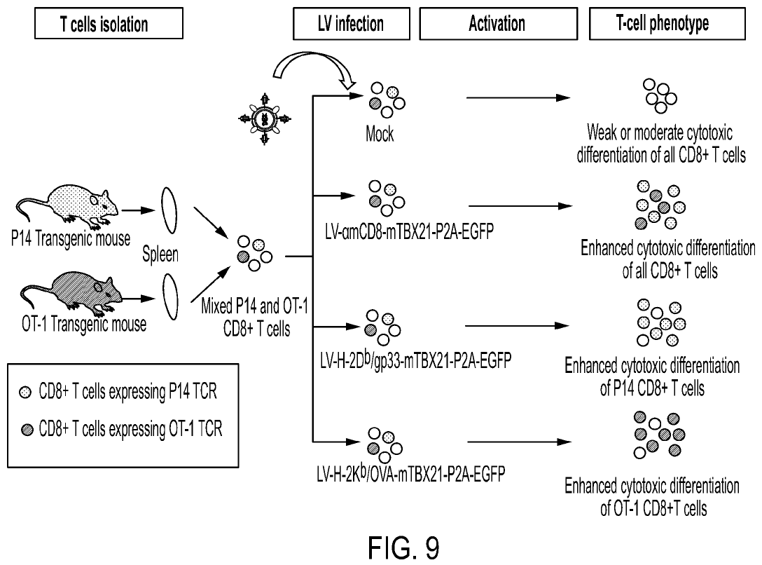

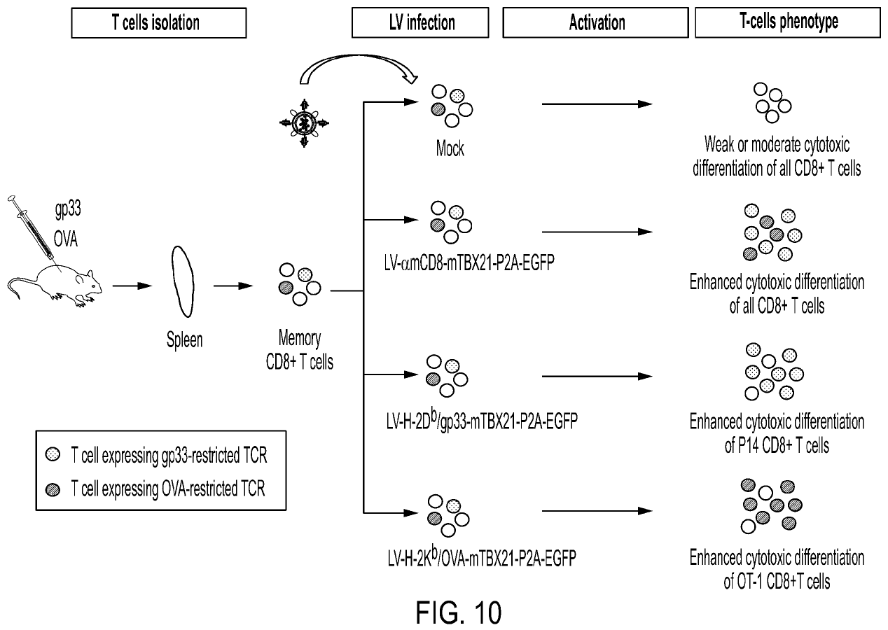

For ex vivo functional assays on mouse primary cells, CD8+ T-cells are isolated from splenocytes of transgenic P14 mice and OT-1 mice and then transduced with either LV-H-2D^{b}/gp33-mTBX21-P2A-LNGFR or LV-H-2K^{b}/OVA-mTBX21-P2A-LNGFR lentiviral particles. Additional controls include transduction without the lentiviral particles (mock) or with lentiviral targeting all CD8+T or all CD4+ T cells by displaying respectively an anti-mouse CD8 monoclonal antibody or an anti-mouse CD4 monoclonal on their surface. After lentiviral transductions, P14 and OT-1 CD8+ T cells are then stimulated with any of the following methods: (i) treatment with phytohemagglutinin (PHA), (ii) treatment with phorbol 12-myristate 13-acetate (PMA), (iii) treatment with both PHA and PMA, (iv) soluble or immobilized anti-CD3/anti-CD28 monoclonal antibodies, (v) activation beads coupled with anti-CD3/anti-CD8 beads, or (vi) antigen-presenting cells (APC) previously pulsed with gp33 and OVA peptides (FIG. 9). An alternative experimental setting consists in immunizing wild type C57Bl/6 mice with either the gp33 or OVA peptide, or other appropriate methods, to expand CD8+ T cells expressing TCRs restricted to gp33 or OVA peptides prior to lentivirus transduction (FIG. 10). Two weeks after immunization, splenocytes containing memory T cells are isolated and transduced with either LV-H-2D^{b}/gp33-mTBX21-P2A-LNGFR or LV-H-2K^{b}/OVA-mTBX21-P2A-LNGFR lentiviral particles, or other control conditions previously described. After lentiviral transductions, splenocytes are stimulated again with the same techniques described for FIG. 9. T-cell phenotype following transduction is evaluated by measuring (i) expression of T cell activation and T cell exhaustion markers such as, but not limited to, CD69 or PD-1 (ii) cytokine (e.g., IL-2, IFNγ, etc.) production, (iii) cell proliferation, (iv) production of cytolytic proteins such as Granzyme B and perforin, (iv) cytotoxic activity proliferation in cocultures with peptide-pulsed target cells and/or (v) other well-known cytotoxic T-cell characteristics.

### Material and Methods

### Plasmids for Lentiviral Vector Production:

The production of lentiviral particles comprising the gp33 peptide in the groove of the MHC class I (H-2D^{b}) molecule, or the OVA peptide in the groove of the MHC class I (H-2K^{b}) on its envelope surface and comprising either a reporter protein (LNGFR), or a regulating transcription factor (TBX21) as the nucleotide of interest is achieved by co-transfection with the following plasmids:
1. Transfer plasmids comprising a nucleotide sequence of interest:
   pLVX-EF1α-GSG-P2A-LNGFR (SEQ ID NO: 66) that encodes the LNGFR reporter protein under control of the ubiquitous EF1α promoter. pLVX-EF1α-P2A-LNGFR was generated by inserting the LNGFR sequence (Milteniy Biotech) sequence into pLVX-EF1α-GSG-P2A-EGFP (SEQ ID NO: 14) derived from the commercial pLVX-EF1α-IRES-Puro plasmid (Clontech/Takara). Alternative reporters include, but are not limited to Thy1.1, EGFP, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-mTBX21-P2A-LNGFR (SEQ ID NO: 30) that comprises a codon-optimized cDNA encoding the canonical isoform of the mouse transcription factor TBX21 (sequences synthesized by IDT, SEQ ID NO: 29) under control of the ubiquitous EF1α promoter and an LNGFR reporter linked to mouse TBX21 via a self-cleaving P2A peptide. pLVX-EF1α-mTBX21-P2A-LNGFR has been generated by inserting the mouse TBX21 cDNA sequence into the pLVX-EF1α-GSG-P2A-LNGFR (SEQ ID NO: 66) backbone plasmid, itself derived from the commercial pLVX-EF1α-IRES-Puro plasmid (Clonetech/Takara). Alternative transfer vector plasmid contains only mouse TBX21 with no reporter, or a different reporter protein, including, but not limited to, Thy1.1, EGFP, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-mTBX21-IRES-LNGFR (SEQ ID NO: 31) is generated by inserting the sequences encoding mouse TBX21 into the pLVX-EF1α-IRES-LNGFR plasmid (SEQ ID NO: 67), itself derived from the commercial pLVX-EF1α-IRES-Puro plasmid (Clonetech/Takara) by replacement of the puromycin resistant sequence with the EGFP sequence.
Alternative non-limiting example of other transcription factors include mouse EOMES and ZEB2 (SEQ ID NO: 32 and 33)
Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contain mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. Packaging plasmids:
   psPAX2 is used as the first packaging plasmid (Addgene plasmid #12260). PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2^{nd} generation packaging plasmids, 3^{rd} generation packaging plasmids, such as pRSV-Rev (Addgene plasmid #12253) and pMDLg-pRRE (Addgene plasmid #12251), Dull et al., J Virol, 72(11):8463-8471 (1998)) or 4^{th} generation packaging system such as Lenti-X® Fourth Generation packaging system (Clontech/Takara). Other modified versions of psPAX2 presenting mutations either in the integrase or the retrotranscriptase enzymes are used too for generating non-integrative versions of those vectors (TABLE 1).
   In some experiments a second packaging plasmid, pRG984-C-Scr-Vpx is used (SEQ ID NO: 34). This plasmids encodes the viral protein Vpx of the Simian Immunodeficience Virus (SIV—Mac251 strain) fused to the membrane targeting domain of the C-Src protein with an extra protease cleavage site (KARVLAEA (SEQ ID NO: 88), initially located between the HIV GAG proteins β27 and β29) for both an efficient incorporation of Vpx in the lentiviral particles and its release in the infected cells, as previously described (Durand et al., J. Virol. 87 (1): 234-242 (2013)). In several hematopoietic cells including T cells, Vpx counteracts the restriction factor SAMHD1 that hampers lentiviral infection at the reverse transcription level by depleting the pool of deoxynucleosides triphosphates (dNTPs) and thus limiting generation of de novo viral DNA from the HIV provirus genome. An alternative plasmid used is pRG984-C-Src-Flag-Vpx that encodes a tagged version of the Vpx protein (SEQ ID NO: 35)
3. Envelope plasmids:
   pRG984-H-2D^{b}/gp33 (SEQ ID NO: 23) and pRG984-H-2K^{b}/OVA (SEQ ID NO: 24) are used as the first envelope plasmid for the production of the LV-H-2D^{b}/gp33-mTBX21-IRES-EGFP and LV-H-2K^{b}/OVA-mTBX21-IRES-EGFP lentiviral particles respectively. The mROR-gp33-β2m-H-2D^{b} and mROR-OVA-β2m-H-2K^{b} cassettes were subcloned into the expression plasmid pRG984 (SEQ ID NO: 25) under the control of the human Ubiquitin C (hUbC) promoter and the β-globin intron.
   pRG984-SINmu (SEQ ID NO: 26) is used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of a control lentiviral particles targeting all CD8+ T cells comprising an anti-mouse CD8 monoclonal antibody (clone YTS156) on its envelope surface and comprising either a reporter protein (LNGFR), or a regulating transcription factor (TBX21) as the nucleotide of interest is achieved by co-transfection with the following plasmids:
1. Transfer plasmids comprising a nucleotide sequence of interest:
   pLVX-EF1α-GSG-P2A-LNGFR (SEQ ID NO: 66) that encodes the LNGFR reporter protein under control of the ubiquitous EF1α promoter. pLVX-EF1α-P2A-LNGFR was generated by inserting the LNGFR sequence (Milteniy Biotech) sequence into pLVX-EF1α-GSG-P2A-EGFP (SEQ ID NO: 14) derived from the commercial pLVX-EF1α-IRES-Puro plasmid (Clontech/Takara). Alternative reporters include, but are not limited to Thy1.1, EGFP, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-mTBX21-P2A-LNGFR (SEQ ID NO: 30) that comprises a codon-optimized cDNA encoding the canonical isoform of the mouse transcription factor TBX21 (sequences synthesized by IDT, SEQ ID NO: 29) under control of the ubiquitous EF1α promoter and an LNGFR reporter linked to mouse TBX21 via a self-cleaving P2A peptide. pLVX-EF1a-mTBX21-P2A-LNGFR has been generated by inserting the mouse TBX21 cDNA sequence into the pLVX-EF1a-GSG-P2A-LNGFR (SEQ ID NO: 66) backbone plasmid, itself derived from the commercial pLVX-EF1α-IRES-Puro plasmid (Clonetech/Takara). Alternative transfer vector plasmid contains only mouse TBX21 with no reporter, or a different reporter protein, including, but not limited to, Thy1.1, EGFP, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-mTBX21-IRES-LNGFR (SEQ ID NO: 31) is generated by inserting the sequences encoding mouse TBX21 into the pLVX-EF1α-IRES-LNGFR plasmid (SEQ ID NO: 67), itself derived from the commercial pLVX-EF1α-IRES-Puro plasmid (Clonetech/Takara) by replacement of the puromycin resistant sequence with the EGFP sequence.
Alternative non-limiting example of other transcription factors include mouse EOMES and ZEB2 (SEQ ID NO: 32 and 33)
   Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contain mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. Packaging plasmids:
   psPAX2 is used as the first packaging plasmid (Addgene plasmid #12260). PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2^{nd} generation packaging plasmids, 3^{rd} generation packaging plasmids, such as pRSV-Rev (Addgene plasmid #12253) and pMDLg-pRRE (Addgene plasmid #12251), Dull et al., J Virol, 72(11):8463-8471 (1998)) or 4^{th} generation packaging system such as Lenti-X® Fourth Generation packaging system (Clontech/Takara). Other modified versions of psPAX2 presenting mutations either in the integrase or the retrotranscriptase enzymes are used too for generating non-integrative versions of those vectors (TABLE 1).
   In some experiments a second packaging plasmid, pRG984-C-Scr-Vpx is used (SEQ ID NO: 34). This plasmids encodes the viral protein Vpx of the Simian Immunodeficience Virus (SIV—Mac251 strain) fused to the membrane targeting domain of the C-Src protein with an extra protease cleavage site (KARVLAEA (SEQ ID NO: 88), initially located between the HIV GAG proteins p27 and p29) for both an efficient incorporation of Vpx in the lentiviral particles and its release in the infected cells, as previously described (Durand et al., J. Virol. 87 (1): 234-242 (2013)). In several hematopoietic cells including T cells, Vpx counteracts the restriction factor SAMHD1 that hampers lentiviral infection at the reverse transcription level by depleting the pool of deoxynucleosides triphosphates (dNTPs) and thus limiting generation of de novo viral DNA from the HIV provirus genome. An alternative plasmid used is pRG984-C-Src-Flag-Vpx that encodes a tagged version of the Vpx protein (SEQ ID NO: 35)
3. Envelope plasmids:
   pRG984-αmCD8(YTS156)-IgG4us (SEQ ID NO: 36) and pRG984-αmCD8(YTS156)-IgK (SEQ ID NO: 37), encoding respectively the heavy chain and the light chain of the anti-mouse CD8 (clone YTS156) monoclonal antibody are used as the first envelope plasmids for the production of the LV-αmCD8-mTBX21-P2A-EGFP and LV-H-αmCD8-mTBX21-P2A-EGFP lentiviral particles respectively. The nucleotide sequences encoding the heavy and light chains of the anti-CD8 monoclonal antibodies were subcloned into the expression plasmid pRG984 (SEQ ID NO: 25) under the control of the human Ubiquitin C (hUbC) promoter and the β-globin intron.
   pBudCE4.1-CD79a/b (SEQ ID NO: 38) is used as the second envelope plasmid. pBudCE4.1-CD79a/b is a bicistronic plasmid that expresses the coding sequences of CD79a and CD79b, the two associated proteins required for the surface expression of antibodies. The coding sequences of CD79a and CD79b were subcloned into the bicistronic expression plasmid pBudCE4.1 (Invitrogen/ThermoFisher, SEQ ID NO: 39), respectively under the control of the CMV and EF1α promoters.
   pRG984-SINmu (SEQ ID NO: 26) is used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of a control lentiviral particles targeting all CD4+ T cells comprising an anti-mouse CD4 monoclonal antibody (clone GK1.5) on its envelope surface and comprising either a reporter protein (LNGFR), or a regulating transcription factor (TBX21) as the nucleotide of interest is achieved by co-transfection with the following plasmids:
1. Transfer plasmids comprising a nucleotide sequence of interest:
   pLVX-EF1α-GSG-P2A-LNGFR (SEQ ID NO: 66) that encodes the LNGFR reporter protein under control of the ubiquitous EF1α promoter. pLVX-EF1α-P2A-LNGFR was generated by inserting the LNGFR sequence (Milteniy Biotech) sequence into pLVX-EF1α-GSG-P2A-EGFP (SEQ ID NO: 14) derived from the commercial pLVX-EF1α-IRES-Puro plasmid (Clontech/Takara). Alternative reporters include, but are not limited to Thy1.1, EGFP, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-mTBX21-P2A-LNGFR (SEQ ID NO: 30) that comprises a codon-optimized cDNA encoding the canonical isoform of the mouse transcription factor TBX21 (sequences synthesized by IDT, SEQ ID NO: 29) under control of the ubiquitous EF1α promoter and an LNGFR reporter linked to mouse TBX21 via a self-cleaving P2A peptide. pLVX-EF1a-mTBX21-P2A-LNGFR has been generated by inserting the mouse TBX21 cDNA sequence into the pLVX-EF1a-GSG-P2A-LNGFR (SEQ ID NO: 66) backbone plasmid, itself derived from the commercial pLVX-EF1α-IRES-Puro plasmid (Clonetech/Takara). Alternative transfer vector plasmid contains only mouse TBX21 with no reporter, or a different reporter protein, including, but not limited to, Thy1.1, EGFP, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-mTBX21-IRES-LNGFR (SEQ ID NO: 31) is generated by inserting the sequences encoding mouse TBX21 into the pLVX-EF1α-IRES-LNGFR plasmid (SEQ ID NO: 67), itself derived from the commercial pLVX-EF1α-IRES-Puro plasmid (Clonetech/Takara) by replacement of the puromycin resistant sequence with the EGFP sequence.
Alternative non-limiting example of other transcription factors include mouse EOMES and ZEB2 (SEQ ID NO: 32 and 33)
   Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contain mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. Packaging plasmids:
   psPAX2 is used as the first packaging plasmid (Addgene plasmid #12260). PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2^{nd} generation packaging plasmids, 3^{rd} generation packaging plasmids, such as pRSV-Rev (Addgene plasmid #12253) and pMDLg-pRRE (Addgene plasmid #12251), Dull et al., J Virol, 72(11):8463-8471 (1998)) or 4^{th} generation packaging system such as Lenti-X® Fourth Generation packaging system (Clontech/Takara). Other modified versions of psPAX2 presenting mutations either in the integrase or the retrotranscriptase enzymes are used too for generating non-integrative versions of those vectors (TABLE 1).
   In some experiments a second packaging plasmid, pRG984-C-Scr-Vpx is used (SEQ ID NO: 34). This plasmids encodes the viral protein Vpx of the Simian Immunodeficience Virus (SIV—Mac251 strain) fused to the membrane targeting domain of the C-Src protein with an extra protease cleavage site (KARVLAEA (SEQ ID NO: 88), initially located between the HIV GAG proteins p27 and p29) for both an efficient incorporation of Vpx in the lentiviral particles and its release in the infected cells, as previously described (Durand et al., J. Virol. 87 (1): 234-242 (2013)). In several hematopoietic cells including T cells, Vpx counteracts the restriction factor SAMHD1 that hampers lentiviral infection at the reverse transcription level by depleting the pool of deoxynucleosides triphosphates (dNTPs) and thus limiting generation of de novo viral DNA from the HIV provirus genome. An alternative plasmid used is pRG984-C-Src-Flag-Vpx that encodes a tagged version of the Vpx protein (SEQ ID NO: 35)
3. Envelope plasmids:
   pRG984-αmCD4(GK1.5)-IgG4us (SEQ ID NO: 68) and pRG984-αmCD4(GK1.5)-IgK (SEQ ID NO: 69), encoding respectively the heavy chain and the light chain of the anti-mouse CD4 (clone GK1.5) monoclonal antibody are used as the first envelope plasmids for the production of the LV-αmCD4-mTBX21-P2A-EGFP and LV-H-αmCD4-mTBX21-P2A-EGFP lentiviral particles respectively. The nucleotide sequences encoding the heavy and light chains of the anti-CD8 monoclonal antibodies were subcloned into the expression plasmid pRG984 (SEQ ID NO: 25) under the control of the human Ubiquitin C (hUbC) promoter and the β-globin intron.
   pBudCE4.1-CD79a/b (SEQ ID NO: 38) is used as the second envelope plasmid. pBudCE4.1-CD79a/b is a bicistronic plasmid that expresses the coding sequences of CD79a and CD79b, the two associated proteins required for the surface expression of antibodies. The coding sequences of CD79a and CD79b were subcloned into the bicistronic expression plasmid pBudCE4.1 (Invitrogen/ThermoFisher, SEQ ID NO: 39), respectively under the control of the CMV and EF1α promoters.

pRG984-SINmu (SEQ ID NO: 26) is used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

### Cell Lines

Adherent HEK 293T/17 cells (ATCC) are cultured in DMEM medium (Gibco/Life Technologies) supplemented with 10% fetal bovine serum and 1% penicillin-streptomycin.

### Splenocytes Isolation and CD8 T Cells Culture

Spleens of adult C57Bl/6, OT-1 or P14 mice are first excised and placed in cold PBS (without calcium or magnesium) supplemented with 2% FBS. Tissues are then homogenized using GentleMACS™ Octo Dissociator (Milteniy) to break apart the spleens in GentleMACS™ C Tubes (Milteniy). Dissociated cells are then centrifuged at 500 g, 4 C for 5 min. After centrifugation the cell pellet is resuspended with gentle vortexing in a suitable volume of ACK lysis buffer (5 ml per spleen, Gibco/Life Technologies) to remove the red blood cells, and incubated for 5 min at room temperature. After incubation the cell suspension is added to 10 ml/spleen of PBS/FBS, and centrifuged at 500 g, 4 C for 5 min. The cell pellet is then resuspended in PBS/FBS and the cell solution was filtered through a 0.7 μM filter (BD Biosciences). The filtered cell suspension is centrifuged again and the subsequent pellet of individualized cells is resuspended one more time in a suitable volume of PBS/FBS.

CD8+ T cells are then isolated using a CD8a+ T cells Isolation Kit (Milteniy)

For the mixed T cell cultures experiment, isolated CD8a+ T cells from wild-type C57Bl/6 and OT-1 mice were combined in culture at a ratio 1:1.

### Antigen Presenting Cell Primary Cultures

A spleen of adult C57Bl/6 mouse is first excised and placed in cold PBS (without calcium or magnesium) supplemented with 2% FBS. Tissues are then homogenized using frosted glass slides (VWR) and centrifuged at 500 g, 4 C for 5 min. After centrifugation the cell pellet is resuspended with gentle vortexing in a suitable volume of ACK lysis buffer (5 ml per spleen, Gibco/Life Technologies) to remove the red blood cells and incubated for 5 min at room temperature. After incubation the cell suspension is added to 10 ml/spleen of PBS/FBS, and centrifuged at 500 g, 4 C for 5 min. The cell pellet is then resuspended in PBS/FBS and the cell solution is filtered through a 0.7 μM filter (BD Biosciences). The filtered cell suspension is centrifuged again and the subsequent pellet of individualized cells is resuspended one more time in a suitable volume of PBS/FBS. Total spleen cells are counted, and global antigen-presenting cells (APC) are sorted using anti-MHC class II Microbeads and MACS® sorting technology (Miltenyi Biotec). After elution, the MHC class II-positive cell fraction is collected and resuspended at 1×10⁶ cells/ml in APC cell medium composed of RPMI 1640 (Gibco/Life Technologies)+1% Horse serum+50 U/ml penicillin (Gibco/Life Technologies)+50 μg/ml streptomycin (Gibco/Life Technologies)+2 Mm L-Glutamin (Gibco/Life technologies) supplemented with 30 ng/ml of IL-4 (Peprotech), 50 ng/ml of GM-CSF (Peprotech) and 1 ng/ml of recombinant mouse TNFα (Peprotech).

Pulsing of APCs with Peptides

APC are then incubated with 10 μg/ml of each peptide (gp₃₃₋₄₁ (KAVYNFATC; SEQ ID NO: 1) or OVA₂₅₇₋₂₆₄ peptide (SIINFEKL; SEQ ID NO: 7)) overnight. The day after, peptide-pulsed APC are harvested and washed before addition at a ratio 1:1 to the different J.RT3-T3.5 derived cell lines, 48 h after infection with the lentiviral particles. J.RT3-T3.5-derived cell lines are then cocultured with the pulsed APC in J.RT3-T3.5 cell culture medium in duplicate with one half for proliferation assay and one half for luciferase assay+ cytokines production assay.

### Lentiviral Particle Production

Lentiviral particles are produced following standard lipofectamine-mediated co-transfection of HEK 293T cells with the respective transfer vector, packaging and envelope plasmids. The day before transfection cells are washed with phosphate buffered saline solution (PBS) once then detached from vessel with TrypLE™ Express (Life Technologies). After neutralization of TrypLE Express with cell medium containing FBS, cells are centrifuged at 1200 rpm for 5 min at 25 C, then resuspended in complete DMEM medium, counted and seeded in 150 mm cell culture dishes at a density of 10×10⁶ cells/plate. On the day of transfection, the cell culture medium is replaced by fresh Opti-MEM medium (Gibco/Life Technologies) supplemented with 25 nM chloroquine (Sigma-Aldrich). The DNA mix is prepared by mixing 60 μg of total DNA 1.5 mL of Opti-MEM with 60 ul of PLUS™ Reagent (Life Technologies). The specific plasmid ratios for the different lentivirus productions are illustrated in TABLE 2. In parallel 100 μl of Lipofectamine® TLX (Life Technologies) is diluted in 1.5 mL of OptiMEM medium. DNA mix is then added to the lipofectamine mix and the new combined solution is incubated at room temperature for 20 minutes before being added directly to the cells dropwise. The culture medium is changed 6 to 8 h after transfection and the cells are then incubated for 48 h at 37 C in an incubator with 5% CO₂ atmosphere. At day 2 post-transfection, cell media containing the lentiviral particles are centrifuged for 10 min at 3000 rpm to remove the debris, then passed through a 0.45 um pore size filter. The filtered supernatants are then treated with 1p g/ml DNAse and 1 mM MgCl2 for 15 minutes at 37° C. to remove residual DNA. For concentrating the lentiviral vectors batch, the supernatants are then ultracentrifuged at 27,100 rpm for 90 min. After ultracentrifugation, pellets are resuspended in a suitable volume of PBS (50 to 100 μl) overnight. The resuspended virus is finally processed through a series of short centrifugations (30 sec at 13500 rpm) to clarify the lentiviral solution of remaining debris. The batches of lentiviral particles are titrated by RT-qPCR using a SYBR® technology-based kit from Clontech/Takara then stocked at −80° C. until use for transduction.

Alternative transfections reagents include polyethylenimine (PEI), calcium chloride or calcium phosphate.

### Lentiviral Transduction of Mouse Primary T Cells

On day of transduction, CD8+ T cells isolated from P14 and OT-1 mice are centrifuged for 5 minutes at 1200 RPM and 25° C. After resuspension in fresh complete T cell medium, cells are counted, seeded in 24-well plates at a density of 100,000 cells/well and mixed with 10 μg/ml Vectofusin®-1 (Miltenyi Biotec) and the suitable amount of virus (standard dose of 25,000 viral copies per cell). Cells are then spinoculated by centrifugating them in presence of the virus at 2000 rpm for 90 min at 30° C. After centrifugation, cells are incubated at 37° C. and 5% C02 for 3 days before fixation and staining/FACS analysis. Alternatively to polybrene, CD8+ T cells are transduced on plates pre-coated with retronectin (Clontech/Takara, 15-40 g/ml) or with LentiBOOST™ (1:100, Sirion Biotech).

Additionally, to optimize transduction efficiency of lentiviral particles in mouse primary T cells, cultures are also treated in some experiments with histone deacetylase inhibitors (HDACi) such as sodium butyrate and Trichostatin-A (TSA).

### Cells Staining and FACS Analysis

Fluorescence-activated cell sorting (FACS) is performed on day 3 after the transduction. Transduced cells are counted and seeded equally into a 96-well V bottom plate. Cells are spun at 2000 rpm at 4 C for 2 min, washed with PBS then spun again. Cells are incubated with Live/Dead® Fixable Near-IR stain (Life Technologies, diluted at 1:10000 in PBS) for 15 min in the dark, washed, and incubated with Fc block (eBioscience, dilution 1:150) in FACS Stain Buffer (BD Biosciences). After being washed twice again with FACS stain buffer, cells are subsequently incubated for 30 min, on ice and in the dark with either of the following tetramers and antibodies: iTag Tetramer/APC-H-2K^{b} OVA® (MBL), iTag Tetramer/APC-H-2D^{b} LCMV®Alexa Fluor®, BV451-conjugated anti-mouse CD8 (Biolegend, 0.25 μg per 10⁶ cells) or BV421 anti-mouse CD8a (clone 53-6.7, Biolegend, 0.25 μg per 10⁶ cells) and BV421 IgG2a, κ Isotype Control (Biolegend, 0.25 μg per 10⁶ cells). Cells are washed one more time with FACS stain buffer, fixed with paraformaldehyde 1%, washed again and finally resuspended in FACS stain buffer. Samples are run for analysis with a BD FACS Canto II analyzer (BD Biosciences).

### Peptide Immunization of Mice

For immunization 1 μg of gp₃₃₋₄₁ (KAVYNFATC; SEQ ID NO: 1) or OVA₂₅₇₋₂₆₄ peptide (SIINFEKL; SEQ ID NO: 7) is diluted in PBS and emulsified 1:1 with either complete Freunds adjuvant (CFA) or incomplete Freunds adjuvant (IFA) using a double syringes system, to reach a final volume of 1 ml of peptide/adjuvant emulsion. 200 ul of the peptide/adjuvant emulsion is then injected subcutaneously in C57Bl/6 mice under isoflurane anaesthesia in 4 different locations (behind each shoulder and hip).

### Proliferation and Cytokine Production Assays

To measure T cell proliferation, on half of the incubated cells, 1 μCi/well [3H]thymidine (Amersham) is added to assay cultures at 48 h after contact with the peptide-pulsed APC. Following incubation for 12-16 h, cultures are harvested onto Unifiter Plates (Packard Instrument). Microscint 20 scintillation fluid (Packard Instrument) is added to each well, and plates are counted on a Scintillation Counter.

For transcription factor activity analysis and cytokines production assay, the other half of the cells is harvested 5 days after contact with the peptide-pulsed APC, centrifuged for 5 min at 300 g, and both cell pellets and supernatants are collected. Cell pellets are processed for RNA extraction for transcriptomics analysis via qPCR, cytokines production is measured by ELISA from the supernatant using the Mouse TH1/TH2 9-Plex Tissue Culture Kit from Meso Scale Diagnostics (MSD).

### Quantification of Cytotoxicity Activity by Flow Cytometry

### Preparation of Target Cells:

Targets cells (autologous B cells) are isolated from splenocytes of OT-1 or P14 mice by using the Mouse B cell s Kit (Milteniy Biotec) according to the manufacturer's instruction and are stimulated for 18 h in the presence of 40 ng/mL of IFN-γ (Peprotech). Targets cells are then counted, split into two tubes and washed in warm PBS once. Half of the target cells are stained with a high concentration (0.2 μM) of CFSE (CFSE_{High}) (Invitrogen/Thermofisher) and the other half with a low concentration (0.02 μM) of CFSE (CFSE_{Low}) in 1 mL of warm PBS for 15 min at 37 C. After the incubation, cells are pelleted and resuspended in 1 mL of warm complete RPMI 1640 (4 mM L-glutamine, and 100 U/mL penicillin and streptomycin, supplemented with 10% FBS) to quench the labeling reaction. Target cells stained with the low concentration of CFSE are pulsed by adding the peptide of interest (gp33 or OVA) at a final concentration of 5 g/mL in the complete RPMI and incubated for 45 min in 5% CO₂ atmosphere at 37 C. Both target cells stained with different concentration of CFSE are then washed twice in complete RPMI, resuspended at a concentration of 2×10⁵ cells/mL each in complete RPMI and mixed with a ratio of 1:1 (CFSE_{High}:CFSE_{Low}).

### Effector Cells

Total CD8 T cells containing the effectors cells are enriched from splenocytes of P14 and OT-1 mice using the Negative Selection Human CD8 T cell isolation Kit (Milteniy Biotec) according to the manufacturer's instruction. CD8 T cells are counted, resuspended in 450 μl in complete T cell medium and serial diluted volume:volume in 225 μl of complete T cell medium from 1:2 to 1:36. From each dilution, 100 μl are seeded in duplicate to round-bottom 96-well plate. The mixed target cells (100 μl) are added to each dilution of effector cells for a final volume of 200 μL. To measure the basal apoptosis, 4 wells are seeded with targets cells alone. Cell mixtures were incubated for 6 h in 5% CO₂ atmosphere at 37 C.

### Flow Cytometry Staining and Acquisition

Cells are transferred to a V-bottom 96 well plate, washed in FACS Stain Buffer (BD Bioscience) and stained with iTag Tetramer/APC-H-2K^{b} OVA® (MBL), iTag Tetramer/APC-H-2D^{b} LCMV®Alexa Fluor® and Live/Dead® Fixable Near-IR stain (ThermoFisher, diluted at 1:10000 in FACS Stain Buffer) and for 15 min in 5% CO₂ atmosphere at 37 C in the dark. Cells are then washed in FACS Stain Buffer before staining with BV421 αCD8a (Biolegend) for 30 min at +4 C in the dark. Cells are washed once with FACS Stain Buffer and resuspended with 2% paraformaldehyde (PFA; Sigma). Acquisition is performed on BD Biosciences LSR II analyzer and all cells are acquired. Post-acquisition data analysis is done with the Flowjo software (version 9.3.1; TreeStar Inc.).

### Example 3: Specific Targeting and Activation of Mouse Primary T Cells Expressing Antigen-Specific T Cell Receptor (TCR) with Retroviral Particles Displaying Antigenic Peptides in MHC Class I Groove on their Surface

This example describes the ability of retroviral vectors derived of gamma-retroviruses (Moloney Murine Leukemia Virus—MMLV and Murine Stem Cell Virus—MSCV) and pseudotyped with MHC class I molecules displaying an antigenic peptide (i.e., epitope) and their use for specifically transducing and modifying the phenotype of mouse primary cells that express an antigen-specific T cell receptor (TCR) that recognizes this peptide.

In particular, this example demonstrates the targeting of mouse primary CD8+ T cells expressing the OT-1 TCR with MMLV and MSCV-derived retroviral particles pseudotyped with a MHC class I molecule (H-2K^{b}) presenting the OVA₂₅₇₋₂₆₄ peptide (OVA, SIINFEKL, SEQ ID NO: 7 and 8) peptide in MHC groove.

More particularly, the ability of retroviral particles carrying the sequence encoding the mouse transcription factor TBX21 (mTBX21, SEQ ID NO: 29) as a nucleotide of interest to trigger activation of mouse naïve CD8+ T cells into CD8+T effector T cells and enhance an antiviral response is tested. TBX21, also known as T-bet, is a T cell-specific transcription factor controlling the expression of IFNγ and primarily known for being involved in the development of the Th1 lineage from naïve CD4+ T cells. In CD8+ T cells, TBX21 expression is induced after TCR and IL-12-mediated activation, and is required for the production of IFNγ, Granzyme B and perforin (Lazarevic et al., Nature Reviews Immunology, 13:777-789 (2013)). TBX21 thus plays a well-established role in effector and memory CD8+ T cells differentiation in association with other transcription factors such as eomesodermin (EOMES) and runt-related transcription factor 3 (RUNX3). Moreover, retroviral overexpression of TBX21 in primary CD8+ T cells has been shown to reduce the expression of the T cell exhaustion marker PD1 and to trigger enhanced CD8+ T cells response in chronic infections models (Kao et al., Nat. Immunol. 12(7): 663-671 (2012)) Targeted overexpression of TBX21 in naïve CD8+ T cell with retroviral particles pseudotyped with MHC class I/peptide complexes thus appears to be a powerful alterative to lentiviral vectors to induce an antigen-specific CD8+ effective response.

A first retroviral particle is generated comprising at its surface (i) H-2K^{b} (MHC class I molecule) displaying the OVA peptide to target the OT-1 TCR (mROR-OVA-β2m-H-2K^{b}, SEQ ID NO: 9) and (ii) a fusogen “SINmu,” as previously described.

Another retroviral particle is generated comprising at its surface (i) H-2D^{b} (MHC Class I molecule) displaying the gp33 peptide to target the OT-1 TCR (mROR-gp33-β2m-H-2D^{b}, SEQ ID NO: 4) and ii) a fusogen “SINmu” as previously described.

These retroviral particles are first generated to carry an EGFP reporter nucleotide of interest (or any other reporter gene including, but not limited to, Thy1.1, LNGFR, DsRed, RFP, YFP, CFP, mCherry, TdTomato, Luciferase, and Lac-Z).

For functional studies, bicistronic retroviral particles pseudotyped with the H-2K^{b}/OVA or the H-2D^{b}/gp33 are used to deliver the murine transcription factor TBX21 in T cells ex vivo. The effects of such treatment on the T cell phenotype are analyzed. Specifically, the ability of retroviral particles displaying the H-2-K^{b}/OVA complex on their surface and comprising the genetic sequence encoding mouse TBX21 (Tbx21, GenBank: AF277994.1, AF27799, AF277992) as a nucleotide of interest to transfer the said nucleotide of interest and modulate T cell phenotype, in this case a cytotoxic phenotype in infected cells is evaluated in primary mouse T cells isolated from transgenic OT-1 mice to expand CD8+ T cells with the OVA-specific OT-1 TCR. Similarly, the ability of retroviral particles displaying the H-2-K^{b}/OVA complex on their surface and comprising the genetic sequence encoding mouse TBX21 as a nucleotide of interest to transfer the said nucleotide of interest and modulate T cell phenotype, is evaluated in primary mouse T cells isolated from transgenic P14 mice (β6; β10-Rag2^{tm1Fwa} Tg(TcrLCMV)327Sdz, Taconic) to expand CD8+ T cells with the gp33-specific P14 TCR.

For ex vivo functional assays on mouse primary cells, CD8+ T-cells are isolated from splenocytes of transgenic P14 mice and OT-1 mice and then transduced with either RV-H-2D^{b}/gp33-mTBX21-P2A-EGFP or RV-H-2K^{b}/OVA-mTBX21-P2A-EGFP retroviral particles. Additional controls include transduction without the retroviral particles (mock) or with retroviral targeting all CD8+T or all CD4+ T cells by displaying respectively an anti-mouse CD8 monoclonal antibody or an anti-mouse CD4 monoclonal on their surface. After retroviral transductions, P14 and OT-1 CD8 T cells are then stimulated with any of the following methods: (i) treatment with phytohemagglutinin (PHA), (ii) treatment with phorbol 12-myristate 13-acetate (PMA), (iii) treatment with both PHA and PMA, (iv) soluble or immobilized anti-CD3/anti-CD28 monoclonal antibodies, (v) activation beads coupled with anti-CD3/anti-CD8 beads, (vi) antigen-presenting cells (APC) previously pulsed with gp33 and OVA peptides or (vii) any other suitable method to activate T cells (FIG. 9). An alternative experimental setting consists in immunizing wild type C57Bl/6 mice with either the gp33 or OVA peptide, or other appropriate methods, to expand CD8+ T cells expressing TCRs restricted to gp33 or OVA peptides prior to lentivirus transduction (FIG. 10). Two weeks after immunization, splenocytes containing memory T cells are isolated and transduced with either RV-H-2D^{b}/gp33-mTBX21-P2A-EGFP or RV-H-2K^{b}/OVA-mTBX21-P2A-EGFP retroviral particles, or other control conditions previously described. After retroviral transductions, splenocytes are stimulated again with the same techniques described for FIG. 9. T-cell phenotype following transduction is evaluated by measuring (i) expression of T cell activation and T cell exhaustion markers such as, but not restricted to, CD69 or PD-1 (ii) cytokines (e.g., IL-2, IFNγ, etc.) production, (iii) cell proliferation, (iv) production of cytolytic proteins such as Granzyme B and perforin, (iv) cytotoxic activity proliferation in cocultures with peptide-pulsed target cells and/or (v) other well-known cytotoxic T-cell characteristics.

### Material and Methods

### Plasmids for Retroviral Vector Production:

The production of retroviral particles comprising the gp33 peptide in the groove of the MHC class I (H-2D^{b}) molecule, or the OVA peptide in the groove of the MHC class I (H-2K^{b}) on its envelope surface and comprising either a reporter protein (EGFP), or a regulating transcription factor (TBX21) as the nucleotide of interest is achieved by co-transfection with the following plasmids:
1. Transfer plasmids comprising a nucleotide sequence of interest:
   pMXS-EF1α-GFP (Cell Biolabs, RTV-601) is a MMLV backbone plasmid that encodes the EGFP reporter protein under control of the ubiquitous promoter EF1α. Alternative reporters include, but are not limited to Thy1.1, LGNFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity.
   pMXS-EF1α-mTBX21-P2A-EGFP (SEQ ID NO: 40) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMXS-mTBX21-P2A-EGFP is generated by inserting the mouse TBX21-P2A-EGFP cassette into the pMXS-EF1-Bsd (Cell Biolabs, RTV-062) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33)
Each of the transfer vectors also contained the following common elements: 5′ MMLV LTR, a primer binding site (PBS), an extended packaging signal, the Rev Response Element (RRE), and a self-inactivating (SIN) MMLV3′ LTR.
Alternatively, the MSCV-derived transfer vector plasmids are used:
   pMSGV-EGFP-PGK-puro (SEQ ID NO: 41) is a MSCV backbone plasmid that encodes the EGFP reporter protein under control of the original MSCV 5′ LTR. pMSGV-EGFP-PGK-puro is generated by inserting the EGFP reporter gene into the pMSGV-PGK puro backbone (Creative Biolabs). Alternative reporters include, but are not limited to, Thy1.1, LNGFR, DsRed, RFP, YFP, CFP, mCherry, TdTomato, Luciferase, and Lac-Z proteins. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity.
   pMSGV-mTBX21-P2A-EGFP-PGK-puro (SEQ ID NO: 42) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMSGV-mTBX21-P2A-EGFP is generated by inserting the mouse TBX21-P2A-EGFP cassette into the pMXS-PGK-puro (Creative Biolabs) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33).
Each of the transfer vectors also contained the following common elements: 5′ MSCV LTR, a primer binding site (PBS), an extended packaging signal, the Rev Response Element (RRE), and a self-inactivating (SIN) MSCV3′ LTR.
2. Packaging plasmids:
   pGag-Pol is used as the packaging plasmid (Addgene plasmid #14887). Gag-Pol is a packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase).
3. Envelope plasmids:
   pRG984-H-2D^{b}/gp33 (SEQ ID NO: 23) and pRG984-H-2K^{b}/OVA (SEQ ID NO: 24) are used as the first envelope plasmid for the production of the LV-H-2D^{b}/gp33-mTBX21-IRES-EGFP and LV-H-2K^{b}/OVA-mTBX21-IRES-EGFP lentiviral particles respectively. The mROR-gp33-β2m-H-2D^{b} and mROR-OVA-β2m-H-2K^{b} cassettes were subcloned into the expression plasmid pRG984 (SEQ ID NO: 25) under the control of the human Ubiquitin C (hUbC) promoter and the β3-globin intron.
   pRG984-SINmu (SEQ ID NO: 26) is used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of a control lentiviral particles targeting all CD8+ T cells comprising an anti-mouse CD8 monoclonal antibody (clone YTS156) on its envelope surface and comprising either a reporter protein (EGFP), or a regulating transcription factor (TBX21) as the nucleotide of interest is achieved by co-transfection with the following plasmids:
1. Transfer plasmids comprising a nucleotide sequence of interest:
   pMXS-EF1α-GFP (Cell Biolabs, RTV-601) is a MMLV backbone plasmid that encodes the EGFP reporter protein under control of the ubiquitous promoter EF1α. Alternative reporters include, but are not limited to, Thy1.1, LNGFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity.
   pMXS-EF1α-mTBX21-P2A-EGFP (SEQ ID NO: 40) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMXS-mTBX21-P2A-EGFP is generated by inserting the mouse TBX21-P2A-EGFP cassette into the pMXS-EF1-Bsd (Cell Biolabs, RTV-062) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33)
Each of the transfer vectors also contained the following common elements: 5′ MMLV LTR, a primer binding site (PBS), an extended packaging signal, the Rev Response Element (RRE), and a self-inactivating (SIN) MMLV3′ LTR.
Alternatively, the MSCV-derived transfer vector plasmids are used:
   pMSGV-EGFP-PGK-puro (SEQ ID NO: 41) is a MSCV backbone plasmid that encodes the EGFP reporter protein under control of the original MSCV 5′ LTR. pMSGV-EGFP-PGK-puro is generated by inserting the EGFP reporter gene into the pMSGV-PGK puro backbone (Creative Biolabs). Alternative reporters include, but are not limited to, Thy1.1, LNGFR, DsRed, RFP, YFP, CFP, mCherry, TdTomato, Luciferase, and Lac-Z proteins. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity.
   pMSGV-mTBX21-P2A-EGFP-PGK-puro (SEQ ID NO: 42) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMSGV-mTBX21-P2A-EGFP is generated by inserting the mouse TBX21-P2A-EGFP cassette into the pMXS-PGK-puro (Creative Biolabs) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33).
Each of the transfer vectors also contained the following common elements: 5′ MSCV LTR, a primer binding site (PBS), an extended packaging signal, the Rev Response Element (RRE), and a self-inactivating (SIN) MSCV3′ LTR.
2. Packaging plasmids:
   pGag-Pol is used as the packaging plasmid (Addgene plasmid #14887). Gag-Pol is a packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase).
3. Envelope plasmids:
   pRG984-αmCD8(YTS156)-IgG4us (SEQ ID NO: 36) and pRG984-αmCD8(YTS156)-IgK (SEQ ID NO: 37), encoding respectively the heavy chain and the light chain of the anti-mouse CD8 (clone YTS156) monoclonal antibody are used as the first envelope plasmids for the production of the LV-αmCD8-mTBX21-P2A-EGFP and LV-H-αmCD8-mTBX21-P2A-EGFP lentiviral particles respectively. The nucleotide sequences encoding the heavy and light chains of the anti-CD8 monoclonal antibodies were subcloned into the expression plasmid pRG984 (SEQ ID NO: 25) under the control of the human Ubiquitin C (hUbC) promoter and the β-globin intron.
   pBudCE4.1-CD79a/b (SEQ ID NO: 38) is used as the second envelope plasmid. pBudCE4.1-CD79a/b is a bicistronic plasmid that expresses the coding sequences of CD79a and CD79b, the two associated proteins required for the surface expression of antibodies. The coding sequences of CD79a and CD79b were subcloned into the bicistronic expression plasmid pBudCE4.1 (Invitrogen/ThermoFisher, SEQ ID NO: 39), respectively under the control of the CMV and EF1α promoters.
   pRG984-SINmu (SEQ ID NO: 26) is used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of a control lentiviral particles targeting all CD4+ T cells comprising an anti-mouse CD8 monoclonal antibody (clone GK1.5) on its envelope surface and comprising either a reporter protein (EGFP), or a regulating transcription factor (TBX21) as the nucleotide of interest is achieved by co-transfection with the following plasmids:
1. Transfer plasmids comprising a nucleotide sequence of interest:
   pMXS-EF1α-GFP (Cell Biolabs, RTV-601) is a MMLV backbone plasmid that encodes the EGFP reporter protein under control of the ubiquitous promoter EF1α. Alternative reporters include, but are not limited to, Thy1.1, LNGFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity.
   pMXS-EF1α-mTBX21-P2A-EGFP (SEQ ID NO: 40) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMXS-mTBX21-P2A-EGFP is generated by inserting the mouse TBX21-P2A-EGFP cassette into the pMXS-EF1-Bsd (Cell Biolabs, RTV-062) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33)
Each of the transfer vectors also contained the following common elements: 5′ MMLV LTR, a primer binding site (PBS), an extended packaging signal, the Rev Response Element (RRE), and a self-inactivating (SIN) MMLV3′ LTR.
Alternatively, the MSCV-derived transfer vector plasmids are used:
   pMSGV-EGFP-PGK-puro (SEQ ID NO: 41) is a MSCV backbone plasmid that encodes the EGFP reporter protein under control of the original MSCV 5′ LTR. pMSGV-EGFP-PGK-puro is generated by inserting the EGFP reporter gene into the pMSGV-PGK puro backbone (Creative Biolabs). Alternative reporters include, but are not limited to, Thy1.1, LNGFR, DsRed, RFP, YFP, CFP, mCherry, TdTomato, Luciferase, and Lac-Z proteins. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity.
   pMSGV-mTBX21-P2A-EGFP-PGK-puro (SEQ ID NO: 42) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMSGV-mTBX21-P2A-EGFP is generated by inserting the mouse TBX21-P2A-EGFP cassette into the pMXS-PGK-puro (Creative Biolabs) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33).
Each of the transfer vectors also contained the following common elements: 5′ MSCV LTR, a primer binding site (PBS), an extended packaging signal, the Rev Response Element (RRE), and a self-inactivating (SIN) MSCV3′ LTR.
2. Packaging plasmids:
   pGag-Pol is used as the packaging plasmid (Addgene plasmid #14887). Gag-Pol is a packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase).
3. Envelope plasmids:
   pRG984-αmCD4(GK1.5)-IgG4us (SEQ ID NO: 68) and pRG984-αmCD8(GK1.5)-IgK (SEQ ID NO: 69), encoding respectively the heavy chain and the light chain of the anti-mouse CD4 (clone GK1.5) monoclonal antibody are used as the first envelope plasmids for the production of the LV-αmCD4-mTBX21-P2A-EGFP and LV-H-αmCD4-mTBX21-P2A-EGFP lentiviral particles respectively. The nucleotide sequences encoding the heavy and light chains of the anti-CD8 monoclonal antibodies were subcloned into the expression plasmid pRG984 (SEQ ID NO: 25) under the control of the human Ubiquitin C (hUbC) promoter and the β-globin intron.
   pBudCE4.1-CD79a/b (SEQ ID NO: 38) is used as the second envelope plasmid. pBudCE4.1-CD79a/b is a bicistronic plasmid that expresses the coding sequences of CD79a and CD79b, the two associated proteins required for the surface expression of antibodies. The coding sequences of CD79a and CD79b were subcloned into the bicistronic expression plasmid pBudCE4.1 (Invitrogen/ThermoFisher, SEQ ID NO: 39), respectively under the control of the CMV and EF1α promoters.

pRG984-SINmu (SEQ ID NO: 26) is used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINmu was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of a control lentiviral particles targeting all murine cells comprising an ecotropic envelope and comprising either a reporter protein (EGFP), or a regulating transcription factor (TBX21) as the nucleotide of interest is achieved by co-transfection with the following plasmids:
1. Transfer plasmids comprising a nucleotide sequence of interest:
   pMXS-EF1α-GFP (Cell Biolabs, RTV-601) is a MMLV backbone plasmid that encodes the EGFP reporter protein under control of the ubiquitous promoter EF1α. Alternative reporters include, but are not limited to, Thy1.1, LNGFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity.
   pMXS-EF1α-mTBX21-P2A-EGFP (SEQ ID NO: 40) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMXS-mTBX21-P2A-EGFP is generated by inserting the mouse TBX21-P2A-EGFP cassette into the pMXS-EF1-Bsd (Cell Biolabs, RTV-062) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33)
Each of the transfer vectors also contained the following common elements: 5′ MMLV LTR, a primer binding site (PBS), an extended packaging signal, the Rev Response Element (RRE), and a self-inactivating (SIN) MMLV3′ LTR.
Alternatively, the MSCV-derived transfer vector plasmids are used:
   pMSGV-EGFP-PGK-puro (SEQ ID NO: 41) is a MSCV backbone plasmid that encodes the EGFP reporter protein under control of the original MSCV 5′ LTR. pMSGV-EGFP-PGK-puro is generated by inserting the EGFP reporter gene into the pMSGV-PGK puro backbone (Creative Biolabs). Alternative reporters include, but are not limited to, Thy1.1, LNGFR, DsRed, RFP, YFP, CFP, mCherry, TdTomato, Luciferase, and Lac-Z proteins. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity.
   pMSGV-mTBX21-P2A-EGFP-PGK-puro (SEQ ID NO: 42) that comprises a codon-optimized cDNA encoding the canonical isoform of the mouse transcription factor TBX21 (sequences synthesized by IDT, SEQ ID NO: 29) under control of the EF1α promoter and an EGFP reporter linked to mouse TBX21 via a self-cleaving P2A peptide. pMSGV-mTBX21-P2A-EGFP is generated by inserting the mouse TBX21-P2A-EGFP cassette into the pMXS-PGK-puro (Creative Biolabs) backbone plasmid. Alternative transfer vector plasmid contains only mouse TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to EF1α, CMV, CMVmax, Ubiquitin C, PGK, CAGG, SFFV, initial MMLV LTR (no other promoter cloned) and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. Alternative non-limiting example of other transcription factors include mouse EOMES and ZEB2 (SEQ ID NO: 32 and 33).
Each of the transfer vectors also contained the following common elements: 5′ MSCV LTR, a primer binding site (PBS), an extended packaging signal, the Rev Response Element (RRE), and a self-inactivating (SIN) MSCV3′ LTR.
2. Packaging plasmids:
   pGag-Pol is used as the packaging plasmid (Addgene plasmid #14887). Gag-Pol is a packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase).
3. pHCMV-EcoEnv (Addgene #15802) as the envelope plasmid encoding the ecotropic receptor glycoprotein (Eco)

### Cell Lines

Adherent HEK 293T/17 cells (ATCC) are cultured in DMEM medium (Gibco/Life Technologies) supplemented with 10% fetal bovine serum and 1% penicillin-streptomycin.

### Splenocytes Isolation and CD8+ T Cells Culture

Spleens of adult C57Bl/6, OT-1 or P14 mice are first excised and placed in cold PBS (without calcium or magnesium) supplemented with 2% FBS. Tissues are then homogenized using GentleMACS™ Octo Dissociator (Milteniy) to break apart the spleens in GentleMACS™ C Tubes (Milteniy). Dissociated cells are then centrifuged at 500 g, 4 C for 5 min. After centrifugation the cell pellet is resuspended with gentle vortexing in a suitable volume of ACK lysis buffer (5 ml per spleen, Gibco/Life Technologies) to remove the red blood cells and incubated for 5 min at room temperature. After incubation the cell suspension is added to 10 ml/spleen of PBS/FBS, and centrifuged at 500 g, 4 C for 5 min. The cell pellet is then resuspended in PBS/FBS and the cell solution was filtered through a 0.7 μM filter (BD Biosciences). The filtered cell suspension is centrifuged again and the subsequent pellet of individualized cells is resuspended one more time in a suitable volume of PBS/FBS.

CD8+ T cells are then isolated using a CD8a+ T cells Isolation Kit (Milteniy)

### Antigen Presenting Cell Primary Cultures

A spleen of adult C57Bl/6 mouse is first excised and placed in cold PBS (without calcium or magnesium) supplemented with 2% FBS. Tissues are then homogenized using frosted glass slides (VWR) and centrifuged at 500 g, 4 C for 5 min. After centrifugation the cell pellet is resuspended with gentle vortexing in a suitable volume of ACK lysis buffer (5 ml per spleen, Gibco/Life Technologies) to remove the red blood cells and incubated for 5 min at room temperature. After incubation the cell suspension is added to 10 ml/spleen of PBS/FBS, and centrifuged at 500 g, 4 C for 5 min. The cell pellet is then resuspended in PBS/FBS and the cell solution is filtered through a 0.7 μM filter (BD Biosciences). The filtered cell suspension is centrifuged again and the subsequent pellet of individualized cells is resuspended one more time in a suitable volume of PBS/FBS. Total spleen cells are counted, and global antigen-presenting cells (APC) are sorted using anti-MHC class II Microbeads and MACS® sorting technology (Miltenyi Biotec). After elution, the MHC class II-positive cell fraction is collected and resuspended at 1×10⁶ cells/ml in APC cell medium composed of RPMI 1640 (Gibco/Life Technologies)+1% Horse serum+50 U/ml penicillin (Gibco/Life Technologies)+50 μg/ml streptomycin (Gibco/Life Technologies)+2 Mm L-Glutamin (Gibco/Life technologies) supplemented with 30 ng/ml of IL-4 (Peprotech), 50 ng/ml of GM-CSF (Peprotech) and 1 ng/ml of recombinant mouse TNFα (Peprotech).

Pulsing of APCs with Peptides

APC are then incubated with 10 μg/ml of each peptide (gp33-41 (KAVYNFATC; SEQ ID NO: 1) or OVA₂₅₇₋₂₆₄ peptide (SIINFEKL; SEQ ID NO: 7; nucleotide (AGTATAATCAACTTTGAAAAACTG; SEQ ID NO: 8)))) overnight. The day after, peptide-pulsed APC are harvested and washed before addition at a ratio 1:1 to the different J.RT3-T3.5 derived cell lines, 48 h after infection with the lentiviral particles. J.RT3-T3.5-derived cell lines are then cocultured with the pulsed APC in J.RT3-T3.5 cell culture medium in duplicate with one half for proliferation assay and one half for luciferase assay+ cytokines production assay.

### Retroviral Particle Production

Retroviral particles are produced following standard lipofectamine-mediated co-transfection of HEK 293T cells with the respective transfer vector, packaging and envelope plasmids. The day before transfection cells are washed with phosphate buffered saline solution (PBS) once then detached from vessel with TrypLE™ Express (Life Technologies). After neutralization of TrypLE Express with cell medium containing FBS, cells are centrifuged at 1200 rpm for 5 min at 25 C, then resuspended in complete DMEM medium, counted and seeded in 150 mm cell culture dishes at a density of 10×10⁶ cells/plate. On the day of transfection, the cell culture medium is replaced by fresh Opti-MEM medium (Gibco/Life Technologies) supplemented with 25 nM chloroquine (Sigma-Aldrich). The DNA mix is prepared by mixing 60 μg of total DNA 1.5 mL of Opti-MEM with 60 ul of PLUS™ Reagent (Life Technologies). The specific plasmid ratios for the different lentivirus productions are illustrated in TABLE 2. In parallel 100 μl of Lipofectamine® TLX (Life Technologies) is diluted in 1.5 mL of OptiMEM medium. DNA mix is then added to the lipofectamine mix and the new combined solution is incubated at room temperature for 20 minutes before being added directly to the cells dropwise. The culture medium is changed 6 to 8 h after transfection and the cells are then incubated for 48 h at 37 C in an incubator with 5% CO₂ atmosphere. At day 2 post-transfection, cell media containing the retroviral particles are centrifuged for 10 min at 3000 rpm to remove the debris, then passed through a 0.45 um pore size filter. The filtered supernatants are then treated with 1p g/ml DNAse and 1 mM MgCl2 for 15 minutes at 37° C. to remove residual DNA. For concentrating the retroviral vectors batches, the supernatants are then ultracentrifuged at 27,100 rpm for 90 min. After ultracentrifugation, pellets are resuspended in a suitable volume of PBS (50 to 100 μl) overnight. The resuspended virus is finally processed through a series of short centrifugations (30 sec at 13500 rpm) to clarify the retroviral solution of remaining debris. The batches of retroviral particles are titrated by RT-qPCR using a SYBR® technology-based kit from Clontech/Takara then stocked at −80° C. until use for transduction.

Alternative transfections reagents include polyethylenimine (PEI), calcium chloride or calcium phosphate.

### Retroviral Transduction of Mouse Primary T Cells

On day of transduction, CD8+ T cells isolated from P14 and OT-1 mice are centrifuged for 5 minutes at 1200 RPM and 25° C. After resuspension in fresh complete T cell medium, cells are counted, seeded in 24-well plates at a density of 100,000 cells/well and mixed with 10 μg/ml Vectofusin®-1 (Miltenyi Biotec) and the suitable amount of retroviral supernatant or concentrated virus (standard dose of 25,000 viral copies per cell). Cells are then spinoculated by centrifugating them in presence of the virus at 2000 rpm for 90 min at 30° C. After centrifugation, cells are incubated at 37° C. and 5% C02 for 3 days before fixation and staining/FACS analysis. Alternatively to polybrene, CD8+ T cells are transduced on plates pre-coated with retronectin (Clontech/Takara, 15-40 μg/ml) or LentiBOOST™ (1:100, Sirion Biotech).

Additionally, to optimize transduction efficiency of retrovrial particles in mouse primary T cells, cultures are also treated in some experiments with histone deacetylase inhibitors (HDACi) such as sodium butyrate and Trichostatin-A (TSA).

### Cells Staining and FACS Analysis

Fluorescence-activated cell sorting (FACS) is performed on day 3 after the transduction. Transduced cells are counted and seeded equally into a 96-well V bottom plate. Cells are spun at 2000 rpm at 4 C for 2 min, washed with PBS then spun again. Cells are incubated with Live/Dead® Fixable Near-IR stain (Life Technologies, diluted at 1:10000 in PBS) for 15 min in the dark, washed, and incubated with Fc block (eBioscience, dilution 1:150) in FACS Stain Buffer (BD Biosciences). After being washed twice again with FACS stain buffer, cells are subsequently incubated for 30 min, on ice and in the dark with either of the following tetramers and antibodies: iTag Tetramer/APC-H-2Kb OVA® (MBL), iTag Tetramer/APC-H-2Db LCMV@Alexa Fluor®, BV451-conjugated anti-mouse CD8 (Biolegend, 0.25 μg per 10⁶ cells) or BV421 anti-mouse CD8a (clone 53-6.7, Biolegend, 0.25 μg per 10⁶ cells) and BV421 IgG2a, κ Isotype Control (Biolegend, 0.25 μg per 10⁶ cells). Cells are washed one more time with FACS stain buffer, fixed with paraformaldehyde 1%, washed again and finally resuspended in FACS stain buffer. Samples are run for analysis with a BD FACS Canto II analyzer (BD Biosciences).

### Peptide Immunization of Mice

For immunization 1 μg of gp₃₃₋₄₁ (KAVYNFATC; SEQ ID NO: 1) or OVA₂₅₇₋₂₆₄ peptide (SIINFEKL; SEQ ID NO: 7) is diluted in PBS and emulsified 1:1 with either complete Freunds adjuvant (CFA) or incomplete Freunds adjuvant (IFA) using a double syringes system, to reach a final volume of 1 ml of peptide/adjuvant emulsion. 200 ul of the peptide/adjuvant emulsion is then injected subcutaneously in C57Bl/6 mice under isoflurane anaesthesia in 4 different locations (behind each shoulder and hip).

### Proliferation and Cytokine Production Assays

To measure T cell proliferation, on half of the incubated cells, 1 μCi/well [³H]thymidine (Amersham) is added to assay cultures at 48 h after contact with the peptide-pulsed APC. Following incubation for 12-16 h, cultures are harvested onto Unifiter Plates (Packard Instrument). Microscint 20 scintillation fluid (Packard Instrument) is added to each well, and plates are counted on a Scintillation Counter.

For transcription factor activity analysis and cytokines production assay, the other half of the cells is harvested 5 days after contact with the peptide-pulsed APC, centrifuged for 5 min at 300 g, and both cell pellets and supernatants are collected. Cell pellets are processed for RNA extraction for transcriptomics analysis via qPCR, cytokines production is measured by ELISA from the supernatant using the Mouse TH1/TH2 9-Plex Tissue Culture Kit from Meso Scale Diagnostics (MSD).

### Quantification of Cytotoxicity Activity by Flow Cytometry

### Preparation of Target Cells:

Targets cells (autologous B cells) are isolated from splenocytes of OT-1 or P14 mice by using the Mouse B cell s Kit (Milteniy Biotec) according to the manufacturer's instruction and are stimulated for 18 h in the presence of 40 ng/mL of IFN-γ (Peprotech). Targets cells are then counted, split into two tubes and washed in warm PBS once. Half of the target cells are stained with a high concentration (0.2 μM) of CFSE (CFSE_{High}) (Invitrogen/Thermofisher) and the other half with a low concentration (0.02 μM) of CFSE (CFSE_{Low}) in 1 mL of warm PBS for 15 min at 37 C. After the incubation, cells are pelleted and resuspended in 1 mL of warm complete RPMI 1640 (4 mM L-glutamine, and 100 U/mL penicillin and streptomycin, supplemented with 10% FBS) to quench the labeling reaction. Target cells stained with the low concentration of CFSE are pulsed by adding the peptide of interest (gp33 or OVA) at a final concentration of 5 g/mL in the complete RPMI and incubated for 45 min in 5% CO₂ atmosphere at 37 C. Both target cells stained with different concentration of CFSE are then washed twice in complete RPMI, resuspended at a concentration of 2×10¹ cells/mL each in complete RPMI and mixed with a ratio of 1:1 (CFSE_{High}: CFSE_{Low}).

### Effector Cells

Total CD8 T cells containing the effectors cells are enriched from splenocytes of P14 and OT-1 mice using the Negative Selection Human CD8 T cell isolation Kit (Milteniy Biotec) according to the manufacturer's instruction. CD8 T cells are counted, resuspended in 450 μl in complete T cell medium and serial diluted volume:volume in 225 μl of complete T cell medium from 1:2 to 1:36. From each dilution, 100 μl are seeded in duplicate to round-bottom 96-well plate. The mixed target cells (100 μl) are added to each dilution of effector cells for a final volume of 200 μL. To measure the basal apoptosis, 4 wells are seeded with targets cells alone. Cell mixtures were incubated for 6 h in 5% CO₂ atmosphere at 37 C.

### Flow Cytometry Staining and Acquisition

Cells are transferred to a V-bottom 96 well plate, washed in FACS Stain Buffer (BD Bioscience) and stained with iTag Tetramer/APC-H-2Kb OVA® (MBL), iTag Tetramer/APC-H-2Db LCMV®Alexa Fluor® and Live/Dead® Fixable Near-IR stain (ThermoFisher, diluted at 1:10000 in FACS Stain Buffer) and for 15 min in 5% CO₂ atmosphere at 37 C in the dark. Cells are then washed in FACS Stain Buffer before staining with BV421 αCD8a (Biolegend) for 30 min at +4 C in the dark. Cells are washed once with FACS Stain Buffer and resuspended with 2% paraformaldehyde (PFA; Sigma). Acquisition is performed on BD Biosciences LSR II analyzer and all cells are acquired. Post-acquisition data analysis is done with the Flowjo software (version 10.2; TreeStar Inc.).

### Example 4: Specific Targeting of Jurkat-Derived T Cells Expressing a CMV Pp65-Restricted T Cell Receptors (TCR) with Lentiviral Particles Displaying HLA-A2-Pp65 on its Surface

Human cytomegalovirus (CMV) is a virus of the Herpesviruses family that can infect humans especially through salivary glands, and trigger serious and life-threatening diseases in immunocompromised individuals, such as HIV-infected persons, patients with cancer, organ transplant recipients or even newborn infants. CMV-related diseases in immunocompromised individuals include CMV-related hepatitis, retinitis, colitis, pneumonitis, esophagitis, polyradiculopathy, transverse myelitis and subacute encephalitis. The lower matrix protein 65 (pp65) of the CMV tegument is abundantly expressed in the cytoplasm and in the nucleus of infected cells and is broadly accepted as the immuno-dominant target of CD8+ T cells in the cell response against CMV infection. One of the most characterized pp65 epitope is the pp65₄₉₅₋₅₀₃ peptide (NLVPMVATV, SEQ ID NO: 43) presented by the MHC class I molecule HLA-A 0201.

This example describes the generation of lentiviral particles pseudotyped with MHC class I molecules displaying the pp65 peptide to target T cells expressing peptide-specific TCRs, which can be used, for example, to selectively upregulate a function of these T cells (e.g., vaccine).

In particular, this example demonstrates the selective targeting by lentiviral particles displaying HLA-A2 (MHC class I molecule) presenting pp65 peptide of the T cells expressing the peptide-specific TCR.

First, a lentiviral particle comprising the human pp65-restricted TCR as a nucleotide of interest was generated and used to transduce Jurkat-derived J.RT3-T3.5/AP1-Luc/hCD28/hCD8αβcells to create a stable immortalized T cell-derived cell line expressing the pp65 TCR (J.RT3-T3.5/AP1-Luc/hCD28/hCD8αβ/pp65 TCR cell line). The pp65-restricted TCR expression cassette (CMVa18-P2A-Vb13-aa, SEQ ID NO: 45) has been synthesized by Integrated DNA Technologies as the association of the pp65 TCRα and TCRβ subunits linked by a P2A self-cleaving peptide as previously described (Stauss H. & Xue S-A, UCL BioMedica PLC, patent WO 2011/039507 A1). Other methods to generate the J.RT3-T3.5/AP1-Luc/mCD28/mCD8αβ/pp65 TCR cell line include nucleofection, electroporation, and transfection of linearized plasmid (using lipofectamine, calcium chloride, calcium phosphate or PEI).

Another lentiviral particle was then generated comprising at its surface (i) HLA-A2 (MHC class I molecule) displaying the immunogenic pp65 peptide to target the pp65-restricted TCR and (ii) fusogen SINmu derived from the mutated Sindbis virus envelope glycoprotein as described in Example 1. This particle was also generated to carry an EGFP reporter gene as a nucleotide of interest. FIG. 11 outlines the general structure of the plasmids used for lentiviral particle production as well as a schematic of the lentiviral particle displaying HLA-A2/pp65 on its surface.

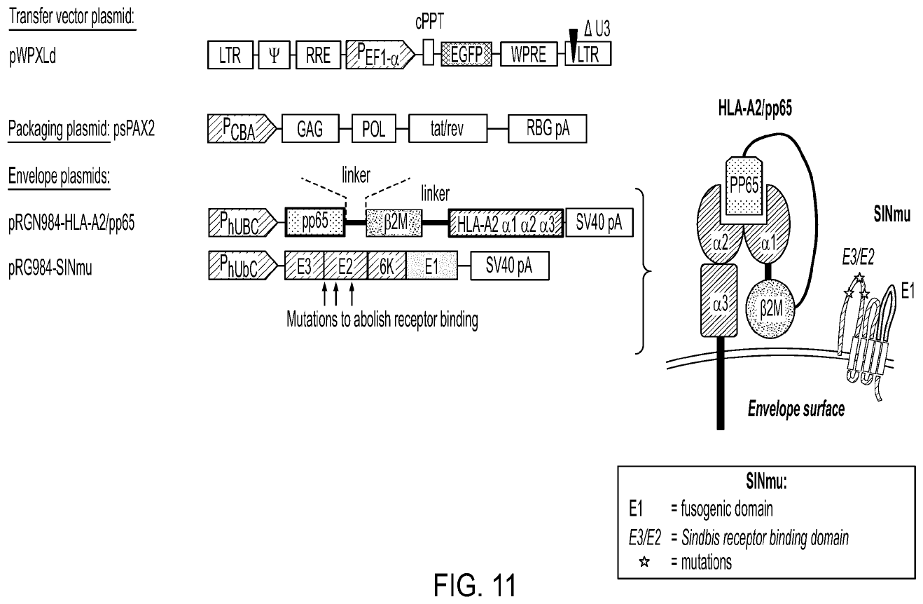

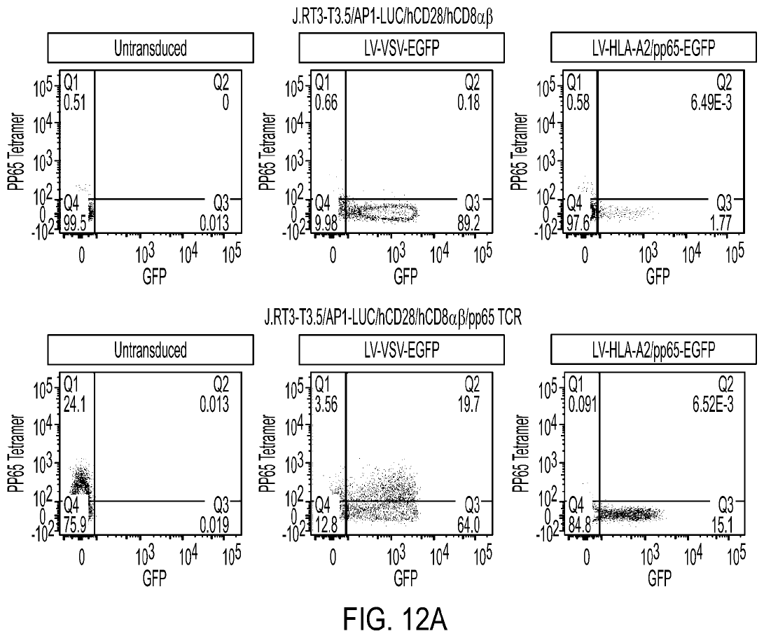

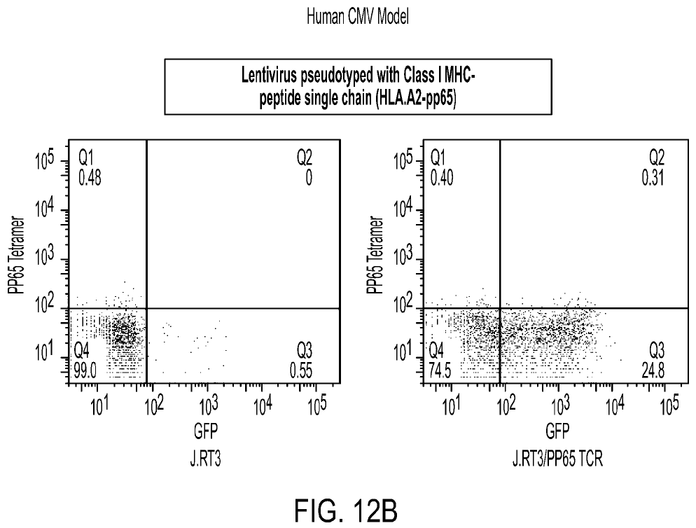

The pp65-restricted TCR-expressing cells J.RT3-T3.5/AP1-luc/hCD28/hCD8αβ were successfully transduced with the HLA-A2.5/pp65-pseudotyped lentiviral particles (LV-HLA-A2/pp65-EGFP) resulting in the expression of EGFP, while control cells not comprising the TCR (J.RT3-T3.5/AP1-luc/hCD28/hCD8a) did not demonstrate EGFP expression (FIG. 12A and FIG. 12B). Other controls included transduction without the lentiviral particles or with pan-tropic lentiviral particles displaying the glycoprotein of the Vesicular Stomatitis Virus (VSV), or the fusogen SINmu alone. These experiments demonstrate that lentiviral particles pseudotyped with a human MHC class I/antigenic peptide complex can specifically target antigen-restricted TCR expressing cells.

For in vitro functional studies, the capacity of lentiviral particles pseudotyped with HLA-A2/pp65 and carrying the human transcription factor TBX21 (SEQ ID NO: 14) as a nucleotide of interest to trigger an enhanced cytotoxic-like phenotype in Jurkat-derived T cells displaying the pp65-restricted TCR is tested.

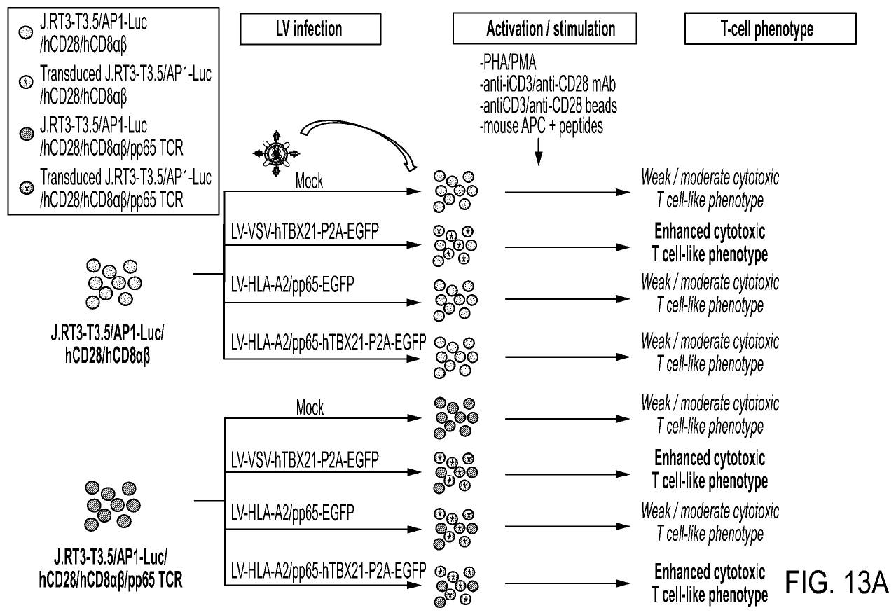

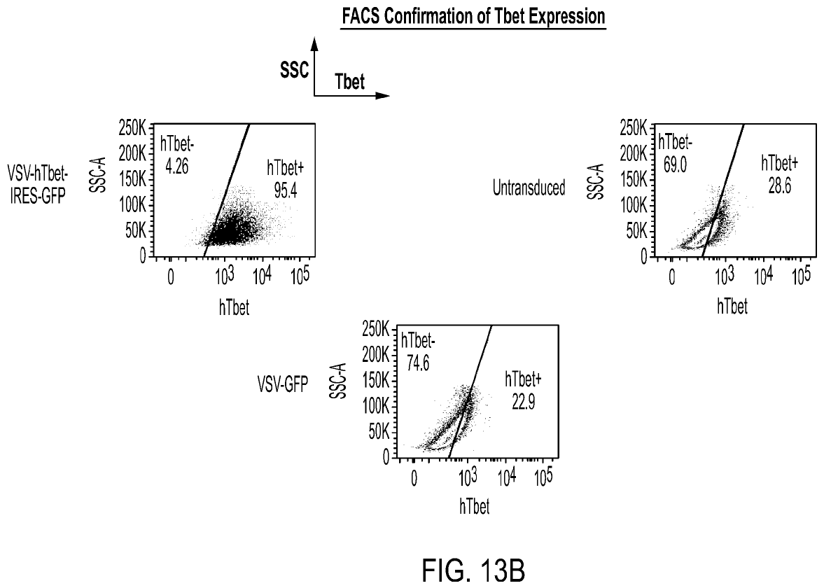

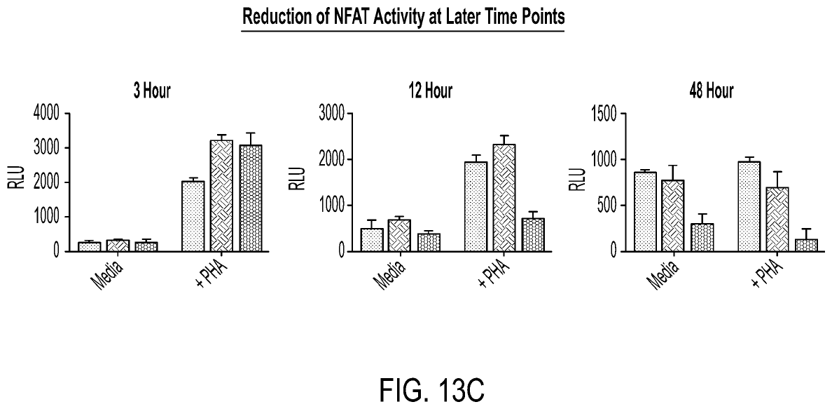

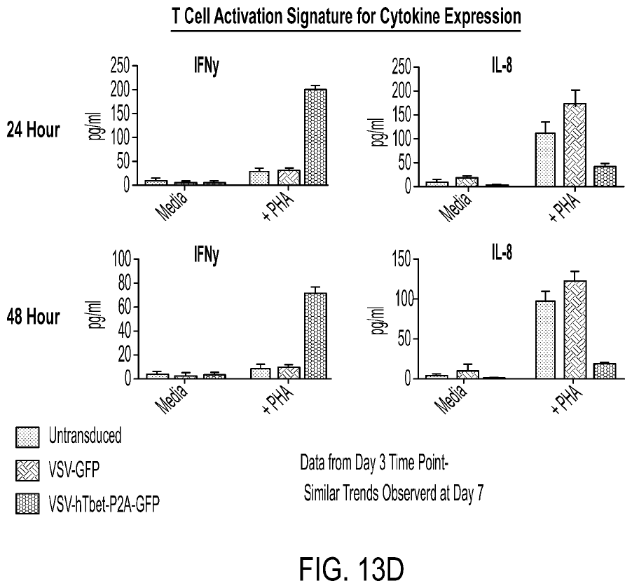

In this model, parental J.RT3-T3.5/AP1-Luc/hCD8ab/hCD28 cells and engineered J.RT3-T3.5/AP1-Luc/hCD28/hCD8αβ/pp65 cells are transduced either with the LV-HLA-A2/pp65-TBX21-IRES-EGFP or LV-HiLA-A2/pp65-EGFP as control (FIG. 13A). Additional control groups may include untransduced cells (Mock) and pan-tropic lentiviral particles (LV-VSV-hTBX21-P2A-EGFP) displaying the VSV-g envelope glycoprotein. After lentiviral transduction, J.RT3-T3.5/AP1-Luc/hCD28/hCD8αβ and J.RT3-T3.5/AP1-Luc/hCD28/hCD8αβ/pp65 cells are then stimulated with any of the following methods: (i) treatment with phytohemagglutinin (PHA), (ii) treatment with phorbol 12-myristate 13-acetate (PMA), (iii) treatment with both PHA and PMA, (iv) soluble or immobilized anti-CD3/anti-CD28 monoclonal antibodies, (v) activation beads coupled with anti-CD3/anti-CD8 beads, (vi) antigen-presenting cells (APC) previously pulsed with gp33 and OVA peptides or (vii) any other suitable method to activate T cells. Phenotype modifications following transduction is evaluated by measuring (i) activity of transcription factors involved in cytokines production (e.g., via measurement of luciferase expression driven by AP-1 promoter, activity of other transcription factors such as NF-κB or NFAT involved in cytokine production during T cell activation, etc.), (ii) cytokine (e.g., IL-2, IFNγ, etc.) production, (iii) cell proliferation, (iv) production of well-described cytotoxic enzymes or proteins such as Granzyme B or perforin, and/or (v) other well-known T-cell characteristics.

In a similar set of experiment (FIGS. 13B-13D), Jurkat/NFAT-Luc cells were transduced with the pan-tropic lentiviral particles LV-VSV-hTBX21-P2A-EGFP. The results show that ectopic expression of T-bet by the pan-tropic lentiviral particles LV-VSV-hTBX21 induces a decrease in NFAT activity but an increase in IFNγ in Jurkat/NFAT-Luc cells.

### Material and Methods

### Plasmids for Lentiviral Vector Production:

The production of lentiviral particles comprising the pp65 peptide in the groove of the human MHC class I (HLA-A2) molecule on its envelope surface and comprising either a reporter protein (EGFP), or a regulating transcription factor (TBX21, or any other T cell transcription factor) as the nucleotide of interest was achieved by co-transfection with the following plasmids:
1. A transfer vector plasmid among the followings:
   pWPXLd (Addgene #12258) that encodes the EGFP reporter protein under control of the ubiquitous EF1α promoter. An alternative plasmid also used was pLVX-EF1α-EGFP-IRES-Puro (SEQ ID NO: 11). pLVX-EF1α-EGFP was generated by inserting the EGFP sequence into the commercial pLVX-EF1α-IRES-Puro plasmid (Clontech/Takara). Alternative reporters include, but are not limited to, Thy 1.1, LNGFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the 3-globin intron sequence to enhance transcription activity.
   pLVX-EF1α-hTBX21-P2A-EGFP (SEQ ID NO: 47) that comprises a codon-optimized cDNA encoding the canonical isoform of the human transcription factor TBX21 (sequences synthesized by IDT, SEQ ID NO: 46) under control of the ubiquitous EF1α promoter and an EGFP reporter linked to human FOXP3 via a self-cleaving P2A peptide. pLVX-EF1α-hTBX21-P2A-EGFP has been generated by inserting the human TBX21 cDNA sequence into the pLVX-EF1α-GSG-P2A-EGFP (SEQ ID NO: 14) backbone plasmid, itself derived from the commercial pLVX-EF1α-IRES-Puro plasmid (Clonetech/Takara). Alternative transfer vector plasmid contains only human TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse or human TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-hTBX21-IRES-EGFP (SEQ ID NO: 48) is generated by inserting the sequences encoding human FOXP3 into the pLVX-EF1α-IRES-EGFP plasmid (SEQ ID NO: 16), itself derived from the commercial pLVX-EF1α-IRES-Puro plasmid (Clonetech/Takara) by replacement of the puromycin resistant sequence with the EGFP sequence. Alternative non-limiting example of other transcription factors include human EOMES and human ZEB2 (SEQ ID NOS: 49 to 50).
   Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contains mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. psPAX2 (Addgene plasmid #12260) was used as the packaging plasmid. PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2nd generation packaging plasmids, 3rd generation packaging plasmids, such as pRSV-Rev (Addgene plasmid #12253) and pMDLg-pRRE (Addgene plasmid #12251), Dull et al., J. Virol, 72(11):8463-8471 (1998)) or 4th generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pRG984-HLA-A2/pp65 (SEQ ID NO: 51) was used as the first envelope plasmid. The pp65-b2m-HLA-A2 single chain nucleotidic sequence was subcloned into expression plasmid pRG984 (SEQ ID NO: 16) under the control of the human Ubiquitin C (hUbC) promoter and the β-globin intron.
4. pRG984-SINmu (SEQ ID NO: 26) was used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINDBISmut was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of control pantropic lentiviral vector displaying the VSV-G envelope glycoprotein on its surface and expressing comprising a nucleotide of interest encoding either a reporter protein (EGFP), or the transcription factor TBX21 (or any other T cell transcription factors including, but not limited to EOMES and ZEB2) as the nucleotide of interest was achieved by co-transfection with the following plasmids:
1. Transfer plasmids comprising a nucleotide sequence of interest:
   pWPXLd (Addgene #12258) that encodes the EGFP reporter protein under control of the ubiquitous EF1α promoter. An alternative plasmid also used was pLVX-EF1α-EGFP-IRES-Puro (SEQ ID NO: 11). pLVX-EF1α-EGFP was generated by inserting the EGFP sequence into the commercial pLVX-EF1α-IRES-Puro plasmid (Clontech/Takara). Alternative reporters include, but are not limited to Thy1.1, LNGFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-hTBX21-P2A-EGFP (SEQ ID NO: 47) that comprises a codon-optimized cDNA encoding the canonical isoform of the human transcription factor TBX21 (sequences synthesized by IDT, SEQ ID NO: 46) under control of the ubiquitous EF1α promoter and an EGFP reporter linked to human FOXP3 via a self-cleaving P2A peptide. pLVX-EF1α-hTBX21-P2A-EGFP has been generated by inserting the human TBX21 cDNA sequence into the pLVX-EF1α-GSG-P2A-EGFP (SEQ ID NO: 14) backbone plasmid, itself derived from the commercial pLVX-EF1α-IRES-Puro plasmid (Clonetech/Takara). Alternative transfer vector plasmid contains only human TBX21 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse or human TBX21 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-hTBX21-IRES-EGFP (SEQ ID NO: 48) is generated by inserting the sequences encoding human FOXP3 into the pLVX-EF1α-IRES-EGFP plasmid (SEQ ID NO: 16), itself derived from the commercial pLVX-EF1α-IRES-Puro plasmid (Clonetech/Takara) by replacement of the puromycin resistant sequence with the EGFP sequence. Alternative non-limiting example of other transcription factors include human EOMES and human ZEB2 (SEQ ID NOS: 49 to 50).
Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contains mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. psPAX2 was used as the packaging plasmid (Addgene plasmid #12260). PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2^{nd} generation packaging plasmids, 3^{rd} generation packaging plasmids, such as pRSV-Rev (Addgene plasmid #12253) and pMDLg-pRRE (Addgene plasmid #12251), Dull et al., J Virol, 72(11):8463-8471 (1998)) or 4^{th} generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pMD2.G (Addgene plasmid #12259) was used as the envelope plasmid displaying the glycoprotein of the Vesicular Stomatitis Virus (VSV).

The production of lentiviral particles LV-VSV-pp65 TCR comprising the pp65-restricted TCR as the nucleotide of interest was achieved by co-transfection of the following plasmids:
1. As transfer vector plasmid: pLVX-EF1α-CMVa18-P2A-Vb13-aa-IRES-hygro (SEQ ID NO: 52) for generation of the LV-VSV-pp65 TCR lentiviral particles.
2. psPAX2 (Addgene plasmid #12260) as the packaging plasmid described above.
3. pMD2.G (Addgene plasmid #12259) as the envelope plasmid comprising the nucleic acid sequence encoding the glycoprotein of the Vesicular Stomatitis Virus (VSV).

### Cell Lines

Adherent HEK 293T/17 cells (ATCC) were cultivated in DMEM medium (Gibco/Life Technologies) supplemented with 10% fetal bovine serum and 1% penicillin-streptomycin.

The suspension cell line J.RT3-T3.5/AP1-luc/hCD28/hCD8αβ (an engineered cell line derived from the J.RT3-T3.5, itself derived from the human lymphoma Jurkat cell line), which does not express any TCRs nor CD3 on its surface, was used to express the pp65-restricted TCR via lentiviral transduction with the LV-VSV-pp65 TCR lentiviral particles. J.RT3-T3.5/AP1-luc/hCD28/hCD8αβ and J.RT3-T3.5/AP1-luc/hCD28/hCD8αβ/pp65 TCR were cultured in RPMI 1640 (Gibco/Life Technologies) supplemented with 10% Fetal Bovine Serum and 1% Penicillin/Streptomycin mix.

### Lentiviral Particle Production

Lentiviral particles were produced following standard lipofectamine-mediated co-transfection of HEK 293T cells with the respective transfer vector, packaging and envelope plasmids. The day before transfection, cells were washed with phosphate buffered saline solution (PBS) once then detached from vessel with TrypLE™ Express (Life Technologies). After neutralization of TrypLE Express with cell medium containing FBS, cells were centrifuged at 1200 rpm for 5 min at 25 C, then resuspended in complete DMEM medium, counted and seeded in 150 mm cell culture dishes at a density of 10×10⁶ cells/plate. On the day of transfection, the cell culture medium was replaced by fresh Opti-MEM medium (Gibco/Life Technologies) supplemented with 25 nM chloroquine (Sigma-Aldrich). The DNA mix was prepared by mixing 60 μg of total DNA 1.5 mL of Opti-MEM with 60 ul of PLUS™ Reagent (Life Technologies). The specific plasmid ratios for the different lentivirus productions are illustrated in TABLE 3. In parallel 100 μl of Lipofectamine® TLX (Life Technologies) was diluted in 1.5 mL of OptiMEM medium. DNA mix was then added to the lipofectamine mix and the new combined solution was incubated at room temperature for 20 minutes before being added directly to the cells dropwise. The culture medium was changed 6 to 8 h after transfection and the cells were then incubated for 48 h at 37 C in an incubator with 5% CO₂ atmosphere. At day 2 post-transfection, cell media containing the lentiviral particles were centrifuged for 10 min at 3000 rpm to remove the debris, then passed through a 0.45 um pore size filter. The filtered supernatants were then treated with 1 μg/ml DNAse and 1 mM MgCl2 for 15 minutes at 37° C. to remove residual DNA. For concentrating the lentiviral vectors batch, the supernatants were then ultracentrifuged at 27,100 rpm for 90 min. After ultracentrifugation, pellets were resuspended in a suitable volume of PBS (50 to 100 μl) overnight. The resuspended lentiviruses were finally processed through a series of short centrifugations (30 sec at 13500 rpm) to clarify the viral solution of remaining debris. The batches of lentiviral particles were titrated by RT-qPCR using a SYBR® technology-based kit from Clontech/Takara then stocked at −80° C. until use for transduction.

Alternative transfections reagents include polyethylenimine (PEI), calcium chloride or calcium phosphate.

### Cell Lines Transduction

On day of transduction, J.RT3-T3.5/AP1-luc/hCD28/hCD8αβ and J.RT3-T3.5/AP1-luc/hCD28/hCD8αβ/pp65 TCR cells were centrifuged for 5 minutes at 1200 RPM and 25° C. After resuspension in fresh complete RPMI medium, cells were counted, seeded in 24-well plates at a density of 100,000 cells/well and mixed with 4 g/ml polybrene and the suitable amount of lentivirus (standard dose of 25,000 viral copies per cell). Cells were then spinoculated by centrifugating them in presence of the virus at 2000 rpm for 90 min at 30° C. After centrifugation, cells were incubated at 37° C. and 5% C02 for 3 days before fixation and staining/FACS analysis.

### Cells Staining and FACS Analysis

Fluorescence-activated cell sorting (FACS) was performed on day 3 after the transduction. Transduced cells were counted and seeded equally into a 96-well V bottom plate. Cells were spun at 2000 rpm at 4 C for 2 min, washed with PBS then spun again. Cells were incubated with Live/Dead® Fixable Near-IR stain (Life Technologies, diluted at 1:10000 in PBS) for 15 min in the dark, washed, and incubated with Fc block (eBioscience, dilution 1:150) in Facs Stain Buffer (BD Biosciences). After being washed twice again with FACS stain buffer, cells were subsequently incubated for 30 min, on ice and in the dark with either of the APC-conjugated iTAg Tetramer®/APC—HLA-A*02:01 CMV pp65 (MBL International). Cells were washed one more time with FACS stain buffer, fixed with paraformaldehyde 1%, washed again and finally resuspended in FACS stain buffer. Samples were run for analysis with a BD FACS Canto II analyzer (BD Biosciences).

### Splenocytes and Antigen Presenting Cell Primary Cultures

Spleens of adult C57Bl/6 mouse was first excised and placed in cold PBS (without calcium or magnesium) supplemented with 2% FBS. Tissues were then homogenized using GentleMACS™ Octo Dissociator (Milteniy) to break apart the spleens in GentleMACS™ C Tubes (Milteniy). Dissociated cells were then centrifuged at 500 g, 4 C for 5 min. After centrifugation the cell pellet was resuspended with gentle vortexing in a suitable volume of ACK lysis buffer (5 ml per spleen, Gibco/Life Technologies) to remove the red blood cells, and incubated for 5 min at room temperature. After incubation the cell suspension was added to 10 ml/spleen of PBS/FBS, and centrifuged at 500 g, 4 C for 5 min. The cell pellet was then resuspended in PBS/FBS and the cell solution was filtered through a 0.7 μM filter (BD Biosciences). The filtered cell suspension was centrifuged again and the subsequent pellet of individualized cells was resuspended one more time in a suitable volume of PBS/FBS. Total spleen cells were counted, and global antigen-presenting cells (APC) were sorted using anti-MHC class II Microbeads and MACS® sorting technology (Miltenyi Biotec). After elution, the MHC class II-positive cell fraction was collected and resuspended at 1×10⁶ cells/ml in APC cell medium composed of RPMI 1640 (Gibco/ThermoFisher)+1% Horse serum (Gibco/ThermoFisher)+100 U/ml penicillin/streptomycin (Gibco/ThermoFisher)+2 Mm L-Glutamin (Gibco/Life technologies) supplemented with 30 ng/ml of IL-4 (Peprotech), 50 ng/ml of GM-CSF (Peprotech) and 1 ng/ml of recombinant mouse TNFα (Peprotech).

Pulsing of APCs with Peptides

APC were then incubated with 10 μg/ml of pp65 peptide, or no peptide as control, overnight. The day after, peptide-pulsed APC were harvested and washed before addition at a ratio 1:1 to the different J.RT3-T3.5 derived cell lines, 48 h after infection with the lentiviral particles. J.RT3-T3.5-derived cell lines were then cocultured with the pulsed APC in J.RT3-T3.5 cell culture medium in duplicate with one half for proliferation assay and one half for luciferase assay+ cytokines production assay.

### Jurkat T Cells Activation Post Transduction

Alternatively, to coculture with peptides-pulsed APCs, J.RT3-T3.5 derived cell lines are activated 48 h after transduction with either phytohemagglutinin (PHA, Sigma, at 5 μg/ml), phorbol 12-myristate 13-acetate (PMA, Sigma, at 2 ug/ml), or a combination of PHA and PMA (5 ug/ml and 2 μg/ml respectively).

Another alternative method to activate J.RT3-T3.5 derived cell lines 48 h after transduction is to use soluble or immobilized anti-CD3 and anti-CD28 monoclonal antibodies (BD Biosciences). Soluble antibodies are added at a concentration of 1 ug/ml. In some experiments plates pre-coated with anti-CD3 antibodies (BD Biosciences) are used in combination with soluble anti-CD28 antibody (BD Biosciences, 1 ug/ml).

Another alternative method to activate J.RT3-T3.5 derived cell lines activating beads coupled with anti-CD3/antiCD28 antibodies (Dynabeads, ThermoFisher). 48 h after transduction, infected J.RT3-T3.5 derived cells are counted, and antiCD3/antiCD28 beads are added to the culture medium at ratio 1:1.

### Proliferation and Cytokine Production Assays

To measure T cell proliferation, on half of the incubated cells, 1 μCi/well [³H]thymidine (Amersham) is added to assay cultures at 48 h after contact with the peptide-pulsed APC. Following incubation for 12-16 h, cultures are harvested onto Unifiter Plates (Packard Instrument). Microscint 20 scintillation fluid (Packard Instrument) is added to each well, and plates are counted on a Scintillation Counter.

For transcription factor activity analysis and cytokines production assay, the other half of the cells is harvested 5 days after contact with the peptide-pulsed APC, centrifuged for 5 min at 300 g, and both cell pellets and supernatants are collected. Cell pellets are processed for luciferase detection assay to measure AP1 activity, and cytokines production is measured by ELISA from the supernatant using the Human TH1/TH2 10-Plex Tissue Culture Kit from Meso Scale Diagnostics (MSD).

### Example 5: Specific Targeting of Human Primary CD4+ T Cells Expressing an Antigen-Restricted T Cell Receptor (TCR) with Lentiviral Particles Displaying Antigenic Peptides in MHC Class II Groove on their Surface

Celiac disease is an autoimmune disorder caused by a reaction to gluten. Most patients with celiac disease carry one of two types of the HLA-DQ protein that belongs to MHC class II (95% carry the isoform DQ2 or DQ8), which bind tightly to gliadin fragments deriving from the gliadin protein which is a component of gluten (GenBank #K03076.1). TCRs for at least two specific peptides with immunogenic epitopes, HLA-DQ2.5-restricted α1-gliadin (QLQPFPQPELPY; SEQ ID NOs: 53 and 54) and HLA-DQ2.5-restricted α2-gliadin (PQPELPYPQPQL; SEQ ID NOs: 55 and 56) are known (Petersen et al., Nature Structural and Molecular Biology, 21(5): 480-488 (2014)).

This example describes generation of lentiviral particles pseudotyped with MHC class II molecules displaying gliadin peptides to target T cells expressing peptide-specific TCRs, which can be used, for example, to selectively downregulate a function of these T cells (e.g., in the context of autoimmunity).

In particular, this example demonstrates the selective targeting by lentiviral particles displaying HLA-DQ2.5 (MHC class II molecule) presenting α1-gliadin-derived peptide of the T cells expressing the peptide-specific TCR.

First, lentiviral transfer vector plasmids encoding respectively the α1-gliadin-restricted TCRα (pLVX-EF1α-380-E48-α1-gliadin-TCRα-IRES-Neo, SEQ ID NO: 57) and the α1-gliadin-restricted TCRβ (pLVX-EF1α-380-E48-α1-gliadin-TCRβ-IRES-hygro, SEQ ID NO: 58) were packaged into lentiviral particles in order to transduce J.RT3-T3.5/AP1-luc/hCD28 cell line and create a stable J.RT3-T3.5-derived cell line expressing the α1-gliadin TCR. Similarly, lentiviral transfer vector plasmids encoding respectively the α2-gliadin-restricted TCRα (pLVX-EF1α-737-60-α2-gliadin-TCRα-IRES-neo, SEQ ID NO: 59) or the α2-gliadin-restricted TCRβ (pLVX-EF1α-737-60 α2 gliadin-TCRβ-IRES-hygro, SEQ ID NO: 60) were generated and used to respectively transduce J.RT3-T3.5/AP1-Luc/hCD28 cell line in order to create a stable J.RT3-T3.5-derived cell line expressing the α2-gliadin TCR.

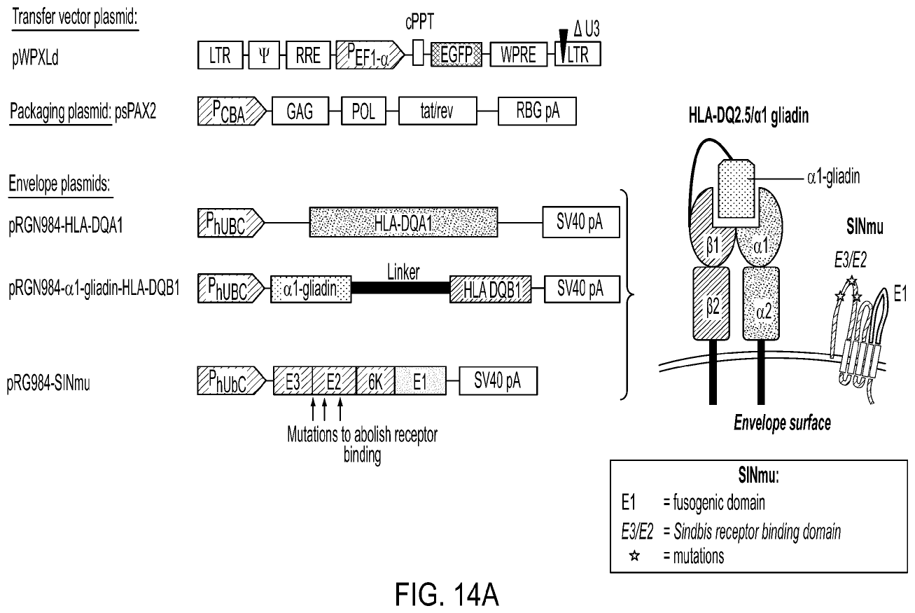

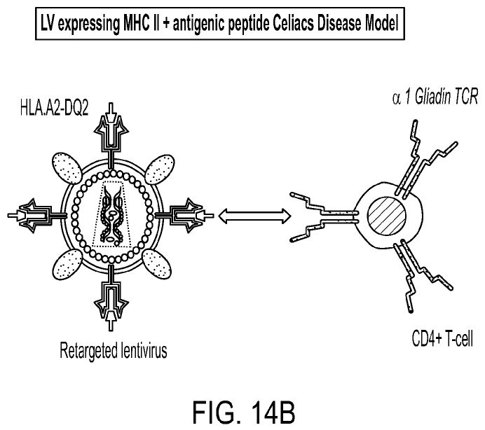

Another lentiviral particle was then generated comprising at its surface (i) HLA-DQ2.5 (MHC class II molecule) displaying the immunogenic α1-gliadin peptide to target the α1-gliadin restricted TCR and (ii) fusogen SINmu derived from the mutated Sindbis virus envelope glycoprotein as describe in Example 1. This particle was also generated to carry an EGFP reporter gene as a nucleotide of interest. FIG. 14A outlines the general structure of the plasmids used for lentiviral particle production as well as a schematic of the lentiviral particle displaying HLA-DQ2.5/α1-gliadin on its surface. FIG. 14B depicts the interaction between the lentiviral particle pseudotyped with HLA-DQ2.5/α1-gliadin and a CD4+ T cell expressing a α1-gliadin TCR.

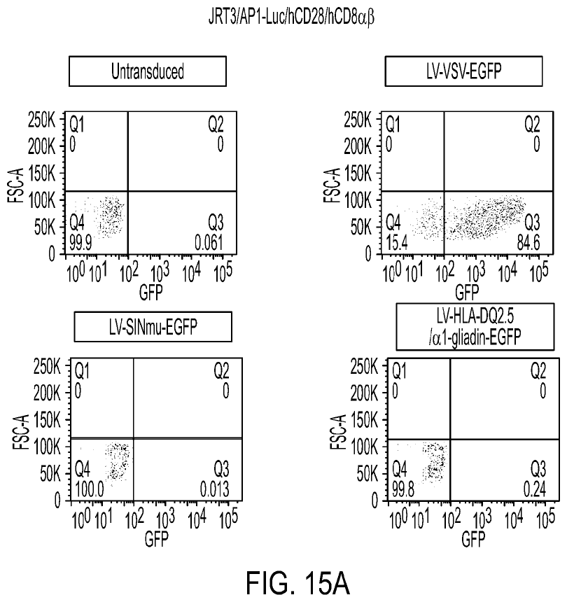

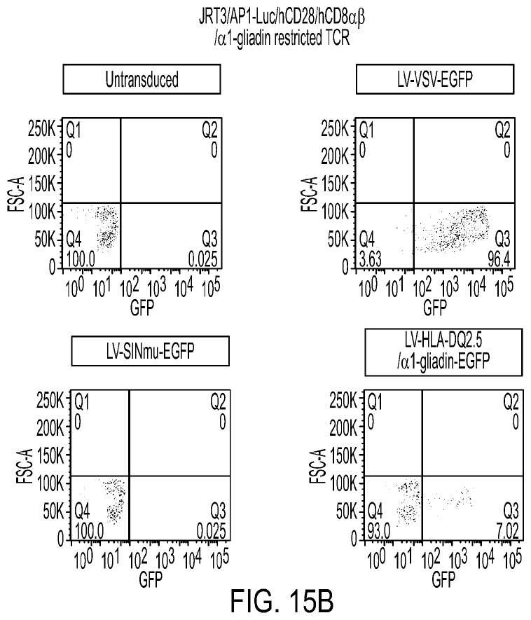

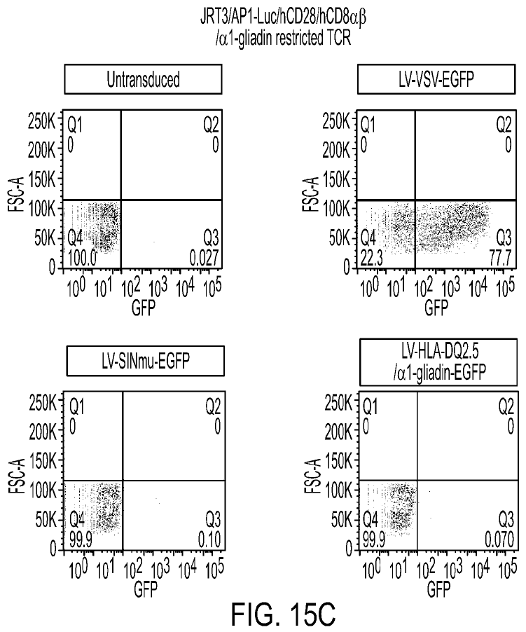

The α1-gliadin-restricted TCR-expressing cells J.RT3-T3.5/AP1-luc/hCD28/380-E48ab-α1-gliadin were successfully transduced with the HLA-DQ2.5/α1-gliadin pseudotyped lentiviral particles (LV-HLA-DQ2.5/α1-gliadin-EGFP) resulting in the expression of EGFP, while control cells not comprising the TCR (J.RT3-T3.5/AP1-luc/hCD28) and control cells expressing α2-gliadin-restricted TCR (J.RT3-T3.5/AP1-luc/hCD28/737-60ab-α2-gliadin) did not demonstrate EGFP expression (FIGS. 15A-15C). Other controls included transduction without the lentiviral particles or with pan-tropic lentiviral particles comprising the glycoprotein of the Vesicular Stomatitis Virus (VSV) or the fusogen SINmu on its surface but no HLA-DQ2.5/α1-gliadin (LV-SINmu-EGFP). These experiments demonstrate the specificity of TCR targeting by the created MHC/peptide pseudotyped lentiviral particles.

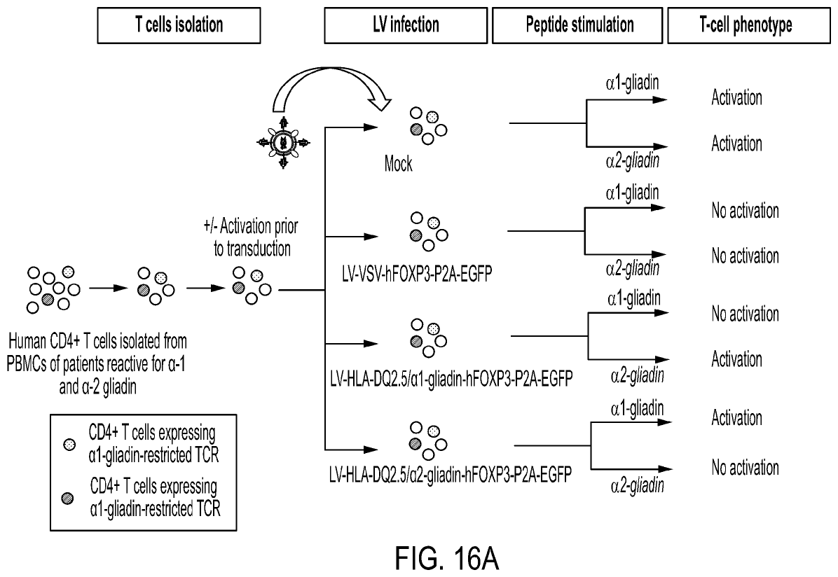

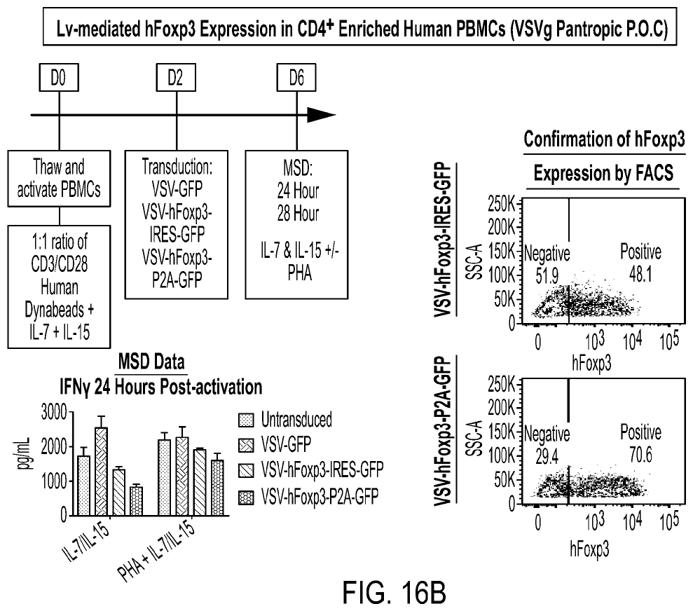

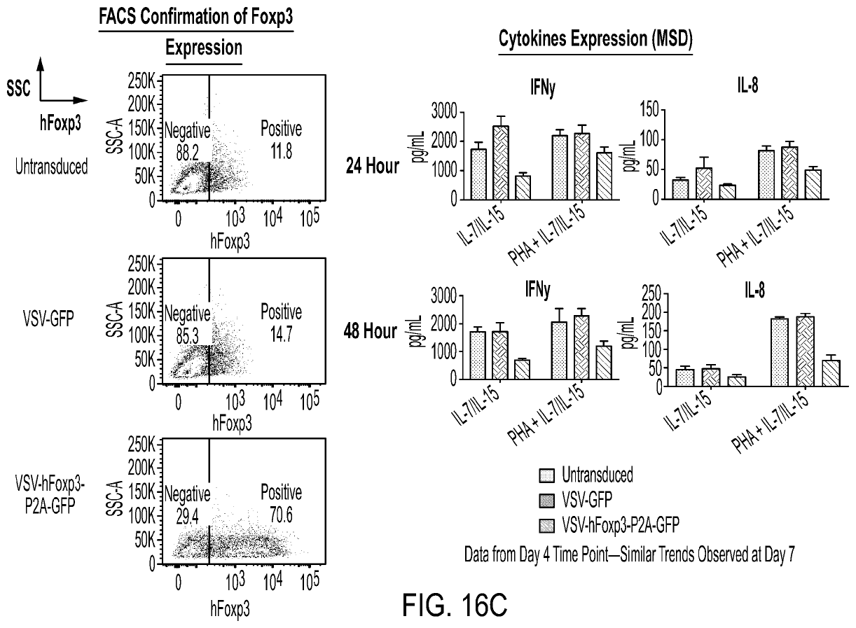

For ex vivo functional studies, the capacity of lentiviral particles pseudotyped with HLA-DQ2.5/α1-gliadin and comprising the human transcription factor FOXP3 as a nucleotide of interest to repress the activation of human CD4+ T cells into Th1 cells after peptide stimulation by inducing a FOXP3-mediated Treg phenotype is evaluated. In this model, CD4+ T cells are isolated from peripheral blood mononuclear cells (PBMC) of patients reactive for α1- and α2-gliadin, and then transduced either with the LV-HLA-DQ2.5/α1-gliadin-hFOXP3-P2A-GFP or LV-HLA-DQ2.5/α2-gliadin-hFOXP3-P2A-GFP lentiviral particles (FIG. 16A). Additional control groups may include untransduced cells (Mock) and pan-tropic lentiviral particles (LV-VSV-hFOXP3-P2A-GFP). Transduced T cells are then stimulated by coculture with either α1-gliadin or α2-gliadin peptide-pulsed APC, also derived from human PBMCs. Phenotype modifications following transduction is evaluated by measuring (i) activity of transcription factors involved in cytokines production (e.g., via measurement of luciferase expression driven by AP-1 promoter, activity of other transcription factors such as NF-κB or NFAT involved in cytokine production during T cell activation, etc.), (ii) cytokine (e.g., IL-2, IFNγ, etc.) production, (iii) cell proliferation, (iv) suppressive activity on CD4+CD25+T responder cells (Tres) proliferation in cocultures models as described in Kim et al., Biochemical and Biophysical Research Communications, 362:44-50 (2007), and/or (v) other well-known T-cell characteristics.

In a similar set of experiments (FIG. 16B), CD4+ enriched human PBMCs were thawed, activated, and treated with beads coupled with anti-CD3/anti-CD8 monoclonal antibodies in the presence of IL-7 and IL-15 on Day 0. On Day 2, cells were transduced with pan-tropic lentiviral particles VSV-GFP or VSV-hFOXP3-IRES-GFP or VSV-hFOXP3-P2A-GFP. On Day 6, cells were stimulated with IL-7, IL-15 with or without PHA. Cells were then evaluated for cytokine production at 24 hour and 48 hour. The results show that the pan-tropic lentiviral particle LV-VSV-hFOXP3 mediate hFOXP3 expression and modulates T cell phenotype in CD4+ enriched human PBMCs. It was further shown that ectopic expression of FOXP3 by the pan-tropic lentiviral particle LV-VSV-hFOXP3 partially induces a Treg-like phenotype in human CD4+ T cells (FIG. 16C).

### Material and Methods

### Plasmids for Lentiviral Vector Production:

The production of lentiviral particles comprising the α1-gliadin peptide in the groove of the MHC class II (HLA-DQ2.5) molecule on its envelope surface and comprising either a reporter protein (EGFP), or a regulating transcription factor (FOXP3 or TBX21 or any other T cell transcription factor) as the nucleotide of interest was achieved by co-transfection with the following plasmids:
1. A transfer vector plasmid among the followings:
   pWPXLd (Addgene plasmid #12258) that encodes the EGFP reporter protein under control of the ubiquitous EF1α promoter. An alternative plasmid also used was pLVX-EF1α-EGFP-IRES-Puro (SEQ ID NO: 11). pLVX-EF1α-EGFP was generated by inserting the EGFP sequence into the commercial pLVX-EF1α-IRES-Puro plasmid (Clontech/Takara). Alternative reporters include, but are not limited to, Thy1.1, LNGFR, DsRed, RFP, YFP, CFP, mCherry, TdTomato, Luciferase, and Lac-Z proteins. Alternative promoters include but are not limited to CMV, CMVmax, Ubiquitin C, PGK, CAGG, SFFV and MSCV, with or without addition of the β-globin intron sequence to enhance transcription activity.
   pLVX-EF1α-hFOXP3-P2A-EGFP (SEQ ID NO: 12) that comprises a codon-optimized cDNA encoding the canonical isoform of the human transcription factor FOXP3 (sequences synthesized by IDT, SEQ ID NO: 13) under control of the ubiquitous EF1α promoter and an EGFP reporter linked to human FOXP3 via a self-cleaving P2A peptide. pLVX-EF1α-hFOXP3-P2A-EGFP has been generated by inserting the human FOXP3 cDNA sequence into the pLVX-EF1α-GSG-P2A-EGFP (SEQ ID NO: 14) backbone plasmid, itself derived from the commercial pLVX-EF1α-IRES-Puro plasmid (Clonetech/Takara). Alternative transfer vector plasmid contains only human FOXP3 with no reporter, or a different reporter protein, including, but not limited to, Thy1.1, LNGFR, DsRed, RFP, YFP, CFP, mCherry, TdTomato, Luciferase, and Lac-Z. Alternative promoters include but are not limited to CMV, CMVmax, Ubiquitin C, PGK, CAGG, SFFV and MSCV, with or without addition of the β-globin intron sequence to enhance transcription activity. Other alternative bicistronic lentiviral vectors also contains other self-cleaving “2A” peptide (T2A, F2A or any other self-cleaving peptide) to express mouse or human FOXP3 (or any other transcription factor) and the EGFP reporter protein (or any other reporter protein) from a same mRNA (Ibrahimi et al. Hum Gene Ther. 20(8):845-60 (2009). Other alternative bicitronic lentiviral vector contains EGFP downstream an Internal Ribosome Entry Site (IRES) element that allows the independent production of both proteins under control of the same promoter. pLVX-EF1α-hFOXP3-IRES-EGFP (SEQ ID NO: 15) is generated by inserting the sequences encoding human FOXP3 into the pLVX-EF1α-IRES-EGFP plasmid (SEQ ID NO: 16), itself derived from the commercial pLVX-EF1α-IRES-Puro plasmid (Clonetech/Takara) by replacement of the puromycin resistant sequence with the EGFP sequence. Alternative non-limiting example of other transcription factors include human IKZF2 (Helios) IKZF4 (EOS), GATA1, IRF4, SATB1, LEF1 (SEQ ID NO: 17 to 22).
Each of the transfer vectors also contained the following common elements: 5′ HIV LTR, a primer binding site (PBS), an encapsidation signal ψ, the Rev Response Element (RRE), the mRNA stabilizing element WPRE, and the ΔU3 HIV 3′ LTR (modified by deletion of the U3 region in order to produce replication-deficient lentiviral particles). Alternative versions of those transfer vector plasmids contains mutations in the PBS, the integrase attachment sites (located in the 5′ and 3′ LTR) or in the 3′ LTR, as illustrated in TABLE 1. Alternative lentiviral backbones plasmids include plasmids containing chimeric LTR/RSV or LTR/CMV promoters for production of lentiviral particles packaging plasmids of third generation.
2. psPAX2 (Addgene plasmid #12260) was used as the packaging plasmid. PsPAX2 is a second generation packaging plasmid that contains the gag gene coding the different structural elements of the lentiviral capsid (i.e., matrix, capsid, and nucelocapsid), and the pol gene coding the different necessary enzymes for the viral genome integration (i.e., retrotranscriptase, protease, and integrase). Alternative packaging plasmids include other 2nd generation packaging plasmids, 3rd generation packaging plasmids, such as pRSV-Rev (Addgene plasmid #12253) and pMDLg-pRRE (Addgene plasmid #12251), Dull et al., J Virol, 72(11):8463-8471 (1998)) or 4th generation packaging system such as Lenti-X® Fourth Generation packaging system (Clontech/Takara). Other modified versions of psPAX2 presenting mutations either in the integrase or the retrotranscriptase enzymes have been used too for generating non-integrative versions of those vectors (TABLE 1).
3. pRG984-HLA-DQA1 (SEQ ID NO: 63) associated either with pRG984-α1-gliadin-HLA-DQB1 (SEQ ID NO: 64) or pRG984-α2-gliadin-HLA-DQB1 (SEQ ID NO: 65) were used as the first envelope plasmid. The HLA-DQA1 and the respective α-1 or α-2-gliadin-HLA-DQB1 cassettes were subcloned into expression plasmid pRG984 (SEQ ID NO: 16) under the control of the human Ubiquitin C (hUbC) promoter and the 3-globin intron (SEQ ID NO: 13).
4. pRG984-SINmu (SEQ ID NO: 26) was used as the second envelope plasmid. pRG984-SINmu expresses a mutated sequence of the Sindbis virus envelope glycoprotein (SINmu) which does not bind to its cognate receptor but maintains its fusogenic properties (Morizono et al., Nature Medicine, 11(30):346-352 (2005); Yang et al., PNAS 103(31):11479-11484 (2006)). pRG984-SINDBISmut was generated by subcloning the SINmu DNA sequence into the pRG984 plasmid backbone. Alternative fusogens comprise Influenza HA glycoprotein, modified VSV glycoprotein, modified Nipah G and F glycoproteins, and modified Measles H and F glycoproteins.

The production of lentiviral particles LV-VSV-α1-gliadinTCR comprising the α1-gliadin-restricted TCR as the nucleotide of interest and lentiviral particles LV-VSV-α2-gliadinTCR comprising the α2-gliadin-restricted TCR as the nucleotide of interest was achieved by co-transfection of the following plasmids:
1. As transfer vector plasmids: pLVX-EF1α-380-E48 α1 gliadin-TCRα-IRES-Neo (SEQ ID NO: 57) and pLVX-EF1α-380-E48 α1 gliadin-TCRβ-IRES-hygro (SEQ ID NO: 58) for generation of the LV-VSV-α1-gliadinTCR lentiviral particles; pLVX-EF1α-737-60 α2 gliadin-TCRα-IRES-neo (SEQ ID NO: 59) and pLVX-EF1α-737-60 α2 gliadin-TCRβ-IRES-hygro (SEQ ID NO: 60) for generation of the LV-VSV-α2-gliadinTCR lentiviral particles
2. psPAX2 (Addgene plasmid #12260) as the packaging plasmid described above.
3. pMD2.G (Addgene plasmid #12259) as the envelope plasmid comprising the nucleic acid sequence encoding the glycoprotein of the Vesicular Stomatitis Virus (VSV).

### Cell Lines

Adherent HEK 293T/17 cells (ATCC) were cultivated in DMEM medium (Gibco/Life Technologies) supplemented with 10% fetal bovine serum and 1% penicillin-streptomycin.

The suspension cell line J.RT3-T3.5/AP1-luc/hCD28 (an engineered cell line derived from the J.RT3-T3.5, itself derived from the human lymphoma Jurkat cell line), which does not express any TCRs nor CD3 on its surface, was used to express the α1-gliadin restricted TCR via lentiviral transduction with the LV-VSV-α1-gliadinTCR lentiviral particles. JRT3-T3.5/AP1-luc/hCD28 was also used to express the α2-gliadin restricted TCR via lentiviral transduction with the LV-VSV-α2-gliadinTCR lentiviral particles. JRT3-T3.5/AP1-luc/hCD28, JRT3-T3.5/AP1-luc/hCD28/α1-gliadinTCR and JRT3-T3.5/AP1-luc/hCD28/α2-gliadinTCR cells were cultured in RPMI 1640 (Gibco/Life Technologies) supplemented with 10% Fetal Bovine Serum and 1% Penicillin/Streptomycin mix.

### Lentiviral Particle Production

Lentiviral particles were produced following standard lipofectamine-mediated co-transfection of HEK 293T cells with the respective transfer vector, packaging and envelope plasmids. The day before transfection, cells were washed with phosphate buffered saline solution (PBS) once then detached from vessel with TrypLE™ Express (Life Technologies). After neutralization of TrypLE Express with cell medium containing FBS, cells were centrifuged at 1200 rpm for 5 min at 25 C, then resuspended in complete DMEM medium, counted and seeded in 150 mm cell culture dishes at a density of 10×10⁶ cells/plate. On the day of transfection, the cell culture medium was replaced by fresh Opti-MEM medium (Gibco/Life Technologies) supplemented with 25 nM chloroquine (Sigma-Aldrich). The DNA mix was prepared by mixing 60 μg of total DNA 1.5 mL of Opti-MEM with 60 ul of PLUS™ Reagent (Life Technologies). The specific plasmid ratios for the different lentivirus productions are illustrated in TABLE 3. In parallel 100 μl of Lipofectamine® TLX (Life Technologies) was diluted in 1.5 mL of OptiMEM medium. DNA mix was then added to the lipofectamine mix and the new combined solution was incubated at room temperature for 20 minutes before being added directly to the cells dropwise. The culture medium was changed 6 to 8 h after transfection and the cells were then incubated for 48 h at 37 C in an incubator with 5% CO₂ atmosphere. At day 2 post-transfection, cell media containing the lentiviral particles were centrifuged for 10 min at 3000 rpm to remove the debris, then passed through a 0.45 um pore size filter. The filtered supernatants were then treated with 1 μg/ml DNAse and 1 mM MgCl₂ for 15 minutes at 37° C. to remove residual DNA. For concentrating the lentiviral vectors batch, the supernatants were then ultracentrifuged at 27,100 rpm for 90 min. After ultracentrifugation, pellets were resuspended in a suitable volume of PBS (50 to 100 μl) overnight. The resuspended lentiviruses were finally processed through a series of short centrifugations (30 sec at 13500 rpm) to clarify the viral solution of remaining debris. The batches of lentiviral particles were titrated by RT-qPCR using a SYBR® technology-based kit from Clontech/Takara then stocked at −80° C. until use for transduction.

Alternative transfections reagents include polyethylenimine (PEI), calcium chloride or calcium phosphate.

### Cell Lines Transduction

On day of transduction, JRT3-T3.5/AP1-Luc/hCD28, JRT3-T3.5/AP1-Luc/hCD28/α1-gliadinTCR and RT3-T3.5/AP1-Luc/hCD28/α2-gliadinTCR cells were centrifuged for 5 minutes at 1200 RPM and 25° C. After resuspension in fresh complete RPMI medium, cells were counted, seeded in 24-well plates at a density of 100,000 cells/well and mixed with 4 μg/ml polybrene and the suitable amount of lentivirus (standard dose of 25,000 viral copies per cell). Cells were then spinoculated by centrifugating them in presence of the virus at 2000 rpm for 90 min at 30° C. After centrifugation, cells were incubated at 37° C. and 5% C02 for 3 days before fixation and staining/FACS analysis.

### Cells Staining and FACS Analysis

Fluorescence-activated cell sorting (FACS) was performed on day 3 after the transduction. Transduced cells were counted and seeded equally into a 96-well V bottom plate. Cells were spun at 2000 rpm at 4 C for 2 min, washed with PBS then spun again. Cells were incubated with Live/Dead® Fixable Near-IR stain (Life Technologies, diluted at 1:10000 in PBS) for 15 min in the dark, washed, and incubated with Fc block (eBioscience, dilution 1:150) in Facs Stain Buffer (BD Biosciences). After being washed twice again with FACS stain buffer, cells were subsequently incubated for 30 min, on ice and in the dark with either of the following antibodies: Alexa Fluor®647-conjugated anti-human TCRαβ (clone IP26, Biolegend, 0.25 μg per 10⁶ cells) or Alexa Fluor® 647 Mouse IgG1, x Isotype Control. Cells were washed one more time with FACS stain buffer, fixed with paraformaldehyde 1%, washed again and finally resuspended in FACS stain buffer. Samples were run for analysis with a BD FACS Canto II analyzer (BD Biosciences).

### PBMC Cultures and Peptide Stimulation

PBMC (ReachBio Research Laboratories) are thawed into RPMI medium supplemented with 10% FBS, 1% Pen-strep mix and cultivated in this medium for 24H after thawing. CD4+ T cells and antigen-presenting cells (APC) are then sorted using, respectively, CD4+ isolation kit and anti-MHC class II Microbeads with MACS® sorting technology (Miltenyi Biotec). After elution, the MHC class II-positive cell fraction is collected and resuspended at 1×10⁶ cells/ml in APC cell medium composed of RPMI 1640 (Gibco/Life Technologies)+1% Horse serum+50 U/ml penicillin (Gibco/Life Technologies)+50 μg/ml streptomycin (Gibco/Life Technologies)+2 Mm L-Glutamin (Gibco/Life technologies) supplemented with 30 ng/ml of IL-4 (Peprotech), 50 ng/ml of GM-CSF (Peprotech) and 1 ng/ml of recombinant human TNFα (Peprotech). APC are then incubated with 10 μg/ml of each peptide overnight. The day after, peptide-pulsed APC were harvested and washed before addition at a ratio 1:1 to the different J.RT3-T3.5 derived cell lines, 48 h after infection with the lentiviral particles. J.RT3-T3.5-derived cell lines were then cocultured with the pulsed APC in J.RT3-T3.5 cell culture medium in duplicate with one half for proliferation assay and one half for luciferase assay+ cytokines production assay.

### Proliferation and Cytokine Production Assays

To measure T cell proliferation, on half of the incubated cells, 1 μCi/well [3H]thymidine (Amersham) is added to assay cultures at 48 h after contact with the peptide-pulsed APC. Following incubation for 12-16 h, cultures are harvested onto Unifiter Plates (Packard Instrument). Microscint 20 scintillation fluid (Packard Instrument) is added to each well, and plates are counted on a Scintillation Counter.

For transcription factor activity analysis and cytokines production assay, the other half of the cells is harvested 5 days after contact with the peptide-pulsed APC, centrifuged for 5 min at 300 g, and both cell pellets and supernatants are collected. Cell pellets are processed for luciferase detection assay to measure AP1 activity, and IL-2 and IFNγ are measured by ELISA from the supernatant.

### Discussion

CD4+ and CD8+ T cells express T cell receptors (TCRs) on their surface that specifically interact with the peptide-loaded MHC molecules (MHC class I molecules for CD8+ T cells and MHC class II molecules for CD4+ T cells). The present inventors pseudotyped recombinant lentiviral particles with MHC molecules displaying specific peptides and showed that the interaction of the pMHC complex with their specific restricted TCR can mediate cell entry leading to the expression of a nucleotide of interest carried by the lentiviral particle. This has been demonstrated for both MHC class I and MHC class II molecules. Using this method, nucleotide of interest can be selectively delivered to lymphocytes carrying specific TCRs, e.g., in order to alter lymphocyte activity.

### Example 6: Induction of Suppressive Phenotype in Mouse Antigen-Specific T Cells by Lentiviral Particles Displaying Antigenic Peptides in MHC Class I Groove on their Surface

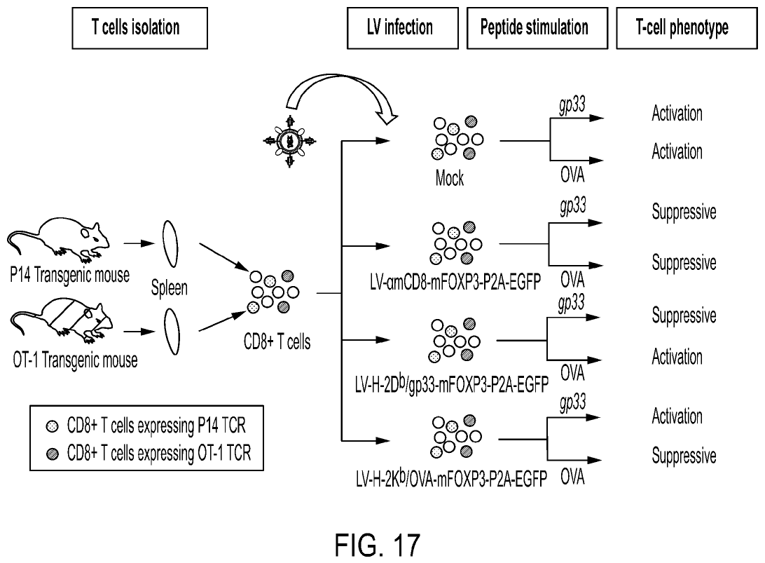

The ability of lentiviral particles presenting the gp33 peptide in the context of mouse MHC I (H2-D^{b}) molecules to transduce T cells specific for an antigen with a modulatory nucleotide of interest is tested ex vivo using the models depicted in FIG. 17.

More particularly, the ability of lentiviral particles carrying the mouse transcription factor FOXP3 (mFoxp3, SEQ ID NO: 61) as a nucleotide of interest to trigger a suppressive phenotype in mouse naïve CD8+ T cells is tested. In addition to the well described CD4+ regulatory described (Tregs), the existence of suppressor CD8+ T cells (Tsups) is emerging, and has been shown to be involved in the pathophysiology of several auto-immune diseases. Among those suppressor CD8+ T cells, a specific CD8+/CD25+/FOXP3+ subset has been identified in both human and mouse, and their capacity to suppress effector T cells has been shown to be equal or even superior as CD4+ Tregs in vitro (Churlaud et al., Frontiers in Immunology, 6(171):1-10 (2015). Therefore, the possibility to also generate antigen-specific CD8+ suppressive T cells by targeted expression of transcription factors constitutes a novel and promising approach for the therapy of auto-immune diseases.

Naïve CD8+ T cells are first isolated from splenocytes of transgenic P14 and OT-1 mice. The isolated CD8+ T cells are then transduced with lentiviral particles displaying the gp33 peptide in the groove of the MHC I (H-2D^{b}) molecule or control OVA peptide in the groove of the MHC I (H-2K^{b}) molecule on its envelope surface and comprising mouse FOXP3 as a nucleotide of interest. Mock or transduced T cells are then re-stimulated with PHA, PMA, anti-CD3/anti-CD8 monoclonal antibodies ot beads, or via APCs pulsed with gp33 or OVA peptides. Restimulated T cells are tested for proliferation via ³H-thymidine assay, activity of cytokines-producing genes, productions of cytokines, and/or activation in a ⁵¹Cr release assay. Expected results are provided in FIG. 17. Alternative approaches include the use of other transcription factor encoding sequences as nucleotides of interest, such as mouse IKZF4, IKZF2, GATA1, IRF4, ELF-1, SATB1 or any other genes responsible of the production of cytokines and/or involved in regulatory/suppressive T cell phenotype.

### Example 7: Ex-Vivo Immortalization of Antigen-Specific T Cells Via Targeted Transduction of Lentiviral Vectors Displaying a MHC Class I/Antigenic Peptide Complex as a Novel Method for Faster Functional Identification of Antigen-Specific TCRs

The identification of antigenic-specific TCRs, and the monitoring of antigen-specific T cells are critical for the study of T cell responses and the development of novel biomarkers and targeted immunotherapies. The most advanced state in the art in this matter uses high-throughput sequencing that can be laborious and cost-effective. Moreover, the sole sequencing of TCRs does not permit identification of the antigen that is recognized by a T cell, and the use of supplemental immune assays (such as multimer binding, proliferation or activation assays) in addition to a TCR repertoire sequencing is necessary to identify the TCRs that are specific for a given antigen.

Here we propose a novel method based on targeted transduction and selection by immortalization of specific TCR expressing T cells with lentiviral vectors expressing a specific antigenic peptide in the groove of a class I MHC.

Using as model the identification of TCRs that are specific for the CMV pp65 peptide, we aim to specifically transduce pp65-restricted TCR expressing cells with a lentivirus displaying the HLA-A2/pp65 complex on its surface and delivering the coding sequence of the human telomerase reverse transcriptase (hTERT) as nucleotide of interest. TERT is as a catalytic subunit of the telomerase enzyme, which allows senescent cells to become immortal by lengthening telomeres in DNA strands. Ectopic overexpression of human TERT (hTERT) from pantropic retroviral vectors has been effective for immortalizing human CD4 and CD8+ T cells (Hooijberg et al. J Immunol. 165:4239-4245 (2000), Rufer et al., Blood, 98(3): 597-604 (2001), Luiten et al., Blood 101(11): 4512-4519 (2203)).

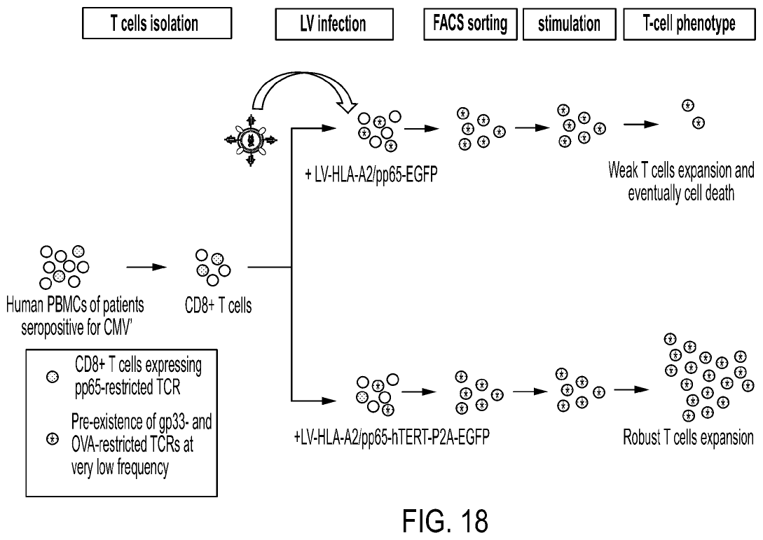

In this model memory human CD8+ T cells are first isolated from patients that are seropositive for CMV. The isolated CD8+ T cells are then transduced with lentiviral particles displaying the pp65 peptide in the groove of the MHC I (HLA-A2) molecule on its surface, and comprising a hTERT-P2A-EGFP cassette as nucleotide of interest. A lentiviral particle displaying the pp65 peptide in the groove of the MHC I (HLA-A2) molecule on its surface and encoding EGFP only is used as a control (FIG. 18). Upon lentiviral transduction, primary CD8+ T cells are kept in culture up to 3 months. After 3 to 4 weeks in culture only cells efficiently transduced and expressing hTERT-P2A-EGFP are surviving and proliferating in comparison to cells transduced only with the EGFP encoding lentiviral particle, and the proportion of EGFP+ reaches more than 90% after 50 days. EGFP+ cells are then sorted and pp65-restricted TCRs can be sequenced.

### Example 8: In Vivo Activation of Gp-33 Specific T Cells by Lentiviral Particles as a Method to Prevent LCMV Infection

For in vivo studies, the ability of lentiviral particles presenting the gp33 peptide in the context of MHC I (H-2D^{b}) molecules to enhance a CD8+ T cell response in order to prevent lymphocytic choriomeningitis virus (LCMV) infection is tested in C57Bl/6 mice (Kyburz, et al. Eur. J. Immunol. 23:1956-1962 (1993)). LCMV is a mouse tropic virus, where the fate of infection depends on the viral strain. Exposure to Armstrong strain results in an acute infection, in which case the infected mice quickly develop a CD8+ T cell response and clearance of the virus in about a week. On the other hand, Clone 13 virus cannot be cleared, and chronic infection is established as T cells become “exhausted” (Zhou et al. Viruses, 4(11): 2650-2669 (2012)). It has been shown that infection of CD8-depleted or MHC class I-deficient mice with the LCMV Armstrong strain that lead to acute infection results in maintenance of high viral titers (Matloubian et al. J. Virol. 68(12):8056-63 (1994)). Thus, since viral infection depends on T cell activity, LCMV is an ideal model to test for modulation of T cell function by in vivo transduction with lentiviral particles presenting the gp33 peptide/MHC class I on their surface in an appropriate context.

Lentiviral particle comprising at its surface either (i) H-2D^{b} (MHC class I molecule) displaying the gp33 peptide or (ii) as a control H-2K^{b} (MHC class I molecule) displaying the OVA₂₅₇₋₂₆₄ peptide (OVA) are generated using the method described in Example 1. Those lentiviral particles are also displaying the fusogen SINmu derived from the mutated Sindbis virus envelope glycoprotein as described above and are generated to carry the transcription factor mouse T-bet as a nucleotide of interest (or any other transcription factor/gene involved in effector and/or memory CD8+ T cell activation).

A cohort of mice is initially injected with the lentiviral particles pseudotyped with the H-2D^{b}/gp33 or H-2K^{b}/OVA complexes and the level of effector and memory CD8+ T cells is monitored at different time points post-injection (from spleen and/or blood). Mock injected mice will serve as negative control group in this experiment. A second cohort of mice are then injected with the lentiviral particles pseudotyped with the H-2D^{b}/gp33 complex and subsequently challenged with the Armstrong strain of LCMV, at post-injection time points that will be determined according to the measurements of effector and memory CD8+ T cells levels from the first experiment. Control groups are constituted with (i) mice not injected with lentiviral particles but infected with LCMV, (ii) mice injected with lentiviral particles pseudotyped with H-2K^{b}/OVA complexes and (iii) mice injected with H-2D^{b}/gp33 lentiviral particles but not infected with LCMV. The LCMV viral burden is analyzed at different time points post-injection by blood collection and post-mortem organs collection to determine the capacity of the lentiviral particles pseudotyped with H-2D^{b}/gp33 to prevent LCMV infection from CD8+ T cells activation.

### Example 9: In Vivo Activation of Mouse Antigen-Specific T Cells by Lentiviral Particles as a Method to Enhance LCMV Infection Clearance

For in vivo studies, the ability of lentiviral particles presenting the gp33 peptide in the context of MHC I (H-2D^{b}) molecules to enhance a T cell response against lymphocytic choriomeningitis virus (LCMV) after infection is tested in C57Bl/6 mice (Kyburz, et al. Eur. J. Immunol. 23:1956-1962 (1993)). More particularly, the in vivo activation of gp33-specific CD8+ T cells infected with H-2D^{b}/gp33-pseudotyped lentiviral particles after (i) gp33 peptide immunization, (ii) acute infection by the LCMV Armstrong strain and/or (iii) chronic infection with LCMV clone 13 strain is compared.

Lentiviral particle comprising at its surface either (i) H-2D^{b} (MHC class I molecule) displaying the gp33 peptide or (ii) as a control H-2K^{b} (MHC class I molecule) displaying the OVA₂₅₇₋₂₆₂ peptide (OVA) are generated using the method described in Example 1. Those lentiviral particles are also displaying the fusogen SINmu derived from the mutated Sindbis virus envelope glycoprotein as described above and are generated to carry the transcription factor mouse T-bet (SEQ ID NO: 15) as a nucleotide of interest (or any other transcription factor/gene involved in effector and/or memory CD8+ T cell activation).

Mice are first inoculated either with the LCMV Armstrong strain, the LCMV Clone 13 strain, immunized with the gp33 peptide (positive control, as described in Example 1) or mock-infected (negative control). The lentiviral particles pseudotyped with the H-2D^{b}/gp33 or H-2K^{b}/OVA complexes produced for this example are then injected 1 day and 7 days after infection with the Armstrong strain, and 1 day, 7 day and 14 days after infection with the Clone 13 strain. The LCMV viral burden will be finally analyzed at different time points post-lentiviral particles injection by blood collection and post-mortem organs collection. At those time points, the levels of effector and memory CD8+ T cells will be measured and compared to the control conditions in order to determine the capacity of the lentiviral particles pseudotyped with H-2D^{b}/gp33 to enhance the T cell response to an LCMV infection.

### Example 10: Enhancement of Gp33 Specific CD8+ T Cell Immunity in a Gp33-Presenting Tumor Model

For in vivo studies, the ability of lentiviral particles presenting the gp33 peptide in the context of MHC I (H-2D^{b}) molecules to enhance a CD8+ T cell response against gp33-presenting tumors is tested in C57Bl/6 mice grafted with B16F10/H-2D^{b}/gp33 cell line that we previously generated as a peptide epitope-presenting melanoma tumor model. B16F10/gp33 melanoma cells have been previously used by other groups as an accurate murine model of non-lymphodepletion adoptive melanoma therapy (Contreras et al. Cancer Immunol Immunother. 65(5):601-611 (2016)). Similarly, we also have generated a MC38/H-2D^{b}/gp33 cell line as a peptide epitope presenting carcinoma tumor model, which will constitute an alternative to B16F10 model for testing this approach.

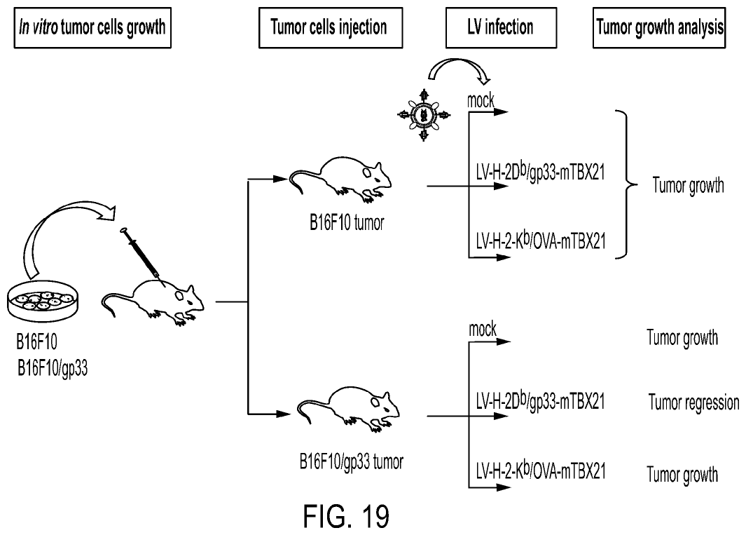

B16F10 and B16F10/H-2D^{b}/gp33 cell lines are first grown in vitro, then subcutaneously injected into C57Bl/6 mice. After tumor cells injection, when tumors are of palpable size, lentiviral particles comprising the gp33 peptide in the groove of the MHC I (H-2D^{b}) or control ovalbumin (OVA) peptide in the groove of the MHC I (H2-K^{b}) molecule on its envelope surface, with or without a gene encoding a T-bet is injected intratumorally. Tumor cell volume is measured. Expected results are provided in FIG. 19. Additionally, tumor, blood and/or spleen may be collected, homogenized in single cell suspensions if necessary, and the level of memory CD8+ T cells in the homogenized samples determined at all those time points.

### Example 11: Development of an Agnostic Multiplexed Platform for Discovering Antigen-Specific CD8 T Cells

The first step in developing T cell-based therapeutics for any disease is the identification of appropriate peptide/MHC (pMHC) targets. The most straightforward way to identifying these targets is to detect them directly from patient tissue samples through methods like mass spectrometry. However, for some diseases, tissue samples may be difficult to acquire in the appropriate quantity and quality for these assays. This is especially true for some viral diseases where patient biopsies are not commonly performed. An alternative approach was proposed which involves identification of virus specific T cells from previously infected donors, with the assumption that these T cells exist because they have previously been presented viral peptides during infection. The antigen-specificity of these T cells can be then determined, and that viral peptide can be assessed for its potential as a target for therapeutics. Dextramer staining has been a common approach to identify antigen-specific T cells, but this requires previous knowledge of which peptides to look for first. Furthermore, dextramers are costly and to produce a large library spanning a viral genome to screen T cells would be impractical.

This Examples describes a platform using fluorescent lentiviruses displaying a library of pMHC for use in identifying virus-specific T cells. Through single cell RNA sequencing, not only the virus specific TCR, but also the cognate viral peptide that the TCR recognizes can be identified. In this Example, this platform was used to identify appropriate BK Virus (BKV) targets for T cell therapies, but this platform can be used for other indications where patient tissue acquisition is difficult.

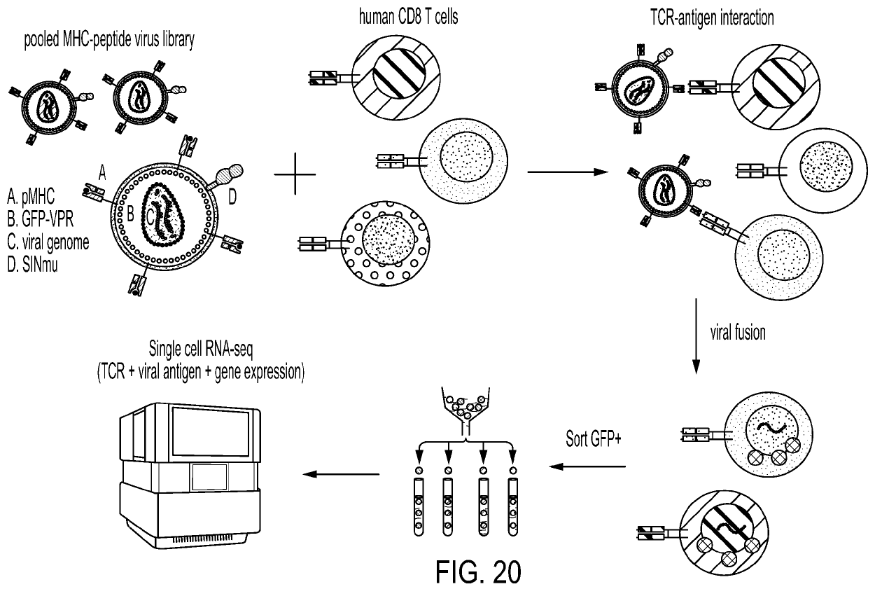

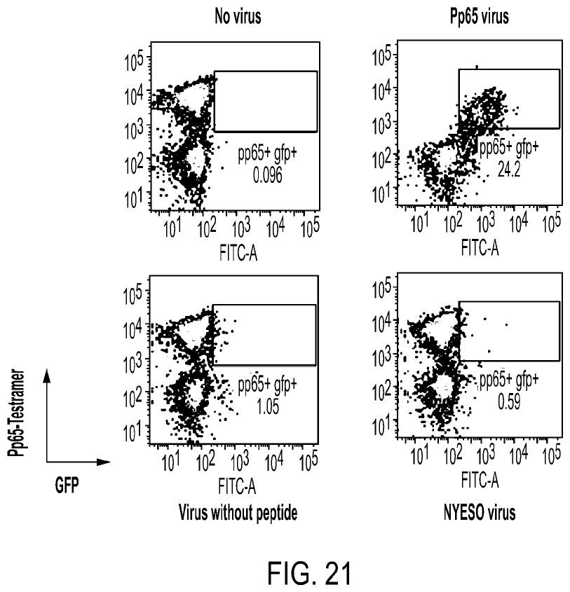

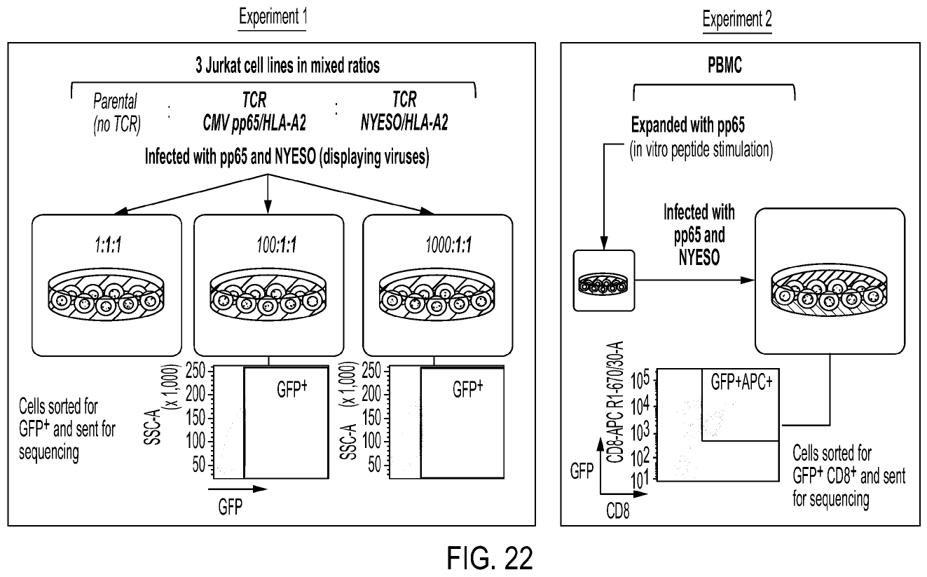

The core of this platform uses lentivirus with several modifications (FIG. 20). The virions have a GFP fusion to the structural protein VPR and are thus fluorescent. Encoded in the viral genome is the sequence for a peptide-MHC-I fusion protein that is presented on the surface of virions. Finally, the virions have a mutated Sindbis fusion protein that lacks the normal receptor binding domain but retains its fusion property. Therefore, the virus will only fuse with CD8 T cells that have a TCR that recognizes the presented pMHC. A library of these lentiviruses presenting different viral peptides was created. Upon binding and fusion, the CD8 T cells that recognize any of these peptides become GFP+ and can be sorted. Single cell RNA sequencing was performed to get the sequence of the TCR, viral peptide (encoded by the viral genome), and gene expression of the T cell. In this manner the TCR can be matched to antigen data in a single step.

CMV pp65 specific CD8 T cells were expanded from human PBMCs in vitro via peptide stimulation. Cells were then mixed with modified lentivirus presenting either pp65/HLA-A2 or NYESO/HLA-A2. Only pp65 specific CD8 T cells (as determined by tetramer stain) become GFP+, indicating that modified lentiviruses are specifically infecting T cells with a TCR recognizing the presented peptide.

To optimize sorting and sequencing of lentivirus-infected cells, two experiments were set up:
(1). Three Jurkat cell lines (no TCR, pp65 TCR, NYESO TCR) were mixed at various ratios and infected with a mixture of pp65/HLA-A2 and NYESO/HLA-A2 displaying lentivirus. After sorting, a ratio of 1:1 for pp65 and NYESO Jurkat cell lines were recovered.
(2). Human PBMCs with pp65 peptide were cultured in vitro and infected with a mixture of pp65/HLA-A2 and NYESO/HLA-A2 displaying lentivirus. After sorting, only CD8 T cells with pp65 viral genomes were recovered.

GFP+ cells were single cell sorted and sent for RNA sequencing (TCR, viral genome, gene expression).

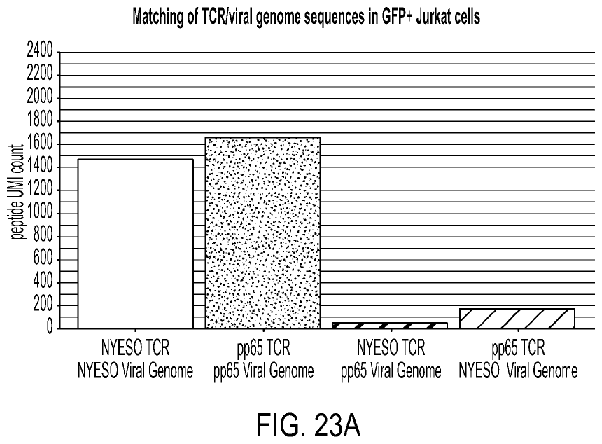

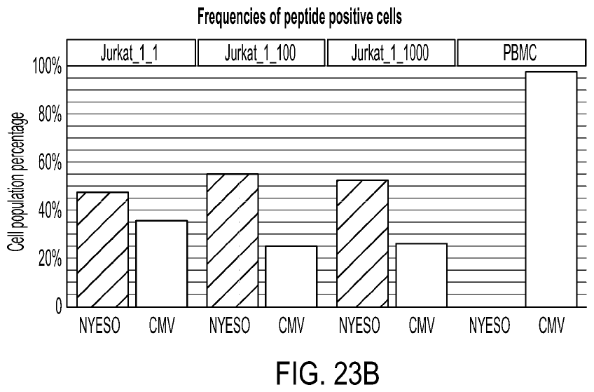

After sorting and sequencing, majority of the recovered Jurkat cells had matching TCR sequences to viral genome (FIG. 23A).

Recovery of GFP+ cells from mixed Jurkat populations showed roughly equal ratios of NYESO and CMV pp65 Jurkat cells. Higher numbers were observed for NYESO Jurkats but this may be due to higher expression of TCR in this cell line (FIG. 23B).

Only CMV pp65 genomes were recovered from GFP+CD8 T cells (FIG. 23B). After sequencing their TCRs, several public pp65 TCRs were found (Table 5), validating this approach.

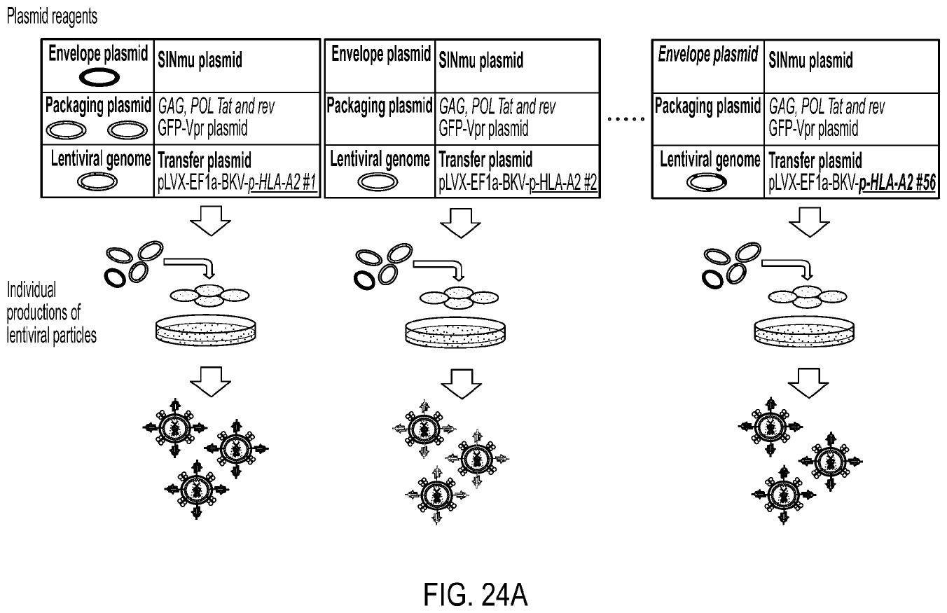

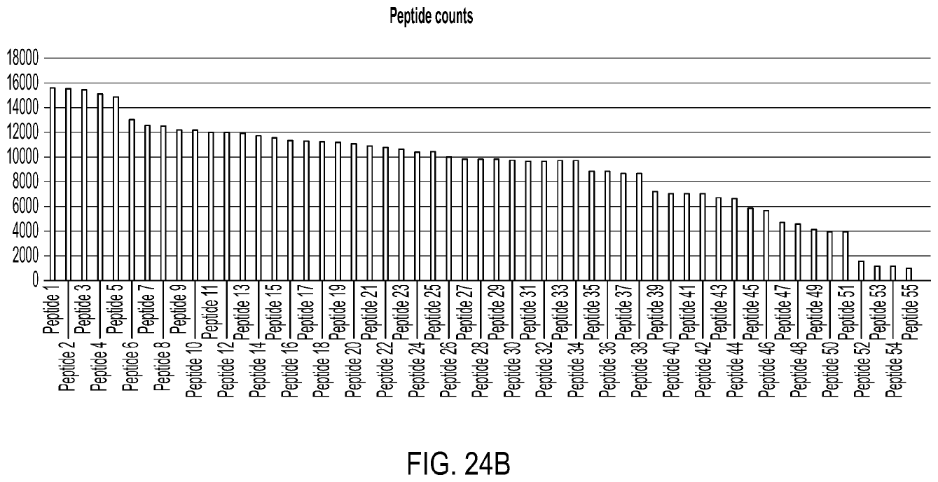

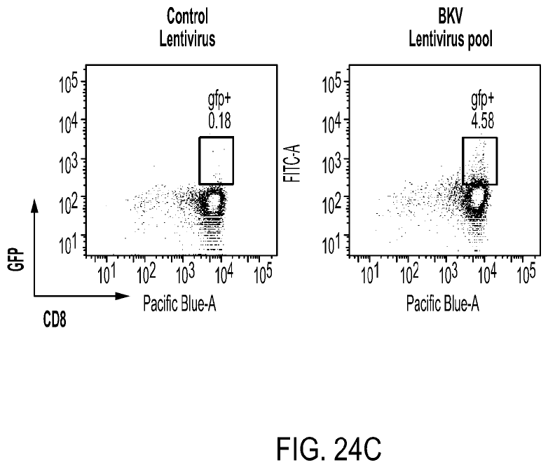

Next, this platform was employed to discover BKV-specific T cells. 56 peptides were picked in the BKV genome that are predicted to bind to HLA-A2 and a lentiviral library that includes these 56 peptides was produced. This was done through 56 separate transfections after which the supernatant was pooled and concentrated together (FIG. 24A).

Sequencing of the library pool revealed that most of the individual 56 different lentiviruses are present in roughly the same ratio (FIG. 2413).

Staining of CD8 T cells from BKV+ donor using BKV lentivirus pool shows GFP+ cells (FIG. 24C).

In conclusion, the platform allowed identification of antigen specific T cells from human donors using labeled fluorescent lentivirus. This platform was validated by identifying CMV pp65-specific CD8 T cells from human PBNCs. 3 KV-specific T cells can next be identified to determine which BKV peptide targets should be pursued for therapeutic development.

The claimed subject matter is not to be limited in scope by the specific embodiments described herein. Indeed, various modifications of the claimed subject matter in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

TABLE 1

| Replication | | Modified | |
|---|---|---|---|
| steps | Plasmid | Regions | Mutation names |
| Reverse | Transfer vector | PBS | PBSnn (non-natural PBS) |
| transcription | plasmid | | dPBS (PBS deletion) |
| | (pLVX) | | |
| | Packaging | Reverse | D443A (RNaseH mutation) |
| | plasmid | Transcriptase | MD182GV (DNA |
| | (psPAX2) | | polymerase mutation) |
| | Transfer vector | 3′ LTR | SV40 (insertion of SV40 |
| | | | poly A before 3'LTR) |
| | plasmid | | d3LTR (Deletion of 3'LTR) |
| | (pLVX) | | SV40dLTR (replacing |
| | | | 3′LTR with SV40 polyA) |
| Integration | Packaging | Zinc Binding | H12A H16A |
| | plasmid | domain | |
| | (psPAX2) | | |
| | Packaging | Catalytic | Single mutants: D64V, |
| | plasmid | domain | D116N, E152A |
| | (psPAX2) | | Double mutants: D64V |
| | | | D116N; D64V N120L; |
| | | | D64V E152A; D116N |
| | | | E152A |
| | Packaging | DNA binding | W235E, K264R |
| | plasmid | domain | |
| | (psPAX2) | | |
| | Transfer vector | Attachment | dAtt |
| | plasmid | site | |
| | (pLVX) | | |

TABLE 2

| | | LV-H-2D^{b}/gp33-EGFP | |
|---|---|---|---|
| | | LV-SINmu-EGFP | |
| | | LV-H-2D^{b}/gp33-mFoxP3 | |
| LV-VSV-P14TCR | | LV-SINmu-mFoxP3 | |
| Transfer plasmid | 25 | Transfer plasmid | 20 |
| Packaging plasmid | 25 | Packaging plasmid | 20 |
| psPAX2 | | | |
| pMD2.G | 10 | pRG984-SINmu | 10 |
| | | pRG984-H-2D^{b}/gp33 | 10 |

TABLE 3

| | | LV-DQ2.5/α1 gliadin-EGFP | |
|---|---|---|---|
| | | LV-SINmu-EGFP | |
| | | LV-DQ2.5/α1 gliadin-hFoxP3 | |
| LV-VSV-DQ2.5/α1-gliadin | | LV-SINmu-hFoxP3 | |
| Transfer plasmid | 25 | Transfer plasmid | 20 |
| Packaging plasmid | 25 | Packaging plasmid | 20 |
| psPAX2 | | | |
| pMD2.G | 10 | pRG984-SINmu | 10 |
| | | pRG984-H-2D^{b}/gp33 | 10 |

TABLE 4

| | | Linker codon- | | Linker codon- | |
|---|---|---|---|---|---|
| Linker amino | SEQ | optimized | SEQ | optimized | SEQ |
| acids | ID | nucleotidic | ID | nucleotidic | ID |
| sequence | NO: | sequence (human) | NO: | sequence (mouse) | NO: |
| GGGS | 74 | GGGGGTGGTTCC | 89 | GGTGGCGGTAGT | 105 |
| GGSG | 75 | GGTGGGTCTGGG | 90 | GGGGGATCTGGT | 106 |
| GSGGS | 73 | GGGTCCGGGGGCTCC | 91 | GGCAGTGGCGGTAGC | 107 |
| GGSGG | 76 | GGTGGGAGCGGTGGT | 92 | GGAGGGAGTGGAGGG | 108 |
| GSGSG | 77 | GGCAGCGGAAGCGGA | 93 | GGGTCTGGCTCAGGC | 109 |
| GSGGG | 78 | GGGAGTGGGGGAGGT | 94 | GGTTCTGGCGGAGGT | 110 |
| GGGSG | 79 | GGTGGGGGAAGTGGA | 95 | GGTGGTGGGAGTGGA | 111 |
| GSSSG | 80 | GGCAGCTCATCTGGT | 96 | GGCTCAAGCAGTGGA | 112 |
| GCGASGGGG | 81 | GGATGTGGTGCATCTG | 97 | GGCTGTGGGGCTAGTG | 113 |
| SGGGGS | | GAGGGGGAGGCTCTG | | GGGGAGGTGGTAGTGG | |
| | | GGGGGGGTGGATCT | | TGGTGGCGGTTCC | |
| GCGASGGGG | 81 | GGGTGTGGTGCTAGT | 98 | GGATGTGGGGCCTCAG | 114 |
| SGGGGS | | GGGGGTGGCGGATCA | | GTGGGGGTGGCAGCGG | |
| | | GGTGGAGGCGGGAGC | | TGGTGGAGGGTCA | |
| GGGGSGGGG | 82 | GGGGGCGGAGGATCT | 99 | GGTGGCGGGGGCTCTG | 115 |
| S | | GGGGGAGGGGGATCA | | GTGGAGGAGGATCT | |
| GGGASGGGG | 83 | GGGGGGGGCGCTTCA | 100 | GGAGGCGGCGCTTCTG | 116 |
| SGGGGS | | GGCGGAGGTGGAAGT | | GGGGCGGGGGTAGTGG | |
| | | GGTGGAGGAGGT | | GGGTGGAGGT | |
| GGGGSGGGG | 84 | GGAGGGGGAGGTTCT | 101 | GGTGGAGGTGGAAGTG | 117 |
| SGGGGS | | GGCGGCGGGGGATCA | | GAGGAGGGGGATCAGG | |
| | | GGAGGCGGTGGGAGC | | CGGAGGCGGGAGC | |
| GGGASGGGG | 85 | GGTGGGGGGGCGTCA | 102 | GGAGGGGGAGCCTCTG | 118 |
| S | | GGTGGAGGCGGAAGT | | GCGGTGGAGGATCA | |
| GGGGSGGGG | 84 | GGCGGCGGAGGTTCT | 103 | GGGGGAGGAGGCAGTG | 119 |
| SGGGGS | | GGTGGGGGTGGCAGT | | GAGGTGGGGGAAGTGG | |
| | | GGAGGAGGAGGCAGC | | TGGAGGGGGGTCT | |
| GGGGSGGGG | 86 | GGAGGTGGAGGTAGT | 104 | GGGGGTGGAGGATCAG | 120 |
| SGGGGSGGG | | GGCGGTGGTGGGTCA | | GAGGCGGTGGTTCTGG | |
| GS | | GGGGGAGGCGGGTCC | | GGGAGGTGGATCCGGC | |
| | | GGTGGCGGTGGGAGT | | GGGGGTGGTAGT | |

TABLE 5

| Sequence | Frequency | Group | Locus |
|---|---|---|---|
| CAVGYGNQFYF (SEQ ID NO: 121) | 26 | PBMC | IR VJ 1 cdr3 |
| CILDNNNDMRF (SEQ ID NO: 122) | 8 | PBMC | IR VJ 1 cdr3 |

1. A recombinant viral particle that is capable of binding to a T cell receptor (TCR), the recombinant viral particle comprising on its surface a TCR-binding molecule.

2. The viral particle of claim 1, wherein the TCR-binding molecule comprises an anti-TCR antibody or portions thereof.

3. A recombinant viral particle that is capable of binding to an antigen-specific T-cell receptor (TCR), the recombinant viral particle comprising on its surface a peptide (p) presented in the context of a major histocompatibility complex (MHC) molecule (pMHC complex).

4. The viral particle of any one of claims 1-3, wherein the particle is capable of binding to an antigen-specific TCR expressed on a surface of a cell.

5. The viral particle of claim 4, wherein the viral particle is capable of infecting the TCR-expressing cell only in the presence of the TCR-binding molecule.

6. The viral particle of any one of claims 1-5, wherein the recombinant viral particle is derived from an enveloped virus.

7. The viral particle of claim 6, wherein the recombinant viral particle further comprises a fusogen on its surface.

8. The viral particle of claim 6 or 7, wherein the pMHC complex and/or the fusogen comprises a transmembrane domain embedded in a lipid envelope of the recombinant viral particle.

9. The viral particle of any one of claims 6-8, wherein one or more of proteins present in the lipid envelope of a wild-type virus corresponding to the recombinant viral particle are absent or mutated so that said recombinant viral particle is not capable of binding to any cell targeted by the wild-type virus in the absence of the TCR-binding molecule.

10. The viral particle of claim 9, wherein all of the proteins which are normally present in the lipid envelope of the wild-type virus are absent.

11. The viral particle of any one of claims 1-5, wherein the recombinant viral particle is derived from a non-enveloped virus.

12. The viral particle of claim 11, wherein the anti-TCR antibody or portions thereof, or the pMHC complex is expressed on the outer surface of a virus capsid of the non-enveloped virus.

13. The viral particle of claim 11, wherein the anti-TCR antibody or portions thereof, or the pMHC complex is attached to the outer surface of the virus capsid by a linker.

14. The viral particle of claim 13, wherein the anti-TCR antibody or portions thereof, or the pMHC complex is attached to the outer surface of the virus capsid by a SpyTag:SpyCatcher system, SpyTag002:SpyCatcher002 system, SpyTag:KTag system, isopeptag:pilin C system, or SnoopTag:SnoopCatcher system.

15. The viral particle of any one of claims 11-14, wherein the non-enveloped virus is derived from a virus of the family Parvoviridae.

16. The viral particle of claim 15, wherein the recombinant viral particle is an adeno-associated virus (AAV) particle.

17. The viral particle of any one of claims 1-16, wherein the viral particle is replication deficient.

18. The viral particle of any one of claims 1-10 and 17, further comprising at least one viral capsomer.

19. The viral particle of any one of claims 1-10 and 17-18, further comprising at least one lentiviral capsomer.

20. The viral particle of any one of claims 1-10 and 17-19, wherein the viral particle is derived from a virus of the family Retroviridae.

21. The viral particle of claim 20, wherein the viral particle is a retroviral particle.

22. The viral particle of claim 20, wherein the viral particle is a lentiviral particle.

23. The lentiviral particle of claim 22, wherein said lentiviral particle does not contain gp120 surface envelope protein and/or gp41 transmembrane envelope protein and wherein said lentiviral particle is not capable of binding to the TCR-expressing cell in the absence of the TCR-binding molecule.

24. The lentiviral particle of claim 23, wherein said lentiviral particle contains a mutant gp120 surface envelope protein and/or a mutant gp41 transmembrane envelope protein and wherein said lentiviral particle is not capable of binding to the cell in the absence of the TCR binding molecule.

25. The viral particle of any one of claims 1-10 and 17-24, wherein the viral particle comprises capsomers from Human Immunodeficiency Virus (HIV).

26. The viral particle of any one of claims 7-10 and 17-25, wherein the fusogen is a protein.

27. The viral particle of any one of claims 7-10 and 17-26, wherein the fusogen is a viral protein or a fragment, mutant or derivative thereof.

28. The viral particle of any one of claims 7-10 and 17-27, wherein the fusogen is heterologous to the virus from which the particle is derived.

29. The viral particle of any one of claims 7-10 and 17-28, wherein the fusogen is selected from the group consisting of a vesiculovirus protein, an alphavirus protein, an orthomyxovirus protein, a paramyxovirus protein, a retrovirus protein, a lentivirus protein, and a fragment, mutant or derivative thereof.

30. The viral particle of any one of claims 7-10 and 17-29, wherein the fusogen is a Sindbis virus glycoprotein or a fragment, mutant or derivative thereof.

31. The viral particle of any one of claims 7-10 and 17-30, wherein the fusogen is a mutated protein which does not bind its natural ligand.

32. The viral particle of any one of claims 7-10 and 17-31, wherein the fusogen comprises the sequence set forth as SEQ ID NO: 5.

33. The viral particle of any one of claims 7-10 and 17-29, wherein the fusogen is a vesicular stomatitis virus G glycoprotein (VSVG) or a fragment, mutant or derivative thereof.

34. The viral particle of any one of claims 7-10 and 17-32, wherein at least one chain of the MHC and the fusogen are comprised within a fusion protein.

35. The viral particle of claim 34, wherein the pMHC complex and the fusogen are comprised within a fusion protein.

36. The viral particle of any one of claims 7-10 and 17-33, wherein at least one chain of the MHC and the antigen are comprised within a fusion protein.

37. The viral particle of any one of claims 7-10 and 17-34, wherein the MHC and the antigen are separated by a linker sequence.

38. The viral particle of claim 37, wherein the linker sequence comprises one or more G₄S repeats (SEQ ID NO: 70).

39. The viral particle of any one of claims 1-38, further comprising a cytotoxic agent.

40. The viral particle of claim 39, wherein the cytotoxic agent is a toxin or a radioactive isotope or a suicide gene.

41. The viral particle of any one of claims 1-40, further comprising a nucleotide sequence of interest.

42. The viral particle of claim 41, further characterized such that, upon infection of the TCR-expressing cell with the viral particle, the nucleotide sequence of interest becomes integrated into the cell genome.

43. The viral particle of claim 41, further characterized such that, upon infection of the TCR-containing cell with the viral particle, the nucleotide sequence of interest does not become integrated into the cell genome.

44. The viral particle of any one of claims 3-43, wherein the MHC comprises (i) a class I MHC polypeptide or a fragment, mutant or derivative thereof, and optionally, (ii) a β2 microglobulin polypeptide or a fragment, mutant or derivative thereof.

45. The viral particle of any one of claims 3-44, wherein the MHC comprises a class I MHC polypeptide linked to a β2 microglobulin polypeptide by a peptide linker.

46. The viral particle of any one of claims 3-45, wherein the MHC comprises a human class I MHC polypeptide selected from the group consisting of HLA-A, HLA-B, HLA-C, HLA-E, HLA-F, and HLA-G.

47. The viral particle of any one of claims 3-46, wherein the MHC comprises a murine class I MHC polypeptide selected from the group consisting of H-2K, H-2D, H-2L, H-2Q, H-2M and H-2T.

48. The viral particle of any one of claims 3-43, wherein the MHC comprises a class II MHC polypeptide or a fragment, mutant or derivative thereof.

49. The viral particle of any one of claims 3-43 and 48, wherein the MHC comprises a and a polypeptides of a class II MHC complex or a fragment, mutant or derivative thereof.

50. The viral particle of any one of claims 3-43 and 48-49, wherein the MHC comprises α and β polypeptides of a class II MHC complex or a fragment, mutant or derivative thereof, and wherein the α and β polypeptides are linked by a peptide linker.

51. The viral particle of any one of claims 3-43 and 48-50, wherein the MHC comprises α and β polypeptides of a human class II MHC complex selected from the group consisting of HLA-DP, HLA-DR, and HLA-DQ.

52. The viral particle of any one of claims 3-43 and 48-50, wherein the MHC comprises α and β polypeptides of a murine H-2A or H-2E class II MHC complex.

53. The viral particle of any one of claims 1-52, comprising a nucleotide sequence of interest that encodes a protein, a peptide, an RNAi molecule, an antisense oligonucleotide, a CRISPR/Cas9 system or derivatives thereof, or a miRNA.

54. The viral particle of any one of claims 1-53, comprising a nucleotide sequence of interest that encodes a molecule toxic to a cell expressing the TCR.

55. The viral particle of any one of claims 1-54, comprising a nucleotide sequence of interest that encodes a molecule that modulates an activity of a cell expressing the TCR.

56. The viral particle of any one of claims 1-55, comprising a nucleotide sequence of interest that encodes a molecule that inhibits an activity of a cell expressing the TCR.

57. The viral particle of any one of claims 1-55, comprising a nucleotide sequence of interest that encodes a molecule that enhances an activity of the TCR-containing cell.

58. The viral particle of any one of claims 1-55, comprising a nucleotide sequence of interest that encodes a reporter molecule or a selectable marker.

59. The viral particle of any one of claims 1-58, comprising a nucleotide sequence of interest under the control of a promoter selected from the group consisting of a viral promoter homologous to the at least one viral element, a viral promoter heterologous to the at least one viral element, a bacterial promoter, a mammalian promoter, an avian promoter, a fish promoter, an insect promoter, and any combination thereof.

60. The viral particle of any one of claims 1-59, comprising a nucleotide sequence of interest under the control of a human promoter.

61. The viral particle of any one of claims 1-59, comprising a nucleotide sequence of interest under the control of a non-human promoter.

62. The viral particle of any one of claims 1-59, comprising a nucleotide sequence of interest under the control of a promoter that is specific to a T cell.

63. The viral particle of any one of claims 1-62, wherein the peptide comprises an antigenic determinant of a protein selected from the group consisting of a self protein associated with an autoimmune disorder, a viral protein, a tumor associated protein, a bacterial protein, a fungal protein, a protozoan parasite protein, a helminth parasite protein, and an ectoparasite protein.

64. The viral particle of claim 63, wherein the peptide comprises an antigenic determinant of a protein associated an inflammatory disorder.

65. The viral particle of claim 62 or claim 63, wherein the peptide comprises an antigenic determinant of a self protein associated with an autoimmune disorder.

66. The viral particle of claim 65, wherein the self protein comprises a gliadin or a fragment thereof.

67. The viral particle of claim 66, wherein the self protein comprises (i) α-gliadin fragment corresponding to amino acids 57-73 or (ii) γ-gliadin fragment corresponding to amino acids 139-153 or (iii) ω-gliadin fragment corresponding to amino acids 102-118.

68. The viral particle of claim 63, wherein the protein is a viral protein.

69. The viral particle of claim 68, wherein the viral protein comprises lymphocytic choriomeningitis virus (LCMV) glycoprotein (gp33), (CMV) pp65 protein, or a fragment thereof.

70. The viral particle of claim 63, wherein the protein is a tumor associated protein.

71. The viral particle of any one of claims 1-70, wherein the TCR is immobilized on a surface.

72. The viral particle of any one of claims 1-71, wherein the TCR is present on a surface of a cell.

73. The viral particle of claim 72, wherein the cell is a lymphocyte.

74. The viral particle of claim 73, wherein the lymphocyte is a T cell.

75. The viral particle of claim 74, wherein the T-cell is a CD4+ T cell.

76. The viral particle of claim 74, wherein the T-cell is a CD8+ T cell.

77. A method of producing a viral particle that is capable of binding to a T-cell receptor (TCR), said method comprising culturing a packaging cell in conditions sufficient for the production of a plurality of viral particles, wherein the packaging cell comprises one or more plasmids comprising (i) one or more viral elements involved in the assembly of the viral particle, and (ii) a nucleotide sequence encoding a TCR-binding molecule.

78. The method of claim 77, wherein the nucleotide sequence (ii) encodes an anti-TCR antibody or portion thereof.

79. A method of producing a viral particle that is capable of binding to an antigen-specific T-cell receptor (TCR), said method comprising culturing a packaging cell in conditions sufficient for the production of a plurality of viral particles, wherein the packaging cell comprises one or more plasmids comprising (i) one or more viral elements involved in the assembly of the viral particle, (ii) a nucleotide sequence encoding a peptide, and (iii) one or more nucleotide sequence(s) encoding a major histocompatibility complex (MHC) molecule, wherein the viral particles present the antigen-derived peptide in the context of the MHC.

80. The method of any one of claims 77-79, wherein the one or more plasmids further comprise a nucleotide sequence encoding a fusogen.

81. The method of any one of claims 77-80, further comprising collecting the viral particles.

82. The method of any one of claims 77-81, comprising one or more of the following steps:
a. clearing cell debris,
b. treating the supernatant containing viral particles with DNase I and MgCl₂,
c. concentrating viral particles, and
d. purifying the viral particles.

83. The method of any one of claims 77-82, wherein the packaging cell further comprises a nucleotide sequence of interest.

84. The method of any one of claims 77-83, wherein the one or more plasmids comprises (a) GAG, (b) POL, and (c) TAT and/or REV lentiviral or retroviral elements.

85. The method of claim 84, wherein the plasmids present in the packaging cell do not comprise a lentiviral or retroviral ENV gene or comprise a mutant non-functional ENV gene.

86. The method of claim 85, wherein the one or more plasmids comprise a mutant lentiviral ENV gene, which does not produce gp120 surface envelope protein or gp41 transmembrane envelope or which encodes a mutant gp120 surface envelope protein and/or a mutant gp41 transmembrane envelope protein and wherein the resulting viral particle is not capable of binding to a target cell in the absence of the TCR-binding molecule.

87. The method of any one of claims 77-86, wherein the one or more viral elements involved in viral assembly encodes Human Immunodeficiency Virus (HIV) component(s).

88. The method of any one of claims 80-87, wherein the fusogen is a protein.

89. The method of any one of claims 80-88, wherein the fusogen is a viral protein or a fragment, mutant or derivative thereof.

90. The method of any one of claims 80-89, wherein the fusogen is heterologous to the virus from which the particle is derived.

91. The method of any one of claims 80-90, wherein the fusogen is selected from the group consisting of a vesiculovirus protein, an alphavirus protein, an orthomyxovirus protein, and a paramyxovirus protein, a retrovirus protein, a lentivirus protein, and a fragment, mutant or derivative thereof.

92. The method of claim 91, wherein the fusogen is a Sindbis virus glycoprotein or a fragment, mutant or derivative thereof.

93. The method of any one of claims 80-92, wherein the fusogen is a mutated protein which does not bind its natural ligand.

94. The method of any one of claims 80-93, wherein the fusogen comprises a sequence set forth as SEQ ID NO: 5.

95. The method of any one of claims 80-94, wherein at least one chain of the MHC and the fusogen are comprised within a fusion protein.

96. The method of any one of claims 80-95, wherein at least one chain of the MHC and the peptide are comprised within a fusion protein.

97. The method of any one of claims 79-96, wherein the MHC and the peptide are separated by a linker sequence.

98. The method of claim 97, wherein the linker sequence comprises one or more G₄S repeats (SEQ ID NO: 70).

99. The method of any one of claims 79-98, wherein the MHC comprises (i) a class I MHC polypeptide or a fragment, mutant or derivative thereof, and optionally, (ii) a β2 microglobulin polypeptide or a fragment, mutant or derivative thereof.

100. The method of any one of claims 79-99, wherein the MHC comprises a class I MHC polypeptide linked to the β2 microglobulin polypeptide by a peptide linker.

101. The method of any one of claims 79-100, wherein the MHC comprises a human class I MHC polypeptide selected from the group consisting of HLA-A, HLA-B, HLA-C, HLA-E, HLA-F, and HLA-G.

102. The method of any one of claims 79-101, wherein MHC comprises a murine class I MHC polypeptide selected from the group consisting of H-2K, H-2D, H-2L, H-2Q, H-2M, and H-2T.

103. The method of any one of claims 79-98, wherein the MHC comprises a class II MHC polypeptide or a fragment, mutant or derivative thereof.

104. The method of any one of claims 79-98 and 103, wherein the MHC comprises α and β polypeptides of a class II MHC complex or a fragment, mutant or derivative thereof.

105. The method of any one of claims 79-98 and 103-104, wherein the MHC comprises α and β polypeptides of a class II MHC complex or a fragment, mutant or derivative thereof that are linked by a peptide linker.

106. The method of any one of claims 79-98 and 103-105, wherein the MHC comprises α and β polypeptides of a human class II MHC complex selected from the group consisting of HLA-DP, HLA-DR, and HLA-DQ.

107. The method of any one of claims 79-98 and 103-106, wherein the MHC comprises α and β polypeptides of a murine H-2A or H-2E class II MHC complex.

108. The method of any one of claims 77-107, wherein the one or more plasmids comprise a nucleotide sequence of interest under the control of a promoter selected from the group consisting of a viral promoter homologous to the at least one viral element, a viral promoter heterologous to the at least one viral element, a bacterial promoter, a mammalian promoter, an avian promoter, a fish promoter, an insect promoter, and any combination thereof.

109. The method of any one of claims 77-108, wherein the one or more plasmids comprise a nucleotide sequence of interest under the control of a human promoter.

110. The method of any one of claims 77-108, wherein the one or more plasmids comprise a nucleotide sequence of interest under the control of a non-human promoter.

111. The method of any one of claims 77-108, wherein the one or more plasmids comprise a nucleotide sequence of interest under the control of a promoter that is specific to a T cell.

112. The method of any one of claims 77-111, wherein the peptide comprises an antigenic determinant of a protein selected from the group consisting of a self protein associated with an autoimmune disorder, a viral protein, a tumor protein, a bacterial protein, a fungal protein, a protozoan parasite protein, a helminth parasite protein, and an ectoparasite protein.

113. The method of any one of claims 79-112, wherein the packaging cell does not comprise any MHCs other than the MHC of the viral particle.

114. A viral particle made by the method of any one of claims 77-113.

115. A pharmaceutical composition comprising the viral particle of any one of claims 1-76 and 114 and a pharmaceutically acceptable carrier or excipient.

116. A pharmaceutical dosage form comprising the viral particle of any one of claims 1-76 and 114.

117. A method for delivering a nucleotide sequence of interest to a cell expressing a T-cell receptor (TCR) comprising contacting the cell with the viral particle according to any one of claims 1-76 and 114 in conditions sufficient for infection of the cell by the viral particle, wherein the TCR-binding molecule binds the TCR and wherein the viral particle comprises the nucleotide of interest.

118. The method of claim 117, wherein the delivery of the nucleotide sequence of interest results in modulating an activity or survival of the TCR-expressing cell.

119. A method for modulating an activity or survival of a cell expressing a T-cell receptor (TCR), comprising expressing in the cell a nucleotide of interest, wherein the cell is infected with the viral particle of any one of claims 1-76 and 114, wherein the TCR-binding molecule binds the TCR, and wherein the viral particle comprises the nucleotide of interest.

120. The method of claim 119, wherein the TCR is antigen specific, and wherein TCR-binding molecule binds the idiotype of the TCR antigen recognition site.

121. The method of any one of claims 117-120, wherein the cell is a lymphocyte.

122. The method of claim 121, wherein the lymphocyte is a T-cell.

123. The method of claim 122, wherein the T-cell is a CD4+ T-cell.

124. The method of claim 122, wherein the T-cell is a CD8+ T-cell.

125. The method of any one of claims 117-124, wherein said contacting is ex vivo.

126. The method of any one of claims 117-124, wherein said contacting is in vivo in a subject.

127. The method of claim 126, wherein the subject is human.

128. The method of any one of claims 117-127, wherein the cell is a mammalian cell.

129. The method of claim 128, wherein the mammalian cell is a human cell.

130. A modified cell comprising the viral particle of any one of claims 1-76 and 114, or one or more portions thereof.

131. The modified cell of claim 130, wherein the cell is a lymphocyte.

132. The modified cell of claim 131, wherein the lymphocyte is a T cell.

133. The modified cell of claim 132, wherein the T-cell is a CD4+ T cell.

134. The modified cell of claim 132, wherein the T-cell is a CD8+ T cell.

135. The modified cell of any one of claims 130-134, wherein the cell is a mammalian cell.

136. The modified cell of claim 135, wherein the mammalian cell is a human cell.

137. A method of treating a disorder in a subject in need thereof comprising administering to said subject an effective amount of the viral particle of any one of claims 1-76 and 114, wherein the viral particle binds to an antigen-specific T-cell receptor (TCR) and wherein the antigen is associated with the disorder.

138. The method of claim 137, wherein the disorder an inflammatory or an autoimmune disorder and wherein the administration inhibits an inflammatory or autoimmune response.

139. The method of claim 137 or claim 138, wherein the disorder is celiac disease or gluten sensitivity.

140. The method of claim 139, wherein the antigen comprises a gliadin or a fragment thereof.

141. The method of claim 139 or 140, wherein the antigen comprises (i) α-gliadin fragment corresponding to amino acids 57-73 or (ii) γ-gliadin fragment corresponding to amino acids 139-153 or (iii) ω-gliadin fragment corresponding to amino acids 102-118.

142. The method of any one of claims 137-141, wherein the viral particle presents a peptide comprising an antigenic determinant of the antigen in the context of a class II MHC.

143. The method of claim 137 or claim 142, wherein the disorder is a tumor and wherein the administration results in enhancement of an anti-tumor immune response.

144. The method of claim 137 or claim 142, wherein the disorder is an infection caused by an infectious agent and wherein the administration results in enhancement of an immune response against the infectious agent.

145. The method of claim 144, wherein the infectious agent is selected from the group consisting of a virus, a bacterium, a fungus, a protozoan, a parasite, a helminth, and an ectoparasite.

146. The method of claim 145, wherein the infectious agent is lymphocytic choriomeningitis virus (LCMV) and the antigen is glycoprotein (gp33) or a fragment thereof.

147. The method of claim 145, wherein the infectious agent is human cytomegalovirus (CMV) and the antigen is lower matrix protein 65 (pp65) or a fragment thereof.

148. The method of any one of claims 137-140 and 143-147, wherein the wherein the viral particle presents a peptide in the context of a class I MHC.

149. The method of any one of claims 137-148, wherein the subject is a mammal.

150. The method of claim 149, wherein the mammal is human.

151. A packaging cell for producing the viral particle of any one of claims 1-76 comprising one or more plasmids comprising (i) one or more viral elements involved in the assembly of the viral particle, (ii) a nucleotide sequence encoding a TCR-binding molecule and optionally (iii) a nucleotide sequence encoding a fusogen.

152. The packaging cell of claim 151, wherein the nucleotide sequence (ii) encodes anti-TCR antibody or portion thereof.

153. A packaging cell for producing the viral particle of any one of claims 3-76 comprising one or more plasmids comprising (i) one or more viral elements involved in the assembly of the viral particle, (ii) a nucleotide sequence encoding a peptide, (iii) one or more nucleotide sequence(s) encoding a major histocompatibility complex (MHC) molecule, and optionally (iv) a nucleotide sequence encoding a fusogen.

154. The packaging cell of claim 153, comprising a nucleotide sequence of interest.

155. The packaging cell of claim 153 or claim 154, wherein the packaging cell does not comprise any MHCs other than the MHC of the viral particle.

156. A library comprising a plurality of viral particles of any one of claims 1-76.

157. The library of claim 156, wherein the viral particles within the library differ in the sequence of the peptides (p) presented in the context of a major histocompatibility complex (MHC) molecule (pMHC complex) on the surface of the viral particles.

158. The library of claim 156 or claim 157, wherein the library comprises at least 10², at least 10³, at least 10⁴, at least 10⁵, at least 10⁶, at least 10⁷, at least 10⁸, at least 10⁹, or at least 10¹⁰ unique viral particles.

159. The library of claim 157 or claim 158, wherein each different peptide (p) sequence within the library is generated based on mass-spectrometry and/or computational analysis and/or validated TCR epitopes.

160. The library of any one of claims 156-159, wherein each viral particle within the library comprises a polynucleotide encoding a reporter protein.

161. The library of any one of claims 156-159, wherein each viral particle within the library comprises a reporter protein.

162. The library of claim 161, wherein the reporter protein is fused to a viral particle protein.

163. The library of claim 162, wherein the viral particle is a lentiviral particle and the viral particle protein is VPR.

164. The library of any one of claims 160-163, wherein the reporter protein is a fluorescent protein.

165. The library of any one of claims 156-164, wherein each viral particle within the library comprises a polynucleotide encoding a pMHC complex and optionally a universal primer binding sequence.

166. The library of claim 165, wherein each viral particle within the library comprises a polynucleotide encoding a pMHC complex and a universal primer binding sequence, wherein the pMHC complex is the same as the pMHC complex which is present on the surface of said viral particle.

167. The library of any one of claims 156-164, wherein each viral particle within the library is a lentiviral particle which comprises a fusogen, wherein said fusogen comprises a Sindbis virus glycoprotein or a fragment, mutant or derivative thereof.

168. The library of claim 167, wherein the fusogen is a mutated Sindbis virus glycoprotein which does not bind its natural ligand.

169. The library of claim 167, wherein the fusogen comprises the sequence set forth as SEQ ID NO: 5.

170. A method for identifying an antigen-specific T-cell receptor (TCR), comprising:
a) contacting a sample comprising TCR-expressing cells, wherein said sample has been isolated from a subject who has been previously exposed to an antigen, with the library of any one of claims 156-169, wherein the library comprises a plurality of viral particles comprising peptides (p) derived from said antigen, wherein said peptides are presented in the context of pMHC complexes on the surface of the viral particles, and wherein the viral particles in the library comprise a reporter protein or a polynucleotide encoding the reporter protein,
b) identifying one or more cells comprising the reporter protein, and
c) determining the sequence(s) of TCR(s) expressed by the one or more cells identified in step (b).

171. The method of claim 170, wherein the TCR is identified using a single cell RNA sequencing.

172. The method of claim 170 or claim 171, wherein TCR-expressing cells are selected from CD8+ T cells, CD4+ T cells, and pan CD3+ T cells.

173. The method of any one of claims 170-172, wherein the sample is isolated peripheral blood mononuclear cells (PBMCs).

174. The method of any one of claims 170-173, wherein prior to step (a) the TCR-expressing cells in the sample are expanded and/or activated in the presence of the same plurality of antigen-derived peptides (p) as the peptides present in the library.

175. The method of any one of claims 170-174, wherein the method further comprises determining the activation state of the one or more cells identified in step (b).

176. The method of claim 175, wherein the activation state of the one or more cells identified in step (b) is determining the expression level of one or more genes selected from IFNγ, granzyme B, 4-1BB, CD28, CD25, CD69, OX40, and CD40L.

177. The method of any one of claims 170-176, wherein each of the viral particles in the library comprises a polynucleotide encoding a pMHC complex and a universal primer binding sequence, wherein the pMHC complex is the same as the pMHC complex which is present on the surface of said viral particle.

178. The method of claim 177, wherein the method further comprises identifying the cognate peptide(s) recognized by the identified TCR(s), by determining the sequence of the peptide(s) contained within the pMHC-encoding polynucleotide(s) in the one or more cells identified in step (b).

179. The method of claim 178, wherein the peptide sequence is determined by sequencing using a primer that binds the universal primer binding sequence.

180. A method for identifying an antigenic peptide recognized by one or more antigen-specific TCR-expressing cells, comprising:
a) contacting a sample comprising TCR-expressing cells, wherein said sample has been isolated from a subject who has been previously exposed to an antigen, with the library of any one of claims 156-169, wherein the library comprises a plurality of viral particles comprising peptides (p) derived from said antigen, wherein said peptides are presented in the context of pMHC complexes on the surface of the viral particles, and wherein the viral particles in the library comprise (i) a reporter protein or a polynucleotide encoding the reporter protein and (ii) a polynucleotide encoding a pMHC complex and a universal primer binding sequence, wherein the pMHC complex is the same as the pMHC complex which is present on the surface of said viral particle,
b) identifying one or more cells comprising the reporter protein, and
c) identifying the peptide(s) encoded by the viral particle(s) which had infected the one or more cells identified in step (b).

181. The method of claim 180, wherein TCR-expressing cells are selected from CD8+ T cells, CD4+ T cells, or pan CD3+ T cells.

182. The method of claim 180 or claim 181, wherein the sample is isolated peripheral blood mononuclear cells (PBMCs).

183. The method of any one of claims 180-182, wherein prior to step (a) the TCR-expressing cells in the sample are expanded in the presence of the same plurality of antigen-derived peptides (p) as the peptides present in the library.

184. The method of any one of claims 180-183, wherein identifying the peptide(s) in step (c) comprises determining the sequence of the peptide(s) contained within the pMHC-encoding polynucleotide expressed by the one or more cells identified in step (b).

185. The method of claim 184, wherein the peptide sequence is determined by sequencing using a primer that binds the universal primer binding sequence.

186. The method of any one of claims 180-185, further comprising identifying the antigenic peptide recognized by the largest number of TCR-expressing cells in samples isolated from a plurality of subjects who have been previously exposed to the antigen.
